(19) United States
(12) Patent Application Publication
(10) Pub. No.: US 2025/0381277 A1
(43) Pub. Date: Dec. 18, 2025

(54) COMPOSITIONS AND METHODS FOR TREATMENT OF NEUROLOGICAL DISORDERS

(71) Applicants: Dana-Farber Cancer Institute, Inc., Boston, MA (US); Icahn School of Medicine at Mount Sinai, New York, NY (US)

(72) Inventors: Harvey Cantor, Boston, MA (US); Yiguo Qiu, Boston, MA (US); Xianli Shen, Boston, MA (US); Michal Schnaider-Beeri, New York, NY (US)

(21) Appl. No.: 18/835,929

(22) PCT Filed: Feb. 1, 2023
(86) PCT No.: PCT/US2023/061750

§ 371 Date: Aug. 5, 2024

Related U.S. Application Data
(60) Provisional application No. 63/306,985, filed on Feb. 4, 2022.
(60) Provisional application No. 63/347,113, filed on May 31, 2022.
(60) Provisional application No. 63/480,398, filed on Jan. 18, 2023.

(51) Int. Cl. A61K 47/64; A61K 9/00; A61K 39/00; A61P 25/28; C07K 16/24; C07K 16/28

(52) U.S. Cl. CPC A61K 47/64; A61K 9/0043; A61P 25/28; C07K 16/24; C07K 16/2845; A61K 2039/505; A61K 2039/543; C07K 2317/622

(57) ABSTRACT

The present invention is directed to engineered brain-penetrating therapeutic compounds, and methods of use thereof. Also disclosed are intranasal administration methods for active agents.

### RELATED APPLICATIONS

This application is a National Stage Entry of PCT Application No. PCT/US2023/61750, which claims priority or the benefit under 35 U.S.C. 119 of U.S. provisional application nos. 63/306,985, filed Feb. 4, 2022; 63/347,113, filed May 31, 2022; and 63/480,398, filed Jan. 18, 2023, the contents each of which are fully incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### GOVERNMENT INTERESTS

This invention was made with government support under Grant No. R01 AI048125 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Apr. 13, 2023, is named 5031461-135-WO1_SL.xml and is 19,948 bytes in size.

### FIELD OF THE INVENTION

This invention is directed to engineered brain-penetrating therapeutic compounds, and methods of use thereof.

### BACKGROUND OF THE INVENTION

Alzheimer's Disease (AD) is a progressive neurological disorder that causes the brain to shrink and brain cells to die. AD is the most common cause of dementia, which is a continuous decline in thinking, behavioral and social skills that affects a person's ability to function independently. Worldwide, approximately 55 million people currently have dementia, and this number is expected to rise to 78 million in 2030 and 139 million in 2050. There are no effective treatments that cure AD or alters the disease process in the brain.

### SUMMARY OF THE INVENTION

Generally, this disclosure relates to compounds that can cross the blood brain barrier (BBB) in a patient. The compounds may include a “payload” or active agent that may be a therapeutic agent, a cytotoxic agent, an imaging agent, and the like. The compounds generally include a moiety for delivery of the active agent across the BBB. In embodiments, the moiety may include one or combinations of a carrier agent, a bridge (e.g., amino acid bridge) and a linker (e.g., a flexible linker).

An aspect of the invention is directed to a therapeutic compound that can cross a blood brain barrier in a patient. In embodiments, the therapeutic compound comprises a therapeutic agent conjugated to a positively charged amino acid bridge, a flexible linker, a carrier agent, or a combination thereof. In embodiments, the therapeutic compound comprises a therapeutic agent conjugated to a a positively charged amino acid bridge, a flexible linker, and a carrier agent. The carrier agent can be configured to cross the blood brain barrier. The amino acid bridge can be configured to cross the blood brain barrier.

Another aspect of the invention includes a moiety for delivery of a therapeutic agent across the blood brain barrier in a patient. In certain embodiments, the moiety comprises a positively charged amino acid bridge, a flexible linker, and a carrier agent. In embodiments, the carrier agent is configured to cross the blood brain barrier via receptor-dependent transcytosis (RDT). In embodiments, the amino acid bridge is configured to cross the blood brain barrier via adsorption-mediated transcytosis (AMT).

In various aspects and embodiments disclosed herein, the positively charged amino acid comprises lysine, arginine, histidine, or a combination thereof. The positively charged amino acid can comprise two consecutive lysine residues.

In various aspects and embodiments disclosed herein, the flexible linker comprises 6-aminohexanoic acid (Ahx).

In certain aspects and embodiments disclosed herein the carrier agent comprises Angiopep-2.

The therapeutic agent can comprise an anti-osteopontin (OPN) antibody. The antibody can be conjugated to a moiety for delivery of a therapeutic agent across the blood brain barrier. In some embodiments, the antibody can be conjugated to the moiety by a MFCO-N-hydroxysuccinimide ester.

An additional aspect includes a pharmaceutical composition for treating a neurodegenerative disease in a patient comprising an anti-osteopontin (OPN) antibody. In embodiments, the pharmaceutical composition comprises a means for crossing the blood brain barrier. In embodiments, the neurodegenerative disease comprises Alzheimer's disease, multiple sclerosis, Parkinson's disease, Amyltrophic Lateral Sclerosis, and the like. In certain embodiments, the means for crossing the blood brain barrier comprises the any of the various moiety embodiments described herein.

In another aspect, the invention includes a method of delivering an agent to the central nervous system of a patient in need thereof. In some embodiments, the method comprises peripherally administering the agent (e.g., a therapeutic agent or moiety as described herein) and permitting the agent to cross the blood brain barrier. In some embodiments, the agent can be administered intravenously. In some embodiments, the agent can be delivered to the brain. In some embodiments, the active agent can be an antibody.

In some embodiments, an active agent (e.g., a therapeutic agent) can be administered using a nasal or intranasal route to deliver the active agent to the brain. In some embodiments, the active agent delivered intranasally may not include a moiety for delivery of the agent across the blood brain barrier. In some embodiments, intranasal delivery of an active agent can result in delivery to the brain without crossing the blood brain barrier. In some embodiments, an active agent delivered intranasally can result in olfactory transfer of the active agent to the brain. In some embodiments, the active agent can be an antibody.

Yet another aspect comprises a method of treating a neurodegenerative disease in a patient. In embodiments, the method comprises administering to the patient any of the pharmaceutical compositions disclosed herein. The neurodegenerative disease can comprise Alzheimer's disease, multiple sclerosis, Parkinson's disease, Amyotrophic Lateral Sclerosis and others. In embodiments, the means for crossing the blood brain barrier comprises any of the various moiety embodiments described herein.

In certain embodiments the therapeutic compound comprises the structure of FIG. 22.

Other objects and advantages of this invention will become readily apparent from the ensuing description.

Other objects and advantages of this invention will become readily apparent from the ensuing description.

### BRIEF DESCRIPTION OF THE FIGURES

FIG. 1A-F shows CD11c⁺ microglia are differentiated from CD11c⁻ precursors upon engulfment of apoptotic neurons (ANs) early in development independent of microglial activation. (A) Brain single cell suspensions of 9-mo old WT mice were generated for validation of microglial CD11c expression by flow cytometry. We first gate on CD11b⁺ cells from single/live cells followed by subsequent gating of CD11b⁺CD45^{low} populations as potential microglia. Almost all the cells in this population (˜99%) are Tmem119⁺, while CCR2 expression is not detectable, indicating that this CD11b⁺CD45^{low} population are microglia. In contrast, CCR2 but not Tmem 119 expression is detected in the CD11b⁺CD45^{high} fraction, i.e., putative macrophage populations. Analysis of the CD11b⁺CD45^{low} microglial population using FMO negative controls for these FACS plots confirms specificity. Brain CD45⁻ cells, mainly containing non-immune cells (neurons, astrocytes, oligodendrocytes, but not microglia) that do not express CD11c were used as negative controls to further validate the specificity of this FACS strategy. (B) The proportion of CD11c⁺ microglia in C57BL/6 (B6) mice during early development and normal aging (n=3) was determined by flow cytometry analysis. CD11c⁺ microglia were initially present at late embryonic day (E18.5), peaked at early postanal day (P5) and gradually declined to marginal levels in young adulthood (3-mon) followed by re-emergence and further expansion during aging. (C) CD11c⁻ microglia were isolated from P5 B6-WT mice by negative section with anti-CD11c magnetic beads (purity >99%) followed by co-incubation×72 h in the presence or absence of pHrodo fluorescent dye-labeled apoptotic neurons (ANs) at a 1:1 ratio. After 72 h incubation, 23% of total CD11c⁻ microglia had ingested ANs (AN⁺), of which 77.4% of these AN⁺ microglia were CD11c⁺. In contrast, the <1% of CD11c⁻ microglia that had not ingested ANs (AN⁻ reflecting 77% of total microglia in culture at 72 h) express CD11c (upper panel). These findings indicate that ˜18% of total CD11c⁻ microglia express CD11c⁺ after incubation with ANs, while 98% of microglia incubated×72 h in the absence of ANs remain CD11c⁻ (lower panel). (D, E) CD11c⁻ microglia isolated from P5 B6-WT mice were incubated in the presence or absence of LPS (10 ng/ml) or Aβ (1 μM)×24 h followed by FACS analysis of microglial activation markers and CD11c expression. Expression of CD11c was assessed in CD86⁺ or MHC II⁺ activated microglia. Despite upregulation of CD86 and MHC II in response to LPS and AD, CD11c⁻ microglial differentiation was not observed in response to these stimuli. FACS plot are representative data from 3 experiments. (F) CD11c⁻ microglia were isolated from P5 B6-WT mice by negative section with anti-CD11c⁻ magnetic beads (purity >99%) followed by co-incubation×72 h in the presence or absence of pHrodo fluorescent dye-labeled ANs at a 1:1 ratio with or without the αVβ3 integrin inhibitor cilengitide (Cil) (10 μM) and/or pan-TAM receptor inhibitor LDC1267 (1 μM). After 72 h incubation, Cilengitide or LDC1267 reduced ANs uptake by CD11c⁻ microglia by ˜50%, and the combination of these two inhibitors further inhibited AN uptake to background levels. Stimulation by ANs induced CD11c expression by ˜20% of initial CD11c⁻ microglia over 72 h, while CD11c expression was not detectable in the absence of AN stimulation during the same period. Inhibition of AN uptake by Cilengitide or LDC1267 reduced acquisition of a CD11c⁺ phenotype by CD11c⁻ progenitors by about 50-75% in each case and inclusion of both inhibitors fully prevented this phenotypic transition (n=3). ****p<0.0001, ***p<0.001, **p<0.01, *p<0.05 by one-way ANOVA with Bonferroni's multiple comparisons test. Data shown as mean±s.e.m.

FIG. 2 shows validation of microglial OPN expression by flow cytometry. Microglial OPN expression was validated by flow cytometry analysis in 9-mo old WT mice using conventional intracellular staining protocols. Microglia were fixed and permeabilized with Intracellular Fixation & Permeabilization Buffer followed by incubation with PE-conjugated anti-OPN Ab (1:10) at 4° C. for 30 min. An isotype control (1:10, PE-conjugated goat IgG) and OPN-KO microglia were used as negative control. Microglia that selectively express the intracellular isoform of OPN (OPN-i-KI) were used as a positive control. WT microglia and OPN-i-KI microglia show similar levels of OPN staining, while staining of microglia from either OPN-KO donors or microglia stained with an isotype control does not yield a detectable signal.

FIG. 3A-H shows the stability of CD11c expression by microglia is regulated by OPN. (A) Flow cytometry analysis of OPN expression in CD11c⁺ versus CD11c⁻ microglia isolated from B6-WT mice at different stages of development and aging (n=3). OPN production was confined to CD11c⁺ microglia during early development and normal aging. ****p<0.0001 by two-way ANOVA with Bonferroni's multiple comparisons test. (B) CD11c⁻ microglia were isolated from P5 B6-WT mice followed by co-incubation×72 h with pHrodo fluorescent dye-labeled apoptotic neurons (ANs) at a 1:1 ratio. CD11c⁻ microglia that engulfed ANs differentiated into CD11c⁺ microglia accompanied by sharply increased expression of OPN (n=3). ****p<0.0001 by two-tailed Student's t test. (C, D) CD11c⁺ microglia (>95% purity) were isolated from P5 and 9-mo old WT and OPN-KO mice followed by incubation×7 d in conventional microglia culture medium (DMEM-F12 with 10% fetal bovine serum+1% penicillin/streptomycin+10 ng/ml M-CSF). In vitro analysis of CD11c⁺ microglial stability was evaluated by comparing the percentage of CD11c⁺ microglia on day 0 with day 7 (percentages were normalized to day 0). CD11c⁺ microglia of P5 WT and 9-mo old WT mice were stable, while OPN-KO CD11c⁺ microglia displayed a significant loss of stability in P5 and 9-mo old WT mice (n=3). ****p<0.0001 by two-way ANOVA with Bonferroni's multiple comparisons test. (E, F) CD11c⁺ microglial stability was assessed using ex vivo organotypic hippocampal slice cultures (OHSC). Freshly prepared OHSC were incubated×24 h with 0.5 mg/ml clodronate liposomes at 35° C. to deplete endogenous microglia. Each microglia-free OHSC was replenished with 4×103 CD11c⁺ microglia isolated from P5 or 9-mo old WT or OPN-KO mice (purity >95%). Representative images displaying CD11c⁺ microglia in hippocampal slices reconstituted with P5 WT or P5 OPN-KO CD11c⁺ microglia at day 7. CD11c⁺ microglia of P5 WT and 9-mo old WT mice stably expressed CD11c, while OPN deficiency reduced CD11c expression by ˜60% (n=3-4). Scale bar: 25 μm. ****p<0.0001, ***p<0.001 by two-way ANOVA with Bonferroni's multiple comparisons test. (G, H) CD11c⁺ microglia isolated from 9-mo old WT and OPN-KO mice were incubated×7 d in vitro or in OHSC in the presence of 1 μM synthetic human Aβ1-42 peptide. CD11c⁺ microglia from 9-mo old WT mice were stable, while OPN deficiency led to a substantial reduction of CD11c phenotype in the presence of AR in vitro and in OHSC, respectively (n=3). ****p<0.0001, **p<0.01 by two-way ANOVA with Bonferroni's multiple comparisons test. Data were shown as mean±s.e.m.

FIG. 4A-B shows OPN deficiency reduces the proportion of CD11c⁺ microglia. (A) The proportion of CD11c⁺ and CD11c⁻ microglia in WT and OPN-KO mice during early development and aging was determined by flow cytometry. OPN deficiency led to a significant reduction of the proportion of CD11c⁺ microglia in P5, 6- and 9-mo old WT mice. An isotype control of CD11c was used as negative control (n=3). **p<0.01, ****p<0.0001 by two-way ANOVA with Bonferroni's multiple comparisons test. (B) The proportion of CD11c⁺ and CD11c⁻ microglia was analyzed in brain cryosections of P5 and 9-mo old WT and OPN-KO mice. OPN-KO mice displayed significantly decreased proportion of CD11c⁺ microglia compared with WT mice (n=3). The proportion of CD11c⁺ or CD11c⁻ microglia was evaluated by the percentage of CD11c⁺ (CD11c⁺Iba-1⁺) or CD11c⁻ microglia (CD11c⁻ Iba-1⁺) within total microglia (Iba-1⁺) in each field. Brain slides incubated without anti-CD11c primary Ab or Tyramide Signal Amplification (TSA) were included as negative controls to exclude non-specific immunofluorescent signals. *p<0.05 by two tailed Student's t test. Data are shown as mean s.e.m.

FIG. 5A-B shows the numbers of CD11c⁺ microglia are comparable in WT and OPN-KO mice at Day 0 in vitro and in OHSC. (A, B) The number of CD11c⁺ microglia of P5, 9-mo old WT mice were compared with age-matched OPN-KO mice by flow cytometry (in vitro) and immunofluorescent staining (in OHSC) at Day 0. The number of CD11c⁺ microglia were similar between P5, 9-mo old WT and OPN-KO mice at Day 0 in vitro and in OHSC (n=3). Statistical analysis was performed by two tailed Student's t test.

FIG. 6 shows the validation of CD11c expression by microglia undergo cold isolation by flow cytometry. Brain single cell suspensions of 9-mo old WT mice was generated by cold isolation for validating microglial CD11c expression. CD11b⁺ cells were gated on single/live cells followed by gating of CD11b⁺CD45^{low} cells as microglia and CD11b⁺CD45^{hi} as macrophages. Large majority of cells in the CD11b⁺CD45^{low} population (95%) are Tmem119⁺, while CCR2 expression is not detectable, indicating that the CD11b⁺CD45^{low} population represents microglia. In contrast, CCR2 but not Tmem119 expression is detected in CD11b⁺CD45^{high} macrophages. Analysis of this CD11b⁺CD45^{low} microglial population with FMO negative control confirmed the specificity of CD11c staining. Brain CD45⁻ cells that mainly contain non-immune cells that do not express CD11c were included as negative controls.

FIG. 7A-G shows the definition of an intrinsic genetic program of CD11c⁺ microglia. (A) Transcriptomic profiling of CD11c⁺ and CD11c⁻ microglia was analyzed in P5 and 9-mo old WT mice by RNA-seq. Venn diagram shows the number of genes expressed in P5 and 9-mo WT CD11c⁺ and CD11c⁻ microglia and genes that are exclusively expressed by each microglial subset. CD11c⁺ microglia-unique genes were identified within those genes that showed a fold change and raw counts in the top 0.15% and negatively-expressed by their CD11c⁻ counterparts. The threshold of negative expression was defined according to raw counts of genes with no expression, e.g., Itgax in CD11c⁻ microglia. A similar strategy was used to identify CD11c⁻ microglial unique genes. (B) The core genetic signature of CD11c⁺ microglia was identified as overlapping unique genes of P5 and 9-mo CD11c⁺ microglia. (C) Heatmap displaying the four CD11c⁺ microglial core genes, including Itgax, Cd209a, Cd209f and Cd36 in P5 and 9-mo CD11c⁺ microglia compared with CD11c⁻ counterparts (FDR<0.05). (D, E) Validation of core genes at the protein level. Core genes of CD11c⁺ microglia were validated at the protein level by flow cytomery analysis. Expression of surface proteins CD36 and CD209a were solely expressed by CD11c⁺ microglia of P5 WT mice and 9-mo old WT mice compared with CD11c⁻ microglia (n=3). ****p<0.0001, **p<0.01 by two-tailed Student's t test. (F) CD11c⁻ microglia isolated from P5 WT mice were incubated×24 h with LPS (10 ng/ml) or Aβ (1 μM) followed by flow cytometry analysis of CD36 and CD209 expression. Protein expression of these core genes was barely detectable in CD86⁺ activated CD11c⁻ microglia in response to activating stimuli. FACS plot are representative of 3 experiments. (G) In vitro differentiation of CD11c⁺ microglia. CD11c⁺ and CD11c⁻ microglia were freshly isolated from P5 WT mice. The CD11c⁺ microglial phenotype was validated by analyzing mRNA expression of representative core genes on day 0. Then CD11c⁻ microglia were incubated×72 h with apoptotic neurons (ANs) to induce CD11c⁻ microglial differentiation into CD11c⁺ microglia. The core genes were validated in CD11c⁺ microglia (differentiated) compared with CD11c⁻ microglia (undifferentiated) on day 3 by Q-PCR. The CD11c⁺ microglial phenotype expressed in CD11c⁺ microglia on day 0 is indicated by upregulation of selected core genes compared with CD11c⁻ microglia. AN-induced CD11c⁻ microglial differentiation is accompanied by induction of a CD11c⁺ microglial genetic program. Expression of core genes was normalized into z-score as shown in the heatmap (n=3), p<0.05.

FIG. 8A-G shows OPN regulates the inherent functions of CD11c⁺ microglia. (A) Microglia isolated from P5 and 9-mo old WT, OPN-KO and OPN-i-KI mice were incubated×1 h with 136 mg pHrodo Green-labeled synaptosomes per 1×10⁵ cells followed by flow cytometry analysis of CD11c⁺ microglial engulfment of synaptosomes. CD11c⁺ microglia from P5, but not 9-mo old WT mice, were highly phagocytic for engulfing synaptosomes in vitro. Deficiency of OPN-s significantly abrogated this function, while OPN-i was not implicated in this process (n=3). ****p<0.0001 by one-way ANOVA with Bonferroni's multiple comparisons test. (B) Ex vivo analysis shows that CD11c⁺ microglia from P5 WT mice, but not 9-mo old WT mice, display robust activity in engulfment of synaptic proteins. P5 WT mice lacking OPN-s, but not OPN-i displayed substantially reduced engulfment of pre-synaptic protein synaptophysin or post-synaptic protein PSD95 by CD11c⁺ microglia (n=3). ****p<0.0001, ***p<0.001 by two tailed Student's t test. (C) CD11c⁺ microglia expressed high levels of proliferation marker Ki-67 early in development (P5). OPN-s, but not OPN-i deficiency decreased Ki-67 expression in CD11c⁺ microglia (n=3). *p<0.05 by one-way ANOVA with Bonferroni's multiple comparisons test. (D) During aging, CD11c⁺ microglia showed relatively low levels of proliferative activity and CD11c⁺ microglial Ki-67 expression was substantially decreased in mice lacking OPN-s (n=3). * WT vs. OPN-KO, #WT vs. OPN-i-KI. ****p<0.0001, *p<0.05, ^{####}p<0.0001, ^{#}p<0.05 by two-way ANOVA with Bonferroni's multiple comparisons test. (E) Microglia isolated from 9-mo old WT mice were incubated×24 h with or without 12.5 μg/ml recombinant mouse OPN (rmOPN) or 10 μM αVβ3 inhibitor (cilengitide) followed by flow cytometry analysis of proliferation marker Ki-67. The rmOPN enhanced Ki-67 expression in CD11c⁺αV3⁺ microglia of 9-mo old WT mice, while this effect was fully abrogated by αVβ3 inhibitor (n=3). **p<0.01, *p<0.05 by one-way ANOVA with Bonferroni's multiple comparisons test. (F) The expression of TNF-α was barely detectable in CD11c⁺ microglia of P5 mice, while its expression gradually increased during aging. The age-dependent increase of TNF-α expression in CD11c⁺ microglia was markedly reduced in WT mice lacking OPN-s, but not OPN-i (n=3). * WT vs. OPN-KO, #WT vs. OPN-i-KI. ****p<0.0001, ^{####}p<0.0001 by two-way ANOVA with Bonferroni's multiple comparisons test. (G) Microglia isolated from 9-mo old WT mice were incubated×24 h with or without 12.5 μg/ml recombinant mouse OPN (rmOPN) or 10 μM αVβ3 inhibitor (cilengitide) followed by flow cytometry analysis of TNF-α expression. Upregulation of TNF-α expression induced by rmOPN was fully abolished by αVβ3 inhibitor in CD11c⁺αVβ3⁺ microglia of 9-mo old WT mice (n=3). **p<0.01 by one-way ANOVA with Bonferroni's multiple comparisons test. Data are shown as mean±s.e.m.

FIG. 9 shows microglial αVβ3 expression gradually increased in WT mice during aging. Representative FACS plots and summary of microglial αVβ3 expression in P5, P30, 3-6-, and 9-mo old WT mice. Microglial αVβ3 expression was gradually increased during aging. Representative plots were from 4 independent experiments.

FIG. 10A-D shows CD11c⁺ microglial core genetic program and the OPN-dependent stable phenotype are retained in the context of 5XFAD disease. (A, B) Selected core genes of CD11c⁺ microglia were validated at protein level by flow cytometry. Protein expression of CD209a and CD36 were solely expressed by CD11c⁻ microglia of 9-month-old 5XFAD mice compared with CD11c⁻ microglia. Isotype controls of CD36 and CD209a were used as negative control. (n=3). ****p<0.0001 by two-tailed Student's t test. (C) CD11c⁺ microglia isolated from 9-mo old 5XFAD or OPN-KO.5XFAD mice were cultured in vitro or in OHSC for 7 days. CD11c⁺ microglia of 9-mo old 5XFAD mice were stable both in vitro and in OHSC, while the stability of CD11′ microglia of OPN-KO.5XFAD mice sharply reduced by ˜40%-80% in vitro and in OHSC, respectively (n=3). ****p<0.0001, ***p<0.001 by two-way ANOVA with Bonferroni's multiple comparisons test. (D) CD11c⁺ microglia isolated from 9-mo old 5XFAD and OPN-KO.5XFAD mice were cultured in vitro or in OHSC in the presence of 1 μM synthetic human Aβ1-42 peptide for 7 days. CD11c⁺ microglia from 9-mo old 5XFAD mice were stable, while OPN deficiency led to a sharp reduction of CD11c⁺ microglial stability in response to Aβ stimulation in vitro and in OHSC (n=3-4). ***p<0.001, **p<0.01 by two-way ANOVA with Bonferroni's multiple comparisons test. Data were shown as mean±s.e.m.

FIG. 11A-L shows OPN deletion diminishes AD pathology and rescues cognitive deficits in 5XFAD mice. (a) Flow cytometry analysis of OPN expression in microglia (CD11b⁺), astrocytes (GFAP⁺) and neurons (MAP2⁺) of 5XFAD mice at different stages of disease development. Microglia are the primary cellular source producing OPN in brains of 5XFAD mice during disease progression (n=3). (b, c) Microglial OPN expression at mRNA level (RT-qPCR) and protein level (flow cytometry). Microglial OPN expression was increased at both mRNA level and protein level in 5XFAD mice compared with age-matched WT mice during disease progression (n=3-4). ****p<0.0001, ***p<0.001, *p<0.05 by two-way ANOVA with Bonferroni's multiple comparisons test. (d) Flow cytometry analysis of OPN expression in CD11c⁺ and CD11c⁻ microglia from 5XFAD mice at different disease stages. OPN production is confined to CD11c⁺ microglia during 5XFAD disease progression (n=3). ****p<0.0001 by two-way ANOVA with Bonferroni's multiple comparisons test. (e) Flow cytometry analysis showing the percentage of CD11c⁺ OPN⁺ microglia was increased in 5XFAD mice compared with age-matched WT mice during disease progression (n=3). ****p<0.0001, **p<0.01 by two-way ANOVA with Bonferroni's multiple comparisons test. (f) Ex vivo analysis of microglial TNF-α expression in WT, 5XFAD and OPN-KO.5XFAD mice during disease progression. OPN-deletion reduced the disease-dependent increasing of microglial TNF-α production in 5XFAD mice (n=3). * WT vs. 5XFAD, ****p<0.0001; ^{#}5XFAD vs. OPN-KO.5XFAD, ^{####}p<0.0001 by two-way ANOVA with Bonferroni's multiple comparisons test. (g, h) Representative immunofluorescent images and quantification of Aβ area in brain cryosections of 5XFAD and OPN-KO.5XFAD mice at different stages of disease. OPN-deficiency resulted in a substantial reduction of total AR plaque area in cortex and hippocampus of 5XFAD mice during disease progression (n=3-4). ****p<0.0001, *p<0.05, ns: not significant by two-way ANOVA with Bonferroni's multiple comparisons test. Scale bar=200 μm. (i) Different forms of Aβ plaque were stained with anti-Aβ (6E10) and Thio-S in brain cryosections of 9-mo old 5XFAD and OPN-KO.5XFAD mice. OPN deficiency resulted in a reduction of percentage of diffuse plaques (6E10⁺ Thio-S⁻) within total plaques (6E10⁺) in 5XFAD mice (n=3). ***p<0.001 by two-tailed Student's t test. (j, k) Representative immunofluorescent images and quantification of dystrophic neurites (labeled with N-terminus APP) per plaque (labeled with Thioflavin-S) in brain sections of 5XFAD and OPN-KO.5XFAD mice. The number of dystrophic neurites were quantified within 25 μm area of each plaque. The number of dystrophic neurites per plaque was significantly reduced in OPN-KO.5XFAD mice compared with 5XFAD mice (n=30-40 plaques from 3 mice per group). ****p<0.0001, ***p<0.001, ns: not significant by two-way ANOVA with Bonferroni's multiple comparisons test. Scale bar=200 μm. (I) Spatial learning and memory (acquisition trials) and cognitive flexibility (reversal trials) was assessed by Water T maze in 9-mo old 5XFAD and OPN-KO.5XFAD mice. Age-matched WT mice were induced as control. The 5XFAD mice lacking OPN showed increased percentage of correct choice in both acquisition and reversal trials (n=3-5, ^{#}: WT vs. 5XFAD, ^{#}p<0.05, ^{##}p<0.01, ^{###}p<0.001; *: 5XFAD vs. OPN-KO.5XFAD, **p<0.01). Statistical analysis was performed by two-way ANOVA with Bonferroni's multiple comparisons test. All data are presented as mean±s.e.m.

FIG. 12A-B shows confirmation of OPN KO in OPN-KO.5XFAD mice. (a) PCR genotyping results validating the successful knock-out of OPN in OPN-KO.5XFAD mice. The 5XFAD mice (OPN^{WT}) show the 300 bp OPN^{WT} band. After crossing with OPN-KO mice, the resulting OPN-KO.5XFAD mice gained 500 bp OPN KO band. All mice show 377 bp 5XFAD transgene band and 324 bp internal positive control IL-2 band. (b) Validation of OPN KO at protein level in microglia of 9-mo old OPN-KO.5XFAD mice. Microglial expression of OPN was undetectable in OPN-KO.5XFAD mice. Isotype control was used as negative control, while microglia from age-matched 5XFAD mice were used as positive control cells. The contour plots were representative results from 3 independent experiments.

FIG. 13 shows the validation of microglial CD11c expression in 5XFAD mice by flow cytometry. Flow cytometry validation of microglial CD11c expression in 9-mo old 5XFAD mice. CD11b⁺ cells were gated on single/live cells followed by the subsequent gating of CD11b⁺CD45^{low} as microglia and CD11b⁺CD45^{hi} as macrophage. Large majority of CD11b⁺CD45^{low} population (˜90%) are Tmem119⁺, while CCR2 expression is not detectable, confirming the CD11b⁺CD45^{low} population as microglia. In contrast, CCR2 but not Tmem 119 expression is detected in CD11 b⁺CD45^{high} macrophages. Analysis of this CD11b⁺CD45^{low} microglial population with FMO negative controls confirms the specificity of CD11c staining. Brain CD45⁻ cells that mainly contain non-immune cells that do not express CD11c was included as negative controls.

FIG. 14A-L shows OPN production by pathogenic CD11c⁺ microglia promote pro-inflammatory responses and inhibits Aβ uptake in 5XFAD mouse brains. (a) Heat map displaying representative differentially expressed genes (DEGs) of CD11c⁺ microglia from 9-mo old 5XFAD mice compared with OPN-KO.5XFAD mice. OPN deletion in CD11c⁺ microglia resulted in a downregulation of inflammatory response related genes (e.g., Tnfrsf9, Il1b, Ifitm1, Ccl17) and an upregulation of phagocytosis related genes (e.g., Trem2, Axl, Mertk, Cd68). Upregulation: log₂ FC>0.5, downregulation: log₂ FC<−0.5, FDR<0.05. (b) The percentage of CD11c⁺ OPN⁺, CD11c⁺ OPN⁻, CD11c⁻OPN⁺ and CD11c⁻OPN⁻ microglial subsets in whole brain of 9-mo old 5XFAD mice was determined by flow cytometry and calculated as the number of each subset within the number of total microglia of the brain (n=3). ****p<0.0001 by one-way ANOVA with Bonferroni's multiple comparisons test. (c) The fraction of CD11c⁺ OPN⁺, CD11c⁺ OPN⁻ and CD11c⁻OPN⁻ microglial subsets were analyzed by immunofluorescent staining in brain cryosections of 9-mo 5XFAD mice. Cortex/hippocampus (C/H): The percentage of each microglial subset was calculated as its number in C/H region out of its total number in whole brain (90 fields from 3 mice were analyzed). Within the C/H region, the percentage of each microglial subset in peri-plaque area (within 25 μm of Aβ plaque core) was calculated as the number of each microglial subset that located in peri-plaque area out of its total number in whole brain (33 fields from 3 mice were analyzed). The percentage of plaque-distal microglia was calculated as the number of each microglial subset that located>25 μm of Aβ plaque core out of its total number in whole brain (33 fields from 3 mice were analyzed). Within the peri-plaque area, Aβ uptake cells of each microglial subset was calculated as the number of Aβ⁺ ingested microglia within the total number of each microglial subset resided in whole brain (33 ROIs from 3 mice were analyzed). ***p<0.001, ns: not significant by one-way ANOVA with Bonferroni's multiple comparisons test. (d) Pie charts summary the fraction of CD11c⁺OPN⁺, CD11c⁺ OPN⁻ and CD11c⁻ OPN⁻ microglial subsets in the 9-mo 5XFAD mice brain. The pie chart was a representative result of 3 independent experiments. (e, f) Representative flow cytometry plots and quantification of TNF-α and TREM2 expression in CD11c⁺ OPN⁺ and CD11c⁺ OPN⁻ microglia of 9-mo old 5XFAD mice. CD11c⁺ OPN⁺ microglia produced substantial amount of TNF-α but showed decreased level of TREM2 expression compared with CD11c⁺ OPN⁻ microglia (n=3). ****p<0.0001, **p<0.01 two-tailed Student's t test. (g) Flow cytometry analysis of TREM2 expression in CD11c⁺TNF-α⁺ vs. CD11c⁺TNF-α⁻ microglia of 9-mo old 5XFAD mice. TREM2 expression was significantly lower in CD11c⁺TNF-α⁺ microglia than CD11c⁺TNF-α⁻ microglia (n=3). ***p<0.001 two-tailed Student's t test. (h) Flow cytometry analysis of αVβ3 expression in CD11c⁺ OPN⁺, CD11c⁺OPN⁻ and CD11c⁻ OPN⁻ microglial subsets in 9-mo old 5XFAD mice. CD11c⁻ OPN⁻ microglia do not express substantial amount of αVβ3, while CD11c⁺ OPN⁺ and CD11c⁺ OPN⁻ microglial subsets express comparable level of αVβ3 (n=3). ****p<0.0001, ns: not significant by one-way ANOVA with Bonferroni's multiple comparisons test. (i) Quantification of Aβ ingested CD11c⁺ microglia in brain cryosections of 9-mo old 5XFAD and OPN-KO.5XFAD mice. Aβ uptake was calculated as the number of Aβ⁺ ingested CD11c⁺ microglia within the total number of peri-plaque CD11c⁺ microglia (24 ROIs from 3 mice per group were analyzed). ****p<0.0001 by two-tailed Student's t test. (j) Flow cytometry analysis of CD11c⁺ microglial TNF-α production in 9-mo old 5XFAD and OPN-KO.5XFAD mice. CD11c⁺ microglial TNF-α expression was substantially decreased in OPN-KO.5XFAD mice compared with age-matched 5XFAD (n=3). ***p<0.001 by two-tailed Student's t test. (k) Flow cytometry analysis showing production of TNF-α in CD11c⁺αVβ3 versus CD11c⁺αVβ3⁻ microglia from 9-mo old 5XFAD and OPN-KO.5XFAD mice. OPN deletion resulted in pronounced reduction of TNF-α in CD11c⁺αVβ3⁺ microglia, but not in CD11c⁺αVβ3⁻ microglia in 5XFAD mice (n=3). ****p<0.0001, ns: not significant by one-way ANOVA with Bonferroni's multiple comparisons test. (1) Flow cytometry analysis of TREM2 expression by CD11c⁺ microglia in 9-mo old 5XFAD mice and OPN-KO.5XFAD mice. OPN deficiency led to a remarkable increase of TREM2 expression in CD11c⁺ microglia of 5XFAD mice (n=3). **p<0.01 by two-tailed Student's t test.

FIG. 15A-B shows immunofluorescent staining of CD11c microglial subsets in brain cryosections of 5XFAD mice. (a) Immunofluorescent signal of microglial CD11c expression was validated in 9-mo old 5XFAD mice. Brain cryosections incubated without anti-CD11c primary Ab or Tyramide Signal Amplification (TSA) reagent were used as negative controls. Scale bar=50 μm. (b) Representative immunofluorescent staining of CD11c⁺ OPN⁺ microglia (CD11c⁺ OPN⁺Iba-1⁺, indicated by cyan arrow), CD11c⁺ OPN⁻ microglia (CD11c⁺Iba-1⁺, indicated by magenta arrow) and CD11c⁻ OPN⁻ microglia (Iba-1⁺, indicated by yellow arrow) in brain of 9-mo old 5XFAD mice. Scale bar=25 μm.

FIG. 16A-E shows microglial expression of TREM2, TNF-α and canonical OPN receptors. (a) Representative flow cytometry plots and quantification of TREM2 mean fluorescence intensity (MFI) in CD11c⁺ OPN⁺ and CD11c⁺ OPN⁻ microglial subsets of 9-mo old 5XFAD mice. CD11c⁺ OPN⁻ microglial subset showed increased percentage of TREM2⁺ cells compared with CD11c⁺ OPN⁺ subset, whereas TREM2 MFI was comparable between these two microglial subsets (n=3). Statistical analysis was performed by two-tailed Student's t test. (b) Representative flow cytometry plots of TNF-α and TREM2 expression in CD11c⁻ OPN⁻ microglial subset of 9-mo old 5XFAD mice. CD11c⁻ OPN⁻ microglia expressed low level of TNF-α and negligible level of TREM2. The plots were representative results from 3 independent experiments. (c) Microglial expression of canonical OPN receptors in 5XFAD mice at different stages of disease. The expression of αVβ3 was gradually increased (n=3), while the expression of CD44 or αVβ5 was barely detectable in microglia of 5XFAD mice during disease progression. The histogram of CD44 and the contour plot of αVβ3 and αVβ5 were the representative results from 3 independent experiments. (d) The impact of OPN-αVβ3 interaction on CD11c⁺ microglial production of TNF-α. Upregulated TNF-α production induced by rmOPN was fully abrogated by αVβ3 inhibitor in CD11c⁺ microglia of 9-mo old OPN-KO.5XFAD mice (n=3). ****p<0.0001 by one-way ANOVA with Bonferroni's multiple comparisons test. (e) Representative flow cytometry plots and quantification of TREM2 MFI in CD11c⁺ microglia of 9-mo old 5XFAD and OPN-KO.5XFAD mice (n=3). OPN-KO.5XFAD mice showed increased percentage of TREM2⁺ microglia within CD11c⁺ microglia compared with 5XFAD mice, while TREM2 MFI was comparable between the two groups. Two-tailed Student's t test was performed for statistical analysis.

FIG. 17 shows the fraction of microglial subsets in 5XFAD mouse brain. Schematic illustration showing that <30% of CD11c⁻ OPN⁻ microglia are enriched in the cortex/hippocampus (C/H) region of 5XFAD mouse brain and only 13% locate in the peri-plaque area. They have low capability in Aβ uptake (˜2%) and express marginal level of TNF-α or TREM2. Majority of CD11c⁺ OPN⁻ microglia (˜70%) reside in the C/H region. They are highly enriched (63%) in the peri-plaque area and may be protective, as most of them (˜60%) in the peri-plaque area can uptake AR but produce negligible levels of TNF-α while express high level of TREM2. In contrast, the CD11c⁺ OPN⁺ microglial subset, which is also significantly enriched (˜60%) in peri-plaque areas, show low levels of Aβ uptake (˜7%) but almost 60% produce TNF-α and express low level of TREM2, supporting that CD11c⁺ OPN⁺ microglia may represent a pathogenic microglial subset.

FIG. 18A-D shows in vitro analysis of OPN-dependent inhibition of TREM2-lysosomal phagocytic pathway. (a) Protocol of in vitro analysis of OPN-dependent inhibition of TREM2-lysosomal phagocytic pathway in 9-mo old 5XFAD and OPN-KO.5XFAD mice. (b) TREM2 expression was significantly higher in CD11c⁺ microglia of OPN-KO.5XFAD mice compared with 5XFAD mice and its expression was sharply reduced in response to rmOPN. The neutralizing anti-OPN Ab abrogated the impact of rmOPN (n=3). ****p<0.0001, ***p<0.001, **p<0.01 by one-way ANOVA with Bonferroni's multiple comparisons test. (c) CD68 expression in CD11c⁺ microglia of OPN-KO.5XFAD mice was higher than 5XFAD mice and its expression was significantly reduced by rmOPN, while anti-OPN Ab fully abolished the impact of rmOPN (n=3). ****p<0.0001, ***p<0.001, by one-way ANOVA with Bonferroni's multiple comparisons test. (d) The MFI of FAM-Aβ 1-42 after 1 h's incubation was determined by flow cytometry in lysosomes of CD11c⁺ microglia (CD11c⁺CD68⁺) and defined as Aβ MFI₁ₕ. MFI of retained FAM-Aβ in lysosomes of CD11c⁺ microglia (CD11c⁺CD68⁺) 24 h after FAM-Aβ₁₋₄₂ withdrawal was determined and defined as Aβ MFI₂₄ₕ. CD11c⁺ microglia of OPN-KO.5XFAD mice showed higher Aβ degradation rate [(Aβ MFI₁ₕ−Aβ MFI₂₄ₕ)/Aβ MFI₁ₕ] than 5XFAD mice, while rmOPN significantly reduced the Aβ degradation rate. The impact of rmOPN was completely blocked by anti-OPN Ab (n=3). **p<0.01, *p<0.05 by one-way ANOVA with Bonferroni's multiple comparisons test. All data are presented as mean±s.e.m.

FIG. 19A-J shows OPN impairs Aβ plaque compaction via suppressing TREM2-lysosomal phagocytic pathway. (a, b) Representative immunofluorescent images and quantification of CD11c⁺TREM2⁺ microglia in brain cryosections of 9-mo old 5XFAD and OPN-KO.5XFAD mice. The proportion of CD11c⁺TREM2⁺ microglia was 2-fold higher in OPN-KO.5XFAD mice compared with 5XFAD mice (n=14 ROIs from 3 mice/group). *p<0.01 by two-tailed Student's t test. Scale bar=50 μm. (c, d) Representative immunofluorescent images and quantification of CD68⁺ area in CD11c⁺ microglia located within 25 μm of Aβ plaque (defined as plaque-associated CD11c⁺ microglia) in 9-mo old 5XFAD and OPN-KO.5XFAD mice. OPN-deficiency led to an increase of CD68⁺ area in CD11c⁺ microglia of 5XFAD mice (n=20 ROIs from 3 mice/group). *p<0.05 by two-tailed Student's t test. Scale bar=25 μm. (e, f) Representative immunofluorescent images and quantification of Cathepsin B⁺ area in plaque-associated CD11c⁺ microglia of 9-mo old 5XFAD and OPN-KO.5XFAD mice. A substantial increase of Cathepsin B⁺ area was observed in CD11c⁺ microglia of OPN-KO.5XFAD compared with 5XFAD (n=22 ROIs from 3 mice/group). ****p<0.0001 by two-tailed Student's t test. Scale bar=25 μm. (g) Representative immunofluorescent images from the brains of 9-mo old 5XFAD mice and OPN-KO.5XFAD mice stained with 6E10 (red) and Thioflavin-S (green) displaying two different forms of Aβ plaques. Total plaques were defined as 6E10⁺. White arrows indicate diffuse plaques (6E10⁺ Thio-S⁻), while compact plaques (6E10⁺ Thio-S⁺) were indicated by yellow arrows. Scale bar=100 μm. (h, i) Quantification of total area (6E10⁺) and compact area (6E10⁺ Thio-S⁺) of each plaque in 3-, 6-, and 9-mo old 5XFAD mice and OPN-KO.5XFAD mice (n=30-40 compact plaques from 3 mice/group). ****p<0.0001, *p<0.05, ns: not significant by two-way ANOVA with Bonferroni's multiple comparisons test. (j) The compactness index of each compact plaque in 3-, 6-, and 9-mo old 5XFAD mice and OPN-KO.5XFAD mice. The compactness index was calculated as 6E10⁺ Thio-S⁺ area/6E10⁺ area (n=30-40 compact plaques from 3 mice/group). ****p<0.0001, **p<0.01, ns: not significant by two-way ANOVA with Bonferroni's multiple comparisons test. All data are presented as mean±s.e.m.

FIG. 20A-I shows increased microglial OPN production positively correlates with AD severity in human AD brains. (a) OPN protein expression in brain (middle frontal gyrus) homogenates of AD patients (CDR≥1, n=11), mild cognitive impairment patients (MCI, CDR 0.5, n=10) and cognitively normal controls (CDR 0, n=11). AD patients showed significantly higher brain OPN protein expression compared to cognitively normal controls, while OPN expression was comparable between MCI and AD patients and between MCI and controls. **p<0.01, ns: not significant by one-way ANOVA with Bonferroni's multiple comparisons test. (b) Positive correlation of brain OPN expression level with CDR scores in AD patients (CDR≥1, n=11), MCI patients (CDR 0.5, n=10) and controls (CDR 0, n=11). r=0.5046, p=0.0032 by Pearson's correlation analysis. (c, d) Representative immunofluorescent images from middle frontal gyrus of AD patients and normal controls stained for Iba-1 (microglia, red), CD11c (green) and OPN (cyan) indicated by white arrows. Percentage of CD11c⁺ OPN⁺ microglia were significantly higher in brains of AD patients (CDR≥1, n=8) compared with MCI (CDR 0.5, n=9) and control subjects (CDR 0, n=5). Each dot represents the average percentage of CD11c⁺ OPN⁺ microglia over total Iba-1⁺ cells in 10-12 fields of each brain sample. Scale bar=25 μm. ****p<0.0001, ns: not significant by one-way ANOVA with Bonferroni's multiple comparisons test. (e) The percentage of CD11c⁺ OPN⁺ microglia in brains positively correlates with CDR scores in AD patients (CDR≥1, n=8), MCI patients (CDR 0.5, n=9) and controls (CDR 0, n=5). r=0.8383, p<0.0001 by Pearson's correlation analysis. (f, g) Brain OPN concentrations and the percentage of CD11c⁺ OPN⁺ microglia positively correlate with neuritc plaque rating (r=0.4919, p=0.0043; r=0.8226, p<0.0001, respectively) by Pearson's correlation analysis. (h, i) Brain OPN level and the percentage of CD11c⁺ OPN⁺ microglia positively correlate with tangle rating (r=0.4884, p=0.0046; r=0.7434, p<0.0001, respectively) by Pearson's correlation analysis. All data are presented as mean±s.e.m.

FIG. 21 shows brain penetration of unmodified vs. modified KK-Ahx-Angiopep2-conjugates under one embodiment.

FIG. 22 shows an exemplary Angipep-2-conjugated anti-OPN/anti-CD11b mAb under one embodiment (SEQ ID NO: 20).

FIG. 23 shows an exemplary model depicting how OPN may function in Alzheimer's Disease. OPN suppression of microglial Aβ plaque compaction and promotion of inflammatory responses contribute to cognitive impairment.

FIG. 24A-C shows exemplary data correlating OPN expression with disease progression in a 5XFAD mouse model. As shown, (A) microglia are the primary cellular source producing OPN in brains of 5XFAD mice during disease progression (n=3). In (B) and (C), microglial OPN expression was increased at both mRNA and protein levels in the mice, compared with age-matched wild-type mice during disuse progression (n=3-4). ****p<0.0001, ***p<0.001, *p<0.05 by two-way ANOVA with Bonferroni's multiple comparisons test. These figures are the same as FIGS. 11A, 11B and 11C, respectively.

FIG. 25A-B shows exemplary data of OPN expression in CD11c⁺ microglia in a 5XFAD mouse model. As shown, (A) OPN production is confied to CD11c⁺ during 5XFAD disease progression (n=3). ****p<0.0001. (B) The proportion of CD11c⁺ OPN⁺ microglia was increased in 5XFAD mice compared with age-matched wild-type mice during disease progression (n=3). ****p<0.0001, **p<0.01 by two-way ANOVA with Bonferroni's multiple comparisons test. These figures are the same as FIGS. 11D and 11E, respectively.

FIG. 26A-F shows exemplary data of OPN deficiency diminishing inflammatory response, plaque areas and diffuseness, neuritic dystrophy and cognitive impairment in a 5XFAD mouse model. As shown, (A) deletion of OPN reduced microglial TNF-α production (n=3, *WT vs. 5XFAD, ^{#}5XFAD vs. OPN^{-/-}. 5XFAD, ****p<0.0001, ^{#####}p<0.0001). (B), (C) OPN deficiency resulted in a reduction of Aβ plaque area and (D) percentage of diffuse plaques (6E10+ Thio-S⁻) in total plaques (6E10+) of 5XFAD mice (n=3). (E) OPN deficiency decreased the number of dystrophic neurites in 5XFAD mice (n=3). (F) Genetical depletion of OPN rescued cognitive impairment of 5XFAD mice (n=3-5, ^{#}: WT vs. 5XFAD, ^{#}p<0.05, ^{##}p<0.01, ^{####}p<0.001, ^{####}p<0.0001; *: 5XFAD vs. OPN^{-/-}.5XFAD, ** p<0.01). Analyzed by two-way ANOVA with Bonferroni's multiple comparisons test. These figures are the same as FIGS. 11F, 11G, 11H, 11I, 11K and 11I, respectively.

FIG. 27A-E shows exemplary data of OPN promoting pro-inflammatory and inhibiting phagocytic responses by CD11c⁺ microglia in 5XFAD mice. (A) OPN deletion in CD11c+ microglia resulted in a downregulation of inflammatory response related genes and an upregulation of phagocytosis related genes in 9-mo old 5XFAD mice as shown by heatmap. (log₂ FC>1 or log₂ FC<−1, FDR<0.05). (B) Number of TNF-α⁻ expressing CD11c⁺ microglia was substantially decreased in OPN^{-/-}.5XFAD mice compared with 5XFAD with disease progression (n=3). ****p<0.0001, ***p<0.001. (C) OPN deletion resulted in pronounced reduction of TNF-α expression in CD11c⁺αVβ3⁺ microglia, but not in CD11c⁺αVβ3− microglia in 5XFAD mice (n=3). ****p<0.0001, *p<0.05 by two-way ANOVA with Bonferroni's multiple comparisons test. (D), (E) In situ analysis of brain cryosections revealed that Aβ engulfed by CD11c⁺ microglia was decreased in 9-mo old 5XFAD compared with OPN^{-/-}.5XFAD mice. Yellow arrow indicates Aβ material that has been engulfed inside the microglia. The ratio of Aβ engulfment was calculated as Aβ⁺ area engulfed by CD11c⁺ microglia (20 CD11c+ microglia from 3 mice). ***p<0.001 by two-tailed Student's t test. FIG. 27A is the same as FIG. 14A.

FIG. 28A-E shows exemplary data of OPN suppressing the TREM2-Axl-lysosomal pathway in CD11c+ microglia from 5XFAD mice. As shown, (A) TREM2 expression in CD11c+vs. CD11c− microglia was analyzed by FACS in 5XFAD mice during disease progression. TREM2 was exclusively expressed by CD11c+ microglia (n=3). **** p<0.0001, *p<0.05. (B) (C) The proportion or CO11c+ TREM2+ microglia was 2-fold higher in OPN^{-/-}.5XFAD mice compared with 5XFAD mice (n=6 sections from 3 mice/group). **p<0.01. (D) OPN-deficiency led to an increase of CD68+ area in plaque-associated CD11c+ microglia in both cortex and hippocampus of 9-mo old 5XFAD mice (Cortex: 30 plaques from 6 sections of 3 mice, hippocampus: 20 plaques from 6 sections of 3 mice). *p<0.05. (E) A substantial increase of Cathepsin B+ area was observed in CD11c+ microglia in both cortex and hippocampus of OPN−/−.5XFAD compared with 5XFAD (Cortex: 22 plaques from 3 mice, hippocampus: 11 plaques from 3 mice). ****p<0.0001, ‘**p<0.01 by two-tailed Student's t test. FIG. 28B is the same as FIG. 19A. FIG. 28C is the same as FIG. 19B. FIG. 28D is the same as FIG. 19D. FIG. 28E is the same as FIG. 19E.

FIG. 29A-D shows exemplary data of OPN inhibiting CD11c+ microglial compaction of Aβ plaques in 5XFAD mice. (A) Brains of 9-mo old 5XFAD mice and OPN^{-/-} 0.5XFAD mice stained with 6E10 (red) and Thioflavin-S (green) displaying two different forms of Aβ plaques. White arrows indicate diffuse plaques (6E10+Thio-S⁻), while compact plaques (6E10+ Thio-S⁻) are indicated by yellow arrows. (B)(C) Quantification of total area (6E10+) and compact area (6E10+Thio-S⁺) of each plaque in 3-, 6-, and 9-mo old 5XFAD and OPN^{-/-}.5XFAD mice (30-40 compact plaques from 3 mice per group). (D) The compactness index of each compact plaque in 3-, 6-, and 9-mo old 5XFAD mice and OPN^{-/-} 0.5XFAD mice. The compactness index was calculated as 6E10⁺ Thio-S⁺ area/6E10⁺ area (30-40 compact plaques from 3 mice per group). ****p<0.0001, **p<0.01 by two-way ANOVA with Bonferroni's multiple compansons test. FIGS. 29A-D are the same as FIGS. 19G-J, respectively.

FIG. 30A-B shows exemplary data correlating brain OPN levels and Alzheimer's Disease severity in human brains. (A) OPN protein expression in middle frontal gyrus homogenates of AD patients (CDR≥1, n=11), mild cognitive impairment patients (MCI, CDR 0.5, n=10) and cognitively normal controls (CDR 0, n=11). Brain OPN protein expression was increased in AD patients compared to normal controls. **p<0.01 by one-way ANOVA with Tukey's multiple comparisons test. (B) Positive correlation of brain OPN expression level with CDR scores in AD patients. MCI patients and controls. r=0.5046, p=0.0032 by Pearson's correlation analysis. FIG. 30A is the same as FIG. 20A. FIG. 30B is the same as FIG. 20B.

FIG. 31A-C shows exemplary data correlating the numbers of CD11c+ OPN+ microglia and Alzheimer's Disease severity in human brains. (A) Middle frontal gyrus of AD patients and controls were stained for Iba-1 (microglia, red), CD11c (green) and OPN (cyan). (B) Percentage of CD11c⁺ OPN⁺ microglia were significantly increased in brains of AD patients (CDR≥1, n=8) compared with MCI (CDR 0.5, n=9) and control subjects (CDR 0, n=5). Each dot represents the average percentage of CD11c⁺ OPN⁺ microglia over total Iba-1⁺ cells in 10-12 fields of each brain sample. HPF: high-power field. ****p<0.0001 by one-way ANOVA with Tukey's multiple comparisons test. (C) The percentage of CD11c⁺ OPN⁺ microglla in brains positively correlates with CDR scores in AD patients. MCI and controls. r=0.8383, p<0.0001 by Pearson's correlation analysis. FIGS. 31A-C are the same as FIGS. 20C-E, respectively.

FIG. 32A-B shows exemplary data correlating brain OPN levels and numbers of CD11c+ OPN+ microglia cells with plaque scores in human brains from Alzheimer's Disease patients. (A)(B) Brain OPN expression (r=0.4919, p=0.0043) and the percentage of CD11c⁺ OPN⁺ microglia (r=0.8226, p<0.0001) positively correlate with plaque rating by Pearson's correlation analysis. FIG. 32A is the same as FIG. 20F. FIG. 32B is the same as FIG. 20G.

FIG. 33A-B shows an exemplary schematic (A) and data (B) for generation and binding activity of an Angiopep-2-conjugated monoclonal antibody. (A) Generation of AF488-labeled Angiopep-2 α-CD11b mAb conjugates (K: lysine). (B) Angiopep-2-conjugated and unconjugated α-CD11b mAb showed similar binding activity (AF488 CD11b⁺ microglia) and mean fluorescence intensity (MFI) after incubation with microglia from 5XFAD mice (n=3).

FIG. 34 shows exemplary data for Angiopep-2 conjugated anti-CD11B monoclonal antibody and brain penetration of the antibody in 5XFAD mice. Angiopep-2-conjugated α-CD11b mAb showed increased brain penetration (AF488⁺ microglia) in brain of 5XFAD mice (n=3), as judged by numbers of surface fluorescent CD11b⁺ microglia. ****p<0.0001 by two-tailed Student's t test.

FIG. 35 shows exemplary data of anti-OPN monoclonal antibody inhibiting microglial TNF-α production in microglial cells from 5XFAD mice. In vitro test: the impact of anti-OPN mAb on microglial TNF-α production: Protocol: Microglia isolated from 9-mo old 5XFAD mice were cultured with anti-OPN mAb (MPIIIBl0(l)) at different concentrations for 24 hours followed by analysis of TNF-α production with flow cytometry. Microglia incubated with isotype control (mouse IgGl) was used as negative control.

FIG. 36 shows an exemplary schematic of OPN-dependent regulation of Aβ breakdown and compaction by microglial cells.

FIG. 37 shows an exemplary schematic of an OPN mechanism of action. A pathogenic OPN-producing CD11c+ microglial subset was identified that promotes the development of AD. The contribution of this CD11c⁺ OPN⁺ microglia to AD pathology and cognitive impairment was defined. Therapies were developed that target OPN in preclinical AD models and improved disease pathology and cognition.

FIG. 38 shows exemplary data of the effects of genetic deletion of OPN on Aβ breakdown and plaque processing in 5XFAD mice.

FIG. 39 shows exemplary data related to OPN in Alzheimer's Disease in 5XFAD mice and humans.

FIG. 40 shows a schematic and data for an embodiment of a therapeutic compound disclosed herein (SEQ ID NO: 20).

FIG. 41 shows an exemplary schematic for embodiments of therapeutic compounds disclosed herein.

FIG. 42A-G shows administration of anti-OPN mAb inhibits microglial proinflammatory responses and ameliorates Aβ plaque pathology. (A) Schematic outline of anti-OPN mAb administration. Weekly intravenous injections (10 mg/kg) of anti-OPN mAb or isotype control (mouse IgG1) into 5XFAD mice starting at 6-mo of age for 1 or 2 months. Microglial proinflammatory responses and Aβ plaque pathology were analyzed after 1-mo or 2-mo treatment. (B) The percentage of CD11c+ microglia and TNF-α+CD11c+ microglia was determined by flow cytometric analysis. Treatment for one month with anti-OPN mAb resulted in a modest reduction of CD11c+ microglia and a 35% reduction in TNF-α expression by CD11c+ microglia compared with age-matched 5XFAD mice treated with isotype-matched IgG1 (n=3). Treatment for two months resulted in a 45% reduction in CD11c+ microglia and 55% decrease in TNF-α production by CD11c+ microglia (n=3-4). **p<0.01, *p<0.05, ns: not significant by two-tailed Student's t test. (C, D) Representative immunofluorescent images from brains of 5XFAD mice after 1-mo or 2-mo treatment with anti-OPN mAb or isotype control. Immunofluorescence is shown after staining with 6E10 (red) and Thioflavin-S (green) to distinguish condensed and diffuse forms of Aβ plaque. Scale bar=100 μm. (E-G) Quantitation of total plaque area (6E10⁺), diffuse plaque area (6E10+Thio-S−) and Compactness Index of plaques in 5XFAD mice after 1-mo or 2-mo treatment with anti-OPN mAb compared with isotype treated mice (n=3). The Compactness Index is calculated as 6E10+Thio-S+ area/6E10+ area (n=9 fields from 3 mice/group). *p<0.05, ns: not significant by two-tailed Student's t test. All data are presented as mean s.e.m.

FIG. 43A-C shows administration of cyclic RGD (Cilengitide) inhibits microglial proinflammatory responses. (A) Schematic outline of cyclic RGD (Cilengitide) administration. Weekly intravenous injection (25 mg/kg) or intranasal delivery (10 mg/kg) of Cilengitide or vehicle control (saline) into 5XFAD mice starting at 6-mo of age for 1 or 2 months. Microglial proinflammatory responses were analyzed after 1-mo or 2-mo treatment. (B) Intranasal (IN) delivery of Cilengitide for 1 month resulted in a decreased tendency of CD11c⁺ microglia and a ˜35% reduction of TNF-α production by CD11c⁺ microglia in 5XFAD mice compared with vehicle (saline) treated control mice (n=3). IV injection of Cilengitide for 1 month reduced the percentage of CD11c⁺ microglia by 50% and decreased CD11c⁺ microglial TNF-α production by ˜50% in 5XFAD mice (n=3). *p<0.5. (C) Intranasal (IN) delivery of Cilengitide for 2 months decreased the percentage of CD11c+ microglia by 35% and resulted in a ˜45% reduction of TNF-α expression by CD11c+ microglia. IV injection of Cilengitide for 2 months reduced the percentage of CD11c+ microglia by 60% and decreased CD11c+ microglial TNF-α production by 55% in 5XFAD mice compared with vehicle (saline) treated control mice (n=3). *p<0.05, **p<0.01, ***p<0.001.

FIG. 44A-C shows intranasal delivery of an anti-CD11b monoclonal antibody (e.g., antibody not containing a conjugated -KK-amino acid bridge-AhX linker-Angiopep-2 moiety). Intranasal delivery of the antibody (10 mg/kg bodyweight) to 6-month-old 5XFAD mice (n=4) was performed at time 0. Antibodies were labeled with the fluorophore, AF488. Observations were obtained at 3 hours after administration. Control was intravenous injection of the same non-conjugated anti-CD11b monoclonal antibody. (A) shows anti-CD11b monoclonal antibody fluorescence (green) in brain for intranasal administration versus control intravenous administration. (B) shows anti-CD11b monoclonal antibody fluorescence (green) in brain for intranasal administration, and also shows immunofluorescence of an anti-Iba-1 antibody (red). Iba-1 is a microglial marker. The merged immunofluorescence (yellow) confirms binding specificity of anti-CD11b monoclonal antibody to microglia. (C) shows data for calculation of anti-CD11b monoclonal antibody penetration into the brain in these studies, as the number of fluorescent microglia per microscope field (μm²×10⁶), for anti-CD11b monoclonal antibody administered via the intranasal route as compared to intravenous administration. The data indicate that intranasal administration of the antibody resulted in about a 10-fold increase in brain penetration compared with intravenous injection.

FIG. 45A-C shows example results related to OPN and inflammasome activation. (A) shows Caspase-1 activation and specificity thereof. Intracellular Caspase-1 activity was measured by bioluminescent assay of microglia from 9-month-old 5XFAD mice. Detection of the specificity of Caspase-1 activity was confirmed by a selective Caspase-1 inhibitor (Ac-YVAD-CHO (SEQ ID NO: 13), 1 μM). Bar plots are representative results from three independent experiments. (B-C) shows the impact of OPN-αVβ3 interaction on microglial Caspase-1 activation (B) and IL-10 production (C). Activation of Caspase-1 and IL1β by provision of rmOPN was blocked following provision of αVβ3 inhibitor to microglia from 9-mo old 5XFAD and OPN-KO.5XFAD mice (n=3). ****p<0.0001, ***p<0.001, **p<0.01, *p<0.05, ns: not significant by one-way ANOVA with Bonferroni's multiple comparisons test. U.D. undetectable.

### DETAILED DESCRIPTION OF THE INVENTION

Microgila cells are a type of macrophage found in the central nervous system (CNS). Disclosed herein is a subset of microglial cells that are CD11c+ and produce osteopontin (OPN) in the brain. CD11c+ microglia can contribute to neuronal synapse elimination by engulfing synaptic proteins in early development and mediate pro-inflammatory responses during aging, which activity is depressed when OPN is absent. CD11c+ OPN− microglial cells engulf Aβ (˜60%), express high levels of TREM2, produce negligible levels of TNF-α, and can be protective. In contrast, only a small proportion of CD11c+ OPN+ microglial cells ingest AD, the cells express low levels of TREM2, and many produce TNF-α. OPN production by these cells can reflect enhanced proinflammatory responses and impaired TREM2-dependent Aβ plaque consolidation in activated lysosomes. CD11c+ OPN+ microglial cells can represent a pathogenic microglial subset and may contribute to symptoms of Alzheimer's Disease (AD). These cells can be a target for therapeutic approaches.

Transportation of materials (e.g., therapeutic agents) from the blood to the brain can be regulated by capillary endothelial cells in the brain, referred to as the blood-brain barrier (BBB). Transport of substances across the BBB is restrictive and selective. Molecules with certain properties can cross the BBB by passive diffusion or active/facilitated transport and transcytosis. However, many substances are excluded from transport across the BBB. In some instances, molecules that normally do not cross the BBB can be caused to cross the BBB using various strategies.

Disclosed herein are approaches for transporting compounds across the blood brain barrier (BBB). The compounds can include a payload or active agent including therapeutic agents, cytotoxic agents, imaging agents and the like. The compounds generally include a moiety for delivering the payload or active agent across the BBB. In some embodiments, the active agent can be conjugated to the moiety. In some embodiments, the payload or active agent may be a therapeutic agent for treating a disease or affliction of the brain. In some embodiments, the therapeutic agent may be an antibody or protein-based therapeutic.

In some embodiments, the moiety for delivering the payload/active agent across the BBB can include a carrier agent. The moiety can include a bridge, which can be an amino acid bridge which can be positively charged (e.g., arginine, histidine and/or lysine residues). The moiety can include a linker, which can be a peptide linker, which can be a flexible linker. The moiety can include one or more of the carrier agents, bridges, linkers, and combinations thereof.

In some embodiments, a disease or affliction of the brain can be treated by the compounds and methods for transporting therapeutic agents across the BBB. These diseases may include inflammatory diseases or cancers. In some embodiments, osteopontin (OPN)-mediated neuroinflammatory diseases like Multiple Sclerosis (MS) and Alzheimer's Disease (AD) may be treated.

In some embodiments, the compounds and methods for transporting therapeutic agents across the BBB may be used to target microglial cells that have OPN (e.g., CD11c+ OPN+ microglial cells). In some embodiments, the therapeutic agent can be an antibody specific for CD11c, OPN or CD11c and OPN. These antibodies can target CD11c+ OPN+ microglial cells. In some embodiments, the therapeutic agent can be an antibody specific for OPN.

Aspects of the invention are drawn to engineered brain-penetrating therapeutic compounds. Aspects of the invention are further drawn to methods for treating neurological disorders, such as Alzheimer's Disease and dementia.

Detailed descriptions of one or more preferred embodiments are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in any appropriate manner.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

Wherever any of the phrases “for example,” “such as,” “including” and the like are used herein, the phrase “and without limitation” is understood to follow unless explicitly stated otherwise. Similarly, “an example,” “exemplary” and the like are understood to be nonlimiting.

The term “substantially” allows for deviations from the descriptor that do not negatively impact the intended purpose. Descriptive terms are understood to be modified by the term “substantially” even if the word “substantially” is not explicitly recited.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Aspects of the invention are drawn to methods of treating neurological disorders. For example, the method comprises administering to a subject a therapeutically effective amount of the composition as described herein. In another example, the neurological disorder can comprise a neurodegenerative disease. In another example, a neurodegenerative disease can comprise Alzheimer's Disease.

As used herein, “treatment” and “treating” can refer to the management and care of a subject for the purpose of combating a condition, disease, or disorder, in any manner in which one or more of the symptoms of a disease or disorder are ameliorated or otherwise beneficially altered. The terms “treat” or “treatment” can also refer to both therapeutic treatment and prophylactic or preventative measures, wherein the object is to prevent or slow down (lessen) an undesired physiological change or disorder. The term can include the full spectrum of treatments for a given condition from which the patient is suffering, such as administration of the active compound for the purpose of: alleviating or relieving symptoms or complications; delaying the progression of the condition, disease or disorder; curing or eliminating the condition, disease or disorder; and/or preventing the condition, disease or disorder, wherein “preventing” or “prevention” can refer to the management and care of a patient for the purpose of hindering the development of the condition, disease or disorder, and includes the administration of the active compounds to prevent or reduce the risk of the onset of symptoms or complications. “Treatment” can also refer to prolonging survival as compared to expected survival if not receiving treatment. Those in need of treatment include those already with the condition or disorder as well as those prone to have the condition or disorder or those in which the condition or disorder is to be prevented. The subject or patient to be treated can be a mammal, such as a human being. Treatment also encompasses any pharmaceutical use of the compositions herein, such as use for treating a disease as provided herein.

In embodiments, the method of preventing or treating neurological disorders in a subject in need thereof comprises administering to the subject a therapeutically effective amount of the composition as described herein.

The term “neurological disorder” refers to any condition of the central or peripheral nervous system of a mammal. The term “neurological disorder” includes a neurodegenerative disease (e.g., Alzheimer's disease, Parkinson's disease, multiple sclerosis and amyotrophic lateral sclerosis), neuropsychiatric diseases (e.g., schizophrenia and anxieties, such as general anxiety disorder). Exemplary neurological disorders include MLS (cerebellar ataxia), Huntington's disease, Down syndrome, multi-infaret dementia, status epilecticus, contusive injuries (e.g. spinal cord injury and head injury), viral infection induced neurodegeneration, (e.g. AIDS, encephalopathies), epilepsy, benign forgetfulness, closed head injury, sleep disorders, depression (e.g., bipolar disorder), dementias, movement disorders, psychoses, alcoholism, post-traumatic stress disorder and the like. “Neurological disorder” also includes any condition associated with the disorder. For instance, a method of treating a neurodegenerative disorder includes methods of treating loss of memory and/or loss of cognition associated with a neurodegenerative disorder. An exemplary method would also include treating or preventing loss of neuronal function characteristic of neurodegenerative disorder. “Neurological disorder” also includes any disease or condition that is implicated, at least in part, in monamine (e.g., norepinephrine) signaling pathways (e.g., cardiovascular disease).

“Alzheimer's disease”, “Alzheimer disease”, or “AD” as used herein is a disease in which cognitive function is impaired gradually over time and includes a symptomatic pre-dementia phase with presentation of mild cognitive impairment (MCI), and a dementia phase, where there is a significant impairment in social or occupational functioning.

“Diagnosis” or “prognosis” as used herein refers to the use of information (e.g., genetic information or data from other molecular tests, biological or chemical information from biological samples, signs and symptoms, physical exam findings, cognitive performance results, etc.) to deduce the most likely outcomes, timeframes, and/or responses to a given treatment for a given disease, disorder, or condition, based on comparisons with a plurality of individuals sharing common nucleotide sequences, symptoms, signs, family histories, or other data relevant to consideration of a patient's health status, or the confirmation of a subject's affliction, e.g., with mild cognitive impairment (MCI) (e.g., cognitive impairment of the Alzheimer's type).

As used interchangeably herein, “subject,” “individual,” or “patient,” can refer to a vertebrate, such as a mammal, for example a human. Mammals can include, but are not limited to, murines, simians, humans, farm animals, sport animals, and pets. The term “pet” includes a dog, cat, guinea pig, mouse, rat, rabbit, ferret, and the like. The term “farm animal” includes a horse, sheep, goat, chicken, pig, cow, donkey, llama, alpaca, turkey, and the like.

The term “administration” can refer to introducing a pharmaceutical composition or formulation as described herein into a subject. One route of administration of the composition is intravenous administration. However, any route of administration, such as oral, topical, subcutaneous, peritoneal, intra-arterial, inhalation, vaginal, rectal, nasal, intranasal, introduction into the cerebrospinal fluid, or instillation into body compartments can be used. While not wishing to be bound by theory, in some embodiments, administration of an active agent that includes a moiety for delivery of the active agent across the blood brain barrier, can result in the active agent crossing the blood brain barrier and entering into the brain.

In some embodiments, nasal or intranasal administration can be used to administer and deliver an active agent to the central nervous system (e.g., brain). In some embodiments, nasal or intranalsal delivery of an active agent can use a liquid nasal spray. While not wishing to be bound by theory, intranasal administration of an active agent can result in olfactory transfer of an active agent to the brain. In some embodiments, nerve cells of the olfactory epithelium, present in the nasal cavity and projecting into the olfactory bulb of the brain, can provide a connection between the brain and the external environment. In some embodiments, an active agent administered intranasally can move along the olfactory nerve cells and enter the brain, generally bypassing the blood-brain barrier. In some embodiments, active agents administered via the intranasal route may not include a moiety for delivery of the active agent across the blood brain barrier.

In some embodiments, an intranasal may result in an active agent entering the blood stream. The active agent may then cross the blood-brain barrier and enter the brain. In some embodiments, these active agents can include a moiety or moiety to facilitate delivery to the brain. Active agents may enter the brain via different mechanisms.

The term “therapeutically effective amount” can refer to that amount of a compound or pharmaceutical composition being administered that will relieve to some extent one or more of the symptoms of the disease or condition being treated, and/or that amount that will prevent, or that will prevent to some extent, one or more of the symptoms of the condition or disease that the subject being treated has or is at risk of developing. In an embodiment, therapeutically effective amount can refer to an amount needed to treat a neurological disorder, such as Alzheimer's Disease, or at least one pathological effect resulting from the presence of a neurological condition in a subject human or animal.

Herein, “active agent” or “payload” refers to substances like therapeutic agents, cytotoxic agents, imaging agents, and the like, that are part of the compounds disclosed herein that are transported across the blood brain barrier (BBB). A “therapeutic agent” refers to a substance that, when administered to a subject, can treat a disease or condition in the subject.

As used herein, “adsorption mediated transcytosis” or “AMT” refers to adsorption of a substance at the luminal surface, and exocytosis of the substance at the abluminal surface of an endothelial cell. Generally, this transport across the endothelial cell involves vesicles.

Herein, “antibody” refers to a molecule or molecules that binds an antigen. Herein, “antibody” generally refers to all types of antibodies, fragments and/or derivatives. Antibodies include polyclonal and monoclonal antibodies of any suitable isotype or isotype subclass. Herein, antibody may refer to, but not be limited to Fab, F(ab′)2, Fab′ single chain antibody, Fv, single chain, mono-specific antibody, bi-specific antibody, tri-specific antibody, multi-valent antibody, chimeric antibody, canine-human chimeric antibody, chimeric antibody, humanized antibody, human antibody, CDR-grafted antibody, shark antibody, nanobody (e.g., antibody consisting of a single monomeric variable domain), camelid antibody (e.g., from the Camelidae family) microbody, intrabody (e.g., intracellular antibody), and/or de-fucosylated antibody and/or derivative thereof. Mimetics of antibodies are also provided. In some embodiments, the antibodies disclosed herein are active agents that are part of the compounds disclosed herein that can cross the blood brain barrier.

As used herein, “blood brain barrier” or “BBB” refers to the cellular block between blood and substances in the blood, and the brain. Generally, the BBB is made up of endothelial and other cells.

As used herein, “bridge” can refer to a part of the compounds or moieties disclosed herein. The bridge can connect other components in the compounds or moieties. In some embodiments, the bridge is an amino acid bridge. An amino acid bridge can refer to one or more amino acids that form peptide bonds and connect at least two components of the compounds or moieties, like therapeutic agents, carrier agents, flexible linkers and the like. In embodiments, a bridge may cross the blood brain barrier. In embodiments, a bridge may cross the blood brain barrier by adsorption-mediated transcytosis (AMT).

As used herein, “carrier agent” can refer to a first substance that facilitates crossing of the blood brain barrier by a second substance, where the second substance generally does not cross the BBB alone. The carrier agent is a part of the compounds or moieties disclosed herein. In embodiments, a carrier agent may cross the blood brain barrier. In embodiments, a carrier agent may cross the blood brain barrier via a specific interaction with a corresponding receptor expressed on cells of the blood brain barrier. In embodiments, a carrier agent by cross the blood brain barrier by receptor-dependent transcytosis (RDT).

As used herein, “compound” refers to one or more active agents connected to one or more moieties. Generally, the active agent and a moiety (e.g., carrier agent, bridge, linker, and combinations thereof) make up the compounds described herein. In some embodiments, the active agent can be conjugated to the moiety. When the active agent is conjugated to the moiety, the “compound” can can include the active agent, the moiety and the conjugate

As used herein, “conjugated to” can refer to chemical attachment of one substance to another substance. Herein, a payload or active agent is generally conjugated to the moiety that crosses the blood brain barrier. “Conjugate” can refer to the chemical attachment connecting the active agent and moiety.

As used herein, “linker” is a part of certain embodiments of the compounds or moieties disclosed herein. In some embodiments, “flexible linker” refers to amino acids, that when added to a protein or polypeptide compound or moiety, can increase flexibility of the protein or polypeptide.

Herein, “moiety” refers to the substance or substances attached to an active agent that facilitates the active agent crossing the BBB. In various embodiments, as described in the application, a moiety may include a carrier agent, a carrier agent plus a bridge, a carrier agent plus a linker, or a carrier agent plus a bridge and a linker.

Herein, “microglial cells” refers to a population of macrophages from the central nervous system (CNS). In brain, these cells can remove damaged and infectious cells.

Herein, “osteopontin” refers to a secreted phosphoprotein encoded by the SPP1 gene in humans and the Spp1 gene in mice.

As used herein, “receptor-dependent transcytosis” or “RDT” can refer to binding of a substance to a receptor on the luminal surface of an endothelial cell and exocytosis of the substance at the abluminal surface. Generally, this transport across the endothelial cell involves vesicles.

Herein, “5XFAD mice” refers to a mouse model that recapitulates the amyloid pathology of Alzheimer's disease (Oakley, Holly, et al. “Intraneuronal β-amyloid aggregates, neurodegeneration, and neuron loss in transgenic mice with five familial Alzheimer's disease mutations: potential factors in amyloid plaque formation.” Journal of Neuroscience 26.40 (2006): 10129-10140).

### Osteopontin (OPN) Antibodies

Unique recombinant monoclonal OPN antibodies are described herein. “Recombinant” as it pertains to polypeptides (such as antibodies) or polynucleotides refers to a form of the polypeptide or polynucleotide that does not exist naturally, a non-limiting example of which can be created by combining polynucleotides or polypeptides that would not normally occur together. “Polypeptide” as used herein can encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids, can refer to “polypeptide” herein, and the term “polypeptide” can be used instead of, or interchangeably with any of these terms. “Polypeptide” can also refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide can be derived from a natural biological source or produced by recombinant technology but is not necessarily translated from a designated nucleic acid sequence. It may be generated in any manner, including by chemical synthesis. As to amino acid sequences, one of skill in the art will readily recognize that individual substitutions, deletions or additions to a nucleic acid, peptide, polypeptide, or protein sequence which alters, adds, deletes, or substitutes a single amino acid or a small percentage of amino acids in the encoded sequence is collectively referred to herein as a “conservatively modified variant”. In some embodiments the alteration results in the substitution of an amino acid with a chemically similar amino acid. Conservative substitution tables providing functionally similar amino acids are well known in the art. Such conservatively modified variants of the anti-OPN antibodies disclosed herein can exhibit increased cross-reactivity to OPN in comparison to an unmodified OPN antibody.

For example, a “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is replaced with another amino acid residue from the same side chain family. In another embodiment, a string of amino acids can be replaced with a structurally similar string that differs in order and/or composition of side chain family members.

The OPN antibodies described herein bind to osteopontin. In one embodiment, the OPN antibodies have high affinity and high specificity for osteopontin. In some embodiments, the ostopontin antibody is a monoclonal antibody, MPIIIB10(1), available from the Developmental Studies Hybridoma Bank (https://dshb.biology.uiowa.edu/MPIIIB10-1), which is reactive against at least human, mouse, rat, dog and chicken ostopontin. Other anti-osteopontin monoclonal antibodies can include at least 7C5H12, 2F10, OT15E4, 4H7, OT12F2, OSP/4589, OT16C12, OT16A12, OTI3C4, or 1E10, (https://www.thermofisher.com/antibody/primary/target/osteopontin), OPN46, (https://www.sigmaaldrich.com/US/en/product/sigma/sab4200018), clone 53 (https://www.enzolifesciences.com/ADI-905-629/osteopontin-monoclonal-antibody-53/) and others. Other anti-osteopontin antibodies can be used.

Some embodiments also feature antibodies that have a specified percentage identity or similarity to the amino acid or nucleotide sequences of the anti-OPN antibodies described herein. For example, “homology” or “identity” or “similarity” refers to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous at that position. A degree of homology between sequences is a function of the number of matching or homologous positions shared by the sequences. For example, the antibodies can have 60%, 70%, 75%, 80%, 85%, 90%, 91%, 92%, 93%, 94%, 95%, 96%, 97%, 98%, 99%, or higher amino acid sequence identity when compared to a specified region or the full length of any one of the anti-OPN antibodies described herein. For example, the antibodies can have 60%, 70%, 75%, 80%, 85%, 90%, 91%, 92%, 93%, 94%, 95%, 96%, 97%, 98%, 99%, or higher nucleic acid identity when compared to a specified region or the full length of any one of the anti-OPN antibodies described herein. Sequence identity or similarity to the nucleic acids and proteins of the present invention can be determined by sequence comparison and/or alignment by methods known in the art, for example, using software programs known in the art, such as those described in Ausubel et al. eds. (2007) Current Protocols in Molecular Biology. For example, sequence comparison algorithms (i.e. BLAST or BLAST 2.0), manual alignment or visual inspection can be utilized to determine percent sequence identity or similarity for the nucleic acids and proteins of the present invention.

Aspects of the invention provide isolated antibodies specific against OPN. The term “isolated” as used herein with respect to cells, nucleic acids, such as DNA or RNA, refers to molecules separated from other DNAs or RNAs, respectively, that are present in the natural source of the macromolecule. The term “isolated” can also refer to a nucleic acid or peptide that is substantially free of cellular material, viral material, or culture medium when produced by recombinant DNA techniques, or chemical precursors or other chemicals when chemically synthesized. For example, an “isolated nucleic acid” can include nucleic acid fragments which are not naturally occurring as fragments and would not be found in the natural state. “Isolated” can also refer to cells or polypeptides which are isolated from other cellular proteins or tissues. Isolated polypeptides can include both purified and recombinant polypeptides.

As used herein, an “antibody” or “antigen-binding polypeptide” can refer to a polypeptide or a polypeptide complex that specifically recognizes and binds to an antigen. An antibody can be a whole antibody and any antigen binding fragment or a single chain thereof. For example, “antibody” can include any protein or peptide containing molecule that comprises at least a portion of an immunoglobulin molecule having biological activity of binding to the antigen. Non-limiting examples a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein. As used herein, the term “antibody” can refer to an immunoglobulin molecule and immunologically active portions of an immunoglobulin (Ig) molecule, i.e., a molecule that contains an antigen binding site that specifically binds (immunoreacts with) an antigen. By “specifically binds” or “immunoreacts with” is meant that the antibody reacts with one or more antigenic determinants of the desired antigen and does not react with other polypeptides.

The terms “antibody fragment” or “antigen-binding fragment”, as used herein, is a portion of an antibody such as F_{(ab′)2}, F_{(ab)2}, F_{ab}′, F_{ab}, Fv, scFv and the like. Regardless of structure, an antibody fragment binds with the same antigen that is recognized by the intact antibody. The term “antibody fragment” can include aptamers (such as spiegelmers), minibodies, and diabodies. The term “antibody fragment” can also include any synthetic or genetically engineered protein that acts like an antibody by binding to a specific antigen to form a complex. Antibodies, antigen-binding polypeptides, variants, or derivatives described herein include, but are not limited to, polyclonal, monoclonal, multispecific, human, humanized or chimeric antibodies, single chain antibodies, epitope-binding fragments, e.g., Fab, Fab′ and F(ab′)₂, Fd, Fvs, single-chain Fvs (scFv), single-chain antibodies, dAb (domain antibody), minibodies, disulfide-linked Fvs (sdFv), fragments comprising either a VL or VH domain, fragments produced by a Fab expression library, and anti-idiotypic (anti-Id) antibodies.

A “single-chain variable fragment” or “scFv” refers to a fusion protein of the variable regions of the heavy (VH) and light chains (VL) of immunoglobulins. A single chain Fv (“scFv”) polypeptide molecule is a covalently linked VH:VL heterodimer, which can be expressed from a gene fusion including VH- and VL-encoding genes linked by a peptide-encoding linker. (See Huston et al. (1988) Proc Nat Acad Sci USA 85(16):5879-5883). In some aspects, the regions are connected with a short linker peptide of ten to about 25 amino acids. The linker can be rich in glycine for flexibility, as well as serine or threonine for solubility, and can either connect the N-terminus of the V_{H} with the C-terminus of the V_{L}, or vice versa. This protein retains the specificity of the original immunoglobulin, despite removal of the constant regions and the introduction of the linker. A number of methods have been described to discern chemical structures for converting the naturally aggregated, but chemically separated, light and heavy polypeptide chains from an antibody V region into an scFv molecule, which will fold into a three-dimensional structure substantially similar to the structure of an antigen-binding site. See, e.g., U.S. Pat. Nos. 5,091,513; 5,892,019; 5,132,405; and 4,946,778, each of which are incorporated by reference in their entireties.

Antibody molecules obtained from humans fall into five classes of immunoglobulins: IgG, IgM, IgA, IgE and IgD, which differ from one another by the nature of the heavy chain present in the molecule. Those skilled in the art will appreciate that heavy chains are classified as gamma, mu, alpha, delta, or epsilon (γ, μ, α, δ, ε) with some subclasses among them (e.g., γ1-γ4). Certain classes have subclasses as well, such as IgG₁, IgG₂, IgG₃ and IgG₄ and others. The immunoglobulin subclasses (isotypes) e.g., IgG₁, IgG₂, IgG₃, IgG₄, IgG₅, etc. are well characterized and are known to confer functional specialization. With regard to IgG, a standard immunoglobulin molecule comprises two identical light chain polypeptides of molecular weight approximately 23,000 Daltons, and two identical heavy chain polypeptides of molecular weight 53,000-70,000. The four chains are joined by disulfide bonds in a “Y” configuration wherein the light chains bracket the heavy chains starting at the mouth of the “Y” and continuing through the variable region. Immunoglobulin or antibody molecules described herein can be of any type (e.g., IgG, IgE, IgM, IgD, IgA, and IgY), class (e.g., IgG1, IgG2, IgG3, IgG4, IgA1 and IgA2) or subclass of an immunoglobulin molecule.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells, or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. The variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. The term “antigen-binding site,” or “binding portion” can refer to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” can refer to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three-dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

The six CDRs present in each antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domains, the FR regions, show less inter-molecular variability. The framework regions largely adopt a n-sheet conformation and the CDRs form loops which connect, and in some cases form part of, the n-sheet structure. The framework regions act to form a scaffold that provides for positioning the CDRs in correct orientation by inter-chain, non-covalent interactions. The antigen-binding domain formed by the positioned CDRs provides a surface complementary to the epitope on the immunoreactive antigen, which promotes the non-covalent binding of the antibody to its cognate epitope. The amino acids comprising the CDRs and the framework regions, respectively, can be readily identified for a heavy or light chain variable region by one of ordinary skill in the art, since they have been previously defined (See, “Sequences of Proteins of Immunological Interest,” Kabat, E., et al., U.S. Department of Health and Human Services, (1983); and Chothia and Lesk, J. Mol. Biol., 196:901-917 (1987)).

Where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a given CDR will vary depending on the sequence and size of the CDR. Those skilled in the art can routinely determine which residues comprise a given CDR given the variable region amino acid sequence of the antibody.

| CDR | Kabat Numbering | Chothia Numbering |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

Kabat et al. defined a numbering system for variable domain sequences that is applicable to any antibody. The skilled artisan can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

As used herein, the terms “nanobody” and “isolated VHH domain” can be used interchangeably and refer to camelid single-domain antibody fragments. A “nanobody” refers to the smallest antigen binding fragment or single variable domain (“VHH”) derived from naturally occurring heavy chain antibody. Nanobodies are derived from heavy chain only antibodies, seen in camelids. In the family of “camelids,” immunoglobulins devoid of light polypeptide chains are found. “Camelids” comprise old world camelids (Camelus bactrianus and Camelus dromedarius) and new world camelids (for example, Lama paccos, Lama glama, Lama guanicoe and Lama vicugna). A nanobody with low specificity binds to multiple different epitopes (or polypeptide regions) via a single antigen binding site or binding domain, whereas a nanobody with high specificity binds to one or a few epitopes (or polypeptide regions) via a single antigen binding site or binding domain.

It should be noted that the term “nanobody,” as used herein in its broadest sense, is not limited to a specific biological source or to a specific method of preparation. For example, the nanobodies hereof can generally be obtained: (1) by isolating the VHH domain of a naturally occurring heavy chain antibody; (2) by expression of a nucleotide sequence encoding a naturally occurring VHH domain; (3) by “humanization” of a naturally occurring VHH domain or by expression of a nucleic acid encoding such a humanized VHH domain; (4) by “camelization” of a naturally occurring VH domain from any animal species, and, for example, from a mammalian species, such as from a human being, or by expression of a nucleic acid encoding such a camelized VH domain; (5) by “camelization” of a “domain antibody” or “Dab” as described in the art, or by expression of a nucleic acid encoding such a camelized VH domain; (6) by using synthetic or semi-synthetic techniques for preparing proteins, polypeptides or other amino acid sequences known per se; (7) by preparing a nucleic acid encoding a nanobody using techniques for nucleic acid synthesis known per se, followed by expression of the nucleic acid thus obtained; and/or (8) by any combination of one or more of the foregoing.

The term “monobody” as used herein, refers to an antigen binding molecule with a heavy chain variable domain and no light chain variable domain. A monobody can bind to an antigen in the absence of light chains and has three CDR regions designated CDRH1, CDRH2 and CDRH3. A heavy chain IgG monobody has two heavy chain antigen binding molecules connected by a disulfide bond. The heavy chain variable domain comprises one or more CDR regions, for example, a CDRH3 region. A “VhH” or “VHH” refers to a variable domain of a heavy chain antibody such as a monobody. A “camelid monobody” or “camelid VHH” refers to a monobody or antigen binding portion thereof obtained from a source animal of the camelid family, including animals with feet with two toes and leathery soles.

The term “DARPin” (designed ankyrin repeat protein) refers to an antibody mimetic protein having high specificity and high binding affinity to a target protein, which is prepared via genetic engineering. DARPin is originated from natural ankyrin protein, and has a structure comprising at least 2 ankyrin repeat motifs, for example, comprising at least 3, 4 or 5 ankyrin repeat motifs. The DARPin can have any suitable molecular weight depending on the number of repeat motifs. DARPin includes a core part that provides structure and a target binding portion that resides outside of the core and binds to a target. The structural core includes a conserved amino acid sequence and the target binding portion includes an amino acid sequence that differs depending on the target. DARPin has target specificity similar to an antibody. Thus, a new form of a bispecific chimeric protein is provided by attaching DARPin to an antibody or antibody fragment, such as an IgG (e.g., IgG1, IgG2, IgG3 or IgG4) antibody, or an scFv-Fc antibody fragment, or the like.

As used herein, the term “affibody” refers to proteins engineered to bind to target proteins or peptides with high affinity, imitating monoclonal antibodies, and are therefore a member of the family of antibody mimetics. Affibodies are composed of a three-helix bundle domain derived from the IgG-binding domain of staphylococcal protein A. The protein domain consists of a 58 amino acid sequence, with 13 randomized amino acids affording a range of affibody variants. Despite being significantly smaller than an antibody (an affibody weighs about 6 kDa while an antibody weighs about 150 kDa), an affibody molecule works like an antibody since its binding site is approximately equivalent in surface area to the binding site of an antibody.

As used herein, the term “epitope” can include any protein determinant that can specifically bind to an immunoglobulin, a scFv, or a T-cell receptor. The variable region allows the antibody to selectively recognize and specifically bind epitopes on antigens. For example, the VL domain and VH domain, or subset of the complementarity determining regions (CDRs), of an antibody combine to form the variable region that defines a three-dimensional antigen-binding site. This quaternary antibody structure forms the antigen-binding site present at the end of each arm of the Y. Epitopic determinants can consist of chemically active surface groupings of molecules such as amino acids or sugar side chains and usually have specific three-dimensional structural characteristics, as well as specific charge characteristics. For example, antibodies can be raised against N-terminal or C-terminal peptides of a polypeptide. More specifically, the antigen-binding site is defined by three CDRs on each of the VH and VL chains (i.e., CDR-H1, CDR-H2, CDR-H3, CDR-L1, CDR-L2 and CDR-L3).

In some embodiments, the antibodies can be directed to osteopontin (OPN). In humans, osteopontin is encoded by the SPP1 gene (secreted phosphoprotein 1). The murine ortholog is Spp1. In some embodiments, the antibodies can be directed to human osteopontin (OPN). Osteopontin generally is secreted. There can be intracellular forms of osteopontin. There may be variants of osteopontin, for example, in certain cancer cells.

In some embodiments, human osteopontin is encoded by a gene having NCBI GenBank Gene ID 6696 (SPP1, secreted phosphoprotein 1). In some embodiments, murine osteopontin is encoded by a gene having NCBI GenBank Gene ID 20750 (Spp1, secreted phosphoprotein 1). In some embodiments osteopontin (from rat) has NCBI GenBank Reference No: AAA41765.1 (317 amino acid residues in length), comprising the amino acid sequence of SEQ ID NO: 2:

MRLAVVCFCLFGLASCLPVKVAEFGSSEEKAHYSKHSDAVATWLKPDP
SQKQNLLAPQNSVSSEETDDFKQETLPSNSNESHDHMDDDDDDDDDGD
HAESEDSVNSDESDESHHSDESDESFTASTQADVLTPIAPTVDVPDGR
GDSLAYGLRSKSRSFPVSDEQYPDATDEDLTSRMKSQESDEAIKVIPV
AQRLSVPSDQDSNGKTSHESSQLDEPSVETHSLEQSKEYKQRASHEST
EQSDAIDSAEKPDAIDSAERSDAIDSQASSKASLEHQSHEFHSHEDKL
VLDPKSKEDDRYLKFRISHELESSSSEVN

As used herein, the terms “immunological binding,” and “immunological binding properties” can refer to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and geometric parameters that equally influence the rate in both directions. Thus, both the “on rate constant” (Kₒₙ) and the “off rate constant” (K_{off}) can be determined by calculation of the concentrations and the actual rates of association and dissociation. (See Nature 361: 186-87 (1993)). The ratio of K_{off}/Kₒₙ allows the cancellation of all parameters not related to affinity, and is equal to the equilibrium binding constant, K_{D}. (See, generally, Davies et al. (1990) Annual Rev Biochem 59:439-473). An antibody of the invention can specifically bind to a OPN epitope when the equilibrium binding constant (K_{D}) is ≤1 μM, ≤10 μM, ≤10 nM, ≤10 pM, or ≤100 pM to about 1 pM, as measured by kinetic assays such as radioligand binding assays or similar assays known to those skilled in the art, such as BIAcore or Octet (BLI). For example, in some embodiments, the K_{D} is between about 1E-12 M and a K_{D} about 1E-11 M. In some embodiments, the K_{D} is between about 1E-11 M and a K_{D} about 1E-10 M. In some embodiments, the K_{D} is between about 1E-10 M and a K_{D} about 1E-9 M. In some embodiments, the K_{D} is between about 1E-9 M and a K_{D} about 1E-8 M. In some embodiments, the K_{D} is between about 1E-8 M and a K_{D} about 1E-7 M. In some embodiments, the K_{D} is between about 1E-7 M and a K_{D} about 1E-6 M. For example, in some embodiments, the K_{D} is about 1E-12 M while in other embodiments the K_{D} is about 1E-11 M. In some embodiments, the K_{D} is about 1E-10 M while in other embodiments the K_{D} is about 1E-9 M. In some embodiments, the K_{D} is about 1E-8 M while in other embodiments the K_{D} is about 1E-7 M. In some embodiments, the K_{D} is about 1E-6 M while in other embodiments the K_{D} is about 1E-5 M. In some embodiments, for example, the K_{D} is about 3 E-11 M, while in other embodiments the K_{D} is about 3E-12 M. In some embodiments, the K_{D} is about 6E-11 M. “Specifically binds” or “has specificity to,” can refer to an antibody that binds to an epitope via its antigen-binding domain, and that the binding entails some complementarity between the antigen-binding domain and the epitope. For example, an antibody is said to “specifically bind” to an epitope when it binds to that epitope, via its antigen-binding domain more readily than it would bind to a random, unrelated epitope.

For example, the OPN antibody can be monovalent or bivalent, and can comprise a single or double chain. Functionally, the binding affinity of the OPN antibody is within the range of 10⁻⁵ M to 10⁻¹² M. For example, the binding affinity of the OPN antibody is from 10⁻⁶M to 10⁻¹²M, from 10⁻⁷M to 10⁻¹²M, from 10⁻⁸M to 10⁻¹²M, from 10⁻⁹ M to 10⁻¹²M, from 10⁻⁵ M to 10⁻¹¹ M, from 10⁻⁶ M to 10⁻¹¹ M, from 10⁻⁷ M to 10⁻¹¹ M, from 10⁻⁸ M to 10⁻¹¹ M, from 10⁻⁹ M to 10⁻¹¹ M, from 10⁻¹⁰ M to 10⁻¹¹ M, from 10⁻⁵ M to 10⁻¹° M, from 10-M to 10⁻¹⁰ M, from 10⁻⁷ M to 10⁻¹⁰ M, from 10⁻⁸ M to 10⁻¹⁰ M, from 10⁻⁹ M to 10⁻¹⁰ M, from 10⁻⁵ M to 10⁻⁹ M, from 10⁻⁶ M to 10⁻⁹ M, from 10⁻⁷ M to 10⁻⁹ M, from 10⁻⁸ M to 10⁻⁹ M, from 10⁻⁵ M to 10⁻⁸ M, from 10⁻⁶ M to 10⁻⁸ M, from 10⁻⁷ M to 10⁻⁸ M, from 10⁻⁵ M to 10⁻⁷ M, from 10⁻⁶ M to 10⁻⁷ M, or from 10⁻⁵ M to 10⁻⁶ M.

A OPN protein, or a derivative, fragment, analog, homolog or ortholog thereof, can be utilized as an immunogen in the generation of antibodies that immunospecifically bind these protein components, e.g., amino acid residues comprising SEQ ID NO: 2. A OPN protein or a derivative, fragment, analog, homolog, or ortholog thereof, coupled to a proteoliposome can be utilized as an immunogen in the generation of antibodies that immunospecifically bind these protein components.

Those skilled in the art will recognize that one can determine, without undue experimentation, if a human monoclonal antibody has the same specificity as a human monoclonal antibody of the invention by ascertaining whether the former prevents the latter from binding to OPN. For example, if the human monoclonal antibody being tested competes with the human monoclonal antibody of the invention, as shown by a decrease in binding by the human monoclonal antibody of the invention, then the two monoclonal antibodies can bind to the same, or to a closely related, epitope.

Another way to determine whether a human monoclonal antibody has the specificity of a human monoclonal antibody of the invention is to pre-incubate the human monoclonal antibody of the invention with the OPN protein, with which it is normally reactive, and then add the human monoclonal antibody being tested to determine if the human monoclonal antibody being tested is inhibited in its ability to bind OPN. If the human monoclonal antibody being tested is inhibited then, in all likelihood, it has the same, or functionally equivalent, epitopic specificity as the monoclonal antibody of the invention. Screening of human monoclonal antibodies of the invention can be also carried out by utilizing OPN and determining whether the test monoclonal antibody is able to neutralize OPN.

Various procedures known within the art can be used for the production of polyclonal or monoclonal antibodies directed against a protein of the invention, or against derivatives, fragments, analogs homologs or orthologs thereof (See, for example, Antibodies: A Laboratory Manual, Harlow E, and Lane D, 1988, Cold Spring Harbor Laboratory Press, Cold Spring Harbor, NY, incorporated herein by reference).

Antibodies can be purified by well-known techniques, such as affinity chromatography using protein A or protein G, which provide primarily the IgG fraction of immune serum. Subsequently, or alternatively, the specific antigen which is the target of the immunoglobulin sought, or an epitope thereof, can be immobilized on a column to purify the immune specific antibody by immunoaffinity chromatography. Purification of immunoglobulins is discussed, for example, by D. Wilkinson (The Scientist, published by The Scientist, Inc., Philadelphia PA, Vol. 14, No. 8 (Apr. 17, 2000), pp. 25-28).

The term “monoclonal antibody” or “mAb” or “Mab” or “monoclonal antibody composition”, as used herein, can refer to a population of antibody molecules that contain only one molecular species of antibody molecule consisting of a unique light chain gene product and a unique heavy chain gene product. For example, the complementarity determining regions (CDRs) of the monoclonal antibody are identical in all the molecules of the population. MAbs contain an antigen binding site that can immunoreact with a given epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is immunized with an immunizing agent to elicit lymphocytes that produce or can produce antibodies that will specifically bind to the immunizing agent. Alternatively, the lymphocytes can be immunized in vitro.

### Active Agents

The compounds disclosed here generally include substances desired to be delivered to the brain and are referred to as active agents or payloads. In some embodiments, active agents can include therapeutic agents, drugs, cytotoxic agents, imaging agents, and the like. In some embodiments, the active agents can include proteins. In some embodiments, the active agents can include antibodies or protein-based therapeutic agents.

The invention also is directed to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Non-limiting examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al, Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See PCT Publication No. WO94/11026, and U.S. Pat. No. 5,736,137).

Those of ordinary skill in the art understand that a large variety of moieties can be coupled to the resultant antibodies or to other molecules of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling can be accomplished by any chemical reaction that will bind the two molecules so long as the antibody and the other moiety retain their respective activities. This linkage can include many chemical mechanisms, for instance covalent binding, affinity binding, intercalation, coordinate binding, and complexation. In one embodiment, binding is covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun. 133: 1335-2549 (1984); Jansen et al., Immunological Reviews 62: 185-216 (1982); and Vitetta et al, Science 238: 1098 (1987)). Non-limiting examples of linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Non-limiting examples of useful linkers that can be used with the antibodies of the invention include: (i) EDC (1-ethyl-3-(3-dimethylamino-propyl) carbodiimide hydrochloride; (ii) SMPT (4-succinimidyloxycarbonyl-alpha-methyl-alpha-(2-pyridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS (-hydroxysulfo-succinimide: Pierce Chem. Co., Cat. #24510) conjugated to EDC.

The linkers described herein contain components that have different attributes, thus leading to conjugates with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, for example, can enhance the stability of carbodimide couplings. Carbodimide couplings (such as EDC) when used in conjunction with sulfo-NHS, forms esters that are more resistant to hydrolysis than the carbodimide coupling reaction alone.

In some embodiments, the active agents can include antibodies or other agents that target certain cell types that are related to a disease or affliction that is being treated (e.g., anti-osteopontin antibodies). In some embodiments, the active agents can target cancer cells or cells related to autoimmune disorders. In some examples, the active agents can target microglial cells. The microglial cells can be CD11c+ OPN+ cells. These cells can be found, for example, in people that have Alzheimer's Disease. In some embodiments, the active agents can antibodies specific for osteopontin (OPN) or CD11.

In some embodiments, the active agent can include integrin inhibitors. In some embodiments, the integrin inhibitor can include αVβ3 inhibitors. αVβ3 inhibitors can include, for example, antibodies like LM609/Avastin, CNTO 95, c7E3, and 17E6. αVβ3 inhibitors can include, for example, antagonists like disintegrins, peptides, and non-peptide molecules (e.g., peptidomimetics, siRNAs).

In some embodiments, the αVβ3 inhibitor can be a cyclic RGD-containing small molecule or derivative thereof. In some embodiments, this can be cilengitide or a derivative thereof. Cilengitide is a head-to-tail cyclized RGD (arg-gly-asp)-containing pentapeptide which can bind to integrin αVβ3 and αVβ5. Cilengitide is designed to compete with the RGD peptide sequence that normally regulates integrin-ligand binding. For example, cilengitide blocks ligation of αVβ3 and αVβ5 integrins to matrix proteins like vitronectin, fibronectin, fibrinogen, von Willebrand factor, osteopontin, and the like.

In some embodiments, the αVβ3 inhibitor can be administered as part of the therapeutic regime in which an anti-osteopontin antibody is administered. In some embodiments, the αVβ3 inhibitor can be administered in combination with an anti-osteopontin antibody. The αVβ3 inhibitor can be conjugated to a bridge, linker, carrier agent or a combination thereof. The αVβ3 inhibitor may not be conjugated to a bridge, linker, carrier agent or a combination thereof.

In this disclosure, active agents are generally attached to a moiety or moieties that provide for the active agents to cross the BBB. The active agent plus the moiety or moieties are referred to as the “compounds” of this disclosure that are delivered across the BBB and to the brain. However, in some embodiments, active agents may not be attached to a moiety or moieties that provide for the active agents to cross the BBB.

### Carrier Agents

Carrier agents can be the moiety or can be part of the moiety or moieties attached to the active agents. In some embodiments, the carrier agent can be directly attached to an active agent. In some embodiments, the carrier agent can be attached to an active agent through a conjugate. In some embodiments, the carrier agent can be attached to a bridge or flexible linker, which is attached to the active agent, directly or through a conjugate.

In some embodiments, carrier agents can facilitate transport of the moiety, or compound containing the active agent and the moiety, across the BBB. In some embodiments, these materials can cross the BBB via receptor-dependent transcytosis (RDT).

In some examples, a carrier agent attached to an active agent may result in the active agent crossing the BBB. In some examples, a first carrier agent by itself may not provide for an active agent to cross the BBB but may provide for the active agent to cross the BBB in combination with one or more second carrier agents or other molecules (other molecules including, for example, a bridge and/or a flexible linker). In some examples, a carrier agent may provide for low level crossing of an active agent through the BBB, which may be increased by combining multiple of the same or different carrier agents.

In some embodiments, a carrier agent can be a peptide. In some embodiments, a carrier agent that is a peptide can mediate crossing of an active agent across the blood brain barrier through interaction of the peptide with its receptor on the blood brain barrier. Angiopep-2 (TFFYGGSRGKRNNFKTEEY; SEQ ID NO: 1) is such a peptide. In some embodiments, the peptide may be a cell-penetrating peptide (CPP). Such CPPs, in some embodiments, can be less than 20 amino acids in length and can contain amino acids with positive charges. CPPs can cross cell membrane bilayers via interaction with the negatively-charged cell membrane.

In embodiments, peptide carrier agents can include D-Lys6-LHRH, Angiopep-2, CNGRCG (SEQ ID NO: 4), PGA, LHRH (SEQ ID NO: 5), DRDDS (spacer; SEQ ID NO: 6), D-γ-E-γ-E-γ-E-E (masking moiety; SEQ ID NO: 7), GSH, HSTPSSP (SEQ ID NO: 8), DSSLFAL (SEQ ID NO: 9) and others (Jafari, Behzad, et al. “Peptide-mediated drug delivery across the blood-brain barrier for targeting brain tumors.” Expert opinion on drug delivery 16.6 (2019): 583-605.). In some embodiments, the carrier agent includes Angiopep-2. Angiopep-2 can have the amino acid sequence, TFFYGGSRGKRNNFKTEEY (SEQ ID NO: 1) or a sequence at least 90% identical to SEQ ID NO: 1.

In some embodiments, peptide carrier agents can include YGRKKRRQRRRPPQQ (TAT; SEQ ID NO: 10), LLIILRRRIRKQAHAHSK (pVEC; SEQ ID NO: 11), RRLSYSRRRF (SynB3; SEQ ID NO: 12) and others.

In some embodiments, a carrier agent can be a peptide of up to 20 or 30 amino acids, positively charged and amphipathic, known as cell-penetrating peptides (Gao, Huile, et al. “Angiopep-2 and activatable cell-penetrating peptide dual-functionalized nanoparticles for systemic glioma-targeting delivery.” Molecular pharmaceutics 11.8 (2014): 2755-2763.).

In some embodiments, a carrier agent can be a cell-targeting peptide (Mousavizadeh, Ali, et al. “Cell targeting peptides as smart ligands for targeting of therapeutic or diagnostic agents: A systematic review.” Colloids and Surfaces B: Biointerfaces 158 (2017): 507-517.).

Generally, any carrier agent can be used in the compositions and methods described herein. In some embodiments, any peptide carrier agent can be used in the compositions and methods described herein.

### Bridge

A bridge can be the moiety or can be part of the moiety or moieties attached to the active agents. In some embodiments, the bridge can be directly attached to an active agent. In some embodiments, the bridge can be attached to an active agent through a conjugate. In some embodiments, the bridge can be attached to a flexible linker or carrier agent, which is attached to the active agent, directly or through a conjugate. In some embodiments, the bridge may be positioned between the active agent and the carrier agent.

In some embodiments, a bridge can facilitate transport of the moiety, or compound containing the active agent and the moiety, across the BBB. In some embodiments, these materials can cross the BBB via adsorption-mediated transcytosis (AMT).

Generally, the bridge can be designed to enhance the ability of the moiety and attached active agent to cross the BBB and be delivered to the brain. In some embodiments, the bridge can be designed to alter the charge of the compound containing the active agent (e.g., the active agent plus the moiety).

In some embodiments, the bridge can include amino acids in the form of a peptide or polypeptide. The bridge can include amino acids that alter the charge of the compound containing the active agent. In some embodiments, amino acids that make up the bridge can be selected to increase or decrease the isoelectric point (pI) of the compound containing the active agent. In some embodiments, the amino acids of the bridge can be selected to increase the pI of the compound containing the active agent. In some embodiments, the amino acids of the bridge can be selected to increase the pI of the compound containing the active agent to alkaline levels. These amino acids may be positively charged (e.g., arginine, lysine, histidine). In some embodiments, the amino acid bridge may include 2, 3, 4, 5, 6 or more consecutive positively charged amino acids. In some embodiments, the bridge may include 2, 3, 4, 5, 6 or more consecutive lysine residues. In some embodiments, the bridge can include 2 consecutive lysine residues.

Increasing the pI of the compound may enhance transport of the moiety, or compound containing the active agent and the moiety, across the BBB. In some embodiments, this transport may occur via adsorption-mediated transcytosis (AMT). The increased positive charge of the compound may enhance interactions between the compound and the surface of endothelial cells that make up the BBB. Generally, the surface of these endothelial cells can be negatively charged.

### Linker

Linkers can be the moiety or can be part of the moiety or moieties attached to the active agents. In some embodiments, the linker can be directly attached to an active agent. In some embodiments, the linker can be attached to an active agent through a conjugate. In some embodiments, the linker can be attached to a carrier agent and/or bridge, which is attached to the active agent, directly or through a conjugate. In some embodiments, the linker may be positioned between the active agent and the carrier agent.

Generally, the linker can be designed to increase flexibility of the moiety and/or compound that includes the moiety and the active agent. In some instances, this flexibility can facilitate solvation of the moiety or compound. In some instances, this flexibility can reduce aggregation of the moiety or compound.

In some embodiments, the linker can refer to amino acid or peptide spacers that separate multiple domains (e.g., active agent, carrier agent) within the compound (e.g., protein or polypeptide) that includes an active agent and a moiety.

In some embodiments, the linker can be a flexible linker. Flexible linker can refer to linkers, that when added to compound or molecule, like a protein or polypeptide, can increase flexibility of the compound or molecule. The flexible linker can be a peptide or polypeptide. Other types of peptide linkers can be rigid linkers or cleavable linkers.

In some embodiments, flexible peptide linkers can include small, polar (e.g., Ser, Thr) or non-polar (e.g., Gly) amino acids. The flexible peptide linkers can have sequences of Gly and Ser residues (e.g., a “GS” linker). An example GS linker amino acid sequence may include (Gly-Gly-Gly-Gly-Ser)ₙ (SEQ ID NO: 14). Other types of flexible linkers may include KESGSVSSEQLAQFRSLD (SEQ ID NO: 15), EGKSSGSGSESKST (SEQ ID NO: 16), (Gly)s (SEQ ID NO: 17), GSAGSAAGSGEF (SEQ ID NO: 18) and (GGGGS)₄ (SEQ ID NO: 19) (Chen, Xiaoying, Jennica L. Zaro, and Wei-Chiang Shen. “Fusion protein linkers: property, design and functionality.” Advanced drug delivery reviews 65.10 (2013): 1357-1369.).

In some embodiments, the flexible linkers can include 2-aminoethoxy acetic acid (AEA), 5-aminovaleric acid (Ava), 8-amino-3,6-dioxaoctanoic acid (PEG2 or AEEA), 12-amino-4,7,10-trioxadodecanoic acid (PEG3), and the like.

In some embodiments, 6-aminohexanoic acid (Ahx) can be used as a flexible peptide linker (Markowska, Agnieszka, Adam Roman Markowski, and Iwona Jarocka-Karpowicz. “The Importance of 6-Aminohexanoic Acid as a Hydrophobic, Flexible Structural Element.” International Journal of Molecular Sciences 22.22 (2021): 12122.).

Generally, any of these flexible linkers can be used in the compounds described herein.

### Conjugates

The moieties, as described herein, may be attached to an active agent using a variety of structures. Herein, these structures may be called “conjugates.” Attaching an active agent to a moiety may be called “conjugating” or “conjugation.” In some instances, when biomolecules are involved, the conjugated structures may be called bioconjugates.

In some embodiments, the conjugate may be chemical. In some embodiments, the conjugate may be non-cleavable or cleavable. The conjugate may be designed to release an active agent under certain stimuli, including environmental stimuli like pH, redox conditions, in the presence of a given enzyme, and the like.

In some embodiments, conjugation or bioconjugation may be performed using “click” chemistry (Hein, Christopher D., Xin-Ming Liu, and Dong Wang. “Click chemistry, a powerful tool for pharmaceutical sciences.” Pharmaceutical research 25.10 (2008): 2216-2230.).

In some embodiments, attachment of an active agent that is a protein (e.g., an antibody) to the moieties disclosed herein may use MFCO-N-hydroxysuccinimide ester.

In some embodiments, a conjugate may include Sulfo-NHS esters, Biotin-NHS-esters, and the like.

### Pharmaceutical Compositions

Antibodies of the invention specifically binding a OPN protein or fragment thereof can be administered for the treatment of a neurological disorder or a neurodegenerative disease in the form of pharmaceutical compositions. Principles and considerations involved in preparing therapeutic pharmaceutical compositions comprising the antibody, as well as guidance in the choice of components are provided, for example, in: Remington: The Science And Practice Of Pharmacy 20th ed. (Alfonso R. Gennaro, et al, editors) Mack Pub. Co., Easton, Pa., 2000; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

A specific dosage and treatment regimen for any given patient will depend upon a variety of factors, including the given antibodies, variant or derivative thereof used, the patient's age, body weight, general health, sex, and diet, and the time of administration, rate of excretion, drug combination, and the severity of the given disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

A therapeutically effective amount of an antibody of the invention can be the amount needed to achieve a therapeutic objective. As noted herein, this can be a binding interaction between the antibody and its target antigen that, in certain cases, interferes with the functioning of the target. The amount required to be administered will furthermore depend on the binding affinity of the antibody for its specific antigen and will also depend on the rate at which an administered antibody is depleted from the free volume other subject to which it is administered. The dosage administered to a subject (e.g., a patient) of the antigen-binding polypeptides described herein is about 0.1 mg/kg to 100 mg/kg of the patient's body weight, between 0.1 mg/kg and 20 mg/kg of the patient's body weight, or 1 mg/kg to 10 mg/kg of the patient's body weight. Human antibodies have a longer half-life within the human body than antibodies from other species due to the immune response to the foreign polypeptides. Thus, lower dosages of human antibodies and less frequent administration can be used. Further, the dosage and frequency of administration of antibodies of the disclosure may be reduced by enhancing uptake and tissue penetration (e.g., into the brain) of the antibodies by modifications such as, for example, lipidation. Common ranges for therapeutically effective dosing of an antibody or antibody fragment of the invention can be, by way of nonlimiting example, from about 0.1 mg/kg body weight to about 50 mg/kg body weight. Common dosing frequencies can range, for example, from twice daily to once a week.

Where antibody fragments are used, the smallest inhibitory fragment that specifically binds to the binding domain of the target protein is useful. For example, based upon the variable-region sequences of an antibody, peptide molecules can be designed that retain the ability to bind the target protein sequence. Such peptides can be synthesized chemically and/or produced by recombinant DNA technology. (See, e.g., Marasco et al, Proc. Natl. Acad. Sci. USA, 90: 7889-7893 (1993)). The formulation can also contain more than one active compound as necessary for the given indication being treated, for example, those with complementary activities that do not adversely affect each other. Alternatively, or in addition, the composition can comprise an agent that enhances its function, such as, for example, a cytotoxic agent, cytokine (e.g., IL-15), chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid allow release of molecules for over 100 days, certain hydrogels release proteins for shorter time periods.

The antibodies or agents of the invention (also referred to herein as “active compounds”), and derivatives, fragments, analogs and homologs thereof, can be incorporated into pharmaceutical compositions suitable for administration. Such pharmaceutical compositions can comprise the antibody or agent and a pharmaceutically acceptable carrier. As used herein, the term “pharmaceutically acceptable carrier” can include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Non-limiting examples of such carriers or diluents include water, saline, ringer's solutions, dextrose solution, and 5% human serum albumin. Liposomes and non-aqueous vehicles such as fixed oils can also be used. The use of such media and agents for pharmaceutically active substances is well known in the art. Except insofar as any conventional media or agent is incompatible with the active compound, use thereof in the compositions is considered. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In embodiments, the composition is sterile and is fluid to the extent that easy syringeability exists. It can be stable under the conditions of manufacture and storage and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents can be included, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. For example, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional ingredient from a previously sterile-filtered solution thereof.

Oral compositions can include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations are apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the given therapeutic effect to be achieved, and the limitations inherent in the art of compounding such an active compound for the treatment of individuals.

The pharmaceutical compositions can be included in a container, pack, or dispenser together with instructions for administration.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention will be further described in the following examples, which do not limit the scope of the invention described in the claims.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### Example 1—Definition of a Mouse Microglial Subset that Regulates Neuronal Development and Pro-Inflammatory Responses in the Brain

### Abstract

Expression of Itgax (encoding the CD11c surface protein) and Spp1 (encoding OPN) have been associated with activated microglia that can develop in healthy brains and some neuroinflammatory disorders. However, whether CD11c and OPN gene expression are a consequence of microglial activation or represents a portion of the genetic program expressed by a stable microglial subset is unknown. Here, we show that OPN production in the brain is confined to a small CD11c⁺ microglial subset that differentiates from CD11c⁻ precursors in perinatal life after uptake of apoptotic neurons. Our analysis indicates that co-expression of OPN and CD11c genes marks a microglial subset that is expressed at birth and persists into late adult life, independent of environmental activation stimuli. Analysis of the contribution of OPN to the intrinsic functions of this CD11c⁺ microglial subset indicates that OPN is required for subset stability and the execution of phagocytic and proinflammatory responses, in part through OPN-dependent engagement of the αVβ3 integrin receptor. Definition of OPN-producing CD11c⁺ microglia as a functional microglial subset provides new insight into microglial differentiation in health and disease.

### Significance Statement

CD11c⁺ microglia enriched for osteopontin gene expression appear at distinct stages of brain development, aging and several neurodegenerative disorders. Whether co-expression of CD11c and OPN results from microglial activation or represent a part of a subset-specific genetic program is unknown. We find that this CD11c⁺ microglial subset is formed before birth upon uptake of apoptotic neurons. Our analysis also indicates that it represents a stable subset that requires OPN to mediate engulfment of synaptic proteins, proliferate and develop a pro-inflammatory phenotype. Definition of OPN-producing CD11c⁺microglia as a specialized microglial subset provides new insight into the contribution of microglial differentiation to brain development and function in health and disease.

### Introduction

CD11c, also termed integrin alpha X (encoded by Itgax) is a defining marker for dendritic cells (DC). When paired with Itgb2, the heterodimeric receptor binds to complement iC3b and mediates phagocytosis (1). A subpopulation of CNS-resident microglia that also expresses CD11c develops early in life and is a feature of microglial development in healthy brains and in murine models of neurodegenerative disease, including Alzheimer's disease (AD) (2-4). Genes expressed by CD11c⁺ microglia include the Spp1 gene, which encodes osteopontin (OPN), a cytokine-like phosphoprotein that is a prominent feature of both protective and pathogenic immune responses in peripheral lymphoid tissues (4-8).

OPN is expressed as a secreted (OPN-s) or intracellular (OPN-i) isoform that originated from a single OPN mRNA precursor (9) after activation of immune cells. Regulation of immune responses by OPN includes promotion of pro-inflammatory responses following ligation of its canonical receptor, the αVβ3 integrin expressed on macrophages (10-12). Production of OPN by DCs also regulates the differentiation of T helper (TH)-cell subsets (8), including TH17 cells, which contribute to the development of murine experimental autoimmune encephalomyelitis (EAE) (13). More recently, microglial production of OPN has been implicated in diverse CNS pathologic disorders, including multiple sclerosis (3), spinal cord injury (14) and neurodegenerative disorders, including AD and Amyotrophic Lateral Sclerosis (ALS) (4, 15). Although CD11c⁺ microglia are a major source of OPN production by activated microglia, whether co-expression of CD11c and OPN is a feature of microglial activation or marks a subset-specific genetic program is not known. This represents a central question in understanding microglial development, since it involves a choice between genetic mechanisms that regulate subset-specific differentiation rather than markers of a transient activation phenotype.

Here, we report that CD11c⁺ OPN-producing microglia represent a small (<5%) subset that differentiates from CD11c OPN⁻ precursors after engulfment of apoptotic neurons (ANs) in perinatal life and represent the sole microglial producers of OPN throughout life. The CD11c⁺ OPN⁺ subset displays a stable phenotype in the steady state and express a core genetic program that is independent of microglial activation. Analysis of the contribution of OPN to CD11c⁺ microglial function indicates that OPN regulates microglial proliferation and the development of a pro-inflammatory phenotype. Definition of OPN-producing CD11c⁺ microglia as a functional subset rather than a transient activation phenotype indicates new approaches to analysis of microglial development and provides insight into the contribution of microglia to normal and dysregulated brain development.

### Results

### CD11c⁺ Microglia are Formed Early in Mouse Brain Development Upon Engulfment of Apoptotic Neurons Independent of Microglial Activation

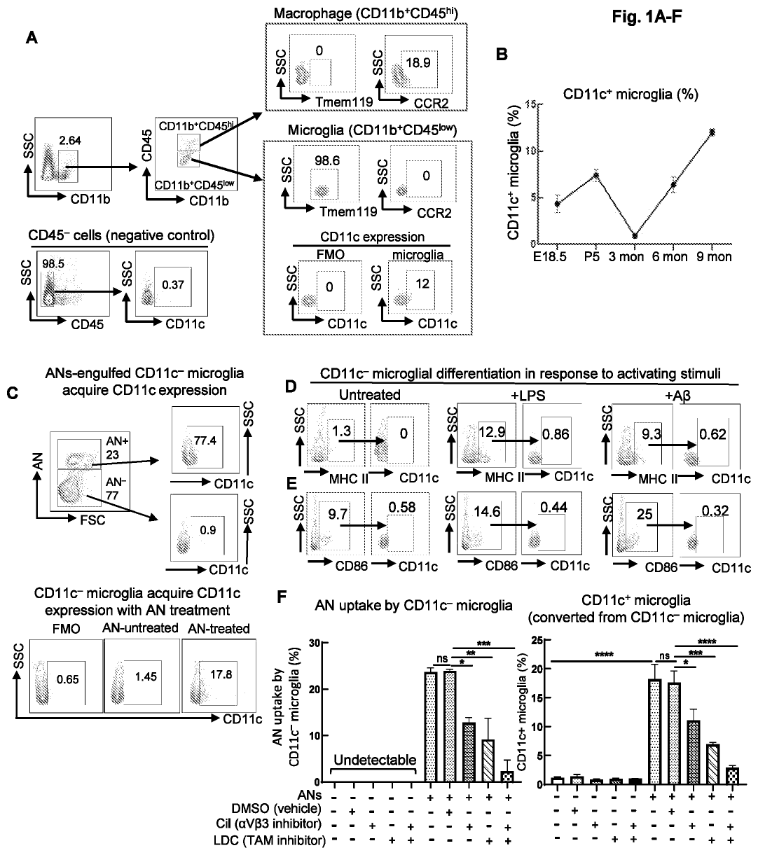

In dendritic cells, the CD11c protein represents an essential part of the iC3b heterodimeric receptor that mediates phagocytosis (1). Microglia that express CD11c are detectable at birth, but decrease dramatically by 3 months of age (3). We therefore traced the development of CD11c⁺ microglia in healthy C57BL/6 mice from prenatal life through adulthood. We first validated microglial CD11c expression by flow cytometry. To distinguish microglia (CD11b⁺CD45^{low}) and macrophages (CD11b⁺CD45^{high}), we used the CCR2 marker expressed by blood-derived macrophages, but not expressed by microglia (3, 16), and a microglia-specific marker Tmem119 (17). The CD11b⁺CD45^{high} subset that expresses CCR2 but not Tmem 119 were confirmed as macrophages, while CD11b⁺CD45^{low} cells that all express Tmem119, but do not express CCR2 were confirmed as microglia. Fluorescence Minus One (FMO) negative control and brain CD45⁻ cells that do not express CD11c were included to confirm the specificity of CD11c staining in CD11b⁺CD45^{low} microglial populations (FIG. 1A). We note that CD11c⁺ microglia arise late in embryogenesis (E18.5) increasing to about 7-8% of total microglia by postnatal day 5 (P5) before receding to almost undetectable levels (<1%) in young adult life. However, CD11c⁺ microglia re-emerge in older (6-9 mos) mice to represent about 10% of total microglia (FIG. 1B).

Since uptake of apoptotic neurons (ANs) may trigger changes in the microglial genetic program that include upregulation of Itgax RNA (encoding CD11c) (18), we asked whether upregulation of CD11c expression by CD11c⁻ precursors was a direct consequence of AN engulfment early in development. We enriched CD11c⁻ microglia by negative isolation with anti-CD11c coated magnetic beads to yield CD11c⁻ microglia (purity >99%) and incubated them with fluorescent-labeled ANs for 72 h followed by a determination of the percentage of CD11c⁻ microglia that expressed CD11c. In FIG. 1C, we observe that the 23% of total CD11c⁻ microglia had ingested ANs (AN⁺), of which 77.4% of these AN⁺ microglia were CD11c⁺. In contrast, of the 77% of CD11c⁻ microglia that had not ingested ANs, <1% expressed CD11c (upper panel, FIG. 1C). These findings also indicate that ˜18% of total CD11c⁻ microglia go on to express CD11c⁺ after incubation with ANs, while <1.5% of the microglia incubated×72 h in the absence of ANs become CD11c⁺ (lower panel, FIG. 1C).

Upregulation of CD11c expression was not simply a consequence of microglial activation after, for example, phagocytosis or inflammation. Purified CD11c⁻ microglia from P5 mice were treated with diverse activating stimuli that mimic CNS inflammatory disorders, including LPS and the amyloid β peptide (Aβ). Although these stimuli provoked marked microglial activation, as judged by increased expression of both CD86 and MHC II (19), activated CD11c⁻ microglia did not upregulate CD11c expression (FIGS. 1D, E). Collectively, these results indicate that the formation of CD11c⁺ microglia following ingestion of ANs at birth is independent of conventional microglial activation.

We then examined the interactions that might promote the formation of CD11c⁺ microglia in neonatal life. Phagocytic receptors expressed by developing microglia that may mediate uptake and clearance of apoptotic cells include TAM (Tyro3, Axl, MerTK) and the integrin αVβ3 receptors (20, 21). We analyze the impact of specific inhibition of αVβ3 and TAM receptors on AN uptake and its impact on acquisition of a CD11c⁺ phenotype by CD11c⁻ microglia. The αVβ3 inhibitor cilengitide (Cil) or the LDC1267 (LDC) inhibitor of pan-TAM receptors reduced AN uptake CD11c⁻ microglia by ˜50%. A combination of these two inhibitors further reduced CD11c⁻ microglial AN uptake to background levels (FIG. 1F, left panel,). Consistent with findings in FIG. 1C, we noted that stimulation by ANs induced CD11c expression by ˜20% of initial CD11c⁻ microglia over 72 h, while CD11c expression was not detectable in the absence of AN stimulation during the same time period. Finally, inhibition of AN uptake by cilengitide or LDC1267 reduced acquisition of a CD11c⁺ phenotype by CD11c⁻ progenitors by about 50-75% in each case and inclusion of both inhibitors fully prevented this phenotypic transition (FIG. 1F, right panel). These findings indicate that engulfment of apoptotic cells may trigger a CD11c⁻ →CD11c⁺ transition following interaction of ANs with microglial receptors that include αVβ3 and TAM.

### The Stability of CD11c Expression by Microglia is Regulated by OPN

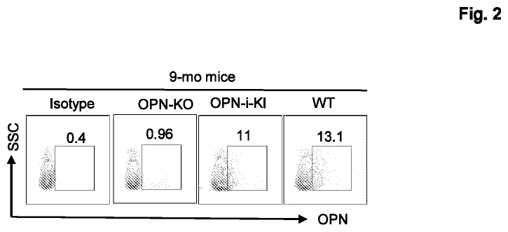

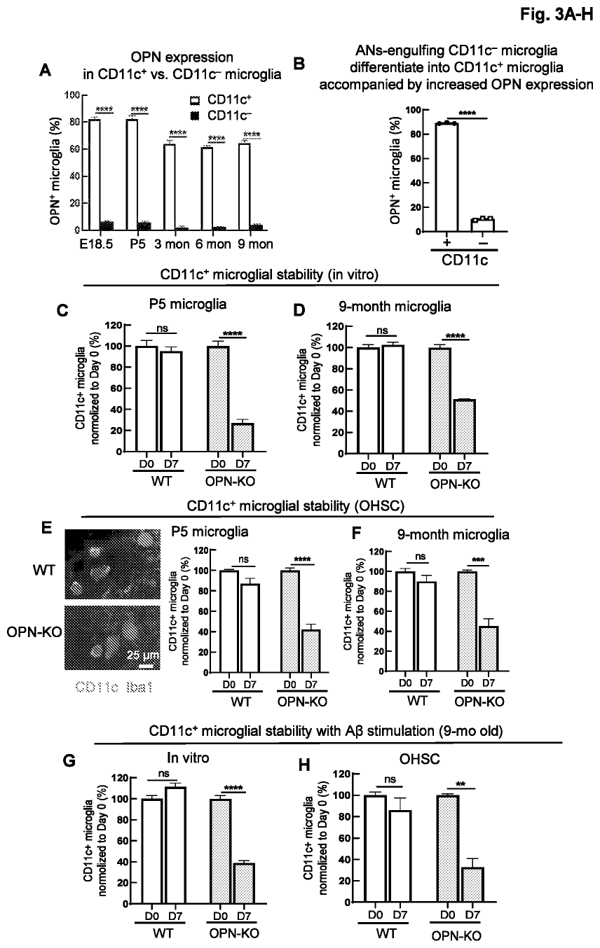

We validated microglial OPN expression by flow cytometry analysis. We used several controls, including an isotype control and, importantly, OPN-KO microglia as negative control cells and microglia that selectively express the intracellular isoform of OPN (OPN-i-KI) as a positive control. WT microglia and OPN-i-KI microglia show similar levels of OPN staining, while staining of microglia from either OPN-KO donors or microglia stained with an isotype control does not yield a detectable signal (FIG. 2). We noted that microglial OPN production is confined to CD11c⁺ microglia and is not significantly expressed by CD11c⁻ microglia (FIG. 3A). Moreover, AN-induced differentiation of CD11c⁻ to CD11c⁺ microglia was also accompanied by a sharp upregulation of OPN production (FIG. 3B).

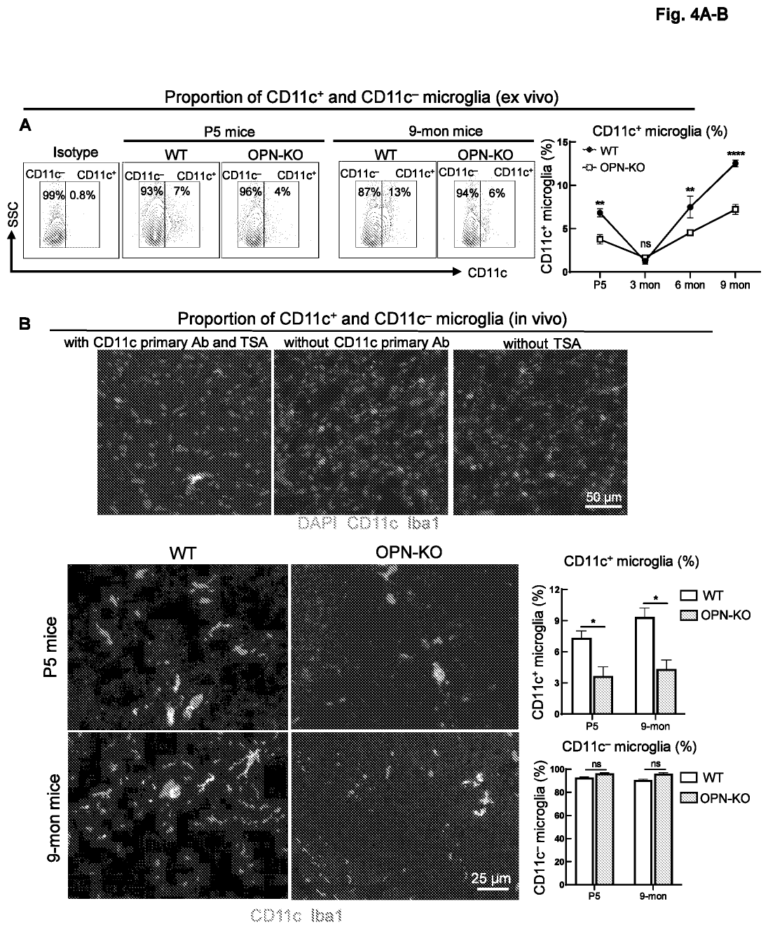

To define the impact of OPN on the CD11c phenotype, we characterized CD11c⁺ microglia from OPN-KO and WT mice during early development and aging. OPN deficiency led to an approximate 50% reduction in the proportions of CD11c⁺ microglia in WT mice at P5, 6- and 9-mos of age, according to flow cytometry analysis (FIG. 4A). In situ analysis of CD11c⁺ and CD11c⁻ microglia in brain cryosections of P5 and 9-mo old WT and OPN-KO mice confirmed reduced properties of CD11c+ microglia from OPN-KO mice (50-75% reduction). CD11c-specific staining was validated using negative controls (FIG. 4B).

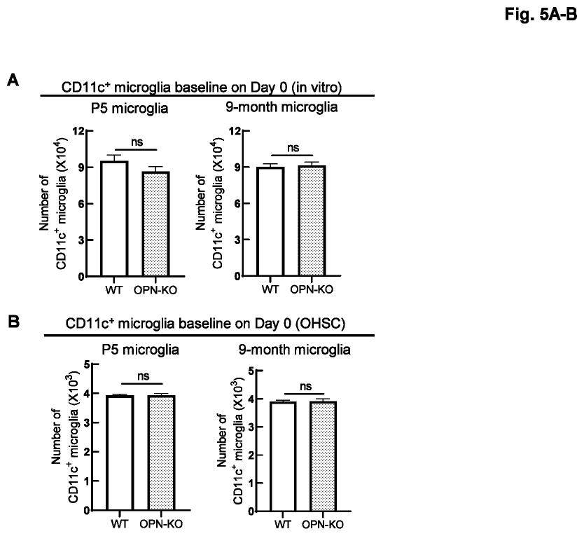

We further defined the contribution of OPN to the stability of CD11c expression by microglia isolated from P5 and 9-mo old WT and OPN-KO mice during a 7 d in vitro culture period. The numbers of CD11c⁺ microglia were comparable between P5, 9-mo old WT and OPN-KO mice at day 0 (FIG. 5A). OPN-deficient neonatal (P5) CD11c⁺ microglia showed a 75% reduction in CD11c expression (FIG. 3C), while OPN-deficient CD11c⁺ microglia from 9-mo old WT mice showed an ˜50% reduction in CD11c expression (FIG. 3D). We examined the stability of the microglial CD11c⁺ phenotype in a more physiological setting using organoid hippocampal slice cultures (OHSC). This system allows replacement of endogenous microglia after depletion of resident microglia from organotypic hippocampal tissue slices. We replenished microglia with CD11c⁺ microglia (>95% CD11c⁺) from WT or OPN-KO P5 and 9-mo old donors. WT and OPN-KO mice showed identical numbers of CD11c⁺ microglia at day 0 in OHSC (FIG. 5B). The WT CD11c⁺ microglia retained their CD11c⁺ phenotype, while the majority of OPN-deficient CD11c⁺ microglia did not (FIGS. 3E, F).

We then assessed the response of CD11c⁺ microglia to Aβ, a pathogenic microglial stimulus that is a prominent feature of age-related neurodegenerative disease. We observed that CD11c⁺ microglia from 9-mo old WT mice stably expressed CD11c after in vitro Aβ stimulation and in OHSC in contrast to OPN-deficient CD11c⁺ microglia, which lost 60-75% of CD11c expression (FIGS. 3G, H). Taken together, these data indicate that stable expression of microglial CD11c may require co-expression of OPN.

### Genetic Profiling of CD11c⁺ Microglia

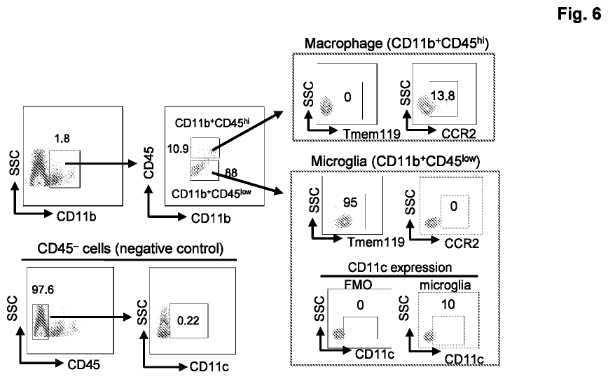

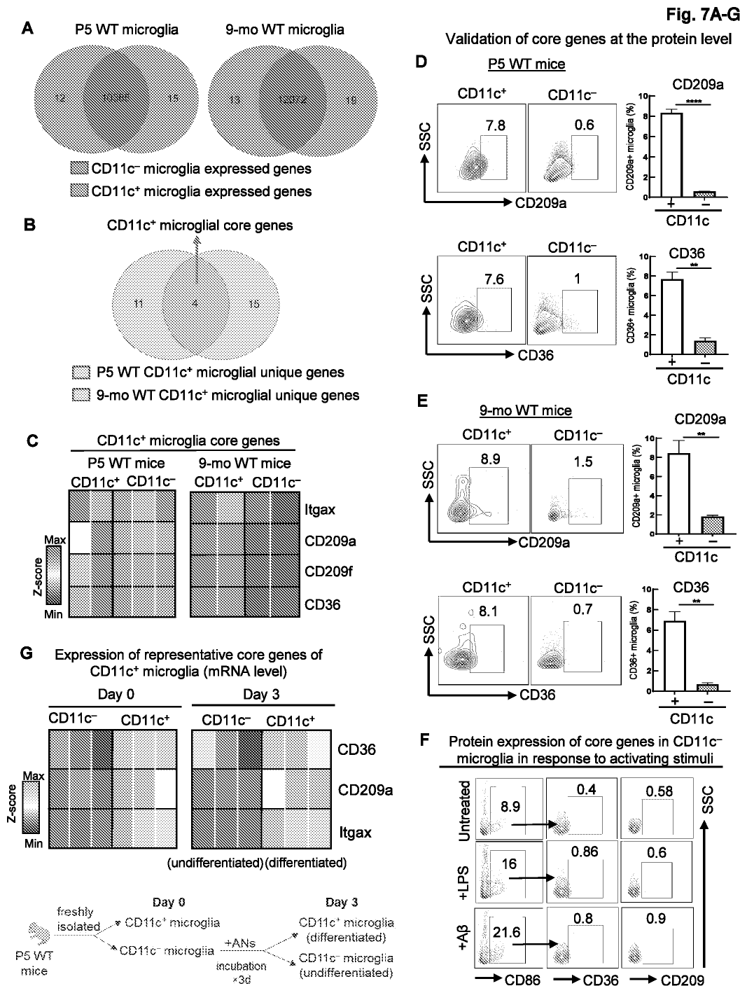

We then asked whether CD11c⁺ microglia represent a distinct subset that expressed a characteristic genetic profile. To that end, we performed RNA-seq analysis of FACS-sorted CD11c⁺ and CD11c⁻ microglia using a cold isolation protocol followed by validation using flow cytometry (FIG. 6). For example, the transcriptomes of CD11c⁺ and CD11c⁻ microglia from neonatal (P5) WT mice overlapped substantially sharing 10,385 genes. However, a small number of genes (12-15) were uniquely expressed by CD11c (12 genes) and about 15 genes were uniquely expressed by CD11c⁺ microglia. Likewise, CD11c⁺ and CD11c⁻ microglia from 9-mo old mice shared 12,072 genes, while 19 genes were solely expressed by CD11c⁺ microglia (FIG. 7A). We identified four genes that were shared by CD11c⁺ microglia by neonatal (P5) and aged (9-mo old) mice (FIG. 7B) and were not expressed by CD11c⁻ microglia from donors of either age (FIG. 7C). These represent genes selectively expressed by CD11c⁺ microglia that are retained from neonatal life to older adulthood in the absence of exogenous inflammatory or infectious stimuli. These genes include CD36, an inflammatory response-modulating molecule (22), and CD209a, a regulator of phagocytic activity (5, 23) and are expressed at both the RNA and protein levels (FIGS. 7D, E). Expression of these proteins by CD11c⁺ microglia was independent of activation stimuli, since deliberate activation of CD11c⁻ microglia did not induce expression of these core proteins (CD209 and CD36) (FIG. 7F). Moreover, these signature genes were upregulated by CD11c⁺ progeny of CD11c precursors after engulfment of ANs, indicating that upregulation of these genes accompanies the formation of this subset in perinatal life (FIG. 7G).

### OPN Regulates Intrinsic Functions of CD11c⁺ Microglia

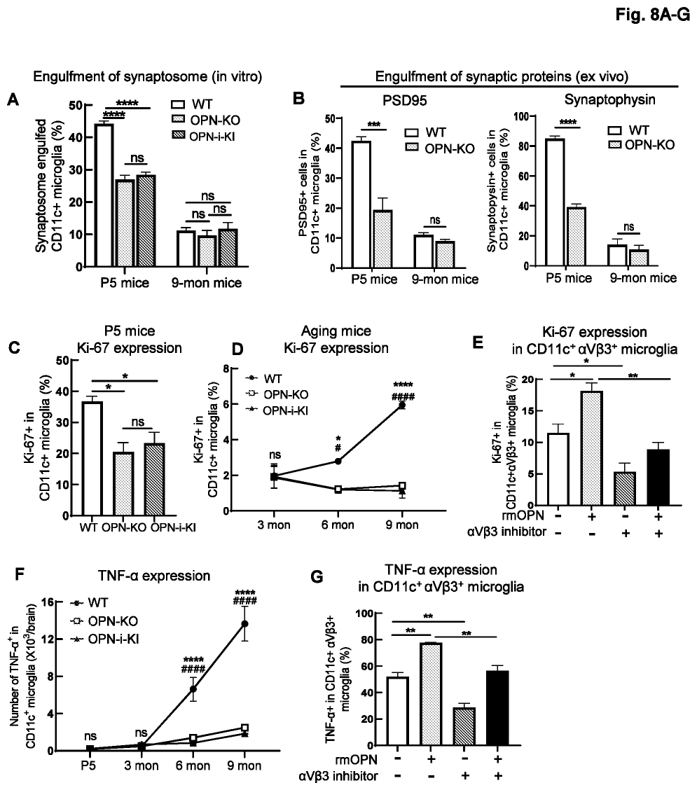

Microglia may contribute to the elimination of excessive neuronal synapses during neonatal brain development (24). We found that CD11c⁺ OPN⁺ microglia from neonatal, but not from 9-mo old brains, were highly phagocytic for synaptosomes (FIG. 8). We asked whether OPN contributed to this microglial function. OPN is expressed as a secreted (OPN-s) or intracellular (OPN-i) isoform that derives from different OPN translational initiation sites (25). We utilized OPN-mutant mice harboring different OPN isoforms to delineate the contribution of OPN isoforms to CD11c⁺ microglial functions. Full OPN deficiency resulted in a ˜45% decrease of synaptosome engulfment by CD11c⁺ microglia. OPN-i was not implicated in this process (FIG. 8A). Similarly, ex vivo analysis revealed that CD11c⁺ OPN⁺ microglia from P5 but not 9-mo old WT mice displayed robust engulfment of synaptic proteins, including synaptophysin and PSD95. Deficiency of both OPN-s and OPN-i and OPN-s alone resulted in a ˜50% reduction of this CD11c⁺ microglial function (FIG. 8B).

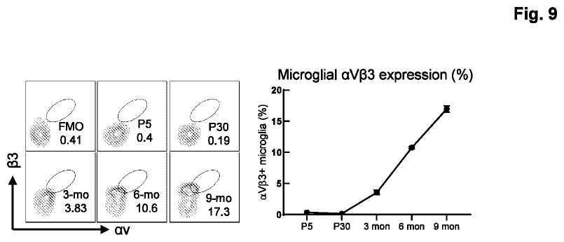

Examination of the contribution of OPN to proliferation of CD11c⁺ microglia revealed that selective deletion of OPN-s was sufficient to substantially reduce proliferation of CD11c⁺ microglia from both newborn and aging WT mice (FIGS. 8C, 8D). We then asked whether an interaction between OPN-s and its canonical receptors might contribute to this CD11c⁺ microglial function. Although the CD44 and αVβ3 OPN receptors (26, 27) are not expressed by microglia at any age, αVβ3 expression gradually increases during aging (FIG. 9). Small molecule-mediated blockade of αVβ3 by the cyclic RGD inhibitor (cilengitide) markedly reduced OPN-dependent Ki-67 expression (FIG. 8E), indicating that OPN-dependent proliferation of CD11c⁺ microglia reflects ligation of the αVβ3 receptor. Moreover, the robust pro-inflammatory response of CD11c⁺ microglia from 9-mo old mice depended on OPN-s expression (FIG. 8F) and reflected engagement of the αVβ3 integrin receptor (FIG. 8G).

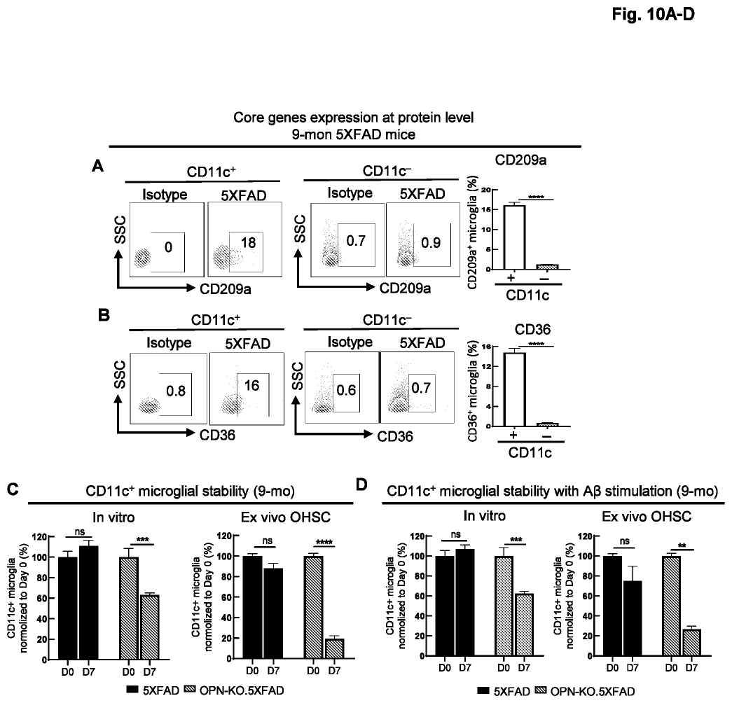

The age-dependent pro-inflammatory phenotype of this OPN-producing CD11c⁺ microglial subset indicates it might contribute to neuroinflammatory disorders. We therefore asked whether the expression of the signature genes of CD11c⁺ OPN⁻ producing microglia might persist in the context of chronic inflammatory stimuli that develop in 5XFAD mice, which recapitulate amyloid pathology of Alzheimer's disease. We observed that CD11c⁺ microglia from OPN-sufficient mice expressed representative core genes CD36 and CD209a at the protein level compared to their CD11c⁻ counterparts in the presence of 5XFAD pathology (FIG. 10A, B). Moreover, OPN production contributed to CD11c⁺ microglial stability, in contrast to strongly reduced stability of CD11c⁺ microglia from age-matched OPN-KO.5XFAD mice (˜40%-80% reduction) in vitro and ex vivo (FIG. 10C). The CD11c phenotype was also stably expressed by CD11c⁺ microglia from 9-mo old 5XFAD mice in the presence of AD, while OPN-deficiency led to 40%-80% reduction in vitro and in OHSC, respectively (FIG. 10D).

### Discussion

Microglial expression of the CD11c receptor and production of OPN have been associated with microglial activation during the development of some neuroinflammatory diseases (3, 4, 7) and in response to exogenous stimuli (14). This may be a consequence of microglial activation or, alternatively, a part of the genetic program of a microglial subset that develops at or before birth and persists into late adulthood. Our studies support the latter view, i.e., CD11c⁺ microglia represent a stable subset programmed to produce OPN rather than a transient activation phenotype.

We identify a small (<5%) OPN-producing CD11c⁺ microglial subset that differentiates from CD11c⁻ at birth upon engulfment of ANs in the absence of external activation stimuli. After their initial formation in perinatal life, CD11c⁺ microglia recede to almost undetectable levels in young adult life before re-emergence in late adult life to constitute 10-15% of total microglia. Both the perinatal CD11c+ microglia and late-adult life CD11c⁺ microglia express a genetic signature that is independent of cellular activation in healthy mice.

Single-cell transcriptomics have described microglial subsets enriched for Itgax (encoding CD11c) at different ages and during development of neurodegenerative disease (4-7), following their description by the Owens group (3), who observed that CD11c⁺ microglial numbers peaked early after birth (P3-5) and were reduced to marginal levels by young adulthood (2-3 months). Our analysis of CD11c⁺ microglia before birth and during normal aging revealed that CD11c⁺ microglia that appear during late embryogenesis (day E18.5) transiently contract before re-emergence and expansion into substantial numbers during normal aging.

Although alterations in the microglial gene transcription program after engulfment of apoptotic cells include upregulation of Itgax gene expression (18, 28), studies of unselected microglial populations have not determined whether this reflects de novo expression of CD11c by CD11c⁻ precursors or expansion of CD11c⁺ microglia from a smaller population. Analysis of the response of isolated CD11c⁻ precursors to ANs revealed that induction of CD11c protein expression following AN engulfment by neonatal CD11c⁻ precursors was accompanied by OPN expression, indicating that this phenotype is a direct consequence of AN-induced differentiation. This view is supported by findings that formation of CD11c⁺ microglia is independent of non-specific microglial activation since deliberate activation of CD11c⁻ precursors by various non-specific stimuli failed to induce expression of CD11c phenotype. It may be relevant that microglia within neonatal brain regions containing high amounts of apoptotic cells express high levels of Itgax/CD11c and phagocytosis-related genes (3, 6, 29, 30). Moreover, injection of apoptotic neurons, but not live neurons, Escherichia coli, or zymosan particles into B6 mouse brain induces a microglial phenotype that includes upregulation of Itgax (CD11c) (18), indicating that microglial induction of CD11c is a tightly controlled response to cellular apoptosis. Without wishing to be bound by theory, conversion of adult microglia later in life can be efficiently promoted by other stimuli including amyloid beta either alone or as a synergistic mixture of apoptotic neurons and amyloid beta.

Expression of the CD11c⁺ phenotype depended on OPN production, as judged from in vitro analyses and after transfer into microglia-free hippocampal tissues. Moreover, analysis of the CD11c⁺ microglial gene profile at birth and late-adult life indicated a persistent genetic signature that was independent of conventional activation stimuli. Although selective expression of these signature genes by CD11c⁺ microglia was not mimicked by deliberate activation of CD11c⁻ microglia, further transcriptomic profiling and single-cell transcriptomic analysis of CD11c⁺ and CD11c⁻ microglia is required for a more detailed genetic description of this subset in healthy brain development and in the face of chronic inflammatory disorders. Expression of a stable phenotype and core genetic program that persists from birth to late adulthood independent of exogenous stimulation indicates that CD11c⁺ microglia can represent a microglial lineage. More direct probing using fate-mapping techniques to trace the genetic history of CD11c⁺ microglia in different reporter mouse models are needed to directly address this question (31, 32).

The genetic program of CD11c⁺ microglia includes genes associated with phagocytosis and inflammation (22, 23), indicating that CD11c⁺ microglia are specialized to execute these microglial functions. Indeed, we note that CD11c⁺ microglia can contribute to neuronal synapse elimination by engulfing synaptic proteins in early development and mediate pro-inflammatory responses during aging, perhaps allowing for elimination of defective or inactive synapses. These functions of the CD11c⁺ microglial subset are regulated by OPN, since engulfment of synaptic proteins by neonatal CD11c⁺ microglia is depressed in the absence of OPN, while the proliferative and pro-inflammatory responses of CD11c⁺ microglia in adult life reflect OPN engagement of αVβ3 integrin receptors. Analysis of OPN mutant mice that specifically express OPN isoforms demonstrated that OPN-s, but not OPN-i, is responsible for these OPN-dependent functions. Promotion of inflammatory responses by CD11c⁺ microglia is reminiscent of the subset of dendritic cells which express high levels of CD11c and carry out OPN-dependent inflammatory responses (33). Our observations that CD11c⁺ microglia from 5XFAD mice express the signature genes at protein level and OPN-dependent phenotype noted in healthy mice can allow definition of their contribution to disease development.

Taken together, we show that CD11c⁺ microglia, the sole producer of OPN in brain, differentiate from CD11c⁻ microglia after uptake of apoptotic neurons at birth. Without being bound by theory, this OPN-producing CD11c⁺ microglial population represents a new subset according to its stable phenotype and expression of a signature gene set at birth and late adult life that is independent of deliberate activation. The pro-inflammatory properties of CD11c⁺ microglia indicate that these cells can contribute to the development of neuroinflammatory diseases including AD, Amyotrophic Lateral Sclerosis, and Parkinson's disease.

### Materials and Methods

Mice. Wild type C57BL/6 (B6) and B6. Cg-Tg (APPSwFlLon,PSEN1*M146L*L286V)6799Vas/Mmjax (5XFAD) mice were obtained from the Jackson Laboratory. Spp1^{flstop} (OPN-KO) and Spp1^{flstop} Ella^{cre} (OPN-i-KI) mice were generated by our lab as previously described (34). OPN-KO.5XFAD were bred by crossing Spp1^{flstop} mice with 5XFAD mice. All the mice were housed in pathogen-free conditions. All experiments were performed in compliance with federal laws and institutional guidelines as approved by the Animal Care and Use Committee of the Dana-Farber Cancer Institute (DFCI).

### Microglial Isolation

Isolation of microglia by MACS. Adult mice were anesthetized with isoflurane and transcardially perfused with ice-cold PBS before brains were removed and minced by scalpels. Tissues were subject to enzymatic dissociation using Collagenase (300 U/ml, Worthington) or Papain (20 U/ml, Worthington) and DNase I (60 U/ml, Worthington) before 30% Percoll gradient was performed to remove myelin, and pelleted cells resuspended in MACS buffer (PBS pH 7.2, 2 mM EDTA and 0.5% BSA). Total microglia were obtained by magnetic isolation using CD11b microbeads (Miltenyi). For isolation of CD11c⁺ and CD11c⁻ microglia, single cell suspensions were incubated with CD11c microbeads (Miltenyi) and cells magnetically bound to columns using MACS were extensively washed before the CD11c⁺ fraction was eluted after lifting the magnetic field. The unbound fraction was then labeled with CD11b microbeads (Miltenyi) and separated using MACS isolation and CD11c⁻ CD11b⁺ cells that were bound to the column were eluted. MACS buffer was used according to manufacturer's protocol. This standard method was used to isolate microglia for all of the experiments except otherwise noted.

Microglia isolation and fluorescence-activated cell sorting for RNA-seq analysis. Single cell suspensions were prepared as described (6). Briefly, mice were anesthetized with isoflurane and transcardially perfused before brains were quickly dissected and minced using a scalpel on ice followed by Dounce homogenization in ice cold HBSS ˜20 times. All tools used were prechilled and all isolation steps were carried out on ice to minimize microglial activation. Cell suspensions were transferred to prechilled 50 ml tubes and passed through a 70 μm cell strainer followed by transfer into a prechilled 15 ml tube and spun down at 500 g×5 min at 4° C. Debris and myelin were then removed using a modified cold Percoll gradient and cell pellets resuspended in 10 ml of ice cold 40% Percoll (Sigma) diluted and then spun ×30 min at 500 g. This approach yielded a microglial pellet at the bottom of the 15 ml tube while Percoll and myelin were removed by vacuum suction. The cell pellet was washed with 10 ml of ice cold HBSS and spun again ×5 min at 500 g at 4° C. All samples were then resuspended in ice cold FACS buffer (0.5% BSA, 1 mM EDTA, in 1× PBS) for staining. Ghost dye Violet 510 (1:1000, Tonbo Biosciences) was used to exclude dead cells. Fc receptors were blocked using CD16/CD32 antibody (1:100, BD Biosciences) to avoid non-specific staining. Single cell suspensions were then stained with anti-CD11b (1:100), anti-CD45 (1:100) and anti-CD11c (1:100) antibodies (Biolegend)×20 min on ice before samples were washed with ice cold FACS buffer and spun down ×5 m at 500 g. Cell pellets were resuspended in 5 ml of ice cold FACS buffer before sorting on a BD FACS Aria II using the 70 μm nozzle with purity mode at ˜10,000 events per second. After sorting, each sample was spun down and cell pellets were immediately stored at −80° C. until further processing.

For P5 mice, 3 biological replicates of CD11c⁺ microglia and CD11c⁻ microglia were sequenced. Samples were pooled from 58 mice. For 9-mo old WT mice, 2 replicates of CD11c⁺ microglia and CD11c⁻ microglia were sequenced. Samples were pooled from 44 mice (22M+22F).

Apoptotic neurons (ANs): induction and labeling for CD11c⁻ microglial differentiation. Primary mouse neurons were prepared from B6 embryos at embryonic day 16.5-17.5. Cerebral hemispheres were isolated and freed from meninges before tissue digestion with 0.25% trypsin in HBSS×15 min at 37° C. followed by titration to obtain single cell preparations. Cell suspensions were filtered through a 70 μm cell strainer and cells centrifuged at 600 g×5 min. Cell density was determined using a hemocytometer, and cells seeded in Neurobasal medium supplemented with 1×B27 and 500 μM GlutaMax (Invitrogen). Half medium was changed every 3 days. To induce apoptosis, primary cultured WT neurons were treated with 300 μM NMDA overnight, before careful detachment from flasks by repeated washes with PBS, followed by centrifugation and the pellet processed for labeling. Neurons (1×10⁷) were carefully resuspended in 1 ml PBS and incubated in darkness ×2 h at room temperature with 100 μg of dissolved labeling dye (pHrodo iFL green, Life Technologies). To block and capture residual dye, cells were diluted with PBS, harvested by centrifugation, resuspended in 1 ml FBS and washed twice with PBS. Total apoptotic cell numbers were determined using Trypan Blue staining.

### Flow Cytometry

Microglial staining for flow cytometry analysis. Microglia were stained with Ghost dye Violet 510 (1:1000, Tonbo Biosciences) to exclude dead cells followed by Fc receptor blocking using CD16/CD32 antibody (1:100, BD Biosciences) to avoid non-specific staining. Appropriate microglial surface markers were used for staining, including anti-CD11b (1:100 Biolegend), anti-CD45 (1:100 Biolegend), anti-CD11c (1:50 Biolegend), anti-CD36 (1:100, Biolegend), anti-CD209a (1:100, Biolegend), anti-αV integrin (1:50, Biolegend), anti-β3 integrin (1:50, Biolegend), anti-CD86 (1:100, Biolegend), and anti-MHC II (1:100, Biolegend), followed by fixation and permeabilization for subsequent intracellular staining with anti-OPN (1:10, R&D Systems), anti-TNF-α (1:50, Biolegend) and intranuclear staining with anti-Ki-67 (1:100, Biolegend).

Validation of microglial OPN expression by flow cytometry analysis. Microglial OPN expression was validated in 9-mo old WT mice using conventional intracellular staining protocols. Microglia were fixed and permeabilized with Intracellular Fixation & Permeabilization Buffer (eBioscience) followed by incubation with PE-conjugated anti-OPN Ab (1:10, Cat. NO. IC808, R&D Systems) at 4° C. for 30 min. An isotype control (1:10, PE-conjugated goat IgG) and OPN-KO microglia were used as negative control. Microglia that selectively express the intracellular isoform of OPN (OPN-i-KI) were used as a positive control.

Validation of microglial CD11c expression by flow cytometry analysis. Brain single cell suspensions from 9-mo old WT mice were stained with Ghost dye Violet 510 (1:1000, Tonbo Biosciences) and anti-CD16/CD32 antibody (1:100, BD Biosciences) followed by anti-CD11b (1:100, Biolegend), anti-CD45 (1:100, Biolegend), anti-CD11c (1:50, Biolegend), anti-TMEME119 (1:200, ABCAM), anti-CCR2 (1:100, Biolegend). CD11b⁺ cells were gated from single/live cells followed by subsequent gating of CD11b⁺CD45^{low} as microglia and CD11b⁺CD45^{hi} as macrophages. To distinguish microglia and macrophages, the CCR2 marker expressed by blood-derived macrophage but not by microglia was used (3, 16). To further distinguish these two cell types, the microglial-specific marker Tmem119 (17) was also included. The CD11b⁺CD45^{high} cells that express CCR2 but not Tmem 119 were confirmed as macrophages, while the CD11b⁺CD45^{low} cells that all express Tmem119, but do not express CCR2 were confirmed as microglia. FMO negative controls were included to confirm the specificity of CD11c staining in CD11b⁺CD45^{low} microglial populations. Brain CD45⁻ cells that mainly contain non-immune cells (e.g., neurons, astrocytes, oligodendrocytes) that do not express CD11c were also included as negative controls to further validate the specificity of this strategy.

Flow cytometry detection of microglial engulfment of synaptic protein. After perfusion, mouse brains were harvested and dissected followed by myelin removal by centrifugation on 30% Percoll gradient. Brain pellets were sequentially stained with Ghost dye Violet 510 (1:1000, Tonbo Biosciences) followed by incubation with microglial surface markers: anti-CD11b, anti-CD45 and anti-CD11c for 30 min. Subsequently, stained samples were fixed and permeabilized using Intracellular Fixation & Permeabilization Buffer (eBioscience). Intracellular staining was performed for pre-synaptic marker anti-Synaptophysin (Invitrogen, 1:100) or post-synaptic marker PSD-95 (Invitrogen, 1:100) followed by staining with Alexa Fluor 488-donkey anti-mouse IgG (H⁺L) secondary antibody (Invitrogen, 1:300). Samples were acquired on CytoFLEX (Beckman Coulter) flow cytometer followed by analysis with FlowJo v10 (Tree star).

Organotypic hippocampal slice cultures. Organotypic hippocampal slice cultures (OHSC) were prepared as described (35). Briefly, hippocampal slices were prepared from newborn (P3-P5) C57BL/6 mice to a thickness of 350 μm before incubation at 35° C. in 5% C02. Microglia were depleted from freshly prepared slice cultures using clodronate liposomes (FormuMax) and freshly prepared OHSC were incubated with 0.5 mg/ml clodronate liposomes ×24 h at 35° C. Subsequently, OHSC were rinsed with warm PBS before replacement of medium (50% MEM, 25% HBSS, 25% normal horse serum, 0.2 mM glutamine, 100 U/ml penicillin, 100 mg/ml streptomycin and 4.5 mg/ml glucose). Microglia-depleted OHSC were maintained for 7 days before experimentation. CD11c⁺ microglia were acutely isolated from P5 or 9-mo old WT and OPN-KO mice or 9-mo old 5XFAD, OPN-KO.5XFAD mice. After isolation, microglia were carefully re-suspended in medium to a final concentration of 2000 cells/μl. Each microglia-free OHSC was replenished with 4000 cells. OHSC reconstituted with CD11c⁺ microglia were incubated in the presence or absence of 2 μl synthetic human Aβ peptide 1-42 (AnaSpec, 15 μM stock)×7 d. Aβ treatment was repeated 4 times every other day, thus each slice was treated with a total of 8 μl of Aβ peptide.

Immunofluorescent staining. Transcardial perfusion by cold PBS was performed on P5 and 9-mo old WT and OPN-KO mice followed by brain removal and fixation in 4% paraformaldehyde (PFA) solution at 4° C. overnight. Then the fixed brains were rinsed with PBS and dehydrated in 30% sucrose at 4° C. overnight. OCT compound (Sakura Finetek) was used to embed the brain tissues and serial sagittal cryosections (10 μm) were cut using Cryostat (Leica). Brain cryosections were permeabilized with PBST buffer (PBS with 0.3% Triton X-100) for 1 h. OHSC slices were fixed in 4% PFA solution ×30 min and permeabilized with PBST buffer ×3 h. After 1 h incubation in blocking solution containing 5% normal donkey serum (Jackson ImmunoResearch Lab) in PBST to prevent non-specific binding, cryosections or OHSC slices were incubated×24 h with appropriate primary antibodies: rabbit anti-Iba-1 (1:1000, WAKO), biotin anti-mouse CD11c (N418, 1:50, Biolegend). To amplify CD11c signals, sections were washed with PBS and incubated with biotin anti-mouse CD11c overnight, followed by incubation with HRP-conjugated streptavidin for 1 h before incubation with Alexa Fluor 488-tyramides for 5 min to generate high density Alexa Fluor 488-labeling of CD11c protein in situ using Tyramide SuperBoost Kit (Invitrogen). Sections were then incubated×1 h with Alexa-594-conjugated donkey anti-rabbit IgG (1:500; Invitrogen). Immunofluorescence of brain slides incubated without anti-CD11c primary Ab or Tyramide Signal Amplification (TSA) were included as negative controls to exclude non-specific immunofluorescent signals. DAPI (Invitrogen) was used as a nuclear counterstain (10 min incubation) before samples were analyzed using an Olympus fluorescence microscope.

### RNA Profiling

RNA Extraction. Total RNA was extracted from FACS-sorted cell pellets using RNeasy Plus Universal Mini Kit, according to manufacturer's instructions (QIAGEN). Library preparations, sequencing reactions and bioinformatic analysis (gene hit counts) were conducted at GENEWIZ, LLC. (South Plainfield, NJ, USA) as follows:

Library Preparation. Extracted RNA samples were quantified using Qubit 2.0 Fluorometer (Life Technologies, Carlsbad, CA, USA) and RNA integrity was checked using Agilent TapeStation 4200 (Agilent Technologies, Palo Alto, CA, USA). PolyA selection strategy: RNA sequencing libraries of CD11c⁺ and CD11c⁻ microglial samples of P5 WT were prepared using a NEBNext Ultra RNA Library Prep Kit for Illumina, per manufacturer's instructions (NEB, Ipswich, MA, USA). Briefly, mRNAs were first enriched with Oligo(dT) beads before enriched mRNAs were fragmented ×15 min at 94° C. First strand and second strand cDNAs were subsequently synthesized and cDNA fragments were end repaired and adenylated at 3′ ends, and universal adapters were ligated to cDNA fragments, followed by index addition and library enrichment by limited-cycle PCR. The sequencing libraries were validated on an Agilent TapeStation (Agilent Technologies, Palo Alto, CA, USA), and quantified using Qubit 2.0 Fluorometer (Invitrogen, Carlsbad, CA) as well as by quantitative PCR (KAPA Biosystems, Wilmington, MA, USA).

Ultra-low input strategy: Due to very limited cell numbers obtained from adult mice. CD11c⁺ and CD11c⁻ microglial samples of 9-mo old WT mice were processed with SMART-Seq v4 Ultra Low Input Kit for Sequencing for full-length cDNA synthesis and amplification (Clontech, Mountain View, CA), and Illumina Nextera XT library for sequencing library preparation. Briefly, cDNA was fragmented and adaptor added using Transposase, followed by limited-cycle PCR to enrich and add index to the cDNA fragments. The final library was assessed with Agilent TapeStation.

HiSeq Sequencing. The sequencing libraries were clustered on flowcell lanes before the flowcell was loaded onto an Illumina HiSeq instrument (4000 or equivalent) per manufacturer's instructions. Samples were sequenced using a 2×150 bp Paired End (PE) configuration and image analysis and base calling were conducted by HiSeq Control Software (HCS). Raw sequence data (.bcl files) generated from Illumina HiSeq was converted into fastq files and de-multiplexed using Illumina's bcl2fastq 2.17 software. One mismatch was allowed for index sequence identification.

RNA-Seq Data Analysis. Mapping and gene counting were performed by GeneWiz. After reviewing the quality of the raw data, sequence reads were trimmed to remove adapter sequences and nucleotides with poor quality using Trimmomatic v.0.36. The trimmed reads were mapped to the Mus musculus reference genome (ENSEMBL) using STAR aligner v.2.5.2b, a splice aligner that detects and incorporates splice junctions to align the entire read sequences. BAM files were generated, and unique gene hit counts were calculated using Counts (Subread package v.1.5.2). Only unique reads that fell within exon regions were counted. Differential expression was considered significant with an FDR-adjusted p value <0.05.

Quantitative PCR. RNA of CD11c⁺ and CD11c⁻ microglia was extracted using RNeasy Plus Universal Mini Kit per manufacturer's instructions (QIAGEN). cDNA was reverse transcribed from 35 ng of RNA and prepared using a High-Capacity cDNA Reverse Transcription Kit (Applied Biosystems) per manufacturer's instructions. Real-time quantitative PCR was performed using the QuantStudio™ 6 Flex Real-Time PCR System (Applied Biosystems) for selected core genes using 5 μl of cDNA, 4.92 μl PowerUp™ SYBR™ Green Master Mix (Applied Biosystems) and 0.08 μl primer (IDT, working concentration: 200 nM) per reaction. The gene expression levels were compared using the ^{ΔΔ}Ct method normalized to β-actin.

### In Vitro Synaptosome Engulfment Assay

Synaptosomes isolation and labeling. Synaptosomes were isolated from WT mice using Syn-PER Synaptic Protein Extraction Reagent (Thermo Scientific), per manufacturer's instruction. For pHrodo labeling, dissolved pHrodo iFL green (Life Technologies) were incubated with synaptosomes on a shaker in PBS×1 h at room temperature protected from light at the ratio of 20 pg pHrodo per 1 mg synaptosomes. Unconjugated pHrodo was removed by washing with PBS before pHrodo-conjugated synaptosomes were resuspended in PBS with 5% DMSO, aliquoted, and stored at −80° C. until use.

Microglial engulfment of synaptosomes. Microglia isolated from P5 and 9-mo old WT, OPN-KO, OPN-i-KI mice were incubated with 136 pg pHrodo Green-labeled synaptosomes per 1×10⁵ cells×1 h followed by staining ×30 min with Ghost dye Violet 510 (1:1000, Tonbo Biosciences) and anti-CD11c (1:50, Biolegend). CD11c⁺ microglial engulfment of synaptosomes was assessed by flow cytometry analysis.

Quantification and statistical analysis. Data in figures are presented as mean s.e.m. Statistical analysis was performed using Prism (GraphPad Prism version 9.0). Quantification of fluorescent microscopy images was performed using ImageJ. Statistical analysis to compare the mean values for multiple groups was performed using GraphPad Prism by one-way or two-way ANOVA with Bonferroni's multiple comparisons test. Comparisons between two groups were analyzed with two-tailed Student's t test. P value <0.05 was considered statistically significant. All of the statistical details of experiments, including the statistical tests used, exact value of sample size can be found in the figure legends.

Data Availability. All protocols and reagents are detailed in the manuscript and supporting information. The RNA-seq data have been deposited in the NCBI Gene Expression Omnibus and are accessible through GEO Series accession number: GSE190713.

### REFERENCES CITED IN THIS EXAMPLE

J. Wu, H. Wu, J. An, C. M. Ballantyne, J. G. Cyster, Critical role of integrin CD11c in splenic dendritic cell capture of missing-self CD47 cells to induce adaptive immunity. Proc Natl Acad Sci USA 115, 6786-6791 (2018).
V. Haage et al., Comprehensive gene expression meta-analysis identifies signature genes that distinguish microglia from peripheral monocytes/macrophages in health and glioma. Acta Neuropathol Commun 7, 20 (2019).
A. Wlodarczyk et al., A novel microglial subset plays a key role in myelinogenesis in developing brain. EMBO J 36, 3292-3308 (2017).
H. Keren-Shaul et al., A Unique Microglia Type Associated with Restricting Development of Alzheimer's Disease. Cell 169, 1276-1290 e1217 (2017).
Q. Li et al., Developmental Heterogeneity of Microglia and Brain Myeloid Cells Revealed by Deep Single-Cell RNA Sequencing. Neuron 101, 207-223 e210 (2019).
T. R. Hammond et al., Single-Cell RNA Sequencing of Microglia throughout the Mouse Lifespan and in the Injured Brain Reveals Complex Cell-State Changes. Immunity 50, 253-271 e256 (2019).
C. Sala Frigerio et al., The Major Risk Factors for Alzheimer's Disease: Age, Sex, and Genes Modulate the Microglia Response to Abeta Plaques. Cell Rep 27, 1293-1306 e1296 (2019).
M. L. Shinohara, J. H. Kim, V. A. Garcia, H. Cantor, Engagement of the type I interferon receptor on dendritic cells inhibits T helper 17 cell development: role of intracellular osteopontin. Immunity 29, 68-78 (2008).
M. L. Shinohara, H. J. Kim, J. H. Kim, V. A. Garcia, H. Cantor, Alternative translation of osteopontin generates intracellular and secreted isoforms that mediate distinct biological activities in dendritic cells. Proc Natl Acad Sci USA 105, 7235-7239 (2008).
R. Patarca et al., Structural and functional studies of the early T lymphocyte activation 1 (Eta-1) gene. Definition of a novel T cell-dependent response associated with genetic resistance to bacterial infection. J Exp Med 170, 145-161 (1989).
S. Ashkar et al., Eta-1 (osteopontin): an early component of type-1 (cell-mediated) immunity. Science 287, 860-864 (2000).
G. F. Weber et al., Phosphorylation-dependent interaction of osteopontin with its receptors regulates macrophage migration and activation. J Leukoc Biol 72, 752-761 (2002).
E. M. Hur et al., Osteopontin-induced relapse and progression of autoimmune brain disease through enhanced survival of activated T cells. Nat Immunol 8, 74-83 (2007).
H. N. Noristani et al., RNA-Seq Analysis of Microglia Reveals Time-Dependent Activation of Specific Genetic Programs following Spinal Cord Injury. Front Mol Neurosci 10, 90 (2017).
I. M. Chiu et al., A neurodegeneration-specific gene-expression signature of acutely isolated microglia from an amyotrophic lateral sclerosis mouse model. Cell Rep 4, 385-401 (2013).
M. Mizutani et al., The fractalkine receptor but not CCR2 is present on microglia from embryonic development throughout adulthood. J Immunol 188, 29-36 (2012).
M. L. Bennett et al., New tools for studying microglia in the mouse and human CNS. Proc Natl Acad Sci USA 113, E1738-1746 (2016).
S. Krasemann et al., The TREM2-APOE Pathway Drives the Transcriptional Phenotype of Dysfunctional Microglia in Neurodegenerative Diseases. Immunity 47, 566-581 e569 (2017).
A. M. Jurga, M. Paleczna, K. Z. Kuter, Overview of General and Discriminating Markers of Differential Microglia Phenotypes. Front Cell Neurosci 14, 198 (2020).
L. Fourgeaud et al., TAM receptors regulate multiple features of microglial physiology. Nature 532, 240-244 (2016).
S. Y. Park, I. S. Kim, Engulfment signals and the phagocytic machinery for apoptotic cell clearance. Exp Mol Med 49, e331 (2017).
E. Grajchen et al., CD36-mediated uptake of myelin debris by macrophages and microglia reduces neuroinflammation. J Neuroinflammation 17, 224 (2020).
D. Schulz, Y. Severin, V. R. T. Zanotelli, B. Bodenmiller, In-Depth Characterization of Monocyte-Derived Macrophages using a Mass Cytometry-Based Phagocytosis Assay. Sci Rep 9, 1925 (2019).
E. Favuzzi et al., GABA-receptive microglia selectively sculpt developing inhibitory circuits. Cell 184, 5686 (2021).
M. Inoue, M. L. Shinohara, Intracellular osteopontin (iOPN) and immunity. Immunol Res 49, 160-172 (2011).
G. F. Weber, S. Ashkar, M. J. Glimcher, H. Cantor, Receptor-ligand interaction between CD44 and osteopontin (Eta-1). Science 271, 509-512 (1996).
E. Ruoslahti, M. D. Pierschbacher, New perspectives in cell adhesion: RGD and integrins. Science 238, 491-497 (1987).
S. R. Anderson et al., Developmental Apoptosis Promotes a Disease-Related Gene Signature and Independence from CSF1R Signaling in Retinal Microglia. Cell Rep 27, 2002-2013 e2005 (2019).
L. D. White, S. Barone, Jr., Qualitative and quantitative estimates of apoptosis from birth to senescence in the rat brain. Cell Death Differ 8, 345-356 (2001).
N. Hagemeyer et al., Microglia contribute to normal myelinogenesis and to oligodendrocyte progenitor maintenance during adulthood. Acta Neuropathol 134, 441-458 (2017).
P. B. Stranges et al., Elimination of antigen-presenting cells and autoreactive T cells by Fas contributes to prevention of autoimmunity. Immunity 26, 629-641 (2007).
L. Madisen et al., A robust and high-throughput Cre reporting and characterization system for the whole mouse brain. Nat Neurosci 13, 133-140 (2010).
E. Kourepini et al., Osteopontin expression by CD103− dendritic cells drives intestinal inflammation. Proc Natl Acad Sci USA 111, E856-865 (2014).
J. W. Leavenworth, B. Verbinnen, J. Yin, H. Huang, H. Cantor, A p85alpha-osteopontin axis couples the receptor ICOS to sustained Bcl-6 expression by follicular helper and regulatory T cells. Nat Immunol 16, 96-106 (2015).
S. Hellwig et al., Forebrain microglia from wild-type but not adult 5×FAD mice prevent amyloid-beta plaque formation in organotypic hippocampal slice cultures. Sci Rep 5, 14624 (2015).

### Example 2

### Example 2—Osteopontin-Producing Microglia Contribute to Alzheimer's Disease

### Summary

Microglia are the resident immune cells in the brain and dysregulated microglial activation is a cardinal feature of Alzheimer's disease (AD) 1. Despite abundant evidence for a critical role in AD, the contribution of this cell type to disease pathology is not well understood. Evidence that microglia can exert a protective effect has come from observations that disease-associated microglia (DAM) can phagocytose amyloid-beta (AD) plaques^{2,3} In contrast, there is evidence that microglia are pathogenic based on observations that microglial elimination prevents Aβ seeding and plaque formation in the 5XFAD mouse model 4. Our studies indicate that the impact of microglia reflects the separate contributions of protective and pathogenic microglial subsets that can be distinguished according to their developmental history and functional phenotype.

Although many disease-associated genes have been identified in murine models of AD, most have not been confirmed in studies of human AD. The Spp1 gene (encoding OPN) is an exception, since it appears to be strongly upregulated by microglia in both animal models of AD and the human disease^{2,5-8}. We have recently characterized a microglial subset according to expression of the CD11c surface marker that is the sole producer of osteopontin (OPN) in healthy murine brain during early development and adult life⁹.

OPN is also expressed by peripheral dendritic cells and macrophages, where it can regulate inflammatory and autoimmune responses¹⁰⁻¹² Steinman and colleagues have implicated OPN in neuroinflammatory and neurodegenerative disorders including Multiple Sclerosis^{13,14} However, the contribution of OPN-producing microglial to neurodegenerative disease is unknown.

Here we define a pathogenic CD11c⁺ OPN⁺ microglial subset which drives AD pathology. Genetic deletion of OPN production by CD11c⁺ microglia in 5XFAD mice inhibits production of inflammatory cytokines and promotes TREM2-dependent microglial uptake of amyloid fibrils and associated lysosomal activation. Targeting this OPN-dependent pathway results in increased lysosomal degradation of Aβ fibrils and extrusion of compacted Aβ protein into brain parenchyma, resulting in a reduction of diffuse Aβ plaques and marked improvement in cognitive function. We extended these murine findings to human disease using clinically and neuropathologically characterized brain tissues from AD patients, mildly cognitively impaired patients, and cognitively normal controls (Mt. Sinai Brain Repository). This analysis indicated that OPN production by human CD11c⁺ microglia correlates closely with the progression of both clinical dementia and AD neuropathology. Collectively, these findings indicate that targeting production of OPN by CD11c⁺ microglia to reduce diffuse toxic plaques and inhibit neuroinflammation represents an effective therapeutic strategy for AD.

### Results

### OPN Contributes to AD Pathology and Cognitive Impairment in 5XFAD Mice

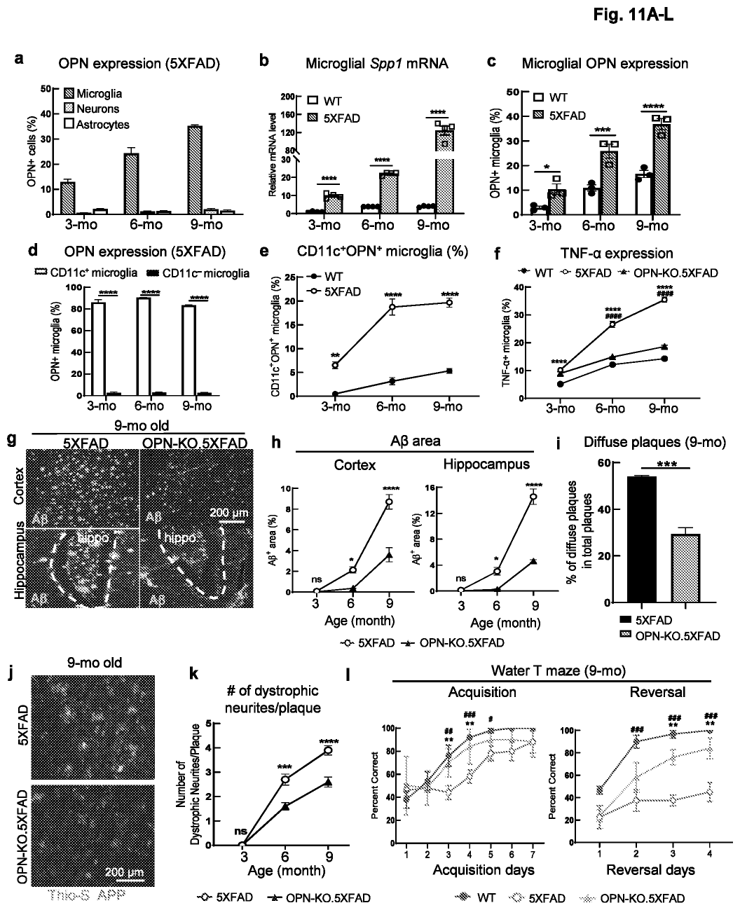

To identify the cellular source of OPN during disease progression in brains of 5XFAD mice, we measured OPN expression by astrocytes, neurons and microglia at 3-, 6- and 9-months of age. Astrocytes and neurons did not produce detectable amounts of OPN, while microglial production of OPN was robust and increased with disease progression (FIG. 11a). There was a 10-20-fold increase in OPN mRNA and a 2-3-fold increase of OPN protein by 9-mo old 5XFAD microglia compared with age-matched B6-WT controls (FIG. 11b, c), indicating microglial OPN production closely parallels disease development.

Microglia that co-express CD11c represent the sole producer of OPN in brains of healthy mice⁹. We observed that microglial OPN production in 5XFAD mice is also confined to this CD11c⁺ microglial subset, which increases dramatically during the initial 6 months of disease progression compared to relatively low levels in healthy age-matched controls (FIGS. 11d, e).

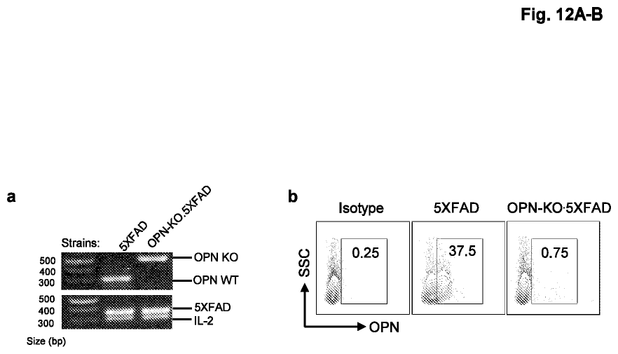

We generated OPN-KO-5XFAD mice after crossing 5XFAD transgenic mice with Spp1^{flstop} (OPN-KO) mice¹⁵ and confirmed OPN deletion at both the genetic and protein levels (FIG. 12). Genetic deletion of OPN in 5XFAD mice resulted in reduction of microglial production of TNF-α to levels similar to age-matched healthy (B6-WT) controls (FIG. 11f). Examination of Aβ plaque area of OPN-KO 5XFAD mice revealed a 3-5-fold reduction in both cortex and hippocampus at 6- and 9-months of age compared to age-matched 5XFAD controls (FIG. 11g, h), indicating that OPN might inhibit Aβ clearance in 5XFAD mice. Analysis of brain cryosections co-stained with anti-Aβ mAb 6E10 (to identify both diffuse and condensed forms of Aβ plaques) and Thioflavin-S (which identifies only β-sheet⁺ Aβ condensed plaques³) also revealed that genetic deletion of OPN substantially reduced the proportion of diffuse plaque development at 9-mo of disease (FIG. 11i).

Neuritic dystrophy, a central element of 5XFAD disease pathology consisting of swollen, bulbous-shaped neurites comprised of dysfunctional axons and terminals expressing the amyloid precursor protein (APP)¹⁶, correlates with the clinical severity of dementia^{17,18}. We noted that the numbers of dystrophic neurites per plaque were reduced by approximately 50% in OPN-KO.5XFAD mice (FIG. 11j, k). Since increased numbers of dystrophic neurites correlates closely with cognitive decline^{19,20} we measured the impact of OPN expression on cognitive function of 9-mo old 5XFAD mice using the Water T maze to assess spatial learning and memory (acquisition trials) and cognitive flexibility (reversal trials). OPN deletion markedly improved these cognitive functions, as indicated by a 40-50% increase in correct choices made by OPN-KO.5XFAD mice in both the acquisition and reversal phases of these trials (FIG. 11l). Collectively, these data indicate that OPN deficiency substantially decreases microglial pro-inflammatory responses, diffuse Aβ plaques and dystrophic neurites, and improves cognitive function in 5XFAD mice.

### OPN Production by the Pathogenic CD11c⁺ Microglia in 5XFAD Brain Promotes Pro-Inflammatory Responses and Inhibits Aβ Uptake

Since CD11c⁺ microglia are the sole cellular producers of OPN in murine brain (FIGS. 1d, 11a-c), the pathogenic impact of OPN can depend on the development and function of this CD11c⁺ microglial subset.

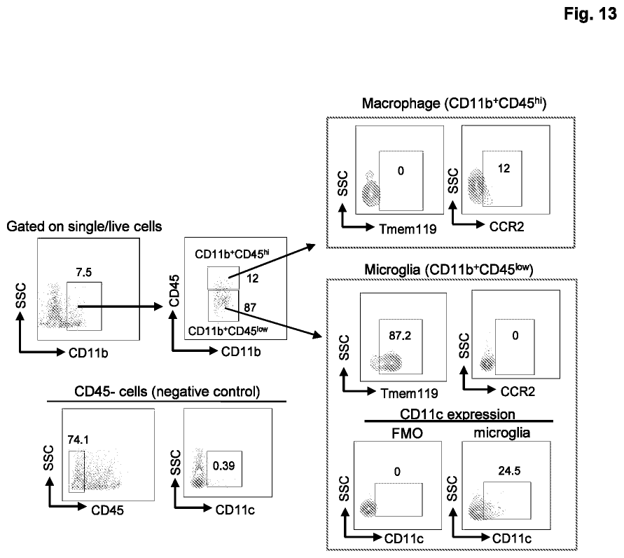

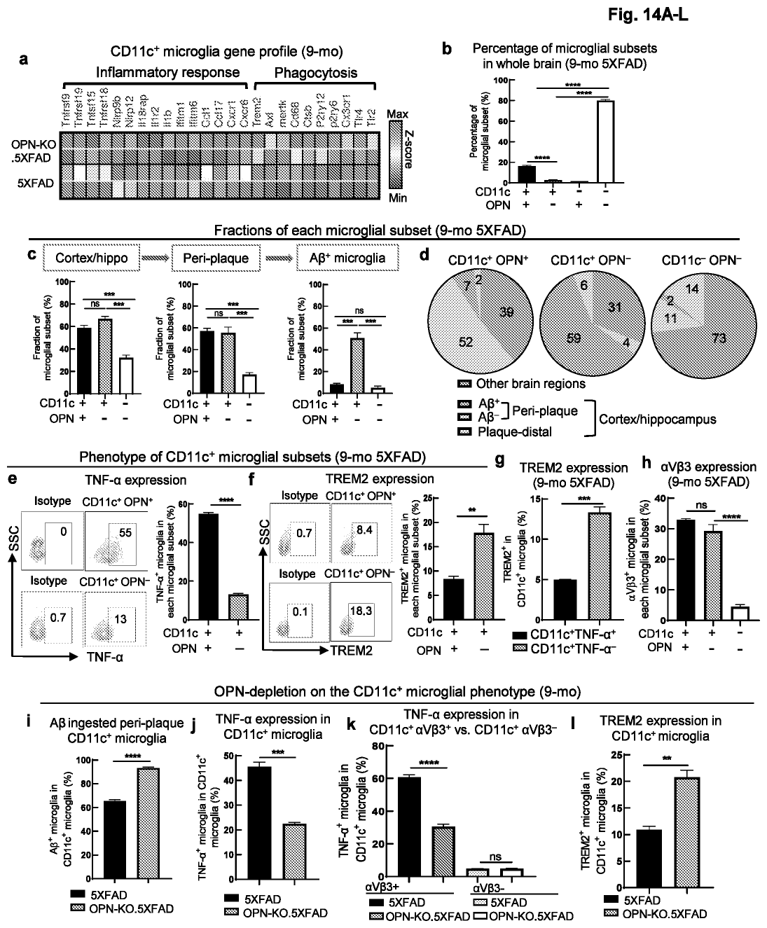

We defined the contribution of OPN to the phenotype of CD11c⁺ microglia in 5XFAD disease according to RNA-Seq analysis of purified CD11c⁺ microglia from 9-mo old 5XFAD mice and OPN-KO.5XFAD mice. We validated the FACS gating of CD11c⁺ microglia in 5XFAD mice by flow cytometry analysis (FIG. 13). OPN deficiency resulted in downregulation of inflammatory response-related genes (e.g., Tnfrsf9,i11b,Cc11) and upregulation of phagocytosis-related genes, including Trem2, MerTk, CD68 and Ctsb (FIG. 14a and Table 1), which includes core components of molecular pathways responsible for Aβ uptake and lysosomal compaction 3. Table 1 includes a list of differentially expressed genes (DEGs) in CD11c⁺ microglia from 9-mo old OPN-KO.5XFAD mice compared with FXFAD mice. There were 2,985 DEGs identified. Gene expression was considered upregulated if log₂ FC>1 or downregulated if log₂ FC, −1. DEGs were considered significant with an FDR-adjusted p value <0.05.

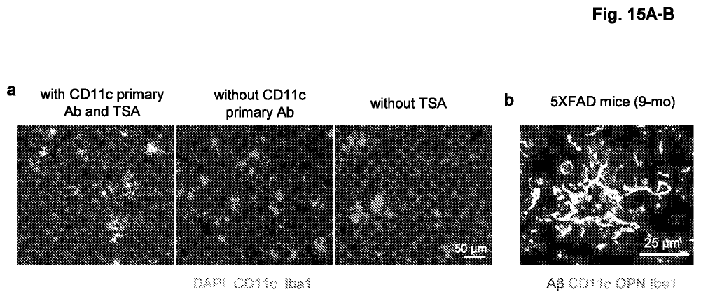

Flow cytometry analysis indicated that the large majority (80%) of microglia in brain from 9-mo old 5XFAD mice expressed neither CD11c nor OPN (double negative, DN), while about 15-20% expressed both (double positive, DP) and less than 5% were CD11c⁺ OPN⁻. We did not detect significant numbers of CD11c⁻ OPN⁺ microglia (FIG. 14b). We then determined the fractions of each microglial subset within the cortex and hippocampus (C/H), i.e., the brain regions most affected by 5XFAD disease, and further evaluated the fractions of each subset that resided within peri-plaque areas (25 mm of the Aβ plaque core)²¹ using brain cryosections of 9-mo old 5XFAD mice. Microglial CD11c-specific immunofluorescent signal was validated using several negative controls before staining and evaluation of CD11c⁺ OPN⁺, CD11c⁺ OPN⁻ and CD11c OPN⁻ microglial subsets (FIG. 15a, b).

Approximately 60% of the CD11c⁺ OPN⁺ (DP) microglial subset resided in C/H regions and virtually all of these DP microglia located within peri-plaque areas. Analysis of Aβ ingestion by these peri-plaque DP microglia revealed that only about 12% contained ingested Aβ (FIG. 14c, d). A substantial (˜70%) fraction of the reciprocal CD11c⁺ subset—CD11c⁺ OPN⁻—also resided within the C/H regions and a majority localized within peri-plaque areas. In this case, however, over 90% of peri-plaque CD11c⁺ OPN⁻ microglia contained ingested Aβ material. In contrast to the CD11c⁺ microglial subsets, the majority (>70%) of the CD11c⁻ OPN⁻ (DN) subset resided in brain regions outside the C/H areas. Analysis of the DN subset within the C/H areas also indicated that, although about half located near or within peri-plaque areas, only about 2% contained Aβ material (FIG. 14c, d).

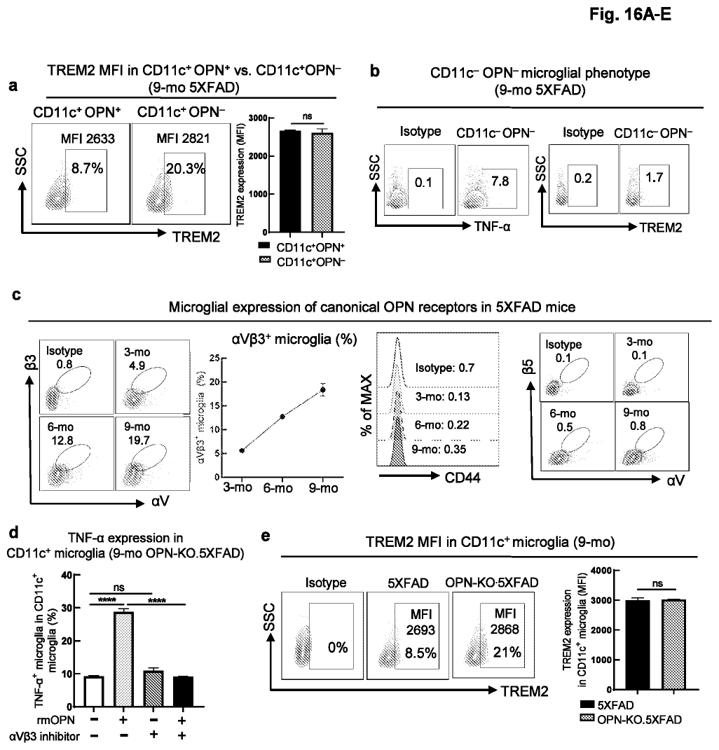

Since both CD11c⁺ microglial subsets closely associate with Aβ plaques (FIG. 14d), we characterized their phenotype in more detail by flow cytometry. CD11c⁺ OPN⁺ (DP) displayed substantial TNF-α production and diminished TREM2 expression, compared to the CD11c*OPN⁻ subset, which expressed low levels of TNF-α but strongly increased proportions of TREM2⁺ microglia (FIG. 14e, f and FIG. 16a). Although CD11c⁻ OPN⁻ (DN) microglia account for ˜80% of whole brain microglia, they do not produce detectable TNF-α and express marginal levels of TREM2 (FIG. 16b). A direct comparison of TREM2 and TNF-α expression by CD11c⁺ microglia revealed that the pro-inflammatory (i.e., TNF-α⁺) CD11c⁺ microglia contained ˜5% TREM2⁺ cells, while ˜15% of TNF-α⁻ microglia were TREM2⁺ (FIG. 14g). These analyses indicate that the pro-inflammatory phenotype of DP CD11c⁺ OPN⁺ microglia is associated with reduced Aβ uptake (FIG. 14c, d), while CD11c⁺ OPN⁻ microglia, which lack a significant pro-inflammatory element, display robust levels of Aβ ingestion.

Since genetic deletion of OPN reduces microglial production of TNF-α to levels found in non-diseased mice (FIG. 1f), we examined the relationship between OPN and the pro-inflammatory TNF-α phenotype. OPN-dependent ligation of macrophage αVβ3 integrin receptor promotes a pro-inflammatory phenotype and reduced uptake of apoptotic debris^{22,23} Microglial expression of the canonical αVβ3 integrin receptor for OPN^{9,24} increased substantially in 5XFAD mice, while other OPN receptors, CD44 and αVβ5, are barely detectable (FIG. 16c). The addition of rmOPN induced TNF-α expression by CD11c⁺ microglia, which was abolished by treatment with cyclic RGD inhibition of the αVβ3 integrin (Cilengitide) (FIG. 16d). We also noted that CD11c OPN microglia expressed negligible amounts of αVβ3, consistent with the virtual absence of pro-inflammatory TNF-α expression by this subset, while the CD11c⁺ subsets (CD11c⁺ OPN⁻ and CD11c⁺ OPN⁺) both expressed higher (>30%) levels of αVβ3 (FIG. 14h). To directly define the contribution of OPN to the phenotype of CD11c⁺ microglia, we examined microglia from OPN-KO.5XFAD mice. We noted that CD11c⁺ microglia from 9-mo old OPN-KO.5XFAD mice displayed substantially increased ingestion of Aβ⁺ material (FIG. 14i) along with a 50% reduction in TNF-α production (FIG. 14j) compared to 5XFAD mice.

In sum, (a) OPN-deficiency leads to reduced proportion of TNF-αC cells in αVβ3⁺ CD11c⁺ microglia and increased proportion of TREM2⁺ microglia and (b) CD11c⁺ microglia lacking the OPN receptor do not produce TNF-α (FIG. 14k, 1 and FIG. 16e). These findings indicate that OPN can promote TNF-α production and reduce TREM2 expression via engagement of its (αVβ3) receptor on CD11c⁺ microglia.

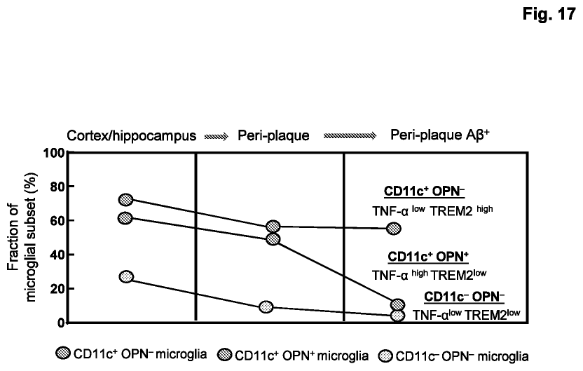

Collectively, these findings reveal the distinct contributions of 3 major microglial subsets to 5XFAD disease. Although the CD11c⁻ OPN⁻ microglial subset represents the major (˜80%) subset in brain, it does not ingest Aβ or produce TNF-α and is not a prominent component of Aβ lesions. In contrast, although CD11c⁺ (both OPN⁺ and OPN⁻) microglia account for a relatively small (˜20%) fraction of microglia in total brain, >60% of these microglia locate within plaque areas. OPN production by these two CD11c⁺ microglial subsets further refines their distinct functional phenotypes within and surrounding plaque areas. Although CD11c⁺ OPN⁺ microglia produce high levels of TNF-α, relatively few contain ingested Aβ or express TREM2, consistent with their pathogenic contribution to disease. In contrast, while CD11c⁺ OPN⁻ microglia do not produce significant levels of TNF-α, they actively engulf Aβ and can exert disease protective rather than a disease-driving effect. (summarized in FIG. 17).

### OPN Inhibits Aβ Plaque Compaction Through Suppression of the TREM2-Lysosomal Phagocytic Pathway

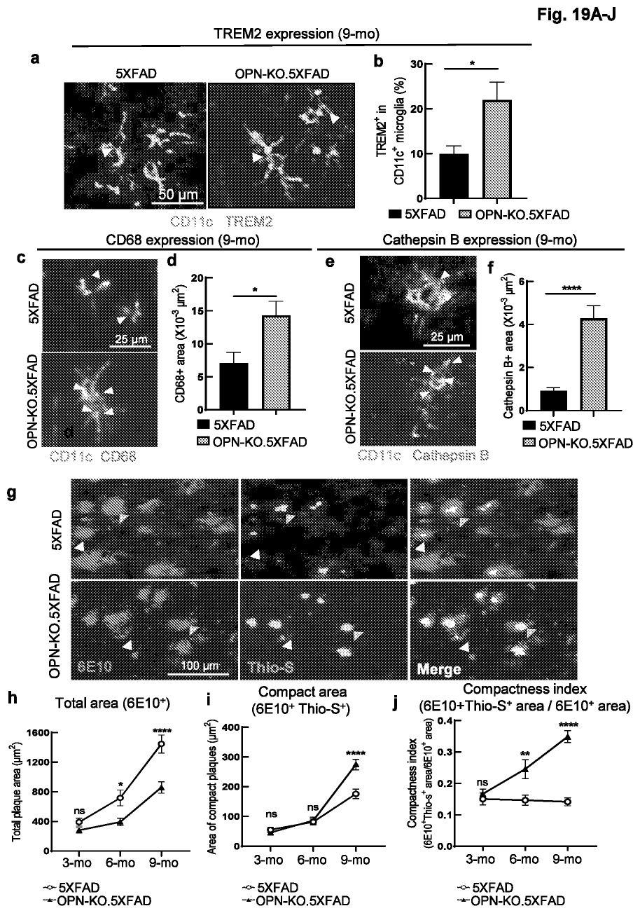

Recent evidence indicates that the TREM2 pathway enhances lysosomal degradation of amyloid fibrils, leading to increased extrusion of condensed AR at the expense of the diffuse form of Aβ plaques in 5XFAD mice^{25,26}. We asked whether the decrease in diffuse plaques we noted earlier in OPN-KO.5XFAD brains (FIG. 11i) reflected increased activation of this TREM2-lysosomal pathway. Analysis of in situ TREM2 expression in brain cryosections confirmed flow cytometry analyses: CD11c⁺ microglia from OPN-KO.5XFAD expressed 2-3-fold more TREM2 compared with 5XFAD mice (FIG. 19a, b). This increase was associated with enhanced lysosomal activation, as judged by increased expression of the CD68 lysosomal activation glycoprotein in microglia from OPN-KO.5XFAD mice compared to age-matched 5XFAD mice (FIG. 19c, d) as well as a 3-4-fold increase in lysosomal expression of cathepsin B, a lysosomal cysteine protease that cleaves and degrades Aβ^{27,28} (FIG. 19e, f). Taken together, these data indicate that OPN-dependent downregulation of the TREM2-phagolysosomal Aβ compaction pathway can account for our findings that genetic deletion of OPN substantially reduces the proportion of diffuse plaques (6E10⁺ Thio-S⁻) within total plaques (6E10⁺) in 9-mo old 5XFAD mice (e.g., FIG. 11i).

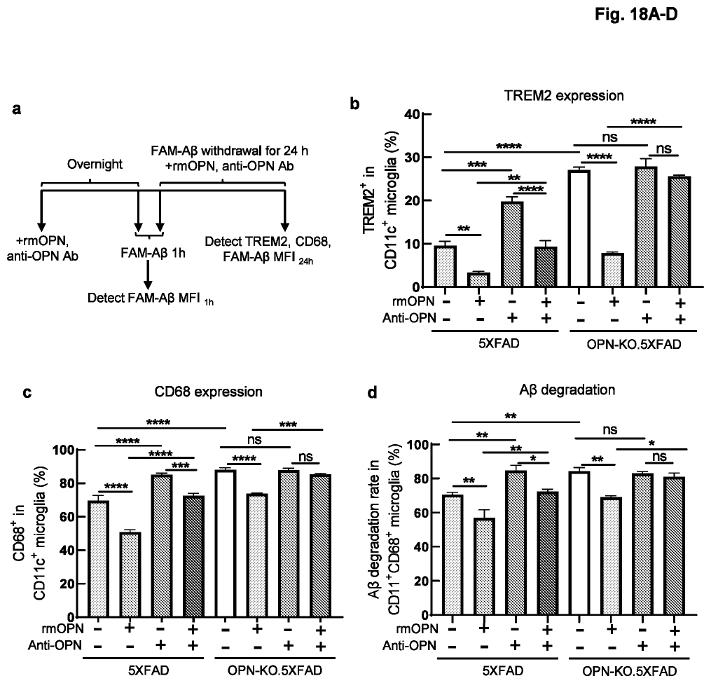

We then asked whether an interaction between recombinant OPN might inhibit TREM2 expression by CD11c⁺ microglia. We found that TREM2 expression wase sharply inhibited by rmOPN, and this reduction was reversed by addition of anti-OPN Ab (FIG. 18a, b). Moreover, rmOPN also inhibited lysosomal activation, as judged by reduced expression of CD68 (FIG. 18c). Suppression of TREM2-lysosomal activation (FIG. 19a-d and FIG. 18a-c) was accompanied by a substantial reduction of Aβ degradation [(Aβ MFI₁ₕ−Aβ MFI₂₄ₕ)/Aβ MFI₁ₕ] in lysosomes of CD11c⁺ microglia that was fully reversed by anti-OPN Ab (FIG. 18d).

To directly test the contribution of OPN to microglial plaque compaction, we distinguished compact plaques from total plaques using Thioflavin-S to identify Aβ⁺ aggregates in a β-sheet conformation. We found that the 40-45% decrease in total plaque area in OPN-KO.5XFAD mice noted in FIG. 3g, h was accompanied by an increase in compact plaque area (FIG. 19i), as judged from a striking increase in plaque compactness index (6E10⁺ Thio-S⁺ area/6E10⁺ area) (FIG. 19j).

Collectively, these data indicate that OPN-mediated inhibition of TREM2-dependent lysosomal activation resulting in impairment of microglial compaction of Aβ plaques and an increase in diffuse plaques, represents a pathogenic pathway driven by OPN⁺CD11c⁺ microglial that can drive 5XFAD disease.

Increased OPN Production by CD11c⁺ Microglia Correlates with Disease Severity and Neuropathology in Brain Tissue from AD Patients

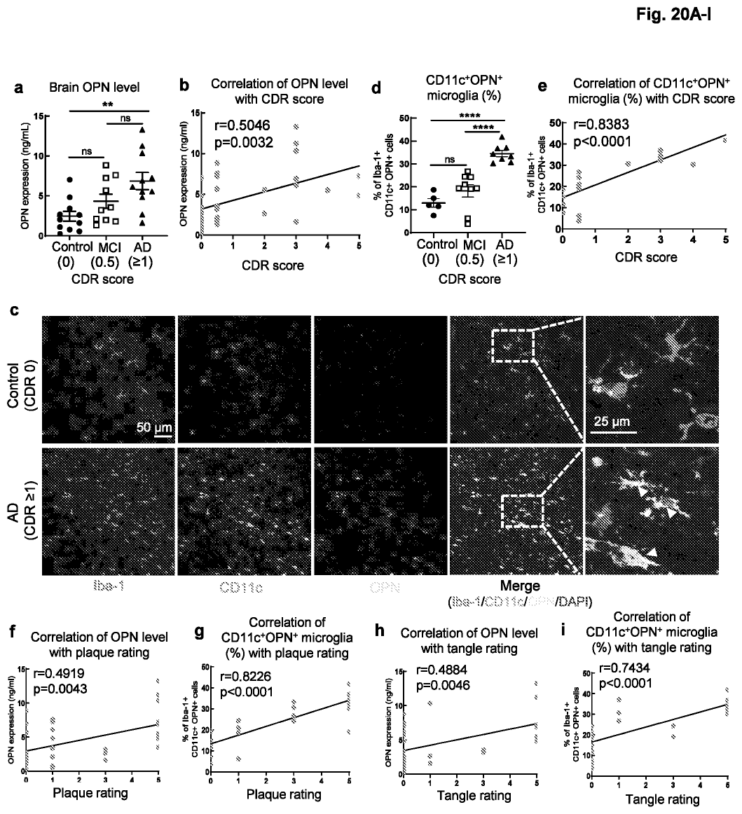

To extend these findings from a murine model of AD to the human disease, we analyzed brain tissue from clinically and neuropathologically confirmed AD patients (cohort characterization in Table 2 and Table 3) whose clinical dementia rating (CDR) was ≥1 at death, as well as patients with mild cognitive impairment (MCI, CDR=0.5) and cognitively normal controls (CDR=0). Measurements of OPN in middle frontal gyrus homogenates revealed a 3-fold increase in OPN expression in AD patient brains compared with that of cognitively normal control subjects. Increased OPN expression in AD patient brains compared with brains of patients with MCI was also noted, but did not reach statistical significance (FIG. 20a). A correlative analysis of brain OPN expression and CDR score indicated that increased OPN expression correlated positively with dementia severity (FIG. 20b).

We then determined the percentage of CD11c⁺ OPN⁺ (DP) microglia by immunofluorescence analysis of human brain sections from the middle frontal gyrus, a region affected early by AD²⁹. Normal controls and MCI subjects displayed similar proportions of CD11c⁺ OPN⁺ microglia (CD11c⁺ OPN⁺Iba-1⁺). We observed a 3-fold increase of CD11c⁺ OPN⁺ microglia in brain sections from AD patients compared with sections from normal controls (FIG. 20c, d). Moreover, the percentage of CD11c⁺ OPN⁺ microglia from AD patients was significantly higher than that from MCI patients (FIG. 20d), indicating that the percentage of CD11c⁺ OPN⁺ microglia represents a more sensitive parameter for differentiation of AD patients from patients who suffer from MCI than simple OPN levels (FIG. 20c). This conclusion is further supported by the very strong correlation of the percentage of CD11c⁺ OPN⁺ microglia with CDR scores (FIG. 20e).

We also determined the correlation between (a) OPN levels and (b) the percentage of CD11c⁺ OPN⁺ microglia in the middle frontal gyrus with neuritic plaques and neurofibrillary tangle density ratings that were determined postmortem by a neuropathologist before sample storage in the Mount Sinai brain repository as described³⁰. We found that higher brain OPN levels correlated with higher neuritic plaque levels (r=0.4919, p=0.0043) and neurofibrillary tangle ratings (r=0.4884, p=0.0046) (FIG. 20f, h). Similar to the associations found with dementia severity, there was a stronger correlation between the percentage of CD11c⁺ OPN⁺ (DP) microglia and the density of neuritic plaques (r=0.8226, p<0.0001) and neurofibrillary tangles (r=0.7434, p<0.0001) (FIG. 20g, i). These findings lend further support to the view that the percentage of CD11c⁺ OPN⁺ microglia can be a sensitive marker of both clinical severity and neuropathology of AD.

### Discussion

Microglial dysregulation characterized by a sustained inflammatory response and impaired Aβ plaque processing can contribute to neuritic dystrophy and cognitive decline in the setting of AD^{1,31} We have previously noted that the small microglial subset expressing CD11c, first defined by the Owens' group, is the sole producer of OPN in brain tissues of healthy mice 9. Here, we report that OPN production is similarly confined to CD11c microglia in the 5XFAD model of AD during disease development. Here, we report that OPN production is similarly confined to CD11c microglia in the 5XFAD model of AD during disease development. OPN production by this CD11c+ OPN⁺ subset can reflect enhanced pro-inflammatory responses and impaired TREM2-dependent Aβ plaque consolidation in activated lysosomes. These findings provide new insight into the molecular mechanisms that drive AD-related cognitive impairment and, without being bound by theory, highlight OPN as a tractable therapeutic target in AD.

Based on expression of CD11c and OPN production, microglia are divisible into 3 major subsets, i.e., CD11c⁺ OPN⁺, CD11c⁺ OPN⁻ and CD11c⁻ OPN-microglia. Although CD11c⁻ OPN⁻ subset accounts for >80% of microglia in the whole brain, <30% reside in the cortex/hippocampus of 5XFAD mouse brain and ˜10% locate within the peri-plaque areas. Without wishing to be bound by theory, since less than 2% of CD11c OPN⁻ microglia ingest Aβ and do not produce appreciable TNF-α, this CD11c⁻ OPN⁻ microglia represents a homeostatic subset that does contribute to 5XFAD pathology. In contrast, dissection of the CD11c⁺ microglia according to expression of OPN identifies a pathogenic and protective subset. Although CD11c⁺ OPN⁻ microglia account for a minor population (˜3%) in whole brain, ˜70% of this subset resides in cortex/hippocampus and the majority (˜65%) of these cells locate in peri-plaque areas. These CD11c⁺ OPN⁻ microglia can be protective, since they robustly engulf AR (˜60%), express high levels of TREM2, and produce negligible levels of TNF-α. In contrast, although the CD11c⁺ OPN⁺ DP microglial subset is also significantly enriched (˜60%) in peri-plaque areas, only ˜5-7% ingest AR while almost 60% produce TNF-α and express low levels of TREM2, supporting the view that CD11c⁺ OPN⁺ microglia represent a pathogenic microglial subset.

Since TREM2 can facilitate microglial uptake of Aβ^{32,33}, strong expression of TREM2 by CD11c⁺ OPN⁻ microglia can contribute to the robust Aβ uptake by these cells. In contrast, weak expression of TREM2 by DP CD11c⁺ OPN⁺ microglia is consistent with low levels of Aβ ingestion. Uptake of Aβ by CD11c⁺ OPN⁻ microglia may also reflect engagement of the αVβ3 receptor by milk fat globule EGF factor 8 (MFG-E8), which binds to phosphatidylserine (PtdSer) molecules that decorate Aβ plaques and can facilitate microglial endocytosis^{3,34,35} Without being bound by theory, an MFG-E8-αVβ3 interaction drives Aβ uptake by CD11c⁺ OPN⁻ microglia, while competition with OPN reduces MFG-E8 binding by CD11c⁺ OPN⁺ DP microglia and enhance TNF-α production by the pathogenic CD11c⁺ OPN⁺ microglial subset.

Collectively, our findings support the view that OPN-deficient CD11c⁺ microglia expressing high levels of TREM2 represent a protective subset that actively ingests Aβ for lysosomal digestion without inducing an inflammatory response. In contrast and without being bound by theory, the OPN-producing CD11c⁺ (DP) microglial subset that fails to effectively take up Aβ produces a strong inflammatory response that drives AD pathology. These findings refine our understanding of the DAM phenotype (which are CD11c⁺), indicating that the protective contribution of DAM² reflects the activities of the CD11c⁺ OPN⁻ subset defined here.

Colonna and colleagues have demonstrated that TREM2 plays an essential role in regulating the microglial interaction with Aβ plaques, perhaps by inducing microglia to surround and alter Aβ plaque structure into more compact form, thereby limiting neuritic darnage^{36,37}. Microglial AR compaction depends in part on a TREM2-dependent phagocytic pathway that internalizes AR into activated lysosomes for digestion³⁸. As a consequence, TREM2 deficiency in both mouse models and AD patients can lead to an increase in AR burden, diffuse plaques, dystrophic neurites and cognitive impairment^{17,26,36,39} The contribution of TAM receptors that can work downstream of TREM2 in this plaque processing pathway comes from observations that TAM receptor-deficient APP/PS1 mice display reduced numbers of dense-core plaques and increased cognitive deficits³. OPN-dependent inhibition of AR phagocytosis and suppression of this TREM2-lysosomal phagocytic pathway is indicated from our transcriptomic analysis of CD11c⁺ microglia that revealed OPN-dependent downregulation of key components in this pathway (Trem2, Axl, Mertk, C68 and Ctsb) and increased expression of TREM2 and CD68 expression by OPN-deficient CD11c⁺ microglia. The substantial reduction of plaque burden in OPN-deficient 5XFAD mice reflects accelerated lysosomal degradation of Aβ, as judged by upregulation of microglial cathepsin B and reductions in diffuse plaques accompanied by an increase in compact plaques.

To date, clinical trials of anti-Aβ antibody have indicated that reduction in plaque load is not accompanied by improved cognitive function. Most recent evidence indicated that microglial deposition of previously ingested and compacted neurotoxic AR fibrils, via exocytosis or microglial death, is essential for the formation of nontoxic dense-core plaques which correlate with rescued cognitive function^{3,40}. Without being boundy by theory, since microglial packing of Aβ fibrils into dense plaques represents a neuroprotective mechanism³, antibodies that target Aβs disaggregate condensed non-toxic AR fibrils into oligomers that increase neurotoxicity⁴¹. Hence, without wishing to be bound by theory, indiscriminate disaggregation of dense plaques does not efficiently ameliorate neurotoxicity and associated cognitive impairment^{42,43}. Without being bound by theory, since OPN deletion increases plaque compactness, substantially reduces dystrophic neurites, and improves cognitive function, targeting OPN represents a more effective therapeutic strategy than current clinical approaches that indiscriminately target plaques in mAb trials. By contrast, OPN deletion favorably alters the ratio of pro-inflammatory to phagocytic microglia and increases TREM2-associated Aβ uptake, leading to increased conversion of neurotoxic Aβ material into non-toxic dense-core plaques.

Not all findings from AD mouse models are applicable to human AD. However, our core findings appear to apply to the human disease. Upregulation of the OPN-encoding gene (Spp1) has been identified as a feature of both AD mouse models^{2,7,44} and patients 8,45,46 Interestingly, an increase in microglial Spp1 gene was not noted in AD patients that carried TREM2 loss-of-function variations (TREM2-R47H and TREM2-R62H)⁴⁴. Without being bound by theory, genetic impairment of the TREM2 pathway obviates the requirement for OPN-dependent suppression of the TREM2 pathway in the development of plaque-related pathology.

Although elevated OPN levels in CSF and plasma samples have been reported in AD patients⁴⁷, the relationship between brain OPN levels and disease severity of AD patients has not been clear. Consistent with our preclinical findings in 5XFAD mice, brain OPN levels and especially the proportion of CD11c⁺ OPN⁺ microglia correlate strongly with neuritic plaque ratings, according to postmortem assessment of brain samples from AD patients. Analysis of human brain samples from well-characterized AD patients and controls also provides evidence that brain OPN levels correlate directly with disease severity according to CDR scores. In addition, the percent of CD11c⁺ OPN⁺ microglia increases with progression and severity of dementia and correlates strongly with CDR scores. The increasing percent of CD11c⁺ OPN⁺ microglia noted by MCI patients and AD patients indicates a sensitive indicator of disease development and OPN production by CD11c⁺ microglia strongly correlates with both deficits and the severity of AD neuropathology. Neurofibrillary tangles, consisting mainly of aggregated hyperphosphorylated tau protein, represent another important pathological hallmark of AD. We noted that brain OPN levels as well as the percent of CD11c⁺ OPN⁺ microglia correlate closely with neurofibrillary tangle ratings, indicating that OPN contributes to tauopathy. These findings indicate that OPN concomitantly regulates multiple disease-escalating factors (i.e., neuritic plaques and neurofibrillary tangles) that contribute to AD pathology. Without being bound by theory, correlation of results in AD mouse models and AD patients indicates OPN as a promising therapeutic target that will successfully translate to the clinic.

Taken together, this report distinguishes a pathogenic subset of microglia from protective microglia by expression of CD11c and production of OPN. Without being bound by theory, the CD11c⁺ OPN⁻ microglial subset are protective, since they efficiently take up Aβ without concomitant TNF-α production and express high level of TREM2, while CD11c⁺ OPN⁺ microglia represent a pathogenic subset which produces substantial amounts of TNF-α and express low levels of TREM2. Without wishing to be bound by theory, the pathogenic impact of this OPN-producing CD11c⁺ microglial subset, including promotion of a pro-inflammatory response and inhibition of a protective TREM2-lysosomal phagocytic pathway, reduces plaque diffusion and toxicity. As summarized above, levels of CD11c⁺ OPN-producing microglia, first defined in the murine 5XFAD model, positively correlate with disease severity and neuropathology in AD patients. Hence, targeting the microglial OPN response can be a more effective therapeutic strategy than current antibody-based approaches that target both forms of amyloid plaque.

### Methods

### Mice

C57BL/6 (B6) and B6. Cg-Tg (APPSwFlLon, PSEN1*M146L*L286V)6799Vas/Mmjax (5XFAD) mice were obtained from the Jackson Laboratory (MMRRC). B6. Spp1^{flstop} (OPN-KO) mice was previously generated by our lab¹⁵ Spp1^{flstop} P mice were crossed with 5XFAD mice to generate OPN-KO.5XFAD mice. Age-matched and sex-balanced mice were used. All the mice were housed in pathogen-free conditions. All experiments were performed in compliance with federal laws and institutional guidelines as approved by the Animal Care and Use Committee of the Dana-Farber Cancer Institute (DFCI).

### Microglial Isolation

Cell isolation by magnetic-activated cell sorting (MACS). Adult mice were anesthetized with isoflurane followed by transcardial perfusion using ice-cold PBS. Brains were then minced and subjected to enzymatic dissociation using Collagenase type IV (300 U/ml, Worthington) or Papain (20 U/ml, Worthington) and DNase I (60 U/ml, Worthington). Myelin was removed by 30% Percoll gradient. MACS buffer (PBS pH 7.2, 2 mM EDTA and 0.5% BSA) was then used to resuspend the pelleted cells. The anti-mouse CD11b microbeads (Miltenyi) were used for magnetic isolation of total microglial population. This method was used to isolate microglia for all of the experiments except otherwise noted. Anti-mouse ACSA-2 microbeads (Miltenyi) were used for isolation of astrocytes. Neurons were negatively enriched using anti-CD11b, anti-ACSA-2 and anti-04 microbeads (Miltenyi) to deplete microglia, astrocytes or oligodendrocytes.

Microglia isolation and fluorescence-activated cell sorting for RNA-seq analysis. Single cell suspension was prepared as previously described^{9,48}. Briefly, mice were anesthetized with isoflurane and transcardially perfused using ice-cold PBS. Brains were quickly dissected and minced using a scalpel on ice followed by Dounce homogenization in ice-cold Hank's balanced salt solution (HBSS). Cell suspension was passed through a 70-μm cell strainer followed by spinning down at 500 g for 5 min at 4° C. Myelin and debris were removed using 10 ml of ice-cold 40% Percoll (Sigma) and spun for 30 min at 500 g before washing with 10 ml of ice-cold HBSS and spun again for 5 min at 500 g at 4° C. All samples were then resuspended in ice-cold FACS buffer (0.5% BSA, 1 mM EDTA, in 1×PBS) for staining. Ghost dye Violet 510 (1:1000, Tonbo Biosciences) and anti-CD16/CD32 antibody (2.4G2, 1:100, BD Biosciences) were used to exclude dead cells and to block Fc receptors, respectively. The single cell suspension was then stained with anti-CD11b (M1/70, 1:100, Biolegend), anti-CD45 (30-F11, 1:100, Biolegend) and anti-CD11c (N418, 1:50, Biolegend) antibodies for 20 min on ice. Samples were then washed with ice-cold FACS buffer and spun down for 5 min at 500 g. The cell pellets were resuspended in 5 ml of FACS buffer then sorted on a BD FACS Aria II using the 70-μm nozzle with purity mode and a sorting speed of approximately 10,000 events per second. After sorting, each sample was spun down, and cell pellets were immediately stored at −80° C. until further processing. For 9-mo old 5XFAD mice, 2 replicates of CD11c⁺ microglia and CD11c⁻ microglia pooled from 44 mice (22 male+22 female) were sequenced. For 9-mo old OPN-KO.5XFAD mice, 2 replicates of CD11c⁺ microglia pooled from 27 mice (13 male+14 female) were sequenced.

### Flow Cytometry

Microglial flow cytometry analysis. The Ghost dye Violet 510 (1:1000, Tonbo Biosciences) and anti-CD16/CD32 antibody (2.4G2, 1:100, BD Biosciences) were used to exclude the dead cells and to avoid non-specific staining, respectively. Microglia were stained with appropriate surface markers including anti-CD11b (M1/70, 1:100 Biolegend), anti-CD45 (30-F11, 1:100 Biolegend), anti-CD11c (N418, 1:50 Biolegend), anti-αV integrin (RMV-7, 1:50, Biolegend), anti-β3 integrin (2C9.G2, 1:50, Biolegend), anti-β5 integrin (KN52, 1:50, eBioscience), anti-CD44 integrin (IM7, 1:100, BD Bioscience), anti-TREM2 (237920, 1:20, Novus Biologicals) followed by fixation and permeabilization with Intracellular Fixation & Permeabilization buffer kit (eBioscience) for the subsequent intracellular staining with anti-OPN (IC808, 1:10, R&D Systems) and anti-TNF-α (MP6-XT22, 1:50, Biolegend). Data were acquired on CytoFLEX LX (Beckman Coulter) and analyzed with FlowJo (Tree Star).

Validation of microglial CD11c expression. Validation of microglial CD11c expression was performed as previously described⁹. Briefly, brain single cell suspension from 9-mo old 5XFAD mice were stained with Ghost dye Violet 510 (1:1000, Tonbo Biosciences) and anti-CD16/CD32 antibody (2.4G2, 1:100, BD Biosciences) followed by incubation with anti-CD11b (M1/70, 1:100, Biolegend), anti-CD45 (30-F11, 1:100, Biolegend), anti-CD11c (N418, 1:50, Biolegend), anti-TMEM119 (106-6, 1:200, ABCAM), anti-CCR2 (SA203G11, 1:100, Biolegend). CD11b⁺ cells were gated from single/live cells followed by subsequent gating of CD11b⁺CD45^{low} as microglia and CD11b⁺CD45^{high} as macrophage. The microglial-specific marker Tmem119⁴⁹ and CCR2 that expressed by blood-derived macrophage, but not expressed by microglia were included to distinguish microglia and macrophage^{50,51} The CD11 b⁺CD45^{high} cells that express CCR2 but not Tmem119 are confirmed as macrophages, while the CD11b⁺CD45^{low} cells that all express Tmem119, but do not express CCR2 are confirmed as microglia. Fluorescence minus one (FMO) negative control was included to confirm the specificity of CD11c staining in CD11b⁺CD45^{low} microglial population. Brain CD45⁻ cells that mainly contain non-immune cells that do not express CD11c was also included as negative controls to further validate the staining specificity.

Real-time quantitative PCR (RT-qPCR). Microglial RNA was extracted using RNeasy Plus Universal Mini Kit per the manufacturer's instructions (QIAGEN). Complementary DNA (cDNA) was then reverse transcribed using 100 ng of RNA with a High-Capacity cDNA Reverse Transcription Kit (Applied Biosystems) according to manufacturer's instructions. Real-time quantitative PCR for Spp1 was performed with the QuantStudio™ 6 Flex Real-Time PCR System (Applied Biosystems) using 5 μl of cDNA, 4.92 μl PowerUp™ SYBR™ Green Master Mix (Applied Biosystems) and 0.08 ul primer (IDT, working concentration: 200 nM) per reaction. The gene expression level of Spp1 was compared using the ^{ΔΔ}Ct method normalized to β-actin.

### Water T-Maze

The animal cognitive tests were conducted in the NeuroBehavior Laboratory, Harvard Institute of Medicine. The Water T-maze (WTM) behavioral paradigm assesses spatial learning and memory by training mice to use the spatial cues in a room to navigate to a hidden platform to escape water. The test also measures cognitive flexibility through a reversal learning procedure in which mice must learn a new location of the hidden platform. The test was performed as previously described⁵². The testing apparatus is a plus maze (each arm 14 cm length, 4.6 cm width) made of clear Plexiglass with each arm designated as north (N), south (S), east (E) or west (W). A divider was placed on the maze to block off the appropriate arm so that the mouse could choose only the E or W arm for escape. Mice are placed in the N or S arms, in a semi-random order, at the start of each trial. The maze was filled with water (25-26° C.) and an escape platform was placed on the E side of the maze submerged about 1 cm below the surface of the water. To ensure that the mice could not see the submerged platform, the water was made opaque by adding white, nontoxic paint. At the start of the trial, the divider was put in place to block off the appropriate arm and mice were carried to the appropriate start point. The experimenter scored a correct or incorrect response for each trial and mice were allowed to stay on the platform for 10 s before being removed. Mice were given 10 trials per day and the percentage correct responses is calculated by averaging correct responses across the 10 trials for each day. Then the platform was moved to the opposite side and the same procedure was repeated for the reversal trial until the mice had learned the new position of the platform.

### RNA-Seq Analysis of CD11c Microglia

RNA extraction. Total RNA was extracted from FACS-sorted cell pellets using RNeasy Plus Universal Mini Kit following manufacturer's instructions (QIAGEN). Library preparations, sequencing reactions and bioinformatic analysis (gene hit counts) were conducted at GENEWIZ, LLC. (South Plainfield, NJ, USA) as follows:

Library preparation. Concentration of extracted RNA was quantified using Qubit 2.0 Fluorometer (Life Technologies, Carlsbad, CA, USA) and RNA integrity was determined using Agilent TapeStation 4200 (Agilent Technologies, Palo Alto, CA, USA). Due to very limited cell numbers of CD11c⁺ microglia that can be obtained from adult mice, microglial samples of 9-mo old 5XFAD and OPN-KO.5XFAD mice were processed with SMART-Seq v4 Ultra Low Input Kit for Sequencing for full-length cDNA synthesis and amplification (Clontech, Mountain View, CA), and Illumina Nextera XT library was used for sequencing library preparation. Briefly, cDNA was fragmented, and adaptor was added using Transposase, followed by limited-cycle PCR to enrich and add index to the cDNA fragments. The final library was assessed with Agilent TapeStation.

HiSeq sequencing. The sequencing libraries were clustered on flowcell lanes. After clustering, the flowcell was loaded on the Illumina HiSeq instrument 4000 according to manufacturer's instructions. Samples were sequenced using a 2×150 bp Paired End (PE) configuration. Image analysis and base calling were conducted by the HiSeq Control Software (HCS). Raw sequence data (.bcl files) generated from Illumina HiSeq was converted into fastq files and de-multiplexed using Illumina's bcl2fastq 2.17 software. One mismatch was allowed for index sequence identification.

RNA-Seq Data Analysis. Mapping and gene counting were performed by GENEWIZ (South Plainfield, NJ, USA). After quality checking of the raw data, sequence reads were trimmed to remove adapter sequences and nucleotides with poor quality using Trimmomatic v.0.36. The trimmed reads were mapped to the Mus musculus reference genome available on ENSEMBL using the STAR aligner v.2.5.2b. The STAR aligner is a splice aligner that detects splice junctions and incorporates them to help align the entire read sequences. BAM files were generated as a result of this step. Unique gene hit counts were calculated by using feature Counts from the Subread package v.1.5.2. Only unique reads that fell within exon regions were counted. Significant differentially expressed genes (DEGs) were then detected using a negative binomial model implemented in the R package edgeR^{53,54}. Gene expression was considered upregulated if log₂ FC>0.5 or downregulated if log₂ FC<−0.5. DEGs were considered significant with a false discovery rate (FDR)-adjusted p value <0.05. FC denotes fold-change of reads per kilobase per million (RPKM) at FDR<0.05.

### Immunofluorescence Staining

Mouse brains were removed after transcardial perfusion and fixed in 4% paraformaldehyde solution (PFA, Electron Microscopy Sciences) at 4° C. overnight. After rinsing with PBS, the fixed brains were dehydrated in 30% sucrose at 4° C. overnight. The brains were then embedded in OCT compound (Sakura Finetek) and serial sagittal frozen sections (10 μm) were cut using Cryostat (CM3050S, Leica).

Brain cryosections were permeabilized with PBS containing 0.1% Triton X100 (PBS-T) for 1 h. After incubation for 1 h in a blocking solution containing 5% normal donkey serum (Jackson ImmunoResearch Lab) in PBS-T to prevent non-specific binding. Then the sections were incubated for 24 h with the appropriate primary antibodies: rabbit anti-Iba-1 (1:1000, WAKO), anti-Amyloid R (6E10, 1:1000, Biolegend), Biotin anti-mouse CD11c (N418, 1:50, Biolegend), goat anti-OPN (AF808, 1:100, R&D), anti-APP (A4, 1:1000, Millipore), sheep anti-TREM2 (AF1729, 1:10, R&D), rat anti-CD68 (FA-11, 1:100, Bio-Rad), goat anti-Cathepsin B (AF965, 1:100, R&D). To amplify CD11c signal, the sections were washed with PBS and incubated for 5 min with Alexa Fluor 488 streptavidin Tyramide SuperBoost Kit (Invitrogen), following the manufacturer's instructions. Sections were then incubated for 1 h with the appropriate secondary antibodies: Alexa-594-conjugated donkey anti-rabbit IgG or Alexa-488-conjugated donkey anti-mouse IgG (1:500; Invitrogen). Brain cryosections incubated without anti-CD11c primary Ab or Tyramide Signal Amplification (TSA) are included as negative controls to exclude non-specific immunofluorescent signals. DAPI (Invitrogen) was used as a nuclear counterstain (10-min incubation). For Thioflavin-S staining, brain cryosections were incubated in filtered 1% aqueous Thioflavin-S for 10 min at room temperature and washed 2×3 min in 80% ethanol and 3 min in 95% ethanol, prior to adding blocking solution. Samples were analyzed under Olympus fluorescence microscope. Quantification of fluorescent microscopy images were performed using ImageJ (NIH).

### In Vitro Microglia Assay

To test the impact of OPN-αVβ3 interaction on CD11c⁺ microglial pro-inflammatory response, microglia were enriched by anti-CD11b microbeads (purity >95%, Miltenyi) from 9-mo old OPN-KO.5XFAD mice and were seeded into 12-well plates at 3×10⁵ cells/well in conventional microglia culture medium (DMEM-F12 with 10% fetal bovine serum+1% penicillin/streptomycin+10 ng/ml recombinant mouse M-CSF). Microglia were pre-incubated with a selective αVβ3 inhibitor, Cilengitide (10 μM) for 1 h followed by the addition of 12.5 pg/ml recombinant mouse OPN (rmOPN) and cultured for 24 h before analysis of CD11c⁺ microglial expression of TNF-α by flow cytometry.

To test the impact of OPN on TREM2-lysosome activation and AR degradation, microglia enriched by anti-CD11b microbeads (purity >95%, Miltenyi) from 9-mo old 5XFAD and OPN-KO.5XFAD mice were seeded into 12-well plates at 3×10⁵ cells/well in conventional microglia culture medium (DMEM-F12 with 10% fetal bovine serum+10% penicillin/streptomycin+10 ng/ml recombinant mouse M-CSF). Microglia were then pre-incubated with anti-OPN Ab (10 μg/ml) for 1 h followed by the incubation with rmOPN (12.5 pg/ml) overnight. Then FAM-labeled Aβ₁₋₄₂ peptide (1 μM) was added into culture for 1 h. Mean fluorescence intensity (MFI) of FAM-Aβ₁₋₄₂ in lysosomes of CD11c⁺ microglia (CD11c⁺CD68⁺) after 1 h's incubation was determined by flow cytometry and defined as Aβ MFI₁ₕ. FAM-Aβ₁₋₄₂ was then withdrawn, and cells were cultured for 24 h in the presence or absence with anti-OPN Ab (10 μg/ml) and/or rmOPN (12.5 μg/ml) before analysis of CD11c⁺ microglial expression of TREM2, CD68 and MFI of retained FAM-Aβ₁₋₄₂ in CD11c⁺CD68⁺ microglia (defined as Aβ MFI₂₄ₕ) by flow cytometry. Aβ degradation rate was calculated as (Aβ MFI₁ₕ−Aβ MFI₂₄ₕ)/Aβ MFI₁ₕ.

### Human Brain Sample Processing and Assessment

Clinical and postmortem diagnoses, brain harvesting procedures, tissue processing and assessment of AD pathology are described in detail elsewhere²⁹. Brain tissue of middle frontal gyrus (a region affected early by AD)²⁹ of clinically and neuropathologically characterized AD patients and controls were obtained from the Mount Sinai Brain Bank through the Neurobiobank (https://neurobiobank.nih.gov/). Three groups of decedents were included in this study: cognitively normal (n=11), mild cognitive impairment (MCI, n=10) and AD patients (n=11). Tables 2, 3 describe this cohort in detail. Cognitive status at death was ascertained using the clinical dementia rating (CDR) scale⁵⁵. The CDR assesses cognitive and functional impairments associated with dementia and provides specific severity criteria for classifying subjects as non-demented (CDR=0), questionably demented (CDR=0.5), or increasing levels of severity of dementia from CDR=1 to CDR=3. Participants with an acute neurological condition such as stroke or traumatic brain injury were excluded.

Plaque and tangle assessment in human brains. Neuritic plaques and neurofibrillary tangles assessment were conducted at the Mount Sinai brain bank according to the Consortium to Establish a Registry for Alzheimer's Disease (CERAD) protocol⁵⁶. Sections from paraffin embedded blocks were variably stained with hematoxylin and eosin, modified Bielschowski, modified thioflavin S, and anti-amyloid (4G8), anti-tau (AD2). All neuropathology data regarding the extent and distribution of neuropathologic lesions were collected in a blinded fashion relative to the subject's dementia status. Each case was assigned a Braak AD-staging score for progression of neurofibrillary neuropathology^{57,58}. In addition, quantitative data regarding the density of neuritic plaques were collected as described³⁰

Human OPN ELISA. For quantification of human brain OPN concentration, 10 mg of human brain frozen tissue were homogenized in 0.3 ml lysis buffer [20 mM Tris-Hcl pH8, 130 mM NaCl, 1% triton X100 and protease inhibitor cocktail (Roche)], kept on ice for 45 min and centrifuged at 13000 g at 4° C. for 20 min. Protein concentration of the brain lysate was measured using a BCA kit (ThermoFisher Scientific) and 10 pg protein was loaded into each well of a human OPN Quantikine ELISA plate (R&D Systems). Procedure was conducted following manufacturer's instructions.

Human brain staining for CD11c⁺ OPN⁺ microglia. Paraffin embedded brain tissue sections, from the middle frontal gyrus, were deparaffinized in xylene, rehydrated in graded ethanol and washed with PBS containing 0.1% Triton X100 (PBS-T). Brain sections were boiled in 10 mM citric acid buffer (pH 6) for antigen retrieval and washed with PBS-T. The endogenous peroxidase activity of the samples was quenched using 3% hydrogen peroxide solution for 60 min, washed and incubated with Zyblack (Zytovision, BS-0002-8) for 30 min to reduce auto fluorescence. Endogenous biotin was blocked with Biotin-Blocking Kit (Invitrogen, E21390). Staining was performed by multiplexing three Tyramide SuperBoost kits (Invitrogen, B40936, B40912, B40923). First, sections were blocked using blocking buffer for 60 min, then incubated with primary antibodies [biotinylated anti-OPN 1:50 (R&D systems, BAF1433), anti CD11c 1:150 (Novus, NBP2-44598) and anti Iba-1 1:500 (Wako, 019-19741)] at 4° C. overnight. After washing, sections were incubated with HRP-conjugated streptavidin for 60 min, washed and incubated with Alexa Fluor™ 647 Tyramide streptavidin reagent for 10 min, followed by reaction stop solution for 5 min. This was followed by superboost kits Alexa Fluor™ 488 tyramid anti-mouse and Alexa Fluor™ 555 tyramid anti-rabbit. Cell nuclei were marked with Hoechst and slides were mounted with Immu-Mount medium (Thermofisher Scientific).

Brain slices of control (n=5), MCI (n=9) and AD (n=8) were coded and 10-12 images were captured under a confocal microscope (Leica DMi8), with the same conditions of lasers intensities, at three separate sequences; laser 408 nm at 5%, lasers 488 nm at 0.1%, laser 638 nm at 6.5% and laser 552 nm at 3% exposures using magnification of X400. Triple positive cells expressing Iba-1, CD11c and OPN were counted, as well as total Iba-1 positive cells at each slice using ImageJ software (NIH). The percent of CD11c⁺ OPN⁺ microglia (CD11c⁺ OPN⁺Iba-1⁺) out of total Iba-1 positive cells, was calculated for each slice and an average was calculated for each brain sample. Analysis was performed by a blind investigator.

### Statistics and Reproducibility

Data are presented as mean±s.e.m. Statistical analysis was performed using GraphPad Prism software version 9.0. Analysis for comparing multiple groups was performed by one-way or two-way ANOVA with Bonferroni's test. Comparisons between two groups were analyzed with two-tailed Student's t test. The correlation analysis was performed using Pearson correlation. P<0.05 was considered to denote significance and expressed as *P<0.05, **P<0.01, ***P<0.001, ****P<0.0001. The statistical details, including the specific statistical tests used in different analyses, as well as the respective sample sizes, are given in the respective figure legends.

### Data Availability

RNA-seq data for CD11c⁺ microglia, CD11c⁻ microglia from 9-mo old 5XFAD and CD11c⁺ microglia from 9-mo old OPN-KO.5XFAD mice have been deposited to NCBI-Gene Expression Omnibus (GEO) under accession number GSE191118. Data are available upon reasonable request from the authors.

### REFERENCES CITED IN THIS EXAMPLE

Heneka, M. T. et al. Neuroinflammation in Alzheimer's disease. Lancet Neurol 14, 388-405, doi:10.1016/51474-4422(15)70016-5 (2015).
Keren-Shaul, H. et al. A Unique Microglia Type Associated with Restricting Development of Alzheimer's Disease. Cell 169, 1276-1290 e1217, doi:10.1016/j.cell.2017.05.018 (2017).
Huang, Y. et al. Microglia use TAM receptors to detect and engulf amyloid beta plaques. Nat Immunol, doi:10.1038/s41590-021-00913-5 (2021).
Spangenberg, E. et al. Sustained microglial depletion with CSF1R inhibitor impairs parenchymal plaque development in an Alzheimer's disease model. Nat Commun 10, 3758, doi:10.1038/s41467-019-11674-z (2019).
Li, Q. et al. Developmental Heterogeneity of Microglia and Brain Myeloid Cells Revealed by Deep Single-Cell RNA Sequencing. Neuron 101, 207-223 e210, doi:10.1016/j.neuron.2018.12.006 (2019).
Safaiyan, S. et al. White matter aging drives microglial diversity. Neuron, doi:10.1016/j.neuron.2021.01.027 (2021).
Sala Frigerio, C. et al. The Major Risk Factors for Alzheimer's Disease: Age, Sex, and Genes Modulate the Microglia Response to Abeta Plaques. Cell Rep 27, 1293-1306 e1296, doi:10.1016/j.celrep.2019.03.099 (2019).
Mathys, H. et al. Single-cell transcriptomic analysis of Alzheimer's disease. Nature 570, 332-337, doi:10.1038/s41586-019-1195-2 (2019).
Shen, X. Q., Y.; Wight, A.E.; Kim, H.-J.; Cantor, H. Definition of a mouse microglial subset that regulates neuronal development and pro-inflammatory responses in the brain. Proc Natl Acad Sci USA, In press (2022).
Patarca, R. et al. Structural and functional studies of the early T lymphocyte activation 1 (Eta-1) gene. Definition of a novel T cell-dependent response associated with genetic resistance to bacterial infection. Journal of Experimental Medicine 170, 145-161 (1989).
Ashkar, S. et al. Eta-1 (osteopontin): an early component of Type 1(cell-mediated) immunity. Science 287, 860-864 (2000).
Shinohara, M. L., Kim, H. J., Kim, J. H., Garcia, V. A. & Cantor, H. Alternative translation of Osteopontin generates intracellular and secreted isoforms that mediate distinct biological activities in dendritic cells. Proc. Natl. Acad. Sci. U.S.A. 105, 7235-7239 (2008).
Hur, E. M. et al. Osteopontin-induced relapse and progression of autoimmune brain disease through enhanced survival of activated T cells. Nat. Immunol 8, 74-83 (2007).
Steinman, L. New targets for treatment of multiple sclerosis. J Neurol Sci 274, 1-4, doi:10.1016/j.jns.2008.06.040 (2008).
Leavenworth, J. W., Verbinnen, B., Yin, J., Huang, H. & Cantor, H. A p85alpha-osteopontin axis couples the receptor ICOS to sustained Bcl-6 expression by follicular helper and regulatory T cells. Nat Immunol 16, 96-106, doi:10.1038/ni.3050 (2015).
Ulrich, J. D. et al. ApoE facilitates the microglial response to amyloid plaque pathology. J Exp Med 215, 1047-1058, doi:10.1084/jem.20171265 (2018).
McKee, A. C., Kosik, K. S. & Kowall, N. W. Neuritic pathology and dementia in Alzheimer's disease. Ann Neurol 30, 156-165, doi:10.1002/ana.410300206 (1991).
Nelson, P. T. et al. Correlation of Alzheimer disease neuropathologic changes with cognitive status: a review of the literature. J Neuropathol Exp Neurol 71, 362-381, doi:10.1097/NEN.0b013e31825018f7 (2012).
Terry, R. D. et al. Physical basis of cognitive alterations in Alzheimer's disease: synapse loss is the major correlate of cognitive impairment. Ann Neurol 30, 572-580, doi:10.1002/ana.410300410 (1991).
Koffie, R. M., Hyman, B. T. & Spires-Jones, T. L. Alzheimer's disease: synapses gone cold. Mol Neurodegener 6, 63, doi:10.1186/1750-1326-6-63 (2011).
McAlpine, C. S. et al. Astrocytic interleukin-3 programs microglia and limits Alzheimer's disease. Nature, doi:10.1038/s41586-021-03734-6 (2021).
Santamaria, M. H. & Corral, R. S. Osteopontin-dependent regulation of Th1 and Th17 cytokine responses in Trypanosoma cruzi-infected C57BL/6 mice. Cytokine 61, 491-498, doi:10.1016/j.cyto.2012.10.027 (2013).
Sakamoto, K. et al. Osteopontin in Spontaneous Germinal Centers Inhibits Apoptotic Cell Engulfment and Promotes Anti-Nuclear Antibody Production in Lupus-Prone Mice. J Immunol 197, 2177-2186, doi:10.4049/jimmunol.1600987 (2016).
Weber, G. F. et al. Phosphorylation-dependent interaction of osteopontin with its receptors regulates macrophage migration and activation. J Leukoc Biol 72, 752-761 (2002).
Wang, S. et al. Anti-human TREM2 induces microglia proliferation and reduces pathology in an Alzheimer's disease model. J Exp Med 217, doi:10.1084/jem.20200785 (2020).
Yuan, P. et al. TREM2 Haplodeficiency in Mice and Humans Impairs the Microglia Barrier Function Leading to Decreased Amyloid Compaction and Severe Axonal Dystrophy. Neuron 92, 252-264, doi:10.1016/j.neuron.2016.09.016 (2016).
Chapman, H. A., Riese, R. J. & Shi, G. P. Emerging roles for cysteine proteases in human biology. Annu Rev Physiol 59, 63-88, doi: 10.1146/annurev.physiol.59.1.63 (1997).
Mueller-Steiner, S. et al. Antiamyloidogenic and neuroprotective functions of cathepsin B: implications for Alzheimer's disease. Neuron 51, 703-714, doi:10.1016/j.neuron.2006.07.027 (2006).
Haroutunian, V., Katsel, P. & Schmeidler, J. Transcriptional vulnerability of brain regions in Alzheimer's disease and dementia. Neurobiol Aging 30, 561-573, doi:10.1016/j.neurobiolaging.2007.07.021 (2009).
Haroutunian, V. et al. Regional distribution of neuritic plaques in the nondemented elderly and subjects with very mild Alzheimer disease. Arch Neurol 55, 1185-1191, doi:10.1001/archneur.55.9.1185 (1998).
Sarlus, H. & Heneka, M. T. Microglia in Alzheimer's disease. J Cin Invest 127, 3240-3249, doi:10.1172/JC190606 (2017).
Zhao, Y. et al. TREM2 Is a Receptor for beta-Amyloid that Mediates Microglial Function. Neuron 97, 1023-1031 e1027, doi:10.1016/j.neuron.2018.01.031 (2018).
Kleinberger, G. et al. TREM2 mutations implicated in neurodegeneration impair cell surface transport and phagocytosis. Sci Transl Med 6, 243ra286, doi:10.1126/scitranslmed.3009093 (2014).
Hanayama, R. et al. Identification of a factor that links apoptotic cells to phagocytes. Nature 417, 182-187, doi:10.1038/417182a (2002).
Kawabe, K., Takano, K., Moriyama, M. & Nakamura, Y. Microglia Endocytose Amyloid beta Through the Binding of Transglutaminase 2 and Milk Fat Globule EGF Factor 8 Protein. Neurochem Res 43, 41-49, doi: 10.1007/s11064-017-2284-y (2018).
Wang, Y. et al. TREM2-mediated early microglial response limits diffusion and toxicity of amyloid plaques. J Exp Med 213, 667-675, doi:10.1084/jem.20151948 (2016).
Joshi, P. et al. TREM2 modulates differential deposition of modified and non-modified Abeta species in extracellular plaques and intraneuronal deposits. Acta Neuropathol Commun 9, 168, doi:10.1186/s40478-021-01263-x (2021).
Parhizkar, S. et al. Loss of TREM2 function increases amyloid seeding but reduces plaque-associated ApoE. Nat Neurosci 22, 191-204, doi:10.1038/s41593-018-0296-9 (2019).
Wang, Y. et al. TREM2 lipid sensing sustains the microglial response in an Alzheimer's disease model. Cell 160, 1061-1071, doi:10.1016/j.cell.2015.01.049 (2015).
Baik, S. H., Kang, S., Son, S. M. & Mook-Jung, I. Microglia contributes to plaque growth by cell death due to uptake of amyloid beta in the brain of Alzheimer's disease mouse model. Glia 64, 2274-2290, doi:10.1002/glia.23074 (2016).
Liu, Y. H. et al. An N-terminal antibody promotes the transformation of amyloid fibrils into oligomers and enhances the neurotoxicity of amyloid-beta: the dust-raising effect. J Neuroinflammation 12, 153, doi:10.1186/s12974-015-0379-4 (2015).
Panza, F., Lozupone, M., Logroscino, G. & Imbimbo, B. P. A critical appraisal of amyloid-beta-targeting therapies for Alzheimer disease. Nat Rev Neurol 15, 73-88, doi:10.1038/s41582-018-0116-6 (2019).
Long, J. M. & Holtzman, D. M. Alzheimer Disease: An Update on Pathobiology and Treatment Strategies. Cell 179, 312-339, doi:10.1016/j.cell.2019.09.001 (2019).
Zhou, Y. et al. Human and mouse single-nucleus transcriptomics reveal TREM2-dependent and TREM2-independent cellular responses in Alzheimer's disease. Nat Med 26, 131-142, doi:10.1038/s41591-019-0695-9 (2020).
Grubman, A. et al. A single-cell atlas of entorhinal cortex from individuals with Alzheimer's disease reveals cell-type-specific gene expression regulation. Nat Neurosci 22, 2087-2097, doi:10.1038/s41593-019-0539-4 (2019).
Lau, S. F., Cao, H., Fu, A. K. Y. & Ip, N. Y. Single-nucleus transcriptome analysis reveals dysregulation of angiogenic endothelial cells and neuroprotective glia in Alzheimer's disease. Proc Natl Acad Sci USA 117, 25800-25809, doi:10.1073/pnas.2008762117 (2020).
Sun, Y. et al. Elevated osteopontin levels in mild cognitive impairment and Alzheimer's disease. Mediators of inflammation 2013, 615745, doi:10.1155/2013/615745 (2013).
Hammond, T. R. et al. Single-Cell RNA Sequencing of Microglia throughout the Mouse Lifespan and in the Injured Brain Reveals Complex Cell-State Changes. Immunity 50, 253-271 e256, doi:10.1016/j.immuni.2018.11.004 (2019).
Bennett, M. L. et al. New tools for studying microglia in the mouse and human CNS. Proc Natl Acad Sci USA 113, E1738-1746, doi:10.1073/pnas.1525528113 (2016).
Mizutani, M. et al. The fractalkine receptor but not CCR2 is present on microglia from embryonic development throughout adulthood. J Immunol 188, 29-36, doi:10.4049/jimmunol.1100421 (2012).
Wlodarczyk, A. et al. A novel microglial subset plays a key role in myelinogenesis in developing brain. EMBO J 36, 3292-3308, doi:10.15252/embj.201696056 (2017).
Ofengeim, D. et al. RIPK1 mediates a disease-associated microglial response in Alzheimer's disease. Proc Natl Acad Sci USA 114, E8788-E8797, doi:10.1073/pnas.1714175114 (2017).
Robinson, M. D., McCarthy, D. J. & Smyth, G. K. edgeR: a Bioconductor package for differential expression analysis of digital gene expression data. Bioinformatics 26, 139-140, doi:10.1093/bioinformatics/btp616 (2010).
McCarthy, D. J., Chen, Y. & Smyth, G. K. Differential expression analysis of multifactor RNA-Seq experiments with respect to biological variation. Nucleic Acids Res 40, 4288-4297, doi:10.1093/nar/gks042 (2012).
Marin, D. B. et al. Reliability and validity of a chronic care facility adaptation of the Clinical Dementia Rating scale. Int J Geriatr Psychiatry 16, 745-750, doi:10.1002/gps.385 (2001).
Mirra, S. S. et al. The Consortium to Establish a Registry for Alzheimer's Disease (CERAD). Part II. Standardization of the neuropathologic assessment of Alzheimer's disease. Neurology 41, 479-486, doi:10.1212/wnl.41.4.479 (1991).
Braak, H. & Braak, E. Neuropathological stageing of Alzheimer-related changes. Acta Neuropathol 82, 239-259, doi:10.1007/BF00308809 (1991).
Braak, H., Alafuzoff, I., Arzberger, T., Kretzschmar, H. & Del Tredici, K. Staging of Alzheimer disease-associated neurofibrillary pathology using paraffin sections and immunocytochemistry. Acta Neuropathol 112, 389-404, doi:10.1007/s00401-006-0127-z (2006).

### Example 3

### Example 3—Features of Angiopep-2 Ab

Although antibody has been modified by numerous methods to address bottlenecks in delivery into various tissues, methods to penetrate the blood-brain-barrier (BBB) currently represent the most difficult obstacle. One approach is based on conjugation of Angiopep-2 peptide to host proteins, including immunoglobulin, to increase penetration across the endothelial lining of the brain. In principle, receptor-dependent transcytosis of Angiopep-2 might facilitate passage of its cargo through the BBB (via an interaction with low-density lipoprotein receptor-related protein 1 [LRP-1]) 1. However, in our hands, this method has not been satisfactory, in terms of its impact on brain penetration and effects on the ability of transcytosed antibody to bind antigen in the brain.

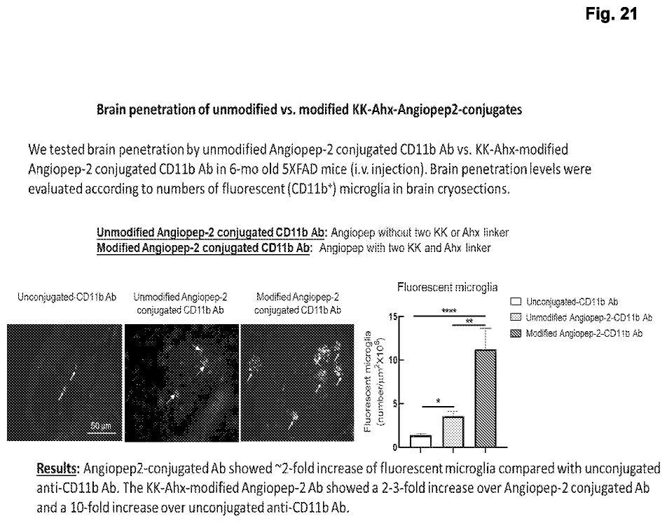

Although several patents use Angiopep-2 to accelerate penetration of small molecules or multiple peptides into the brain, we were able to identify only one patent (U.S. Pat. No. 9,161,988) that uses Angiopep-2 Ab-conjugates to enhance BBB penetration. We tested this method according to brain penetration by tagged Angiopep-conjugated Ab and the antigen binding ability of the penetrated Ab (neither of these issues are addressed in the above-mentioned U.S. Pat. No. 9,161,988). We noted relatively weak activity (FIG. 21). We reasoned that modifying the positive-charge of Angiopep2-Ab might enhance the interaction with negatively-charged endothelial cells lining the brain. Without being bound by theory, increasing the flexibility of the Angiopep-2-Ab conjugation bridge decreases its aggregation potential and render the conjugated Ab more functional. These considerations resulted in an approach to this problem that embodies the features discussed below. In short, we designed a KK modification and Ahx linker to enhance brain penetration of Angiopep2-conjugated mAb.

First, we add two lysines (-kk-, pI 9.74) to the bridge between Ab and angiopep-2 to increase the isoelectric point (pI) of the conjugated antibody (to alkaline levels). Without being bound by theory, this enhances penetration into the BBB via adsorption-mediated transport (AMT), which depends on interactions between the negative-charged surface of endothelial cells and the positive-charged substituted ligand.

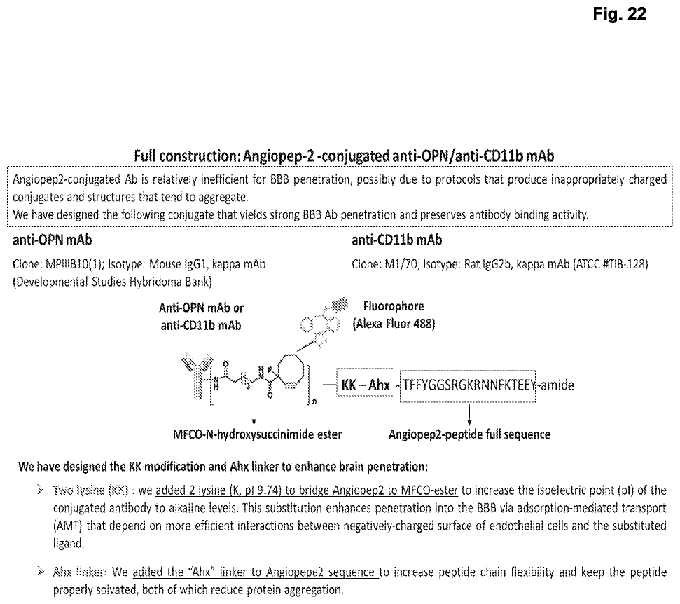

Second, we add an Ahx linker to Angiopep2 sequence. Ahx can increase the flexibility of peptide chains and keep them appropriately solvated to prevent aggregation and associated reduction in receptor-mediated transcytosis (RMT) and functional penetration by Angiopep-2 Ab (FIG. 22).

### REFERENCE CITED IN THIS EXAMPLE

Demeule, M. et al. Involvement of the low-density lipoprotein receptor-related protein in the transcytosis of the brain delivery vector angiopep-2. J Neurochem 106, 1534-1544, doi:10.1111/j.1471-4159.2008.05492.x (2008).

### Example 4

### Example 4—Development of Engineered Brain-Penetrating Monoclonal Antibody (mAb) targeting Osteopontin (OPN) for Alzheimer's disease therapy

Recent clinical trials of anti-amyloid-D (AD) antibodies have reported significant reductions in plaque load without appreciable cognitive improvement. These disappointing results have accelerated efforts to identify and validate new drug targets that may halt or reverse disease progression and cognitive decline. An alternative approach is indicated from the recent division of Aβ plaques into two subsets: a diffuse, invasive subset of neurotoxic plaques that impairs cognition and a dense subset that may represent non-toxic products of microglial processing and compaction of AR fibrils (Huang et al., 2021; Parhizkar et al., 2019; Yuan et al., 2016). If microglial packing of AR fibrils into dense plaques is a neuroprotective mechanism, generalized disaggregation of dense plaques by antibody may be counterproductive. These considerations also indicate that therapeutic enhancement of microglial plaque compaction may represent a more effective therapeutic strategy than current approaches that indiscriminately target plaques in mAb trials.

We have defined the Spp1 gene (encoding Osteopontin, OPN) as a new and tractable target based on preclinical studies using the 5XFAD model of Alzheimer's disease (AD) and analysis of a panel of well-characterized human brain tissues. We also have developed strategies to test the therapeutic potential of a blood-brain-barrier (BBB)-penetrating antibody specific for OPN. This anti-OPN mAb may improve cognitive function in 5XFAD mice by reducing the neurotoxic diffuse Aβ plaques and microglia inflammatory response.

### Definition of OPN as a Therapeutic Target for AD:

Although many disease-associated genes have been identified in murine models of AD, most have not been confirmed from studies of human AD. The Spp1 gene (encoding OPN) is a notable exception since it appears to be strongly upregulated by microglia in both animal models of AD and the human disease. Our recent studies indicate that genetic deletion of OPN in 5XFAD mice substantially reduces inflammatory microglia, amyloid-beta (AD) plaque area, and markedly improves cognitive function. Our mechanistic studies indicate that targeting OPN de-represses a TREM2-dependent pathway responsible for microglial uptake of Aβ fibrils, lysosome digestion and extrusion into brain parenchyma (Huang et al., 2021; Keren-Shaul et al., 2017; Parhizkar et al., 2019; Yuan et al., 2016) to reduce dissemination of toxic Aβ oligomers throughout the brain.

We have used clinically and neuropathologically characterized brain tissue from AD patients and controls (Mt. Sinai Brain Bank) to test the validity of these murine findings in the human disease. OPN production by human microglia correlates closely with both AD severity and pre-mortem assessment of cognitive impairment, as judged from a comparison of brain material from AD patients and healthy controls (in collaboration with Dr. Michal Beeri, Mt. Sinai). Increasing numbers of OPN-producing microglia from AD brain sections closely correlate with progressive dementia, as measured by the Clinical Dementia Rating (CDR) scale. Based on these studies, blockade of OPN may decrease proinflammatory microglia, promotes lysosomal degradation and associated detoxification of Aβ plaque, thereby to improve cognitive function.

### Generation and Testing of a Monoclonal Anti-OPN Ab Engineered to Enhance Brain Penetration:

We have produced a fluorophore-labeled (AF488) mAb after conjugation of monoclonal anti-OPN Ab (MPIIIB10) to chemically-modified Angiopep-2 peptide (Ang2pep). Ang2pep mediates receptor-dependent transcytosis and passage through the blood-brain barrier (BBB) via an interaction with low-density lipoprotein receptor-related protein 1 (LRP-1) expressed by brain endothelial cells. This strategy will allow OPN blockade for therapeutic purpose in vivo.

What Features Distinguish the Disclosed Approach?What Improvements or Advantages does the Disclosed Approach have Over Existing Approaches?

Our approach is based on a more precise definition of the contribution of OPN, which has been identified as part of the genetic signature in both animal models of AD and the human disease. Monoclonal Ab targeting of OPN may reduce AR pathology and improve cognitive function, in contrast to current AR-based approaches that indiscriminately target all Aβ plaques. We have engineered a brain-penetrating anti-OPN mAb to selectively target neurotoxic Aβ plaques for proof-of-principle studies and as a foundation for development of a new and effective antibody-based AD therapy.

Several features of OPN enhance the clinical feasibility of targeting this protein by mAb. OPN is an extracellular protein secreted by microglia that is easily accessed by mAb. OPN production is generally associated with pathogenic rather than protective responses. Expression of OPN in peripheral tissues contributes to several chronic disorders, including atherosclerotic and cardiovascular disease, autoimmune disease and cancer growth and metastasis, and genetic or antibody-based reduction of OPN ameliorates pathology in these clinical settings. In contrast, genetic deletion or reduction of OPN does not suppress general immune response.

What Applications of the Disclosed Approach are Envisioned?how would Those Applications Work?

OPN expression substantially increases proinflammatory microglia and dampens lysosomal degradation and associated detoxification of Aβ plaque. Efficient targeting of OPN by anti-OPN mAb is applicable to AD and other OPN-mediated neuroinflammatory diseases such as Multiple Sclerosis (MS).

Since OPN expression is upregulated in microglia during disease progression and continuing AD, antibody-based neutralization of OPN is during the window of time when OPN-mediated effects are most destructive and pathogenic.

What Other Approaches May be Used in Combination with the Disclosed Approach?

The following approaches may be used for additional enhancement of anti-OPN mAb brain penetration:
1. Cell-penetrating peptides (CPPs) are a group of short peptides, consisting of amphipathic and/or cationic sequences that allow for crossing the cell membranes. For example, trans-activator of transcription peptide (TAT) is an effective CPP which has been shown to effectively transport various therapeutic reagents into brains of murine models with different CNS disorders. Positively-charged TAT may facilitate the interactions with negatively-charged cell membranes at the BBB and trigger the internalization through adsorption-mediated transcytosis (AMT). This represents a receptor/transporter-independent pathway distinct from the Ang2pep pathway. If indicated, we will construct an Ang2pep-TAT dual-conjugated mAb to determine whether Ab-conjugates that exploit both receptor-mediated transcytosis (RMT) and adsorption-mediated transcytosis (AMT) pathways will allow increased penetration and higher Ab concentration in brain tissue.
2. A two-stage injection regimen can be used to maximize brain penetration of anti-OPN mAb. Unconjugated (“cold”) anti-OPN mAb can be injected at time 0, which will allow occupation of available OPN and Fc receptors expressed by peripheral tissues before injection of Angiopepe2-conjugated (“hot”) anti-OPN mAb thus to enhance brain penetration of this conjugated “hot” Ab.

### Describe Additional Research Plans.

We have found that genetic deletion of OPN in 5XFAD mouse model inhibits microglial production of inflammatory cytokines and promotes TREM2-dependent microglial uptake of amyloid fibrils and associated lysosomal activation, resulting in increased condensation and extrusion of non-toxic Aβ plaques. These changes are accompanied by marked improvements in cognitive function. We have engineered a brain penetrating peptide conjugated anti-OPN mAb to test its therapeutic effects in vivo use mouse models of AD. Analysis of brain penetration and therapeutic activity of conjugated anti-OPN mAb will depend on a comparison between OPNWT.5XFAD mice and OPN−/−. 5XFAD mice to distinguish specific anti-OPN binding to brain OPN from non-specific uptake of anti-OPN mAb (MPIIIB10).

To analyze brain penetration of anti-OPN mAb, brains and major peripheral organs (spleen, kidney, liver, etc.) of 5XFAD and OPN−/−.5XFAD mice injected with conjugated or unconjugated Ab will be harvested and the intensity of AF488 fluorophore will be measured using a fluorescent microplate reader to obtain raw fluorescence units (RFUs). Antibody concentrations will be quantified as percent of injected dose per gram tissue (% ID/g): % ID/g=Measured RFUs per gram brain (or tissue)/Total injected RFUs. In addition, a brain capillary depletion assay will be performed to distinguish Ang2pep-mediated transcytosis of Ab into brain parenchyme from binding to brain microvasculature, according to fluorophore (AF488) intensity in capillary-enriched fractions vs. parenchymal fractions of brain homogenates at different time points after Ab injection. Binding specificity of conjugated Ab at the optimized dose will also be determined by counter staining of OPN brain sections from 5XFAD mice and OPN−/−.5XFAD mice with a second fluorophore after injections of conjugated Ab.

The therapeutic activity of conjugated anti-OPN mAb using an optimized dosing regimen will be evaluated for its impact on Aβ plaque pathology and neuroinflammation according to Aβ plaque numbers/area, plaque compaction and number of TNF-α+ microglia. Aβ plaque load will be examined by immunofluorescence of hippocampal and cortical sections with anti-Aβ Ab 6E10 and confirmed by Aβ positron emission tomography (PET) imaging according to the mean PET standard uptake value ratio (SUVR) composite of score 18F-Florbetapir tracer that binds to Aβ plaques. Microglial lysosomal activation (CD68 lysosomal activation protein and cathepsin B enzyme) will be assessed by immunofluorescence of mouse brain cryosections. The impact of anti-OPN mAb on neuropathology will be determined according to the numbers of dystrophic neurites with labeled anti-APP Ab and, if indicated from above analysis, will be further confirmed by an examination of cognitive function using Water T maze and Novelty Y maze.

### REFERENCES CITED IN THIS EXAMPLE

Huang, Y., Happonen, K.E., Burrola, P.G., O'Connor, C., Hah, N., Huang, L., Nimmerjahn, A., and Lemke, G. (2021). Microglia use TAM receptors to detect and engulf amyloid beta plaques. Nat Immunol.
Keren-Shaul, H., Spinrad, A., Weiner, A., Matcovitch-Natan, O., Dvir-Szternfeld, R., Ulland, T.K., David, E., Baruch, K., Lara-Astaiso, D., Toth, B., et al. (2017). A Unique Microglia Type Associated with Restricting Development of Alzheimer's Disease. Cell 169, 1276-1290 e1217.
Parhizkar, S., Arzberger, T., Brendel, M., Kleinberger, G., Deussing, M., Focke, C., Nuscher, B., Xiong, M., Ghasemigharagoz, A., Katzmarski, N., et al. (2019). Loss of TREM2 function increases amyloid seeding but reduces plaque-associated ApoE. Nat Neurosci 22, 191-204.
Yuan, P., Condello, C., Keene, C.D., Wang, Y., Bird, T.D., Paul, S.M., Luo, W., Colonna, M., Baddeley, D., and Grutzendler, J. (2016). TREM2 Haplodeficiency in Mice and Humans Impairs the Microglia Barrier Function Leading to Decreased Amyloid Compaction and Severe Axonal Dystrophy. Neuron 92, 252-264.

### Example 5

### Example 5—Development of Engineered Brain-Penetrating Monoclonal Antibody (mAb) to Target Osteopontin (OPN) for Alzheimer's Disease Therapy

An example strategy for developing an engineered brain-penetrating monoclonal antibody to target OPN for Alzheimer's disease therapy is shown below.

In the working model, OPN suppression of microglial Aβ plaque compaction and promotion of inflammatory responses contribute to cognitive impairment. The definition of OPN as a therapeutic target for Alzheimer's disease (AD) comes from: i) data from 5XFAD mouse model, and ii) data from AD patients and controls (Mount Sinai brain bank). Also, a monoclonal anti-OPN Ab was engineered to enhance brain penetration.

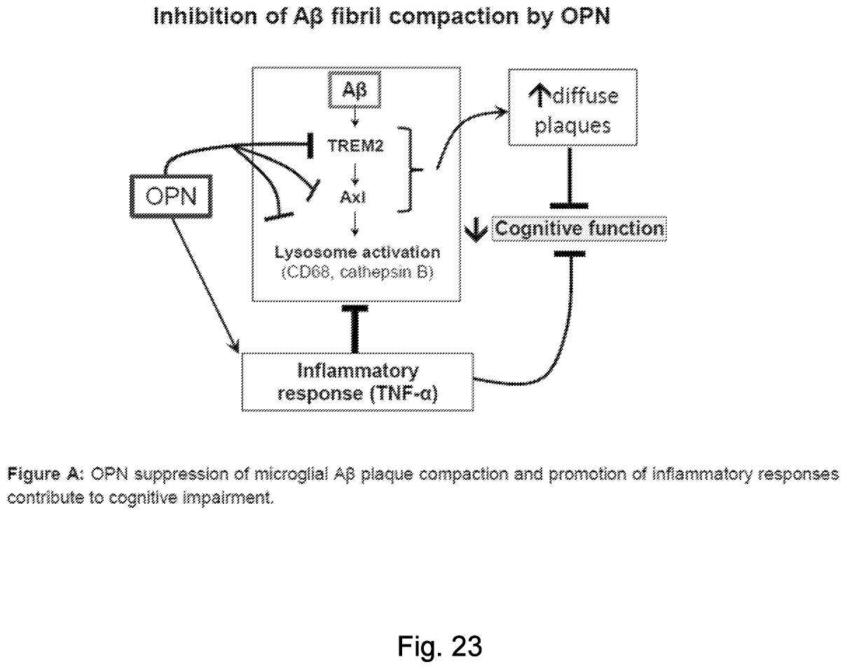

An example working model of microglial OPN expression is shown in FIG. 23. In the model, OPN expression promotes microglial proinflammatory response (TNF-α) and inhibits TREM2/Axl/lysosomal phagocytic pathway, thereby dampening lysosomal degradation and Aβ plaque compaction, which ultimately leads to cognitive impairment. The OPN-based platform described herein targets diffuse Aβ plaques and microglial proinflammatory responses to simultaneously inhibit two disease-escalating factors.

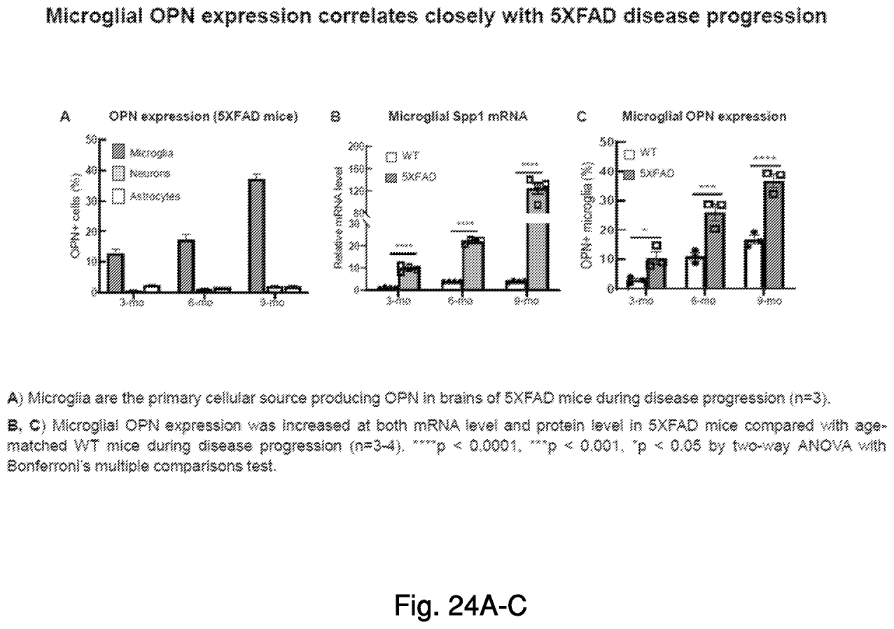

Microglia are the primary cellular source producing OPN in brains of 5XFAD mice. Microglial expression of OPN was increased at both the mRNA and protein levels in 5XFAD mice as compared with age-matched WT mice with disease progression, as shown in FIG. 24.

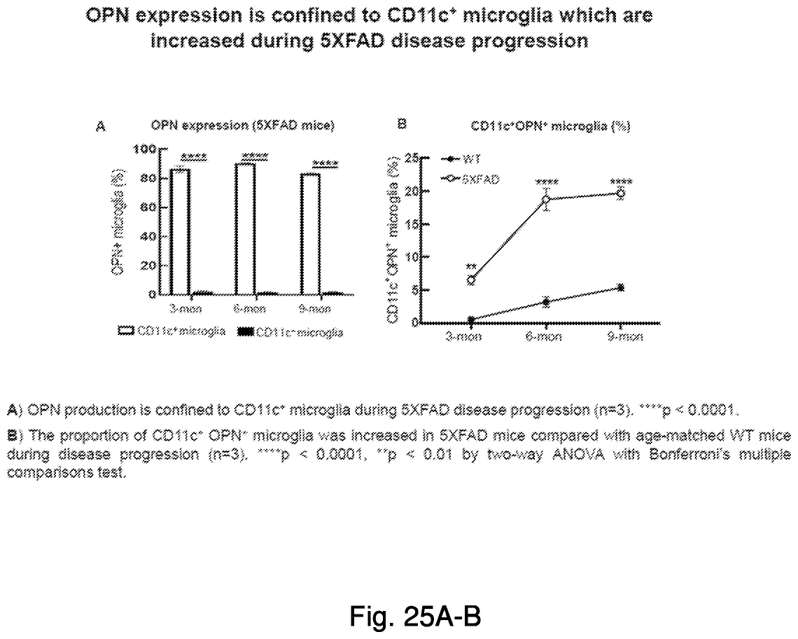

OPN expression is confined to CD11c⁺ microglia in brains of 5XFAD mice. The percentage of CD11c⁺ OPN⁺ microglia was substantially increased in 5XFAD mice compared with age-matched WT mice during disease progression (FIG. 25).

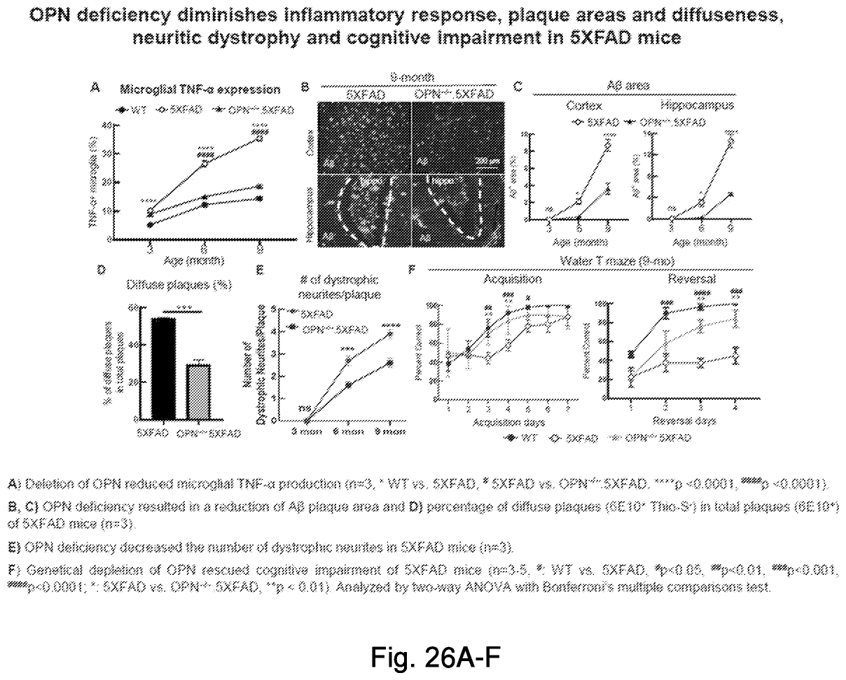

Genetic depletion of OPN reduced microglial TNF-α production, total Aβ plaque area, the ratio of diffuse plaques and the numbers of distrophic neurites, resulting in rescue of cognitive impairment (FIG. 26).

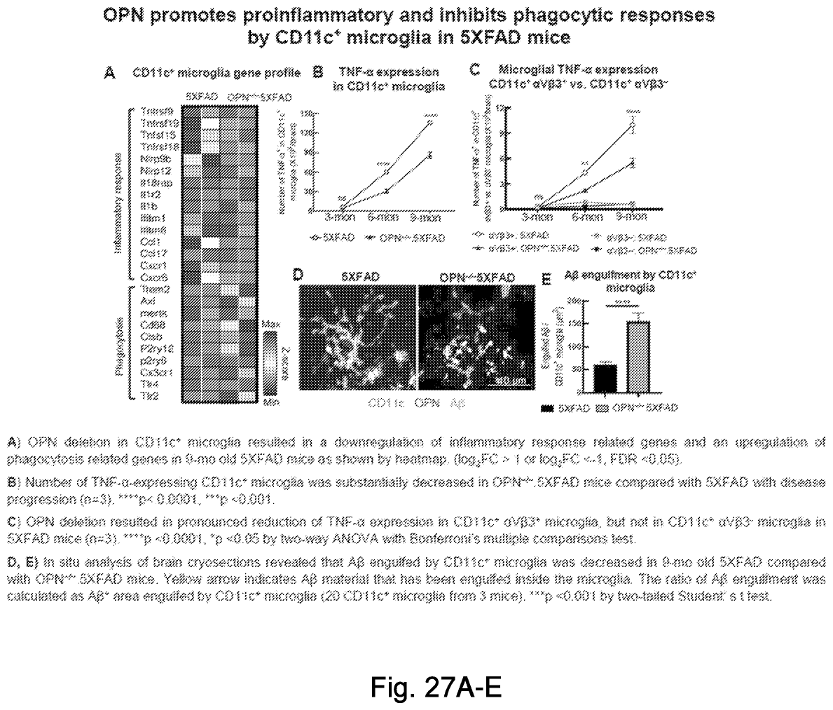

Transcriptomic analysis of CD11c⁺ microglia revealed that OPN-deletion resulted in downregulation of proinflammatory response-related genes and upregulation of phagocytosis-related genes. Genetic deletion of OPN reduced TNF-α production by αVβ3 CD11c⁺ microglia, but not by αVβ3⁻ CD11c⁺ microglia, indicating that OPN promotes microglial proinflammatory responses via its interation with the αVβ3 integrin receptor. Moreover, in-situ analysis of 5XFAD mice brain cryosections showed that OPN-deletion increased microglial engulfment of Aβ (FIG. 27).

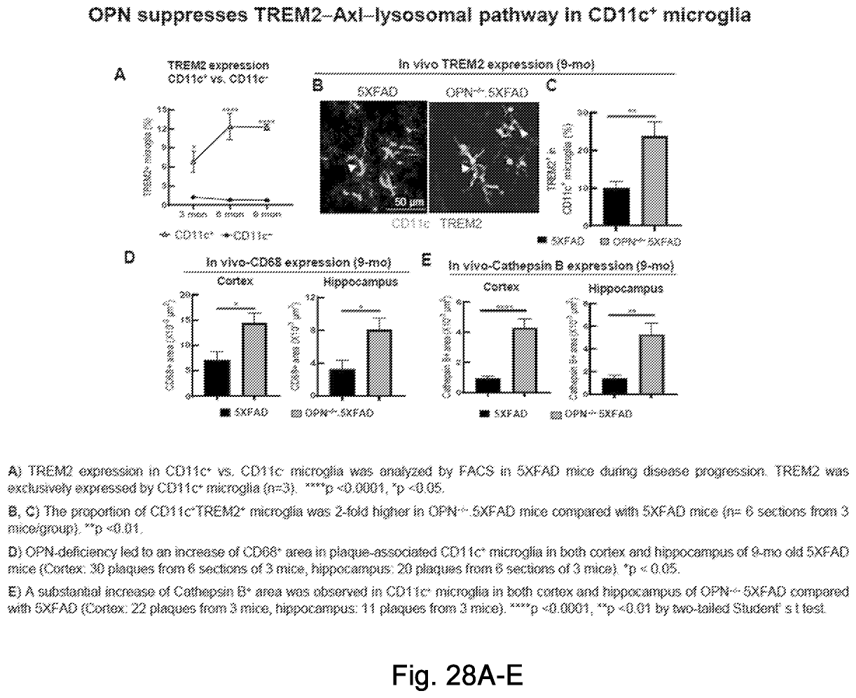

TREM2 is exclusively expressed by CD11c⁺ microglia in brains of 5XFAD mice. Genetic deletion of OPN led to an increase of TREM2 expression by CD11c⁺ microglia and activation of lysosome as judged from increased CD11c⁺ microglial expression of CD68 and cathepsin B in OPN-KO.5XFAD mice compared with 5XFAD mice, indicating that OPN suppresses the TREM2-lysosomal phagocytic pathway in CD11c⁺ microglia of 5XFAD mice (FIG. 28).

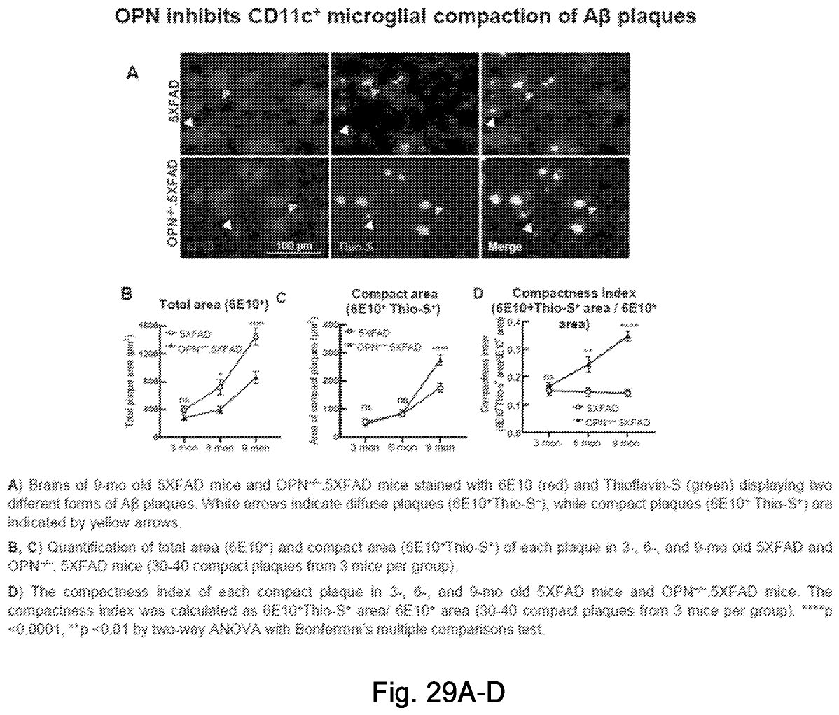

OPN-deficiency resulted in a substantial reduction of total area of Aβ plaques (6E10⁺) and an increase of compact plaque areas (6E10⁺ Thio-S⁺), as judged by a striking upregulation of compactness index of plaques (6E10⁺ Thio-S⁺ area/6E10⁺ area), indicating OPN inhibits CD11c⁺ microgial compaction of Aβ plaques (FIG. 29).

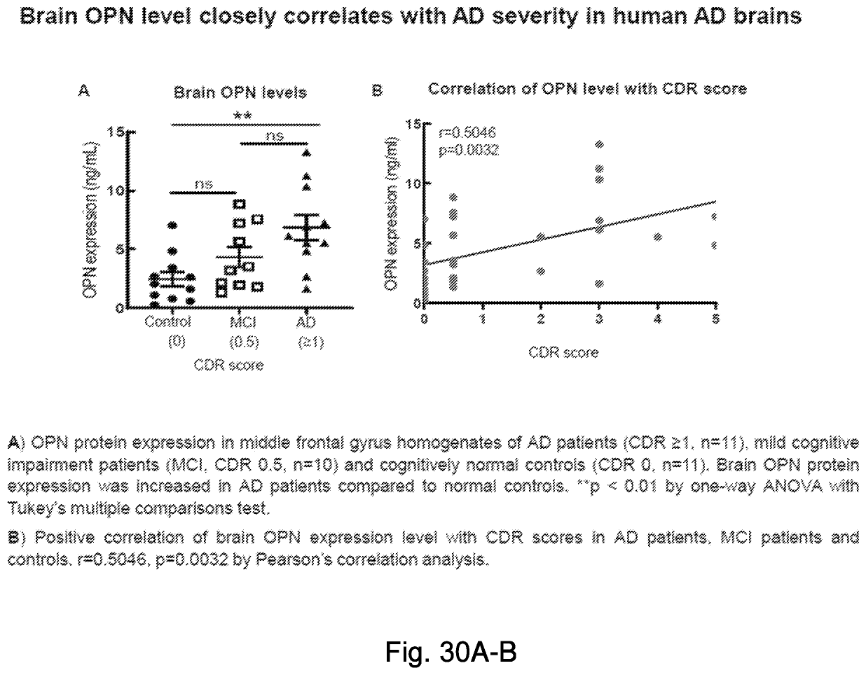

Measurements of OPN in middle frontal gyrus homogenates revealed a 3-fold increase in OPN expression in AD patient brains compared with those of cognitively normal control subjects. Increased OPN expression in AD patient brains compared with brains of patients with MCI was also noted but did not reach statistical significance. A correlative analysis of brain OPN expression and CDR score indicated that increased OPN expression correlated positively with dementia severity (FIG. 30).

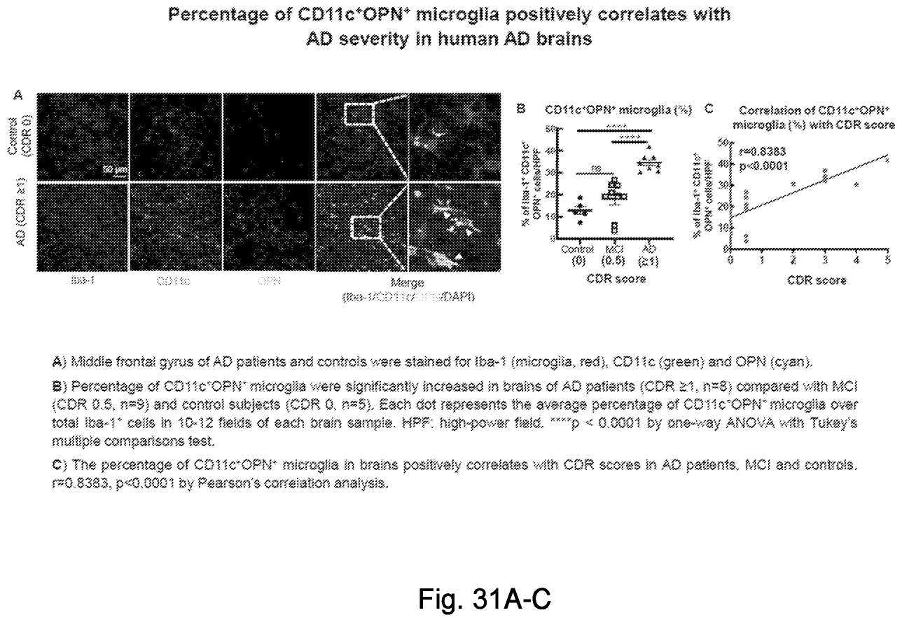

We determined the percentage of CD11c+ OPN+(double positive, DP) microglia by immunofluorescence analysis of human brain sections from the middle frontal gyrus, a region affected early by AD. Normal controls and MCI subjects displayed similar proportions of CD11c+ OPN+ microglia (CD11c+ OPN+Iba-1+). We observed a 3-fold increase of CD11c+ OPN+ microglia in brain sections from AD patients compared with sections from normal controls. Moreover, the percentage of CD11c+ OPN+ microglia from AD patients was significantly higher than that from MCI patients, indicating that the percentage of CD11c+ OPN+ microglia represents a sensitive parameter for differentiation of AD patients from patients who suffer from MCI. This conclusion is further supported by the very strong correlation of the percentage of CD11c+ OPN+ microglia with CDR scores (FIG. 31).

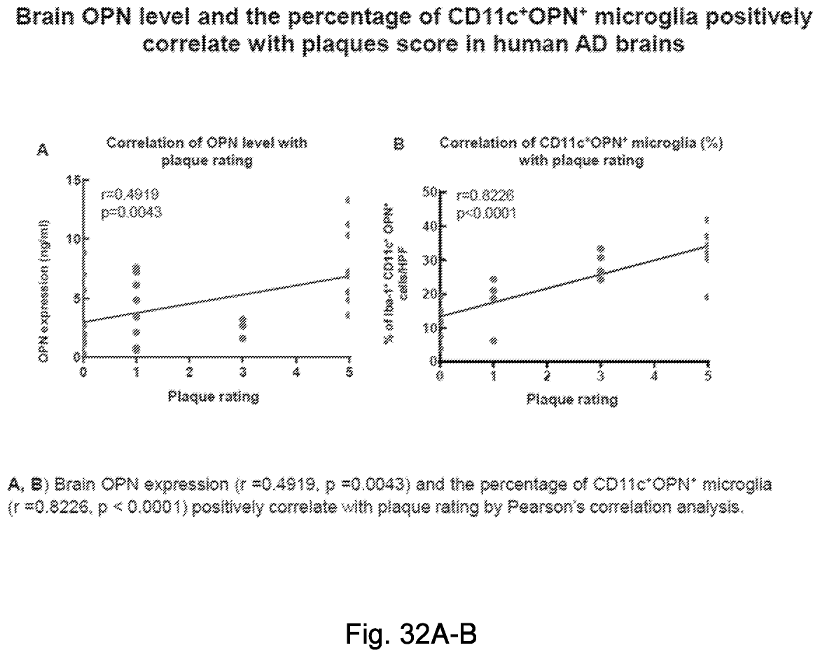

Higher brain OPN levels correlated with higher neuritic plaque levels (r=0.4919, p=0.0043). Similar to the associations found with dementia severity, there was a stronger correlation between the percentage of CD11c+ OPN+(DP) microglia and the density of neuritic plaques (r=0.8226, p<0.0001). These findings lend further support to the view that the percentage of CD11c+ OPN+ microglia may be a sensitive marker of both clinical severity and neuropathology of AD (FIG. 32).

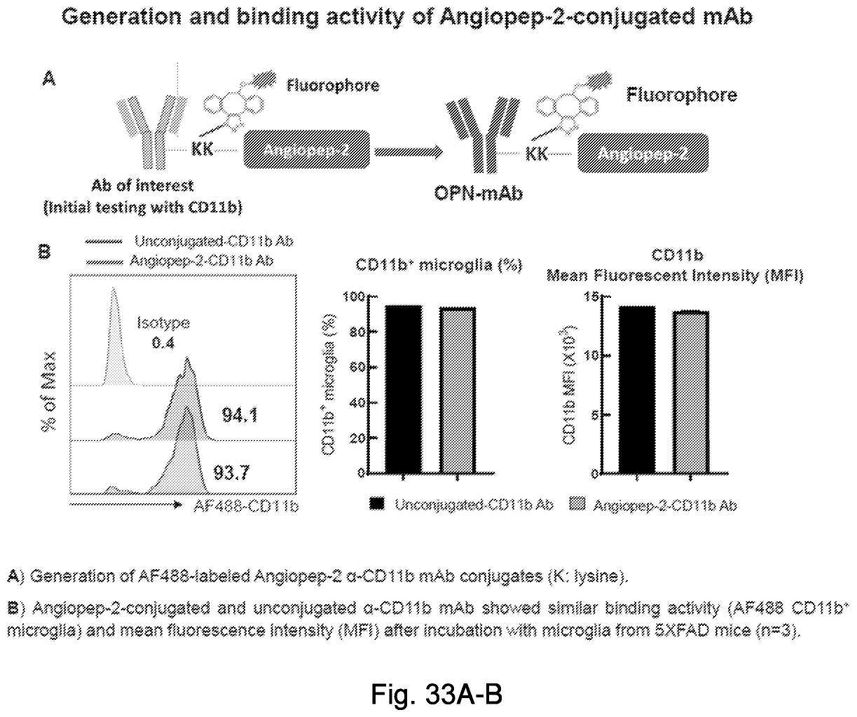

We conjugated Angiopep2 to anti-CD11b mAb and tested the binding activity of conjugated and unconjugated mAb. Angiopep2-conjugated and unconjugated anti-CD11b mAb showed similar binding activity (AF488+ microglia) and mean fluorescent intensity (MFI) after incubation with microglia from 5XFAD mice, indicating that conjugation does not alter binding activity of mAb (FIG. 33).

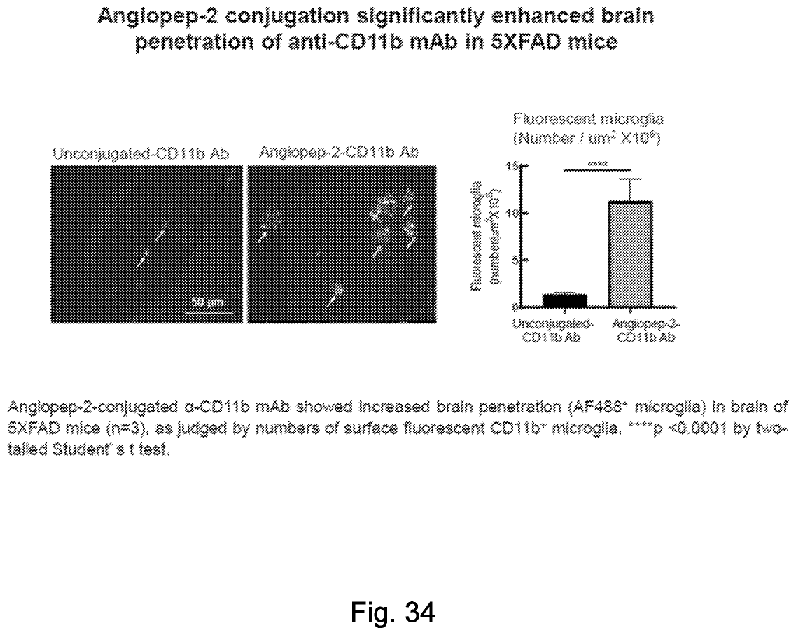

We conjugated a modified Angiopep2 peptide (containing -KK- amino acid bridge and AhX linker) to an anti-CD11b mAb. In vivo testing displayed efficient brain penetration of conjugated anti-CD11b mAb. The Angiopep-2 conjugated Ab showed a 10-fold increase over unconjugated anti-CD11b Ab (FIG. 34).

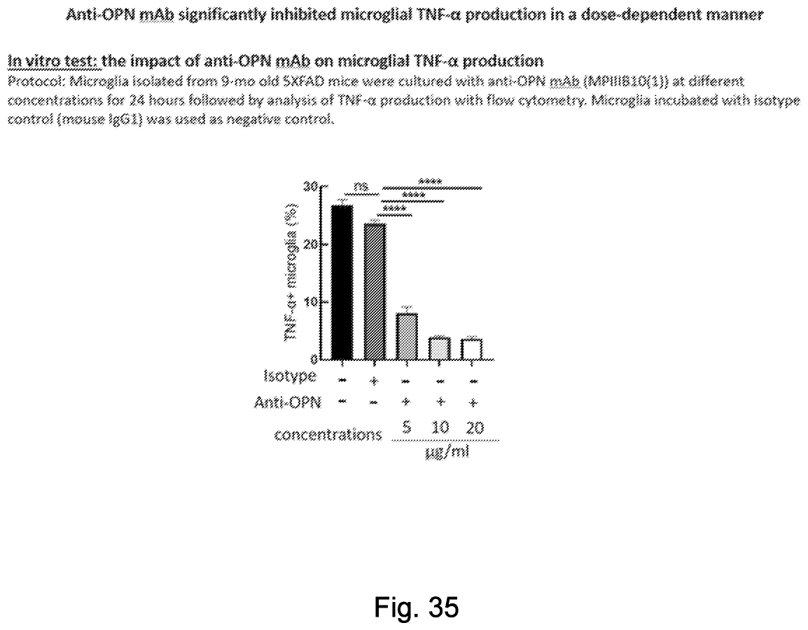

Microglia isolated from 9-mo old 5XFAD mice were incubated with anti-OPN mAb (MPIIIB10) at increasing concentrations (5, 10, 20 μg/ml) for 24 hours followed by flow cytometric analysis of TNF-α production by CD11c⁺ microglia. Microglia incubated with isotype control (mouse IgG1) were included as a negative control. Anti-OPN mAb blockade resulted in dose-dependent inhibition of TNF-α production by CD11c⁺ microglia (FIG. 35).

### Example 6

### Example 6—Exemplary Approach to Treatment of Alzheimer's Disease

Our approach to treatment of Alzheimer's disease is based, in part, on the following:

An unmet need: i) Dementia/Alzheimer's affects 55 million people (2020) and 130 affected people are projected by 2050; ii) No effective treatments. Anti-Aβ antibodies (e.g., Aducanumab) reduce plaques without cognitive improvement ($55K/year); and iii) Long-term care impact on families, healthcare systems and society.

OPN-based therapeutics: Engineered brain-penetrating anti-OPN mAb selectively removes toxic plaques and improves cognitive function.

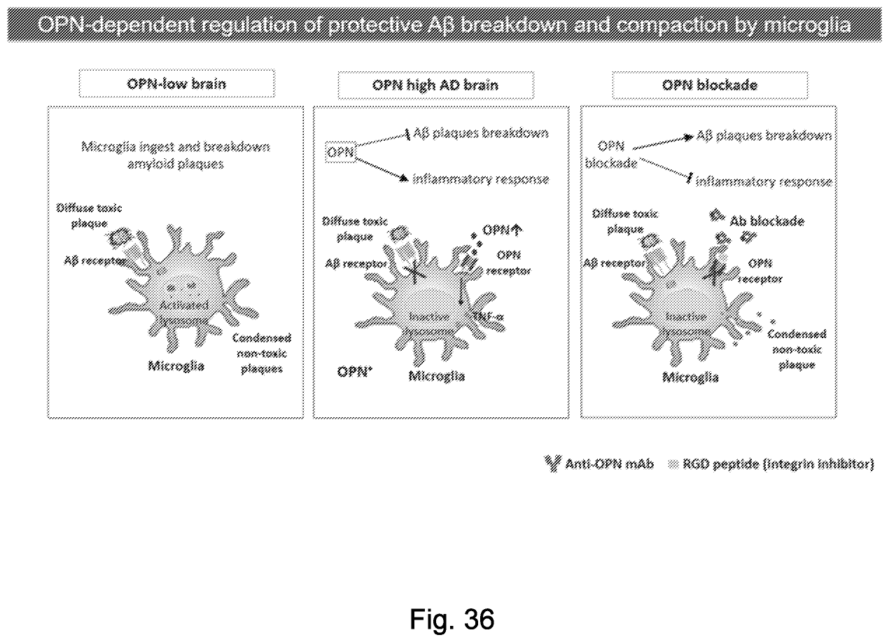

The schematic (FIG. 36) shows OPN-dependent regulation of protective A breakdown and compaction by microglia. In OPN-low brain, microglia ingest and breakdown Aβ plaques in activated lysosomes followed by extrusion of non-toxic compacted plaques into the microenviroment. In OPN high AD brain, OPN inhibits microglial uptake and compaction of toxic diffuse plaques, while promoting proinflammatory responses (TNF-α production). OPN blockade with anti-OPN mAb or integrin inhibitor inhibits microglial proinflammatory responses and enhances microglial uptake and compaction of diffuse plaques (FIG. 36).

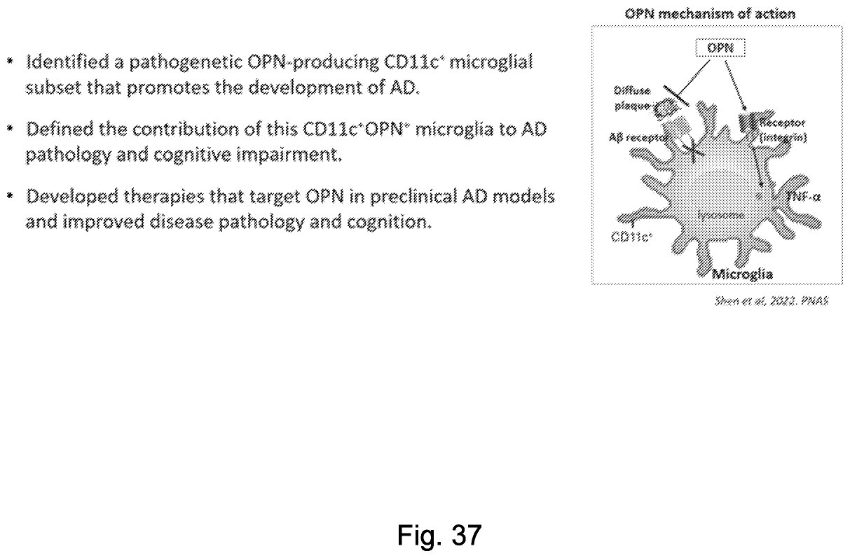

The schematic in FIG. 37 shows an example OPN mechanism of action: We identified a pathogenic OPN-producing CD11c⁺ microglial subset that promotes proinflammatory responses and inhibits microglial uptake/compaction of Aβ plaques thereby contributing to AD pathology and cogonitive impairment (FIG. 37).

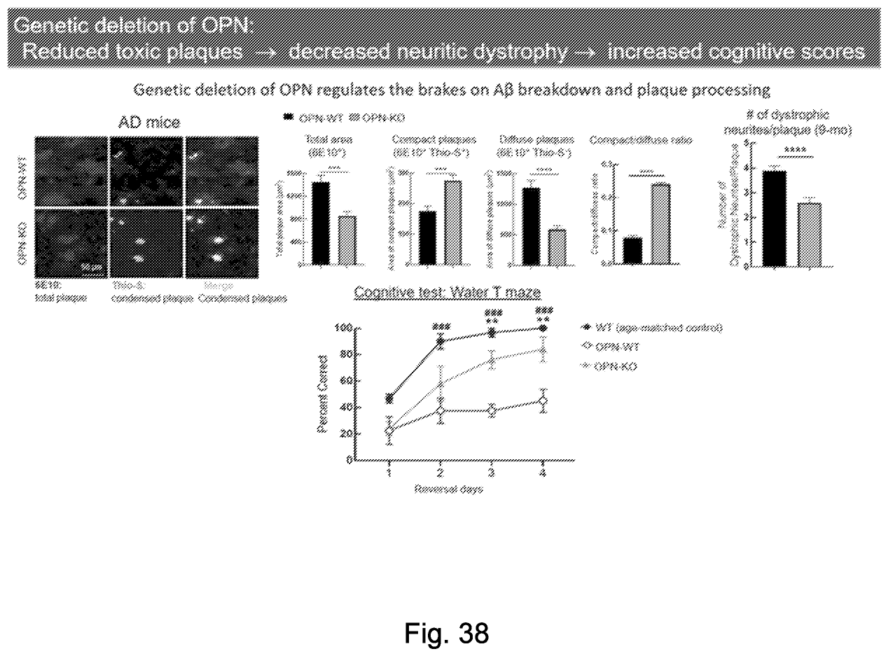

Genetic deletion of OPN leads to reduced neurotoxic plaques (6E10⁺ Thio-S⁻) and decreased numbers of dystrophic neurites, which ultimately rescued cognitive impairment of 5XFAD mice (FIG. 38).

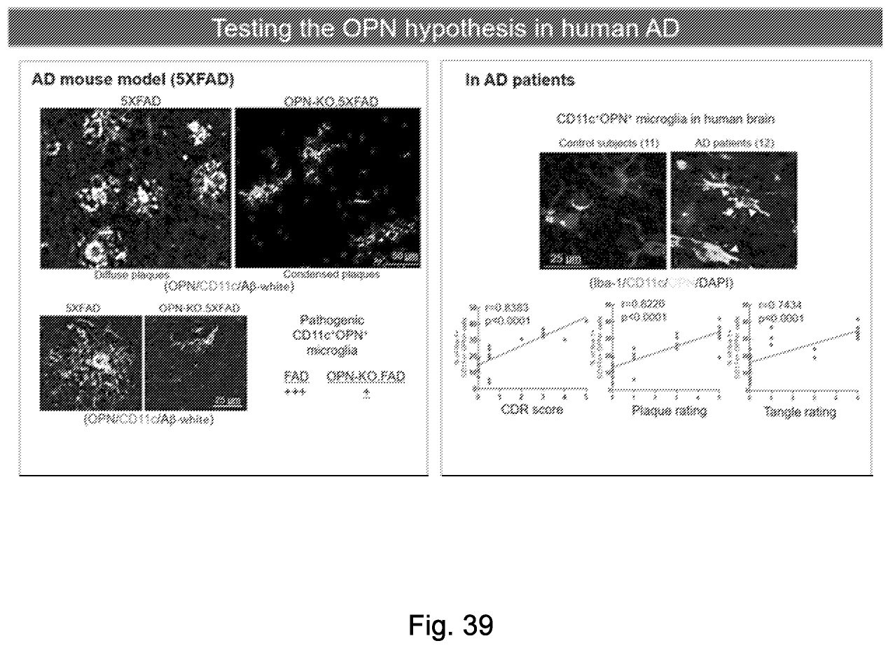

In the AD mouse model (5XFAD mice), OPN deficiency led to a substantial reduction of toxic diffuse plaques and a marked reduction of the pathogenic CD11c⁺ OPN+ microglia. These findings were validated in AD patients using clinically and neuropathogenically defined brain samples. The percentage of this pathogenic CD11c⁺ OPN⁺ microglia highly correlated with Clinical Dementia Rating (CDR) scale, neuritic plaque rating and tau tangle rating (FIG. 39).

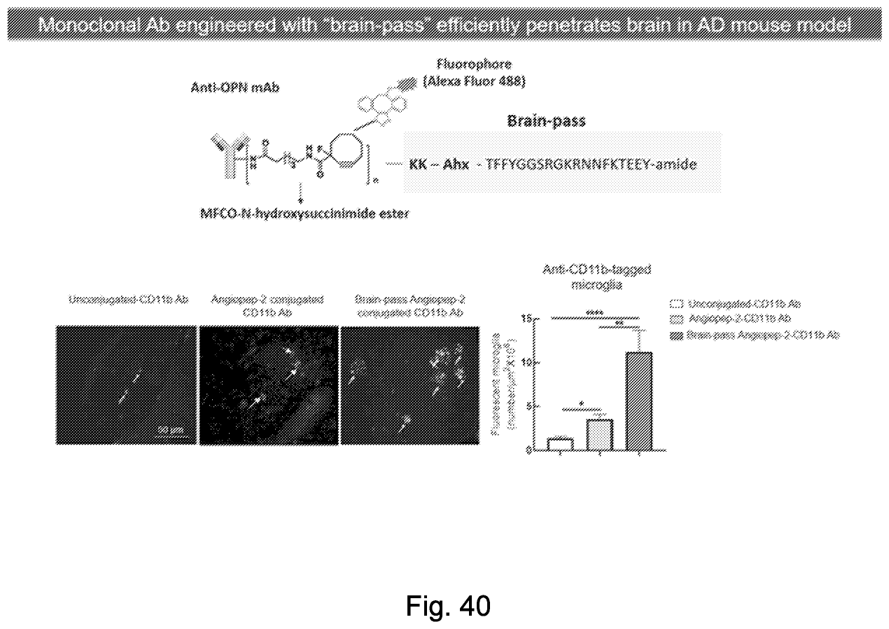

We conjugated a brain-pass Ab containing Angiopep2 peptide, -KK- amino acid bridge and AhX linker to an anti-CD11b mAb. In vivo testing displayed efficient brain penetration of brain-pass conjugated anti-CD11b mAb. Angiopep2-conjugated Ab showed ˜2-fold increase of fluorescent microglia compared with unconjugated anti-CD11b Ab. The KK-Ahx-modified Angiopep-2 Ab showed a 2-3-fold increase over Angiopep-2 conjugated Ab and a 10-fold increase over unconjugated anti-CD11b Ab (FIG. 40).

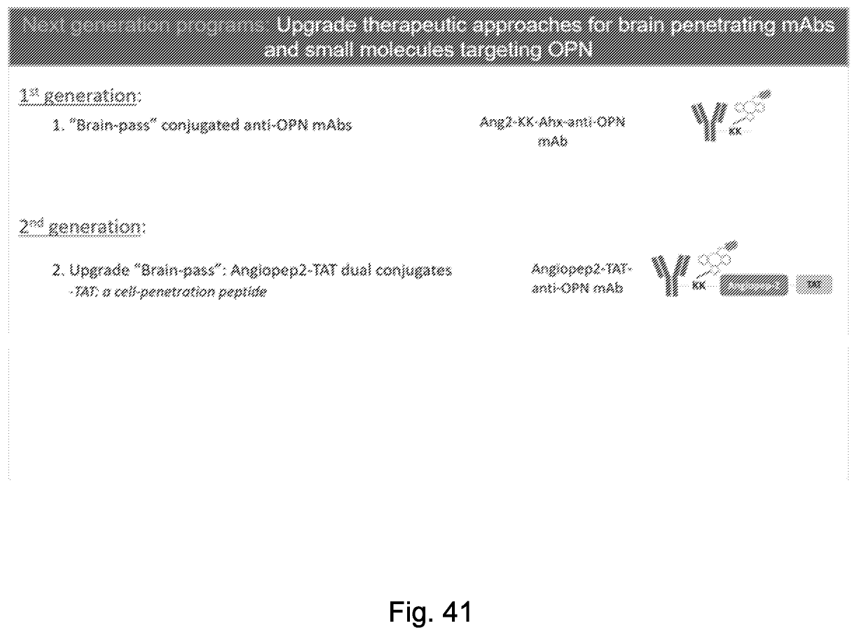

The first generation of brain-pass conjugated anti-OPN mAb entailed the conjugation of modified Angiopep2 (containing -KK- bridage and AhX linker) to anti-OPN mAb. Another conjugation strategy can generate Angiopep2-TAT dual conjugates which may further increase brain penetration of anti-OPN mAb (FIG. 41).

### Example 7

### Example 7—Administration of Anti-OPN mAb Inhibits Microglial Proinflammatory Responses and Ameliorates A6 Plaque Pathology

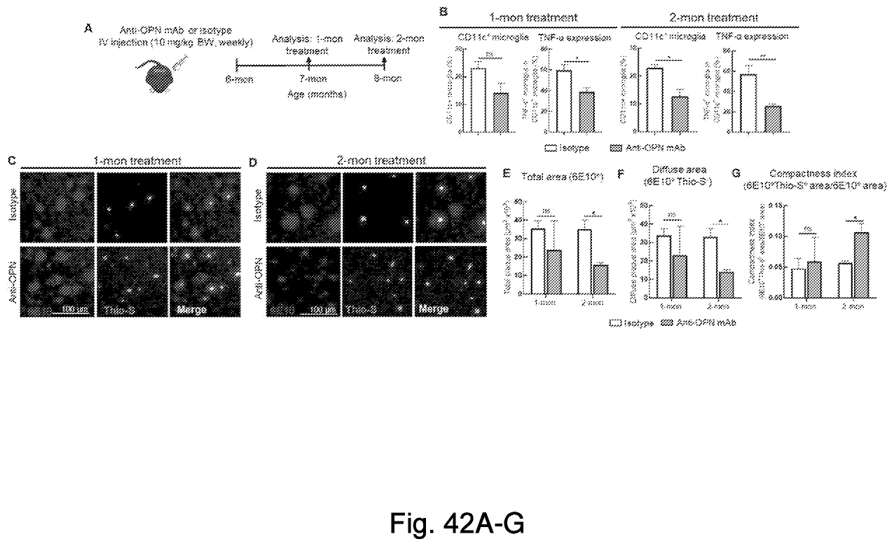

Weekly administration of anti-OPN to 5XFAD mice were given to 6-mo old mice, continuing for 1 or 2 months. Microglial proinflammatory responses and Aβ plaque pathology were analyzed after 1-mo or 2-mo of treatment (FIG. 42A).

Treatment for one month resulted in a modest reduction, and for two months resulted in a 45% reduction in CD11c+ microglia compared to control mice. Treatment for one month resulted in a 35% decrease, and for two months resulted in a 55% decrease in TNF-α expression by CD11c+ microglia compared to isotype-matched control mice (FIG. 42B).

Representative immunofluorescent images from brains of 5XFAD mice after 1-mo or 2-mo (FIGS. 42C and D). The first column of images in (C) and (D) were stained with 6E10 to identify both diffuse and condensed forms of Aβ plaques. The second column of images were stained with Thioflavin-S to identify only 0-sheet⁺ Aβ condensed plaques. The third column shows merged images of the first and second columns. Quantitation of total plaque area, diffuse plaque area and Compactness Index of plaques after 1-mo and 2-mo of treatment is shown in (E-G).

### Example 8

### Example 8—Administration of Cyclic RGD (Cilengitide) Inhibits Microglial Proinflammatory Response

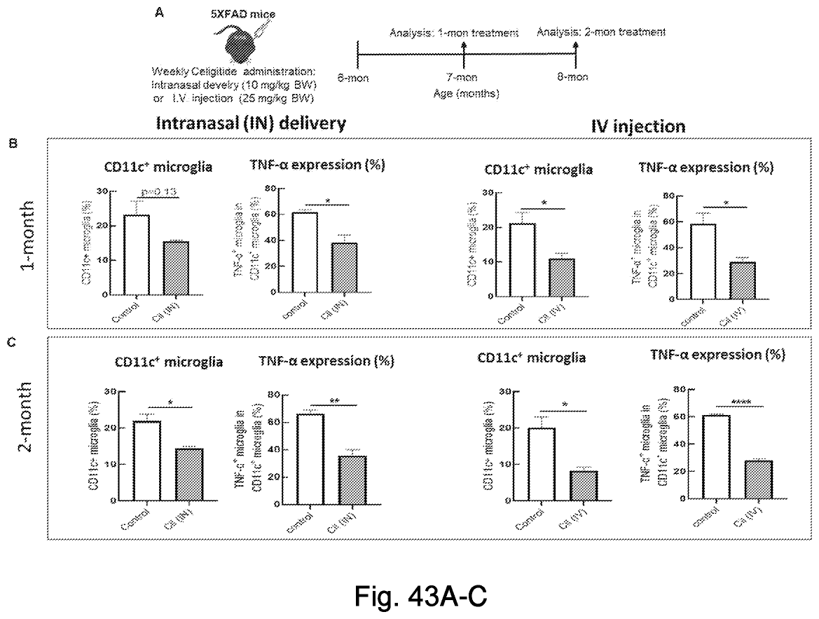

Weekly administration of cyclic RGD (Cilengitide) was given intravenously or intranasally to 6-mo old mice, continuing for 1 or 2 months. Microglial proinflammatory responses and Aβ plaque pathology were analyzed after 1-mo or 2-mo of treatment (FIG. 43A).

Intranasal Cilengitide for one month resulted in a decrease, and for two months resulted in a 35% reduction of CD11c+ microglia compared to control mice. Intranasal Cilengitide for one month resulted in an approximate 35% decrease, and for two months resulted in an approximate 45% decrease in TNF-α expression by CD11c+ microglia compared to control mice (FIGS. 43B and C).

Intravenous Cilengitide for one month resulted in an 50% decrease, and for two months resulted in an 60% reduction of CD11c+ microglia compared to control mice. Intravenous Cilengitide for one month resulted in an approximate 50% decrease, and for two months resulted in a 55% decrease in TNF-α expression by CD11c+ microglia compared to control mice (FIGS. 43B and C).

### Example 9

### Example 9—Intranasal Administration of Antibody

Generally, administration of the Angiopep-2-conjugated antibody in many of the previously-described studies was via the intravenous route. Without wishing to be bound by theory, because osteopontin is present in multiple tissues, those tissues can act as a “sink” for anti-OPN antibodies when the antibodies are administered systemically (e.g., intravenously).

In the studies described in this example, intranasal administration of conventional antibodies (i.e., antibodies not conjugated to a moiety like Angiopep-2 for delivery of an active/therapeutic agent) was tested as a way to increase delivery of the antibodies to the brain, possibly be avoiding the “sink” effect that occurs with intravenous administration of the Angiopep-2-conjugated antibodies. While not wanting to be bound by theory, intranasal delivery was tested as a way to bypass the blood brain barrier by using olfactory transfer to deliver agents to the brain.

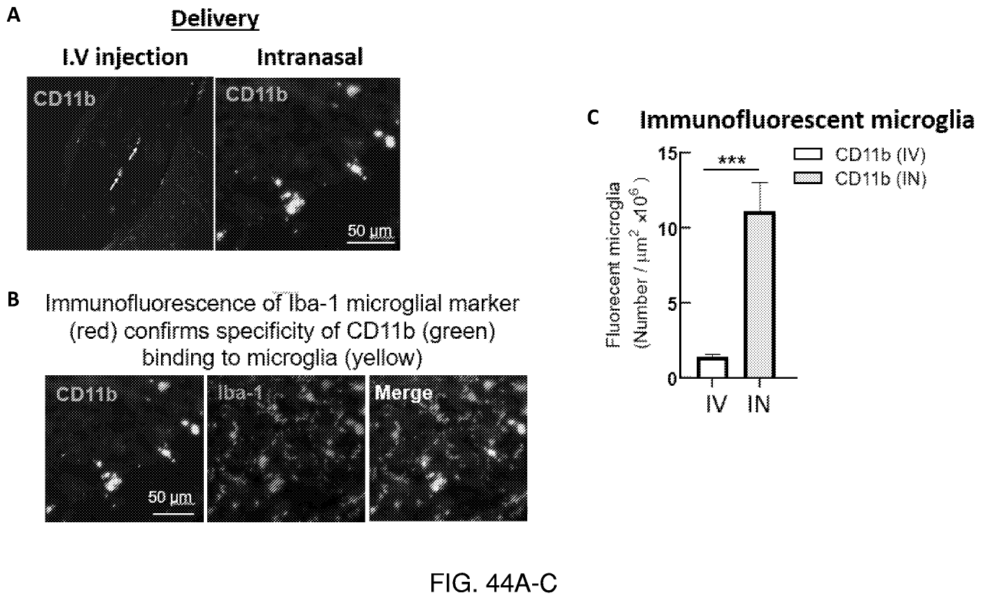

FIG. 44A-C shows intranasal delivery of conventional unconjugated anti-CD11b monoclonal antibody. Intranasal delivery of the antibody (10 mg/kg bodyweight) to 6-month-old 5XFAD mice (n=4) was performed at time 0. Antibodies were labeled with the fluorophore, AF488. Observations were obtained at 3 hours after administration. Control was intravenous injection of anti-CD11b monoclonal antibody (also unconjugated). (A) shows anti-CD11b monoclonal antibody fluorescence (green) in brain for intranasal administration versus control intravenous administration. (B) shows anti-CD11b monoclonal antibody fluorescence (green) in brain for intranasal administration, and also shows immunofluorescence of an anti-Iba-1 antibody (red). Iba-1 is a microglial marker. The merged immunofluorescence (yellow) confirms binding specificity of anti-CD11b monoclonal antibody to microglia. (C) shows data for calculation of anti-CD11b monoclonal antibody penetration into the brain in these studies, as the number of fluorescent microglia per microscope field (μm²×10⁶), for anti-CD11b monoclonal antibody administered via the intranasal route as compared to intravenous administration. The data indicate that intranasal administration of the antibody resulted in about a 10-fold increase in brain penetration compared with intravenous injection.

### Example 10

### Example 10—OPN Affects on Microglial Inflammasome Activation

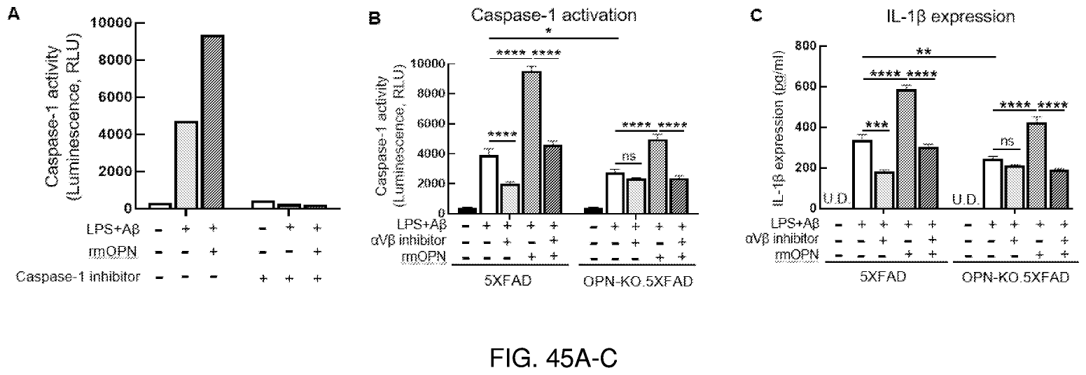

Activation of the NLRP3 (NLR family pyrin domain containing 3) inflammasome represents a major neuroinflammatory component of Alzheimer's Disease (AD) pathology in both human and mouse models of AD. We analyzed the impact of OPN on microglial inflammasome activation in vitro according to Caspase-1 activity and IL-1β production. Microglial intracellular Caspase-1 activity was determined using a bioluminescent assay which specifically detects Caspase-1 activation, confirmed by a selective Caspase-1 inhibitor (FIG. 45A). We observed that recombinant mouse OPN (rmOPN) substantially increased Caspase-1 activity and IL-10 production in LPS+Aβ-stimulated microglia from both 9-mo 5XFAD and OPN-KO.5XFAD mice. This OPN-dependent promotion of inflammasome activation was blocked by the αVβ3 inhibitor (FIGS. 45B-C), indicating that OPNdependent promotion of microglial inflammasome activation can reflect its interaction with αVβ3 integrin.

In these studies, microglia isolated from 9-mo old 5XFAD and OPN-KO.5XFAD mice were seeded into 96-well plates at 6×10⁴ cells/well in conventional microglia culture medium (DMEM-F12 with 10% fetal bovine serum+1% penicillin/streptomycin+10 ng/ml recombinant mouse M-CSF). To induce inflammasome activation, microglia were primed with 100 ng/ml LPS for 3 h followed by stimulation with 10 μM AD1-42 fibrils overnight in the presence or absence of 12.5 pg/ml rmOPN. The αVβ3 inhibitor (Cilengitide, Selleck, 10 μM) were added into culture 1 h before the addition of rmOPN. Microglial intracellular Caspase-1 activity was analyzed by the bioluminescent Caspase-Glo® 1 Inflammasome Assay Kit (Promega) per the manufacturer's instruction. The detection specificity of Caspase-1 activity was validated using a selective Caspase-1 inhibitor (Ac-YVAD-CHO (SEQ ID NO: 13), 1 μM) included in the kit. Culture medium was collected for quantitative determination of microglial IL-10 production by mouse IL-10 DuoSet ELISA kit (R&D Systems) according to the manufacturer's instructions.

### EQUIVALENTS

Those skilled in the art will recognize, or be able to ascertain, using no more than routine experimentation, numerous equivalents to the specific substances and procedures described herein. Such equivalents are considered to be within the scope of this invention and are covered by the following claims.

TABLE 1
DEGs of CD11c+ microglia in 9-mo OPN-KO.5XFAD vs 5XFAD

| Gene_id | Gene_name | Log₂FC | Adj. P-Value |
|---|---|---|---|
| ENSMUSG00000046101 | Mcmdc2 | −2.50058094 | 7.28E−04 |
| ENSMUSG00000099032 | Tcf24 | −2.16020373 | 0.021189951 |
| ENSMUSG00000067851 | Arfgef1 | −1.05319033 | 0.006621008 |
| ENSMUSG00000048960 | Prex2 | −3.98784847 | 2.25E−04 |
| ENSMUSG00000016918 | Sulf1 | −2.68142127 | 0.013470105 |
| ENSMUSG00000025938 | Slco5a1 | −3.18311037 | 0.003972101 |
| ENSMUSG00000025935 | Tram1 | 1.26195282 | 5.81E−04 |
| ENSMUSG00000025940 | Tmem70 | 1.09557966 | 0.004924841 |
| ENSMUSG00000025777 | Gdap1 | −3.7602 | 0.001076152 |
| ENSMUSG00000025931 | Paqr8 | −2.8007581 | 2.70E−05 |
| ENSMUSG00000026155 | Smap1 | −1.26728041 | 0.009358187 |
| ENSMUSG00000073725 | Lmbrd1 | 1.35383524 | 4.24E−04 |
| ENSMUSG00000033569 | Adgrb3 | −3.61556617 | 0.003837507 |
| ENSMUSG00000026117 | Zap70 | −3.09235193 | 0.002457105 |
| ENSMUSG00000026083 | Eif5b | −1.03953557 | 0.003045655 |
| ENSMUSG00000037138 | Aff3 | −3.77462821 | 0.003355591 |
| ENSMUSG00000048814 | Lonrf2 | −3.71033574 | 0.003759636 |
| ENSMUSG00000026080 | Chst10 | 1.15062283 | 0.006049416 |
| ENSMUSG00000026074 | Map4k4 | −1.13631965 | 0.001773083 |
| ENSMUSG00000026073 | Il1r2 | −3.21263267 | 2.06E−07 |
| ENSMUSG00000070942 | Il1rl2 | 1.1407167 | 0.001446014 |
| ENSMUSG00000026070 | Il18r1 | −1.75714052 | 0.046472266 |
| ENSMUSG00000026068 | Il18rap | −1.89561966 | 3.30E−04 |
| ENSMUSG00000041945 | Mfsd9 | 2.35026253 | 0.014805434 |
| ENSMUSG00000060424 | Pantr1 | −3.7959586 | 0.00282162 |
| ENSMUSG00000041907 | Gpr45 | −3.7486185 | 0.003959971 |
| ENSMUSG00000008136 | Fhl2 | −2.59191983 | 0.029726741 |
| ENSMUSG00000066877 | Nck2 | −3.40625012 | 9.40E−04 |
| ENSMUSG00000026047 | Poglut2 | 1.179563 | 0.001636767 |
| ENSMUSG00000026043 | Col3a1 | −7.41136407 | 2.81E−04 |
| ENSMUSG00000026042 | Col5a2 | −4.48121252 | 0.001924207 |
| ENSMUSG00000025993 | Slc40a1 | 1.93043523 | 1.24E−05 |
| ENSMUSG00000025986 | Slc39a10 | 1.13085832 | 0.001867849 |
| ENSMUSG00000026109 | Tmeff2 | −2.84142321 | 0.011516747 |
| ENSMUSG00000018417 | Myo1b | 1.20642545 | 0.001624852 |
| ENSMUSG00000062939 | Stat4 | −1.72484824 | 0.001358501 |
| ENSMUSG00000002881 | Nab1 | −1.31430777 | 0.009145981 |
| ENSMUSG00000101501 | Rps27a-ps1 | −3.81209399 | 0.001923241 |
| ENSMUSG00000025978 | Rftn2 | −1.61972631 | 0.001375408 |
| ENSMUSG00000038079 | Tmem237 | 1.15113658 | 0.008534924 |
| ENSMUSG00000049439 | Cyp20a1 | 1.3184099 | 5.18E−04 |
| ENSMUSG00000026014 | Raph1 | −1.48940961 | 0.003108462 |
| ENSMUSG00000025969 | Nrp2 | 1.13878019 | 0.002304931 |
| ENSMUSG00000025964 | Adam23 | −3.0439534 | 0.001559271 |
| ENSMUSG00000025956 | Mettl21a | 1.095942 | 0.002857998 |
| ENSMUSG00000015222 | Map2 | −5.72023731 | 4.08E−06 |
| ENSMUSG00000050296 | Abca12 | −4.53524226 | 0.001766377 |
| ENSMUSG00000026193 | Fn1 | −1.86354126 | 4.03E−04 |
| ENSMUSG00000026187 | Xrcc5 | 1.09300376 | 0.006745389 |
| ENSMUSG00000026185 | Igfbp5 | −3.76982301 | 7.16E−05 |
| ENSMUSG00000061815 | Rufy4 | −2.6233751 | 0.009529372 |
| ENSMUSG00000026180 | Cxcr2 | −3.1660611 | 1.86E−06 |
| ENSMUSG00000064272 | Gpbar1 | −1.73316082 | 0.014526654 |
| ENSMUSG00000026177 | Slc11a1 | 1.31903024 | 8.25E−04 |
| ENSMUSG00000026176 | Ctdsp1 | −1.4255523 | 0.001051108 |
| ENSMUSG00000026173 | Plcd4 | −1.97519523 | 0.010386937 |
| ENSMUSG00000026205 | Slc23a3 | −2.3725772 | 0.021273975 |
| ENSMUSG00000049339 | Retreg2 | 1.25977525 | 0.009179317 |
| ENSMUSG00000033124 | Atg9a | 1.11218725 | 0.012364574 |
| ENSMUSG00000026235 | Epha4 | −2.58096533 | 0.001503064 |
| ENSMUSG00000054702 | Ap1s3 | −2.73752561 | 0.024887572 |
| ENSMUSG00000026249 | Serpine2 | 2.16725923 | 2.90E−05 |
| ENSMUSG00000026142 | Rhbdd1 | 1.42771238 | 3.03E−04 |
| ENSMUSG00000036766 | Dner | −4.89654934 | 0.001652026 |
| ENSMUSG00000026223 | Itm2c | 1.11899291 | 0.001946963 |
| ENSMUSG00000079445 | B3gnt7 | 1.18203937 | 0.009826339 |
| ENSMUSG00000026255 | Efhd1 | −2.95389581 | 0.002427007 |
| ENSMUSG00000026259 | Ngef | −4.06884683 | 0.003338868 |
| ENSMUSG00000090124 | Ugt1a7c | 1.64554854 | 8.53E−05 |
| ENSMUSG00000079429 | Mroh2a | −1.31378454 | 0.010992722 |
| ENSMUSG00000055013 | Agap1 | −1.57321431 | 0.001002088 |
| ENSMUSG00000026269 | Rnpepl1 | −1.34625882 | 8.89E−04 |
| ENSMUSG00000014602 | Kif1a | −1.56168938 | 0.008119087 |
| ENSMUSG00000034107 | Ano7 | 1.3025151 | 0.03591337 |
| ENSMUSG00000026278 | Bok | −3.01654709 | 0.015646791 |
| ENSMUSG00000026285 | Pdcd1 | 1.31711511 | 4.24E−04 |
| ENSMUSG00000103973 | BC055308 | −1.88385956 | 0.035336498 |
| ENSMUSG00000097814 | Panct2 | −1.68780703 | 0.048770054 |
| ENSMUSG00000026335 | Pam | −2.34531762 | 0.007828587 |
| ENSMUSG00000056536 | Pign | 1.07229885 | 0.003877632 |
| ENSMUSG00000044340 | Phlpp1 | −1.30886819 | 0.007437857 |
| ENSMUSG00000057329 | Bcl2 | −1.07574861 | 0.002198053 |
| ENSMUSG00000092572 | Serpinb10 | −3.0379288 | 2.09E−04 |
| ENSMUSG00000026312 | Cdh7 | −2.31044639 | 0.047582926 |
| ENSMUSG00000038702 | Dsel | 1.82222487 | 0.007777274 |
| ENSMUSG00000070695 | Cntnap5a | −4.11253418 | 0.003995266 |
| ENSMUSG00000026380 | Tfcp2l1 | −8.47975373 | 6.20E−05 |
| ENSMUSG00000026383 | Epb41l5 | −1.63895709 | 0.00425131 |
| ENSMUSG00000026384 | Ptpn4 | −1.13588602 | 0.047230049 |
| ENSMUSG00000036975 | Tmem177 | 1.06998544 | 0.005886533 |
| ENSMUSG00000050777 | Tmem37 | 1.32080897 | 0.003278617 |
| ENSMUSG00000026385 | Dbi | −1.04621814 | 0.017924466 |
| ENSMUSG00000003721 | Insig2 | 1.0295938 | 0.003711253 |
| ENSMUSG00000026342 | Slc35f5 | 1.26243906 | 0.001335589 |
| ENSMUSG00000045382 | Cxcr4 | −1.32746876 | 0.009131581 |
| ENSMUSG00000026409 | Pfkfb2 | 1.01902661 | 0.024308752 |
| ENSMUSG00000016528 | Mapkapk2 | −1.21665755 | 0.001083091 |
| ENSMUSG00000042349 | Ikbke | −1.95958257 | 0.001133746 |
| ENSMUSG00000004552 | Ctse | 1.23153315 | 0.004963319 |
| ENSMUSG00000026434 | Nucks1 | −1.25900945 | 6.34E−04 |
| ENSMUSG00000042066 | Tmcc2 | −1.63017142 | 0.007982823 |
| ENSMUSG00000048174 | Tmem81 | −1.14937066 | 0.045858304 |
| ENSMUSG00000051998 | Lax1 | −2.74768214 | 0.008267608 |
| ENSMUSG00000026463 | Atp2b4 | −2.9492644 | 5.93E−04 |
| ENSMUSG00000020423 | Btg2 | 1.24087334 | 0.002591736 |
| ENSMUSG00000064246 | Chil1 | −2.68473934 | 2.66E−05 |
| ENSMUSG00000031506 | Ptpn7 | −1.6549023 | 3.20E−04 |
| ENSMUSG00000026424 | Gpr37l1 | −3.95109855 | 0.00353856 |
| ENSMUSG00000041889 | Shisa4 | −3.40495554 | 3.90E−04 |
| ENSMUSG00000009418 | Nav1 | −1.31509388 | 5.54E−04 |
| ENSMUSG00000026421 | Csrp1 | −1.74862908 | 0.001512866 |
| ENSMUSG00000041801 | Phlda3 | −3.40193658 | 3.02E−06 |
| ENSMUSG00000026411 | Tmem9 | 1.21772266 | 0.001709422 |
| ENSMUSG00000056268 | Dennd1b | −1.0156403 | 0.003877747 |
| ENSMUSG00000033952 | Aspm | −1.25009262 | 0.033323763 |
| ENSMUSG00000070594 | Cfhr4 | 2.20723433 | 0.002565812 |
| ENSMUSG00000033898 | Cfhr2 | 1.87177073 | 0.011649047 |
| ENSMUSG00000026365 | Cfh | 2.24517631 | 3.62E−05 |
| ENSMUSG00000026358 | Rgs1 | 1.6830029 | 1.09E−04 |
| ENSMUSG00000026357 | Rgs18 | −1.46391965 | 0.001775846 |
| ENSMUSG00000056220 | Pla2g4a | 1.15241112 | 0.001529074 |
| ENSMUSG00000097754 | Ptgs2os2 | −3.92248142 | 0.003030119 |
| ENSMUSG00000006014 | Prg4 | −1.7979708 | 0.022857371 |
| ENSMUSG00000023150 | Ivns1abp | 1.63989789 | 2.08E−04 |
| ENSMUSG00000043019 | Edem3 | 1.41818846 | 5.65E−04 |
| ENSMUSG00000026482 | Rgl1 | 1.40535203 | 0.003878003 |
| ENSMUSG00000042751 | Nmnat2 | −7.69123453 | 4.68E−04 |
| ENSMUSG00000026479 | Lamc2 | −2.56021034 | 0.008780338 |
| ENSMUSG00000042671 | Rgs8 | −4.31902705 | 8.45E−04 |
| ENSMUSG00000015484 | Fam163a | −3.89223371 | 0.001305008 |
| ENSMUSG00000060519 | Tor3a | 1.57821771 | 6.55E−05 |
| ENSMUSG00000026589 | Sec16b | 1.72516008 | 0.006012225 |
| ENSMUSG00000004031 | Brinp2 | −5.73655218 | 2.78E−04 |
| ENSMUSG00000026701 | Prdx6 | −1.1516848 | 0.001052719 |
| ENSMUSG00000000817 | Fasl | −2.74749439 | 0.005575903 |
| ENSMUSG00000078190 | Dnm3os | −3.90026614 | 0.006064428 |
| ENSMUSG00000040181 | Fmo1 | −2.1351986 | 0.015343915 |
| ENSMUSG00000102117 | Rpsa-ps1 | −1.48328987 | 0.013858704 |
| ENSMUSG00000026581 | Sell | −1.74560121 | 5.15E−05 |
| ENSMUSG00000026580 | Selp | −3.31205934 | 0.001245546 |
| ENSMUSG00000026579 | F5 | −2.56599289 | 6.26E−04 |
| ENSMUSG00000040918 | Slc19a2 | 1.08214772 | 0.01605233 |
| ENSMUSG00000026573 | Xcl1 | −4.55128142 | 7.64E−06 |
| ENSMUSG00000040848 | Sft2d2 | 1.16795636 | 0.001660034 |
| ENSMUSG00000040713 | Creg1 | 1.38630127 | 0.001245817 |
| ENSMUSG00000005763 | Cd247 | −1.86866643 | 0.018567074 |
| ENSMUSG00000026564 | Dusp27 | 1.61939631 | 0.001320427 |
| ENSMUSG00000060568 | Fam78b | −2.54942398 | 0.014936352 |
| ENSMUSG00000026558 | Uck2 | −1.12495355 | 0.012941631 |
| ENSMUSG00000052534 | Pbx1 | −1.61351237 | 3.90E−04 |
| ENSMUSG00000038530 | Rgs4 | −4.3419568 | 0.001428877 |
| ENSMUSG00000026674 | Ddr2 | −4.35347421 | 0.002757289 |
| ENSMUSG00000102418 | Sh2d1b1 | −1.31054423 | 0.001469687 |
| ENSMUSG00000042800 | Spata46 | 1.62298099 | 0.045932394 |
| ENSMUSG00000038463 | Olfml2b | 1.67484713 | 1.34E−04 |
| ENSMUSG00000070524 | Fcrlb | −1.05572445 | 0.033134359 |
| ENSMUSG00000038421 | Fcrla | −2.32611544 | 0.041551408 |
| ENSMUSG00000059498 | Fcgr3 | 1.25699183 | 0.002818244 |
| ENSMUSG00000038370 | Pcp4l1 | −1.70950441 | 0.016432715 |
| ENSMUSG00000103711 | Tstd1 | −1.20817146 | 0.045308125 |
| ENSMUSG00000038235 | F11r | 1.42442099 | 5.24E−04 |
| ENSMUSG00000004709 | Cd244a | −1.34515602 | 6.89E−04 |
| ENSMUSG00000038179 | Slamf7 | −1.72636642 | 5.46E−05 |
| ENSMUSG00000015316 | Slamf1 | 1.60569589 | 0.001768797 |
| ENSMUSG00000038147 | Cd84 | 1.15662759 | 0.003926749 |
| ENSMUSG00000015314 | Slamf6 | 1.3779496 | 8.78E−04 |
| ENSMUSG00000003458 | Ncstn | 1.7079076 | 7.58E−05 |
| ENSMUSG00000007097 | Atp1a2 | −4.0168309 | 2.97E−05 |
| ENSMUSG00000044708 | Kcnj10 | −1.42666657 | 0.006465906 |
| ENSMUSG00000037995 | Igsf9 | −1.1110902 | 0.013425067 |
| ENSMUSG00000005338 | Cadm3 | −2.34549095 | 0.026900442 |
| ENSMUSG00000090222 | Ifi203-ps | −3.05232561 | 7.84E−04 |
| ENSMUSG00000090272 | Mndal | −1.76129853 | 4.35E−05 |
| ENSMUSG00000039997 | Ifi203 | −1.55199925 | 1.83E−04 |
| ENSMUSG00000028354 | Fmn2 | −4.03971867 | 0.00284756 |
| ENSMUSG00000039783 | Kmo | −1.93754577 | 1.41E−04 |
| ENSMUSG00000078184 | Rbm8a2 | −1.74819139 | 0.02794589 |
| ENSMUSG00000038936 | Sccpdh | 1.13372563 | 0.001728119 |
| ENSMUSG00000026490 | Cdc42bpa | −1.31528192 | 0.014005561 |
| ENSMUSG00000026499 | Acbd3 | 1.0496233 | 0.005405524 |
| ENSMUSG00000038793 | Lefty1 | 1.05958551 | 0.016784149 |
| ENSMUSG00000026519 | Tmem63a | 1.39420277 | 5.70E−04 |
| ENSMUSG00000038776 | Ephx1 | 1.60415843 | 1.84E−04 |
| ENSMUSG00000022995 | Enah | −3.456293 | 2.13E−04 |
| ENSMUSG00000038576 | Susd4 | −5.11461134 | 0.001048396 |
| ENSMUSG00000079164 | Tlr5 | 1.31276217 | 0.00680296 |
| ENSMUSG00000030768 | Disp1 | 1.49739433 | 0.001058986 |
| ENSMUSG00000037624 | Kcnk2 | −4.62012872 | 0.001462235 |
| ENSMUSG00000026605 | Cenpf | −1.0749542 | 0.008389157 |
| ENSMUSG00000010175 | Prox1 | 1.23092395 | 0.00539559 |
| ENSMUSG00000026628 | Atf3 | 1.60829909 | 7.86E−04 |
| ENSMUSG00000026627 | Pacc1 | 1.53549959 | 1.13E−04 |
| ENSMUSG00000026626 | Ppp2r5a | −1.69295459 | 4.61E−05 |
| ENSMUSG00000037375 | Hhat | 1.62887441 | 0.001868378 |
| ENSMUSG00000026639 | Lamb3 | −2.63781727 | 0.003194459 |
| ENSMUSG00000016194 | Hsd11b1 | −2.33413712 | 9.50E−04 |
| ENSMUSG00000009633 | G0s2 | −2.95085776 | 2.72E−06 |
| ENSMUSG00000016494 | Cd34 | 1.86476302 | 4.13E−05 |
| ENSMUSG00000016481 | Cr1l | 1.09727661 | 0.004244453 |
| ENSMUSG00000050530 | Fam171a1 | 1.47578695 | 0.030552449 |
| ENSMUSG00000015619 | Gata3 | −3.25865021 | 0.004809411 |
| ENSMUSG00000025780 | Itih5 | −3.75941969 | 4.34E−04 |
| ENSMUSG00000026770 | Il2ra | −1.96900717 | 0.047186708 |
| ENSMUSG00000026728 | Vim | −1.65801154 | 7.98E−05 |
| ENSMUSG00000003418 | St8sia6 | 1.06030966 | 0.003234331 |
| ENSMUSG00000036949 | Slc39a12 | 1.11751475 | 0.026787012 |
| ENSMUSG00000026748 | Plxdc2 | 2.13382971 | 1.71E−05 |
| ENSMUSG00000053702 | Nebl | −3.68835285 | 7.47E−05 |
| ENSMUSG00000026986 | Hnmt | 1.26663602 | 0.006742162 |
| ENSMUSG00000044103 | Il36g | −3.45220084 | 3.16E−05 |
| ENSMUSG00000026981 | Il1rn | −1.39958454 | 0.001378479 |
| ENSMUSG00000026974 | Zmynd19 | −1.67296114 | 0.016785418 |
| ENSMUSG00000036833 | Pnpla7 | 1.18511861 | 0.002376326 |
| ENSMUSG00000059555 | Tor4a | 1.22743811 | 0.001549785 |
| ENSMUSG00000026958 | Dpp7 | 1.35177661 | 4.43E−04 |
| ENSMUSG00000015094 | Npdc1 | −7.3504545 | 3.05E−04 |
| ENSMUSG00000015090 | Ptgds | −1.41215665 | 0.005731755 |
| ENSMUSG00000015083 | C8g | 1.28016521 | 0.017362278 |
| ENSMUSG00000087679 | Tmem250-ps | −1.31980122 | 0.016869197 |
| ENSMUSG00000036327 | Qsox2 | 1.20694248 | 0.014075876 |
| ENSMUSG00000014867 | Surf4 | 1.07437875 | 0.001951702 |
| ENSMUSG00000009214 | Mymk | 1.53587025 | 0.001461864 |
| ENSMUSG00000009216 | Fam163b | −7.36264481 | 4.32E−04 |
| ENSMUSG00000026835 | Fcnb | −4.40446925 | 2.33E−07 |
| ENSMUSG00000026833 | Olfm1 | −3.71835046 | 3.82E−07 |
| ENSMUSG00000035829 | Ppp1r26 | 1.12277153 | 0.038034213 |
| ENSMUSG00000026829 | Gbgt1 | 2.12410046 | 2.27E−04 |
| ENSMUSG00000039787 | Cercam | 1.49067722 | 0.004164719 |
| ENSMUSG00000079484 | Phyhd1 | 1.02039778 | 0.002346739 |
| ENSMUSG00000075419 | Dolk | 1.00417047 | 0.005400639 |
| ENSMUSG00000026858 | Miga2 | 1.29328894 | 0.001725458 |
| ENSMUSG00000026856 | Dolpp1 | 1.02829229 | 0.00350447 |
| ENSMUSG00000000194 | Gpr107 | 1.01338621 | 0.003930708 |
| ENSMUSG00000076441 | Ass1 | −1.26281452 | 0.009345046 |
| ENSMUSG00000051373 | Plpp7 | 1.85447605 | 5.63E−04 |
| ENSMUSG00000039262 | Prrc2b | −1.22103176 | 0.002404507 |
| ENSMUSG00000039254 | Pomt1 | 1.38283324 | 0.001036415 |
| ENSMUSG00000039195 | Bbln | −1.42377782 | 0.002122516 |
| ENSMUSG00000026822 | Lcn2 | −2.36813858 | 6.89E−06 |
| ENSMUSG00000079442 | St6galnac4 | 1.46459456 | 1.50E−04 |
| ENSMUSG00000026814 | Eng | 1.84765103 | 5.18E−05 |
| ENSMUSG00000009563 | Tor2a | 1.0263633 | 0.00258015 |
| ENSMUSG00000026791 | Slc2a8 | 1.17494787 | 0.001109577 |
| ENSMUSG00000038860 | Garnl3 | 1.01005769 | 0.003966669 |
| ENSMUSG00000004105 | Angptl2 | −1.58803435 | 0.030044788 |
| ENSMUSG00000026864 | Hspa5 | 1.2428156 | 0.001785702 |
| ENSMUSG00000026875 | Traf1 | −2.30957667 | 1.47E−05 |
| ENSMUSG00000026879 | Gsn | −1.39639272 | 3.20E−04 |
| ENSMUSG00000047250 | Ptgs1 | 1.74246353 | 6.44E−05 |
| ENSMUSG00000026915 | Strbp | −1.00135065 | 0.029377442 |
| ENSMUSG00000000247 | Lhx2 | −7.24396192 | 8.27E−04 |
| ENSMUSG00000085201 | Nr6a1os | 1.36929686 | 0.001835873 |
| ENSMUSG00000049252 | Lrp1b | −2.40526403 | 0.009895216 |
| ENSMUSG00000049744 | Arhgap15 | −1.31789262 | 3.67E−04 |
| ENSMUSG00000026764 | Kif5c | −2.85220256 | 0.003253012 |
| ENSMUSG00000017144 | Rnd3 | −1.91762173 | 1.66E−04 |
| ENSMUSG00000017412 | Cacnb4 | −1.14620007 | 0.041423821 |
| ENSMUSG00000036053 | Fmnl2 | −1.16839179 | 0.011541278 |
| ENSMUSG00000026826 | Nr4a2 | −2.32208915 | 8.34E−04 |
| ENSMUSG00000026830 | Ermn | −3.92936933 | 0.001165621 |
| ENSMUSG00000026832 | Cytip | −1.91058123 | 3.48E−05 |
| ENSMUSG00000026980 | Ly75 | −1.62332124 | 0.014650735 |
| ENSMUSG00000035000 | Dpp4 | −1.99922291 | 3.01E−05 |
| ENSMUSG00000044647 | Csrnp3 | −7.2604715 | 4.10E−04 |
| ENSMUSG00000064329 | Scn1a | −4.77717481 | 0.001313859 |
| ENSMUSG00000027030 | Stk39 | −2.97271054 | 0.00103326 |
| ENSMUSG00000034738 | Nostrin | −1.37203689 | 0.025792139 |
| ENSMUSG00000005233 | Spc25 | −1.30271095 | 0.011217664 |
| ENSMUSG00000027068 | Dhrs9 | −2.64725565 | 0.001823434 |
| ENSMUSG00000049044 | Rapgef4 | −4.92212631 | 1.92E−04 |
| ENSMUSG00000041762 | Gpr155 | 2.03944929 | 3.23E−05 |
| ENSMUSG00000075284 | Wipf1 | −1.19866984 | 0.001784305 |
| ENSMUSG00000056486 | Chn1 | −3.67637558 | 1.55E−04 |
| ENSMUSG00000075272 | Ttc30a2 | 2.24599172 | 0.007684757 |
| ENSMUSG00000042359 | Osbpl6 | −3.26420477 | 0.001292873 |
| ENSMUSG00000002732 | Fkbp7 | 1.01363762 | 0.023789452 |
| ENSMUSG00000002733 | Plekha3 | −1.57017764 | 0.008143613 |
| ENSMUSG00000051747 | Ttn | −1.6984017 | 0.006905758 |
| ENSMUSG00000044033 | Ccdc141 | −2.62528516 | 0.007546432 |
| ENSMUSG00000042272 | Sestd1 | −1.38437464 | 0.010512602 |
| ENSMUSG00000027009 | Itga4 | −2.02633334 | 1.34E−05 |
| ENSMUSG00000075256 | Cerkl | −1.84761548 | 0.015859928 |
| ENSMUSG00000034701 | Neurod1 | −3.9491287 | 0.00296501 |
| ENSMUSG00000027002 | Nckap1 | −2.35537303 | 0.004720383 |
| ENSMUSG00000048388 | Fam171b | −2.53200133 | 0.006120025 |
| ENSMUSG00000027082 | Tfpi | 1.53055136 | 2.37E−04 |
| ENSMUSG00000050043 | Tmx2 | 1.47066573 | 8.03E−04 |
| ENSMUSG00000002100 | Mybpc3 | −1.54056178 | 0.025250883 |
| ENSMUSG00000002103 | Acp2 | 1.32431301 | 3.49E−04 |
| ENSMUSG00000027257 | Pacsin3 | −1.50175944 | 0.014861516 |
| ENSMUSG00000027249 | F2 | −3.28109729 | 0.00730124 |
| ENSMUSG00000027239 | Mdk | −1.76709654 | 0.014096258 |
| ENSMUSG00000027221 | Chst1 | 1.00489556 | 0.007569665 |
| ENSMUSG00000075028 | Prdm11 | −1.09652289 | 0.046612528 |
| ENSMUSG00000027217 | Tspan18 | 2.10192647 | 2.60E−05 |
| ENSMUSG00000027198 | Ext2 | 1.69157582 | 5.31E−05 |
| ENSMUSG00000027195 | Hsd17b12 | 1.06683066 | 0.003575897 |
| ENSMUSG00000005089 | Slc1a2 | −5.19346117 | 1.62E−07 |
| ENSMUSG00000005087 | Cd44 | −1.96197901 | 1.08E−05 |
| ENSMUSG00000012350 | Ehf | −2.387934 | 0.00114335 |
| ENSMUSG00000032724 | Abtb2 | −2.2560512 | 0.001714933 |
| ENSMUSG00000032679 | Cd59a | 3.0123975 | 3.13E−04 |
| ENSMUSG00000027173 | Depdc7 | 1.78235713 | 8.19E−05 |
| ENSMUSG00000005973 | Rcn1 | 1.02254898 | 0.009453681 |
| ENSMUSG00000027134 | Lpcat4 | −1.44607715 | 0.001416042 |
| ENSMUSG00000057378 | Ryr3 | −2.06551395 | 0.019064416 |
| ENSMUSG00000027210 | Meis2 | −6.13924861 | 1.75E−04 |
| ENSMUSG00000027347 | Rasgrp1 | −1.96857948 | 0.001243213 |
| ENSMUSG00000045838 | Ccdc9b | −1.5992847 | 0.028677019 |
| ENSMUSG00000074916 | Chst14 | 2.11391841 | 9.10E−05 |
| ENSMUSG00000081413 | Uqcrh-ps2 | −1.79020248 | 0.028046878 |
| ENSMUSG00000027315 | Spint1 | 1.50906227 | 2.00E−04 |
| ENSMUSG00000034226 | Rhov | −1.95541337 | 0.01998689 |
| ENSMUSG00000027304 | Rtf1 | −1.08251605 | 0.005871104 |
| ENSMUSG00000027259 | Adal | 1.05771653 | 0.015442999 |
| ENSMUSG00000027254 | Map1a | −3.06524325 | 0.004840347 |
| ENSMUSG00000000308 | Ckmt1 | −2.90223409 | 0.003860119 |
| ENSMUSG00000027248 | Pdia3 | 1.16820366 | 0.002604677 |
| ENSMUSG00000027238 | Frmd5 | −3.05617048 | 0.006476474 |
| ENSMUSG00000060227 | Golm2 | 1.11397325 | 0.025294715 |
| ENSMUSG00000027224 | Duoxa1 | 2.219279 | 8.93E−04 |
| ENSMUSG00000033213 | AA467197 | −2.34628624 | 2.10E−04 |
| ENSMUSG00000068394 | Cep152 | 1.34269872 | 3.49E−04 |
| ENSMUSG00000060131 | Atp8b4 | −2.66805046 | 4.97E−06 |
| ENSMUSG00000027360 | Hdc | −3.13291021 | 1.60E−06 |
| ENSMUSG00000027366 | Sppl2a | 1.12201807 | 0.00122756 |
| ENSMUSG00000027368 | Dusp2 | −1.46495101 | 0.001197723 |
| ENSMUSG00000046338 | Gpat2 | 3.8623375 | 5.76E−04 |
| ENSMUSG00000027375 | Mal | −4.34994624 | 0.001907016 |
| ENSMUSG00000014361 | Mertk | 1.47557002 | 8.41E−04 |
| ENSMUSG00000027399 | Il1a | 2.06373367 | 7.81E−06 |
| ENSMUSG00000027398 | Il1b | −1.60130297 | 1.98E−04 |
| ENSMUSG00000037902 | Sirpa | 1.75101952 | 4.02E−04 |
| ENSMUSG00000027400 | Pdyn | 3.38879619 | 0.00558516 |
| ENSMUSG00000027401 | Tgm3 | −3.21091727 | 0.00245461 |
| ENSMUSG00000027329 | Spef1 | −1.53199611 | 0.047174729 |
| ENSMUSG00000068267 | Cenpb | −1.25662806 | 0.002402057 |
| ENSMUSG00000037523 | Mavs | 1.1288664 | 0.001158759 |
| ENSMUSG00000037514 | Pank2 | 1.20724122 | 0.016313126 |
| ENSMUSG00000048911 | Rnf24 | 1.69323217 | 0.00328322 |
| ENSMUSG00000027340 | Slc23a2 | 1.45216925 | 4.52E−04 |
| ENSMUSG00000058793 | Cds2 | 1.11689373 | 0.001409298 |
| ENSMUSG00000027357 | Crls1 | −1.22758691 | 0.02184041 |
| ENSMUSG00000034723 | Tmx4 | 1.56162016 | 1.03E−04 |
| ENSMUSG00000051177 | Plcb1 | −3.82959903 | 2.88E−05 |
| ENSMUSG00000039913 | Pak5 | −3.57701259 | 0.006513594 |
| ENSMUSG00000027273 | Snap25 | −4.6386005 | 7.25E−06 |
| ENSMUSG00000027276 | Jag1 | −1.71943118 | 0.00853791 |
| ENSMUSG00000077714 | Snord17 | −1.55568744 | 0.010095316 |
| ENSMUSG00000074743 | Thbd | −1.36404119 | 0.001975129 |
| ENSMUSG00000027435 | Cd93 | −1.16856231 | 0.006421827 |
| ENSMUSG00000027447 | Cst3 | 1.11518942 | 0.017401555 |
| ENSMUSG00000068129 | Cst7 | 1.21421469 | 0.016296473 |
| ENSMUSG00000032046 | Abhd12 | 1.67983006 | 2.60E−04 |
| ENSMUSG00000068115 | Ninl | 1.03581196 | 0.020435858 |
| ENSMUSG00000027463 | Slc52a3 | −4.17248811 | 0.001742383 |
| ENSMUSG00000000359 | Rem1 | −1.53753261 | 0.042431484 |
| ENSMUSG00000068040 | Tm9sf4 | 1.4678742 | 2.28E−04 |
| ENSMUSG00000027489 | Necab3 | −2.28915954 | 0.009912957 |
| ENSMUSG00000038467 | Chmp4b | −1.04769219 | 0.022789486 |
| ENSMUSG00000038383 | Pigu | 1.19463756 | 0.001938992 |
| ENSMUSG00000074652 | Myh7b | −2.56358077 | 0.006469155 |
| ENSMUSG00000038312 | Edem2 | 1.32084238 | 4.34E−04 |
| ENSMUSG00000005881 | Ergic3 | 1.16972658 | 0.001731844 |
| ENSMUSG00000038085 | Cnbd2 | 1.02364929 | 0.015847144 |
| ENSMUSG00000067818 | Myl9 | −7.60670246 | 1.98E−04 |
| ENSMUSG00000062175 | Tgif2 | 1.20084642 | 0.023541943 |
| ENSMUSG00000027639 | Samhd1 | −1.05268314 | 0.002331313 |
| ENSMUSG00000027642 | Rpn2 | 1.63374883 | 1.25E−04 |
| ENSMUSG00000067786 | Nnat | −3.69636759 | 9.60E−05 |
| ENSMUSG00000016024 | Lbp | −2.17550164 | 0.002040083 |
| ENSMUSG00000037754 | Ppp1r16b | −3.7052973 | 1.29E−04 |
| ENSMUSG00000053141 | Ptprt | −5.33824434 | 3.85E−04 |
| ENSMUSG00000048486 | Fitm2 | 1.27002024 | 0.015631037 |
| ENSMUSG00000017707 | Serinc3 | 1.54931833 | 0.001236645 |
| ENSMUSG00000017697 | Ada | −1.04172994 | 0.028273518 |
| ENSMUSG00000017002 | Slpi | −3.78132942 | 1.06E−07 |
| ENSMUSG00000098489 | Mir7678 | −3.39709524 | 0.00869587 |
| ENSMUSG00000017734 | Dbndd2 | −1.94982173 | 6.91E−04 |
| ENSMUSG00000001403 | Ube2c | −1.27335827 | 0.003622621 |
| ENSMUSG00000017760 | Ctsa | 1.73566534 | 2.48E−04 |
| ENSMUSG00000017737 | Mmp9 | −1.47615755 | 3.17E−04 |
| ENSMUSG00000017740 | Slc12a5 | −4.0090248 | 4.40E−04 |
| ENSMUSG00000017652 | Cd40 | −1.01414336 | 0.011834671 |
| ENSMUSG00000027670 | Ocstamp | −2.76427804 | 0.001524995 |
| ENSMUSG00000027661 | Slc2a10 | 2.98519197 | 0.005435791 |
| ENSMUSG00000085274 | BC046401 | −1.41424739 | 0.041560779 |
| ENSMUSG00000002718 | Cse1l | 1.02996979 | 0.002972948 |
| ENSMUSG00000017929 | B4galt5 | −1.88967639 | 2.54E−04 |
| ENSMUSG00000056501 | Cebpb | −2.22799638 | 8.20E−05 |
| ENSMUSG00000047907 | Tshz2 | −6.60520644 | 0.001149495 |
| ENSMUSG00000013523 | Bcas1 | −5.20313905 | 4.49E−06 |
| ENSMUSG00000074570 | Cass4 | 1.00342315 | 0.011462497 |
| ENSMUSG00000027514 | Zbp1 | −1.11467334 | 0.002759627 |
| ENSMUSG00000016256 | Ctsz | 1.33699317 | 0.003683767 |
| ENSMUSG00000078866 | Zfp970 | 1.6703992 | 0.013988057 |
| ENSMUSG00000027525 | Phactr3 | −4.55264478 | 9.96E−05 |
| ENSMUSG00000060445 | Sycp2 | −2.79948288 | 0.003177276 |
| ENSMUSG00000039117 | Taf4 | −1.38875289 | 0.002533831 |
| ENSMUSG00000039108 | Lsm14b | −1.44932332 | 0.02170608 |
| ENSMUSG00000038963 | Slco4a1 | 1.38299565 | 3.85E−04 |
| ENSMUSG00000038848 | Ythdf1 | −1.01869579 | 0.009849784 |
| ENSMUSG00000016356 | Col20a1 | −2.5899695 | 0.026544186 |
| ENSMUSG00000027580 | Helz2 | −1.36721838 | 0.005059245 |
| ENSMUSG00000027581 | Stmn3 | −4.70239486 | 9.02E−04 |
| ENSMUSG00000027582 | Zgpat | 1.01248273 | 0.004809743 |
| ENSMUSG00000085028 | Slc2a4rg-ps | −1.57568834 | 0.002993917 |
| ENSMUSG00000031146 | Plp2 | −1.71934385 | 2.39E−04 |
| ENSMUSG00000031149 | Praf2 | 1.02168404 | 0.025090283 |
| ENSMUSG00000009731 | Kcnd1 | 1.46490081 | 7.16E−04 |
| ENSMUSG00000031156 | Slc35a2 | 1.00374562 | 0.004971419 |
| ENSMUSG00000039278 | Pcsk1n | −4.47319089 | 5.65E−04 |
| ENSMUSG00000031169 | Porcn | 1.12843699 | 0.017128325 |
| ENSMUSG00000058254 | Tspan7 | 1.15752798 | 0.001224634 |
| ENSMUSG00000031007 | Atp6ap2 | 1.15946435 | 0.006175532 |
| ENSMUSG00000083364 | Llph-ps2 | −1.44903228 | 0.037473623 |
| ENSMUSG00000040229 | Gpr34 | 2.05055826 | 8.15E−04 |
| ENSMUSG00000040147 | Maob | −3.89139841 | 1.97E−05 |
| ENSMUSG00000037347 | Chst7 | 1.48434034 | 1.89E−04 |
| ENSMUSG00000037341 | Slc9a7 | 1.62898331 | 5.13E−04 |
| ENSMUSG00000037315 | Jade3 | 1.19286156 | 0.0440305 |
| ENSMUSG00000037217 | Syn1 | −5.59461206 | 6.99E−04 |
| ENSMUSG00000001128 | Cfp | −1.84261236 | 2.15E−05 |
| ENSMUSG00000017057 | Il13ra1 | 1.33250608 | 4.85E−04 |
| ENSMUSG00000006373 | Pgrmc1 | 1.14148047 | 0.002793778 |
| ENSMUSG00000016534 | Lamp2 | 1.6158449 | 3.86E−04 |
| ENSMUSG00000048970 | C1galt1c1 | 1.33876466 | 0.003530693 |
| ENSMUSG00000005696 | Sh2d1a | −2.93756818 | 0.005123436 |
| ENSMUSG00000037010 | Apln | −3.06390775 | 0.004287296 |
| ENSMUSG00000036985 | Zdhhc9 | 1.21270403 | 7.18E−04 |
| ENSMUSG00000085396 | Firre | 1.12699682 | 0.008944673 |
| ENSMUSG00000036109 | Mbnl3 | −2.02387737 | 0.008841095 |
| ENSMUSG00000073176 | Zfp449 | 1.41438108 | 0.004360714 |
| ENSMUSG00000035967 | Ints6l | 1.03480116 | 0.004887175 |
| ENSMUSG00000023092 | Fhl1 | −3.19955793 | 0.008730095 |
| ENSMUSG00000031138 | F9 | 1.63802551 | 0.001240776 |
| ENSMUSG00000090546 | Cdr1 | −4.47672052 | 4.96E−04 |
| ENSMUSG00000073139 | Tmem185a | 1.28761402 | 0.001034201 |
| ENSMUSG00000035776 | Cd99l2 | 1.04045122 | 0.003865467 |
| ENSMUSG00000015217 | Hmgb3 | −2.55328952 | 0.001251839 |
| ENSMUSG00000031385 | Plxnb3 | 1.30900719 | 0.0051604 |
| ENSMUSG00000002007 | Srpk3 | 1.31451383 | 4.07E−04 |
| ENSMUSG00000002014 | Ssr4 | 1.06675153 | 0.01848383 |
| ENSMUSG00000031391 | L1cam | −1.38371231 | 0.001552048 |
| ENSMUSG00000031397 | Tktl1 | −4.55061797 | 5.12E−04 |
| ENSMUSG00000019087 | Atp6ap1 | 1.33930412 | 8.03E−04 |
| ENSMUSG00000032806 | Slc10a3 | 1.53318508 | 3.83E−04 |
| ENSMUSG00000031399 | Fam3a | 1.51645735 | 1.74E−04 |
| ENSMUSG00000016382 | Pls3 | 1.06703608 | 0.004956622 |
| ENSMUSG00000081049 | Rps24-ps3 | −1.38018799 | 0.021125971 |
| ENSMUSG00000025666 | Tmem47 | −3.97162904 | 7.79E−05 |
| ENSMUSG00000045103 | Dmd | −3.99708507 | 3.89E−04 |
| ENSMUSG00000035246 | Pcyt1b | −1.82211308 | 0.03094223 |
| ENSMUSG00000025656 | Arhgef9 | −1.61425283 | 0.008875065 |
| ENSMUSG00000031210 | Gpr165 | 2.52421225 | 9.33E−05 |
| ENSMUSG00000046532 | Ar | −4.62989894 | 0.001410151 |
| ENSMUSG00000031217 | Efnb1 | 1.35585257 | 0.003572544 |
| ENSMUSG00000019359 | Gdpd2 | −6.72036375 | 9.32E−04 |
| ENSMUSG00000042903 | Foxo4 | −1.71346713 | 0.022548266 |
| ENSMUSG00000031304 | Il2rg | −1.01971212 | 0.006909116 |
| ENSMUSG00000031302 | Nlgn3 | −3.48534106 | 0.003300118 |
| ENSMUSG00000049191 | Rtl5 | −1.95150167 | 0.004256412 |
| ENSMUSG00000079480 | Pin4 | −1.01644805 | 0.021836712 |
| ENSMUSG00000082229 | Nap1l2 | −3.30100368 | 0.002557891 |
| ENSMUSG00000033906 | Zdhhc15 | −1.42694754 | 0.048234351 |
| ENSMUSG00000033777 | Tlr13 | 1.22485069 | 0.001068078 |
| ENSMUSG00000052821 | Cysltr1 | 1.0872729 | 0.0063218 |
| ENSMUSG00000050921 | P2ry10 | −1.76361652 | 0.002122987 |
| ENSMUSG00000054293 | P2ry10b | 1.17095666 | 0.015268984 |
| ENSMUSG00000073008 | Gpr174 | −3.65144811 | 0.004279211 |
| ENSMUSG00000031245 | Hmgn5 | −1.39634905 | 0.003203971 |
| ENSMUSG00000031246 | Sh3bgrl | −1.20747056 | 0.003415162 |
| ENSMUSG00000031253 | Srpx2 | −6.74629782 | 0.001050148 |
| ENSMUSG00000000223 | Drp2 | −8.29597708 | 6.82E−05 |
| ENSMUSG00000033460 | Armcx1 | −3.44292989 | 0.003524922 |
| ENSMUSG00000049047 | Armcx3 | 1.05167456 | 0.006655531 |
| ENSMUSG00000072964 | Bhlhb9 | 1.08709738 | 0.004043883 |
| ENSMUSG00000072966 | Gprasp2 | 1.41779561 | 0.001414914 |
| ENSMUSG00000048040 | Arxes2 | −1.98073412 | 0.036601459 |
| ENSMUSG00000042750 | Bex2 | −4.86808219 | 1.25E−04 |
| ENSMUSG00000044550 | Tceal3 | −4.55129277 | 1.47E−04 |
| ENSMUSG00000031425 | Plp1 | −2.88885555 | 2.15E−04 |
| ENSMUSG00000031428 | Zcchc18 | −2.38090456 | 0.003676692 |
| ENSMUSG00000052854 | Nrk | −3.47407606 | 0.008141578 |
| ENSMUSG00000031438 | Rnf128 | 2.32052397 | 1.29E−05 |
| ENSMUSG00000000266 | Mid2 | −3.7922236 | 0.004569347 |
| ENSMUSG00000090122 | Kcne1l | −7.24697062 | 4.09E−04 |
| ENSMUSG00000042225 | Ammecr1 | −1.72529304 | 0.01535226 |
| ENSMUSG00000041688 | Amot | −3.65910393 | 3.05E−04 |
| ENSMUSG00000025270 | Alas2 | 1.53091291 | 0.006739249 |
| ENSMUSG00000045180 | Shroom2 | −4.81924321 | 4.86E−04 |
| ENSMUSG00000047238 | Mageh1 | 1.15543728 | 0.044379758 |
| ENSMUSG00000041658 | Rragb | −2.91738662 | 0.001344836 |
| ENSMUSG00000025289 | Prdx4 | 1.51959738 | 5.99E−04 |
| ENSMUSG00000041552 | Ptchd1 | 1.03220571 | 0.005635839 |
| ENSMUSG00000031309 | Rps6ka3 | −1.19689416 | 0.020417506 |
| ENSMUSG00000041020 | Map7d2 | −3.85608857 | 0.005245503 |
| ENSMUSG00000031303 | Map3k15 | −1.38872228 | 0.025869327 |
| ENSMUSG00000031292 | Cdkl5 | −2.69219257 | 0.005857175 |
| ENSMUSG00000031377 | Bmx | −2.83924182 | 1.79E−04 |
| ENSMUSG00000031379 | Pir | −2.71694176 | 0.007667052 |
| ENSMUSG00000031382 | Asb11 | −2.6151698 | 4.05E−04 |
| ENSMUSG00000047757 | Fancb | 1.16966187 | 0.032306579 |
| ENSMUSG00000031342 | Gpm6b | −4.00860938 | 2.17E−07 |
| ENSMUSG00000044583 | Tlr7 | 1.24022258 | 0.001581979 |
| ENSMUSG00000031355 | Arhgap6 | −1.30341672 | 0.028262229 |
| ENSMUSG00000031352 | Hccs | 1.0512529 | 0.007146274 |
| ENSMUSG00000081137 | BC022960 | 2.31097308 | 0.004352671 |
| ENSMUSG00000095562 | | −2.72796466 | 0.011832548 |
| ENSMUSG00000069125 | Rps24-ps2 | −4.25499159 | 0.003251775 |
| ENSMUSG00000027499 | Pkia | −2.44833134 | 0.002615165 |
| ENSMUSG00000027500 | Stmn2 | −3.31077119 | 0.006827902 |
| ENSMUSG00000040289 | Hey1 | −2.94385491 | 0.001995594 |
| ENSMUSG00000027533 | Fabp5 | −1.70567399 | 2.02E−04 |
| ENSMUSG00000062515 | Fabp4 | −1.8291627 | 9.60E−04 |
| ENSMUSG00000027562 | Car2 | −2.7296667 | 2.97E−04 |
| ENSMUSG00000095788 | Sirpb1a | −2.04899034 | 6.10E−04 |
| ENSMUSG00000095028 | Sirpb1b | −1.59539118 | 0.003929605 |
| ENSMUSG00000074677 | Sirpb1c | −1.93647643 | 2.77E−04 |
| ENSMUSG00000039519 | Cyp7b1 | −2.11427558 | 0.04057876 |
| ENSMUSG00000063887 | Nlgn1 | −3.08001038 | 0.006449367 |
| ENSMUSG00000027698 | Nceh1 | 1.24684852 | 6.56E−04 |
| ENSMUSG00000039304 | Tnfsf10 | 1.09931752 | 0.014040495 |
| ENSMUSG00000027695 | Pld1 | 1.03657121 | 0.006030112 |
| ENSMUSG00000027692 | Tnik | −1.39247977 | 0.024882508 |
| ENSMUSG00000037730 | Mynn | 1.09123834 | 0.001992359 |
| ENSMUSG00000037661 | Gpr160 | 1.00690085 | 0.00429994 |
| ENSMUSG00000037643 | Prkci | 1.25146755 | 0.005918078 |
| ENSMUSG00000091091 | Kcnmb3 | −2.98571862 | 0.00447506 |
| ENSMUSG00000027669 | Gnb4 | −1.80938941 | 0.008970748 |
| ENSMUSG00000105265 | Sox2ot | −3.112086 | 0.001688335 |
| ENSMUSG00000037325 | Bbs7 | 1.13895355 | 0.00707894 |
| ENSMUSG00000037211 | Spry1 | 1.3188526 | 0.001557357 |
| ENSMUSG00000025759 | Mfsd8 | 1.04765498 | 0.034008196 |
| ENSMUSG00000025762 | Larp1b | −1.85824898 | 0.009483756 |
| ENSMUSG00000049100 | Pcdh10 | −4.3772834 | 0.001431635 |
| ENSMUSG00000090919 | Pabpc4l | −3.34551026 | 0.009969482 |
| ENSMUSG00000027737 | Slc7a11 | −2.63937442 | 2.27E−05 |
| ENSMUSG00000023087 | Noct | −2.42140636 | 2.94E−04 |
| ENSMUSG00000074604 | Mgst2 | −2.38210835 | 3.57E−04 |
| ENSMUSG00000048332 | Lhfp | −5.91630497 | 2.00E−04 |
| ENSMUSG00000042997 | Nhlrc3 | 1.23382821 | 0.001225752 |
| ENSMUSG00000027744 | Stoml3 | −2.80636594 | 0.021920733 |
| ENSMUSG00000027750 | Postn | 1.65609674 | 0.001536624 |
| ENSMUSG00000036632 | Alg5 | 1.21295768 | 9.44E−04 |
| ENSMUSG00000036615 | Rfxap | 1.67763891 | 5.48E−04 |
| ENSMUSG00000027797 | Dclk1 | −4.06438932 | 1.56E−05 |
| ENSMUSG00000027803 | Wwtr1 | −3.33622449 | 1.59E−04 |
| ENSMUSG00000036503 | Rnf13 | 1.03467026 | 0.009114092 |
| ENSMUSG00000027805 | Pfn2 | −2.07662475 | 1.67E−04 |
| ENSMUSG00000036432 | Siah2 | −1.37327861 | 0.001301286 |
| ENSMUSG00000036362 | P2ry13 | 2.48184651 | 1.94E−05 |
| ENSMUSG00000036353 | P2ry12 | 2.53616223 | 4.44E−05 |
| ENSMUSG00000036334 | Igsf10 | 1.94410272 | 0.003538221 |
| ENSMUSG00000036894 | Rap2b | −1.54287063 | 9.31E−04 |
| ENSMUSG00000036885 | Arhgef26 | −3.67876898 | 2.28E−04 |
| ENSMUSG00000027822 | Slc33a1 | 1.40847086 | 0.002867996 |
| ENSMUSG00000027775 | Mfsd1 | 1.246799 | 0.001594578 |
| ENSMUSG00000027777 | Schip1 | −2.79599017 | 0.009584917 |
| ENSMUSG00000027784 | Ppm1l | 1.74976417 | 0.002063457 |
| ENSMUSG00000043300 | B3galnt1 | 1.61344774 | 1.67E−04 |
| ENSMUSG00000027790 | Sis | −4.71487095 | 0.001196384 |
| ENSMUSG00000027956 | Tmem144 | 1.58449023 | 1.27E−04 |
| ENSMUSG00000028015 | Ctso | 1.74199295 | 1.71E−04 |
| ENSMUSG00000033910 | Gucy1a1 | −3.16446932 | 0.002514149 |
| ENSMUSG00000033900 | Map9 | −1.52922452 | 0.046643768 |
| ENSMUSG00000027995 | Tlr2 | 1.2992531 | 0.001020996 |
| ENSMUSG00000028076 | Cd1d1 | 1.11024953 | 0.008831224 |
| ENSMUSG00000015852 | Fcrls | 1.67723364 | 2.40E−04 |
| ENSMUSG00000059994 | Fcrl1 | 1.43424337 | 6.44E−04 |
| ENSMUSG00000028073 | Pear1 | 1.29718655 | 0.002915058 |
| ENSMUSG00000028071 | Sh2d2a | −3.84673006 | 3.67E−05 |
| ENSMUSG00000004892 | Bcan | −3.70741157 | 8.60E−04 |
| ENSMUSG00000097428 | AW047730 | −8.2501525 | 2.75E−04 |
| ENSMUSG00000001418 | Glmp | 1.22574408 | 7.57E−04 |
| ENSMUSG00000028064 | Sema4a | −1.02757716 | 0.003375184 |
| ENSMUSG00000028051 | Hcn3 | 1.63222764 | 0.048887818 |
| ENSMUSG00000028049 | Scamp3 | 1.06518734 | 0.001812752 |
| ENSMUSG00000032657 | Fam189b | −1.23967225 | 0.016935076 |
| ENSMUSG00000028048 | Gba | 1.51035051 | 1.61E−04 |
| ENSMUSG00000028047 | Thbs3 | −3.33792078 | 0.001300999 |
| ENSMUSG00000028041 | Adam15 | 1.14290137 | 0.002120364 |
| ENSMUSG00000028042 | Zbtb7b | −1.04571351 | 0.008583603 |
| ENSMUSG00000042572 | Ube2q1 | −1.06859101 | 0.01118871 |
| ENSMUSG00000027939 | Nup210l | −1.42485193 | 0.007463081 |
| ENSMUSG00000027935 | Rab13 | −1.81593848 | 0.016625613 |
| ENSMUSG00000052310 | Slc39a1 | −1.49722395 | 0.012774632 |
| ENSMUSG00000042390 | Gatad2b | −1.13769579 | 0.001365755 |
| ENSMUSG00000027932 | Slc27a3 | −2.32711781 | 7.51E−04 |
| ENSMUSG00000044080 | S100a1 | −1.09804244 | 0.013759612 |
| ENSMUSG00000074457 | S100a16 | −4.3044753 | 2.52E−06 |
| ENSMUSG00000001020 | S100a4 | −3.19344474 | 4.41E−05 |
| ENSMUSG00000001025 | S100a6 | −2.85759898 | 4.07E−06 |
| ENSMUSG00000056054 | S100a8 | −2.9088026 | 3.58E−05 |
| ENSMUSG00000056071 | S100a9 | −2.83202798 | 1.16E−05 |
| ENSMUSG00000081640 | Rplp0-ps1 | −1.88570069 | 0.033519352 |
| ENSMUSG00000027907 | S100a11 | −2.67432223 | 1.22E−06 |
| ENSMUSG00000041959 | S100a10 | −1.92465608 | 2.74E−05 |
| ENSMUSG00000028150 | Rorc | −4.19126183 | 0.003539714 |
| ENSMUSG00000005628 | Tmod4 | −2.21636115 | 0.006700752 |
| ENSMUSG00000015714 | Cers2 | 1.35802657 | 2.94E−04 |
| ENSMUSG00000038642 | Ctss | 1.43798792 | 0.004197772 |
| ENSMUSG00000015850 | Adamtsl4 | −1.22814041 | 0.043386001 |
| ENSMUSG00000038550 | Ciart | 1.19461025 | 0.002017387 |
| ENSMUSG00000015947 | Fcgr1 | 1.2067204 | 0.002427196 |
| ENSMUSG00000038393 | Txnip | 1.17736673 | 0.002194411 |
| ENSMUSG00000049288 | Lix1l | −1.79233827 | 0.012168324 |
| ENSMUSG00000105144 | Rpl21-ps11 | −2.47669041 | 0.017494 |
| ENSMUSG00000028100 | Nudt17 | −3.17154708 | 0.001142837 |
| ENSMUSG00000038304 | Cd160 | −2.49125604 | 0.008395761 |
| ENSMUSG00000028096 | Gpr89 | 1.55713758 | 2.43E−04 |
| ENSMUSG00000043468 | Adam30 | −1.54627699 | 0.044015338 |
| ENSMUSG00000008763 | Man1a2 | 1.0090537 | 0.002652804 |
| ENSMUSG00000027863 | Cd2 | −2.12456552 | 0.003570934 |
| ENSMUSG00000042035 | Igsf3 | −3.58620342 | 0.003043363 |
| ENSMUSG00000033147 | Slc22a15 | −2.09181549 | 5.20E−04 |
| ENSMUSG00000027860 | Vangl1 | 1.93485808 | 0.002511014 |
| ENSMUSG00000027858 | Tspan2 | −1.92799908 | 0.003578008 |
| ENSMUSG00000087119 | Atg4a-ps | −2.08049321 | 0.021796651 |
| ENSMUSG00000027848 | Olfml3 | 1.97299785 | 1.22E−04 |
| ENSMUSG00000008730 | Hipk1 | −1.08718503 | 0.008442688 |
| ENSMUSG00000027843 | Ptpn22 | −1.43920691 | 5.30E−04 |
| ENSMUSG00000032913 | Lrig2 | 1.06146036 | 0.003490461 |
| ENSMUSG00000032902 | Slc16a1 | 1.5466816 | 3.56E−04 |
| ENSMUSG00000002228 | Ppm1j | −1.01627029 | 0.026676254 |
| ENSMUSG00000045576 | St7l | 1.06740675 | 0.002363173 |
| ENSMUSG00000062127 | Cttnbp2nl | 1.28894854 | 7.50E−04 |
| ENSMUSG00000000562 | Adora3 | 1.42913077 | 3.72E−04 |
| ENSMUSG00000040809 | Chil3 | −2.93142664 | 3.34E−06 |
| ENSMUSG00000063779 | Chil4 | −2.23516353 | 0.004391438 |
| ENSMUSG00000040747 | Cd53 | 1.17092181 | 0.006538259 |
| ENSMUSG00000047959 | Kcna3 | 2.46903238 | 0.002684607 |
| ENSMUSG00000040724 | Kcna2 | −4.67062338 | 0.001019385 |
| ENSMUSG00000027893 | Ahcyl1 | −1.24908252 | 7.11E−04 |
| ENSMUSG00000068762 | Gstm6 | −6.43074561 | 0.001418032 |
| ENSMUSG00000058135 | Gstm1 | −1.70762373 | 3.90E−04 |
| ENSMUSG00000068740 | Celsr2 | −2.87219318 | 0.019437886 |
| ENSMUSG00000027884 | Clcc1 | 1.35080945 | 4.63E−04 |
| ENSMUSG00000040322 | Slc25a24 | −1.79420233 | 4.63E−04 |
| ENSMUSG00000033721 | Vav3 | −2.25935047 | 5.79E−05 |
| ENSMUSG00000027966 | Col11a1 | −2.76844433 | 0.008509749 |
| ENSMUSG00000045092 | S1pr1 | 1.79970263 | 3.86E−05 |
| ENSMUSG00000054414 | Slc30a7 | 1.289153 | 5.00E−04 |
| ENSMUSG00000027963 | Extl2 | 1.3436833 | 8.02E−04 |
| ENSMUSG00000089911 | Mfsd14a | 1.18545852 | 0.001243991 |
| ENSMUSG00000027957 | Slc35a3 | 1.2383517 | 7.92E−04 |
| ENSMUSG00000044667 | Plppr4 | 1.82707649 | 0.006472746 |
| ENSMUSG00000053931 | Cnn3 | −2.0962581 | 8.22E−05 |
| ENSMUSG00000028127 | Abcd3 | 1.14026886 | 0.001896091 |
| ENSMUSG00000053965 | Pde5a | −2.99439058 | 0.002946866 |
| ENSMUSG00000039701 | Usp53 | −1.09069971 | 0.016434748 |
| ENSMUSG00000050315 | Synpo2 | −3.83583586 | 0.005105164 |
| ENSMUSG00000106258 | Rpl7a-ps7 | −1.70644603 | 0.003869866 |
| ENSMUSG00000041220 | Elovl6 | −1.24936633 | 0.036390858 |
| ENSMUSG00000027994 | Mcub | −1.9666567 | 6.18E−05 |
| ENSMUSG00000019232 | Etnppl | −5.74131408 | 5.56E−05 |
| ENSMUSG00000050931 | Sgms2 | −2.9396538 | 5.24E−04 |
| ENSMUSG00000040998 | Npnt | 2.11525694 | 6.72E−06 |
| ENSMUSG00000045328 | Cenpe | −1.51495764 | 0.002990807 |
| ENSMUSG00000028164 | Manba | 1.30759836 | 4.01E−04 |
| ENSMUSG00000053897 | Slc39a8 | 1.74616473 | 5.80E−04 |
| ENSMUSG00000037922 | Bank1 | 1.11798902 | 0.001703206 |
| ENSMUSG00000028159 | Dapp1 | 1.11530768 | 0.001219712 |
| ENSMUSG00000052430 | Bmpr1b | −3.0575387 | 0.005756 |
| ENSMUSG00000028266 | Lmo4 | −1.21412302 | 0.002631607 |
| ENSMUSG00000040151 | Hs2st1 | 1.05003458 | 0.016282378 |
| ENSMUSG00000028195 | Ccn1 | 1.16063026 | 0.003570781 |
| ENSMUSG00000028194 | Ddah1 | −4.19028691 | 4.63E−05 |
| ENSMUSG00000028189 | Ctbs | 1.31872868 | 0.001911772 |
| ENSMUSG00000036745 | Ttll7 | −5.22985384 | 0.001138098 |
| ENSMUSG00000028184 | Adgrl2 | 1.54966252 | 5.21E−04 |
| ENSMUSG00000025437 | Usp33 | 1.25505112 | 0.00100924 |
| ENSMUSG00000039047 | Pigk | 1.13857215 | 0.001490364 |
| ENSMUSG00000005493 | Msh4 | −3.76276005 | 0.002300226 |
| ENSMUSG00000053870 | Fpgt | 1.04904146 | 0.0049749 |
| ENSMUSG00000040037 | Negr1 | −1.67872963 | 0.006676707 |
| ENSMUSG00000040016 | Ptger3 | 1.84600916 | 0.003181321 |
| ENSMUSG00000028173 | Wls | 1.40138943 | 3.87E−04 |
| ENSMUSG00000028232 | Tmem68 | 1.19530692 | 0.00475114 |
| ENSMUSG00000041272 | Tox | −7.40528205 | 3.15E−04 |
| ENSMUSG00000041261 | Car8 | −3.39295134 | 0.005585756 |
| ENSMUSG00000028207 | Asph | 1.54440633 | 1.72E−04 |
| ENSMUSG00000028214 | Gem | 1.58025388 | 7.47E−05 |
| ENSMUSG00000049488 | Tmem67 | 1.37056884 | 0.001358504 |
| ENSMUSG00000055963 | Triqk | 1.57621462 | 0.040418567 |
| ENSMUSG00000028238 | Atp6v0d2 | −4.21434556 | 3.67E−08 |
| ENSMUSG00000073987 | Ggh | 1.0826768 | 0.004925785 |
| ENSMUSG00000040410 | Fbxl4 | 1.31203921 | 5.64E−04 |
| ENSMUSG00000095139 | Pou3f2 | −3.44942306 | 0.004034391 |
| ENSMUSG00000040387 | Klhl32 | −2.67663821 | 0.008089261 |
| ENSMUSG00000040359 | Ufl1 | 1.08503438 | 0.001876963 |
| ENSMUSG00000040520 | Manea | 1.88100254 | 1.01E−04 |
| ENSMUSG00000028289 | Epha7 | −2.26379912 | 0.024368664 |
| ENSMUSG00000040183 | Ankrd6 | −1.6735687 | 0.043623411 |
| ENSMUSG00000028278 | Rragd | −1.32463522 | 9.42E−04 |
| ENSMUSG00000028293 | Slc35a1 | 1.1559441 | 0.005942033 |
| ENSMUSG00000073910 | Mob3b | −1.8322729 | 7.24E−04 |
| ENSMUSG00000028435 | Aqp3 | −1.79245974 | 0.022320137 |
| ENSMUSG00000028445 | Enho | −2.72255638 | 0.003418979 |
| ENSMUSG00000028469 | Npr2 | −4.40779774 | 0.002570824 |
| ENSMUSG00000028476 | Reck | 1.47750541 | 0.04219111 |
| ENSMUSG00000028480 | Glipr2 | −1.88547147 | 1.16E−04 |
| ENSMUSG00000048232 | Fbxo10 | 1.18303613 | 0.017048655 |
| ENSMUSG00000035539 | Ccdc180 | −2.85906032 | 6.82E−04 |
| ENSMUSG00000039813 | Tbc1d2 | −1.55814766 | 0.032487514 |
| ENSMUSG00000039774 | Galnt12 | 2.15674328 | 1.07E−05 |
| ENSMUSG00000007613 | Tgfbr1 | 1.57012427 | 0.001015771 |
| ENSMUSG00000039740 | Alg2 | 1.3761513 | 9.13E−04 |
| ENSMUSG00000039693 | Msantd3 | −2.59294967 | 0.012614936 |
| ENSMUSG00000039634 | Zfp189 | 1.24470201 | 0.004585944 |
| ENSMUSG00000039611 | Pgap4 | −2.05786099 | 0.031385502 |
| ENSMUSG00000003032 | Klf4 | −1.11025565 | 0.001550058 |
| ENSMUSG00000038668 | Lpar1 | −4.65949182 | 7.04E−04 |
| ENSMUSG00000028381 | Ugcg | −1.0080563 | 0.008436354 |
| ENSMUSG00000066150 | Slc31a1 | 1.16201697 | 0.001817317 |
| ENSMUSG00000028391 | Wdr31 | −2.62370535 | 0.006326264 |
| ENSMUSG00000039196 | Orm1 | −3.46022434 | 6.31E−05 |
| ENSMUSG00000028359 | Orm3 | −1.56309743 | 0.031344488 |
| ENSMUSG00000050395 | Tnfsf15 | −3.44761439 | 0.008471378 |
| ENSMUSG00000039005 | Tlr4 | 1.78466475 | 2.96E−05 |
| ENSMUSG00000028351 | Brinp1 | −6.96404163 | 7.71E−04 |
| ENSMUSG00000028402 | Mpdz | −1.86949551 | 0.044411485 |
| ENSMUSG00000008575 | Nfib | −3.60573244 | 0.004454905 |
| ENSMUSG00000028488 | Sh3gl2 | −4.73474808 | 5.87E−05 |
| ENSMUSG00000038368 | Focad | 1.04370085 | 0.025587858 |
| ENSMUSG00000008489 | Elavl2 | −4.75364567 | 0.001177449 |
| ENSMUSG00000052684 | Jun | 1.0233299 | 0.043304885 |
| ENSMUSG00000028572 | Hook1 | −2.31335323 | 0.042888498 |
| ENSMUSG00000015224 | Cyp2j9 | −3.43860252 | 2.84E−04 |
| ENSMUSG00000073792 | Alg6 | 1.43779278 | 0.001737772 |
| ENSMUSG00000028527 | Ak4 | −2.75550128 | 6.67E−04 |
| ENSMUSG00000035212 | Leprot | 1.06287235 | 0.004901099 |
| ENSMUSG00000057722 | Lepr | −3.52107765 | 0.006700033 |
| ENSMUSG00000035069 | Oma1 | 1.06370196 | 0.001932015 |
| ENSMUSG00000028519 | Dab1 | −3.29314458 | 1.52E−04 |
| ENSMUSG00000034926 | Dhcr24 | −1.59983649 | 0.008609008 |
| ENSMUSG00000061887 | Ssbp3 | −1.03067529 | 0.029070782 |
| ENSMUSG00000028618 | Tmem59 | 1.29202075 | 0.001423829 |
| ENSMUSG00000028617 | Lrrc42 | −1.06075817 | 0.018280576 |
| ENSMUSG00000028613 | Lrp8 | −1.57333132 | 0.036352409 |
| ENSMUSG00000034557 | Zfyve9 | −1.2912005 | 0.00370371 |
| ENSMUSG00000028557 | Rnf11 | −1.09879992 | 0.012536683 |
| ENSMUSG00000028545 | Bend5 | 1.03288902 | 0.025419646 |
| ENSMUSG00000070868 | Skint3 | −2.42664729 | 4.84E−04 |
| ENSMUSG00000085399 | Foxd2os | −1.65069659 | 0.011279543 |
| ENSMUSG00000034171 | Faah | −2.36606785 | 0.019586588 |
| ENSMUSG00000028681 | Ptch2 | −2.73298023 | 5.84E−04 |
| ENSMUSG00000006390 | Elovl1 | 1.29525771 | 0.001042849 |
| ENSMUSG00000028645 | Slc2a1 | 1.32048247 | 3.45E−04 |
| ENSMUSG00000028637 | Ccdc30 | −2.43339504 | 0.020708215 |
| ENSMUSG00000070803 | Cited4 | −2.44219806 | 0.003818156 |
| ENSMUSG00000043207 | Zmpste24 | 1.37818051 | 2.24E−04 |
| ENSMUSG00000028657 | Ppt1 | 1.43044061 | 9.38E−04 |
| ENSMUSG00000028656 | Cap1 | 1.30120435 | 0.002797153 |
| ENSMUSG00000028655 | Mfsd2a | −4.58939949 | 0.001775088 |
| ENSMUSG00000046093 | Hpcal4 | −4.20304419 | 2.60E−04 |
| ENSMUSG00000028648 | Ndufs5 | −1.42771442 | 7.54E−04 |
| ENSMUSG00000028859 | Csf3r | 1.18593673 | 0.001603469 |
| ENSMUSG00000042616 | Oscp1 | −1.13140406 | 0.015331618 |
| ENSMUSG00000050212 | Eva1b | 1.03641707 | 0.007598893 |
| ENSMUSG00000042489 | Clspn | −1.80943863 | 0.010358962 |
| ENSMUSG00000028820 | Sfpq | −1.28778683 | 4.58E−04 |
| ENSMUSG00000042408 | Zmym6 | 1.20178032 | 0.002421046 |
| ENSMUSG00000062545 | Tlr12 | 1.44535168 | 0.001106606 |
| ENSMUSG00000028789 | Azin2 | −1.39495239 | 0.027139535 |
| ENSMUSG00000028785 | Hpca | −4.41254123 | 8.02E−04 |
| ENSMUSG00000050390 | C77080 | −4.04809946 | 0.001611669 |
| ENSMUSG00000000409 | Lck | −1.38747713 | 0.014085713 |
| ENSMUSG00000050493 | Fam167b | 1.74552909 | 1.85E−04 |
| ENSMUSG00000028773 | Fabp3 | −1.54011706 | 5.31E−04 |
| ENSMUSG00000066043 | Phactr4 | −1.14986201 | 0.005735173 |
| ENSMUSG00000028874 | Fgr | −1.12831538 | 0.001498005 |
| ENSMUSG00000028854 | Slc9a1 | 1.23446598 | 0.003671234 |
| ENSMUSG00000047281 | Sfn | −1.80819448 | 0.028654719 |
| ENSMUSG00000037553 | Zdhhc18 | −1.38862137 | 0.003704888 |
| ENSMUSG00000003644 | Rps6ka1 | 1.04657266 | 0.004568005 |
| ENSMUSG00000037348 | Paqr7 | 1.12932555 | 0.001466247 |
| ENSMUSG00000078521 | Aunip | −2.27641887 | 0.014234906 |
| ENSMUSG00000070691 | Runx3 | −2.50084326 | 9.41E−06 |
| ENSMUSG00000028801 | Stpg1 | −1.93640232 | 0.03468578 |
| ENSMUSG00000062157 | Ifnlr1 | −2.08129168 | 0.011859467 |
| ENSMUSG00000028673 | Fuca1 | 1.12059689 | 0.001290167 |
| ENSMUSG00000018983 | E2f2 | −1.94815756 | 8.89E−04 |
| ENSMUSG00000036995 | Asap3 | 1.44776119 | 0.00120219 |
| ENSMUSG00000001604 | Tcea3 | −2.62213872 | 4.53E−04 |
| ENSMUSG00000051351 | Zfp46 | 1.05580297 | 0.01506832 |
| ENSMUSG00000036940 | Kdm1a | −1.23364624 | 0.002185359 |
| ENSMUSG00000036905 | C1qb | 1.31166842 | 0.003821899 |
| ENSMUSG00000036896 | C1qc | 1.51283871 | 0.001364223 |
| ENSMUSG00000028757 | Ddost | 1.19148097 | 0.001249505 |
| ENSMUSG00000083326 | Rpl38-ps1 | −1.6900071 | 0.003568641 |
| ENSMUSG00000046447 | Camk2n1 | −1.38771123 | 0.007101825 |
| ENSMUSG00000041120 | Nbl1 | 1.77392588 | 4.06E−04 |
| ENSMUSG00000078517 | Emc1 | 1.63681603 | 1.96E−04 |
| ENSMUSG00000041025 | Iffo2 | −1.60079331 | 0.002551632 |
| ENSMUSG00000040945 | Rcc2 | −1.34939766 | 5.69E−04 |
| ENSMUSG00000025330 | Padi4 | −3.38325155 | 1.16E−05 |
| ENSMUSG00000036622 | Atp13a2 | 1.59158491 | 0.004007955 |
| ENSMUSG00000028919 | Arhgef19 | −7.69381568 | 4.00E−04 |
| ENSMUSG00000040761 | Spen | −1.09520249 | 0.003419132 |
| ENSMUSG00000040697 | Dnajc16 | 1.12675578 | 0.005683783 |
| ENSMUSG00000040616 | Tmem51 | −1.75436815 | 4.97E−05 |
| ENSMUSG00000073728 | Tmem51os1 | −2.78558067 | 0.003844765 |
| ENSMUSG00000040606 | Kazn | −3.35739847 | 0.003191408 |
| ENSMUSG00000028602 | Tnfrsf8 | −1.98904517 | 0.020926663 |
| ENSMUSG00000070583 | Fv1 | 2.02445912 | 0.00964896 |
| ENSMUSG00000019055 | Plod1 | 1.92083096 | 2.03E−05 |
| ENSMUSG00000041556 | Fbxo2 | −6.21095204 | 7.14E−05 |
| ENSMUSG00000047719 | Ubiad1 | 1.06113172 | 0.003686001 |
| ENSMUSG00000028989 | Angptl7 | 4.56845325 | 3.25E−08 |
| ENSMUSG00000006442 | Srm | 1.00994232 | 0.03285713 |
| ENSMUSG00000028975 | Pex14 | 1.03080418 | 0.003165449 |
| ENSMUSG00000044700 | Tmem201 | 1.75087845 | 3.30E−04 |
| ENSMUSG00000028980 | H6pd | 1.34259603 | 0.026049743 |
| ENSMUSG00000028976 | Slc2a5 | 1.3965736 | 4.19E−04 |
| ENSMUSG00000039838 | Slc45a1 | −4.76823351 | 0.001088593 |
| ENSMUSG00000028965 | Tnfrsf9 | −3.94272893 | 1.48E−04 |
| ENSMUSG00000047777 | Phf13 | 1.09965994 | 0.007360063 |
| ENSMUSG00000028937 | Acot7 | −1.29818977 | 0.002390886 |
| ENSMUSG00000039662 | Icmt | 1.74670538 | 0.003460135 |
| ENSMUSG00000005045 | Chd5 | −3.2396336 | 0.001630948 |
| ENSMUSG00000028931 | Kcnab2 | −2.49254594 | 1.54E−04 |
| ENSMUSG00000029064 | Gnb1 | −2.1831637 | 5.19E−04 |
| ENSMUSG00000073680 | Tmem88b | −5.66084507 | 4.09E−04 |
| ENSMUSG00000029070 | Mxra8 | 3.66712074 | 5.53E−06 |
| ENSMUSG00000050796 | B3galt6 | 1.16395292 | 0.006972379 |
| ENSMUSG00000029076 | Sdf4 | 1.0060629 | 0.003842097 |
| ENSMUSG00000041954 | Tnfrsf18 | −2.41528741 | 5.34E−04 |
| ENSMUSG00000035692 | Isg15 | −1.24187936 | 0.005009894 |
| ENSMUSG00000095567 | Noc2l | 1.19226639 | 0.031189352 |
| ENSMUSG00000096351 | Samd11 | 3.33540785 | 1.59E−04 |
| ENSMUSG00000007415 | Gatad1 | −1.46522224 | 0.003478342 |
| ENSMUSG00000105893 | Wdr46-ps | −3.32312486 | 0.003210019 |
| ENSMUSG00000015653 | Steap2 | −3.04690768 | 0.004625546 |
| ENSMUSG00000012428 | Steap4 | −2.97934412 | 0.004513384 |
| ENSMUSG00000042476 | Abcb4 | 1.6030298 | 8.84E−04 |
| ENSMUSG00000056004 | Elapor2 | −2.93314186 | 0.005283893 |
| ENSMUSG00000061601 | Pclo | −4.90524904 | 0.001212485 |
| ENSMUSG00000040003 | Magi2 | −1.77447518 | 0.032671003 |
| ENSMUSG00000028771 | Ptpn12 | −1.55444223 | 0.0206406 |
| ENSMUSG00000064280 | Ccdc146 | −1.96117801 | 0.02875157 |
| ENSMUSG00000042453 | Reln | −3.74190387 | 7.60E−04 |
| ENSMUSG00000105987 | Al506816 | 1.76674311 | 2.92E−05 |
| ENSMUSG00000028973 | Abcb8 | 1.15544886 | 0.003421228 |
| ENSMUSG00000023353 | Agap3 | −1.11773351 | 0.004780909 |
| ENSMUSG00000038181 | Chpf2 | 1.36705236 | 7.63E−04 |
| ENSMUSG00000086446 | Prkag2os1 | −2.25549359 | 0.006758454 |
| ENSMUSG00000038072 | Galnt11 | 1.01112262 | 0.00901834 |
| ENSMUSG00000062372 | Otof | −2.13756822 | 0.036810193 |
| ENSMUSG00000049265 | Kcnk3 | −1.90494668 | 0.043599005 |
| ENSMUSG00000029175 | Slc35f6 | 1.45810444 | 1.62E−04 |
| ENSMUSG00000038828 | Tmem214 | 1.3624008 | 5.77E−04 |
| ENSMUSG00000029163 | Emilin1 | −1.02960676 | 0.045583427 |
| ENSMUSG00000006641 | Slc5a6 | 2.05497895 | 1.21E−04 |
| ENSMUSG00000013622 | Atraid | 1.2427805 | 0.002583526 |
| ENSMUSG00000107355 | Al839979 | −2.38775625 | 0.00145905 |
| ENSMUSG00000029135 | Fosl2 | −1.55770939 | 3.16E−04 |
| ENSMUSG00000014956 | Ppp1cb | −1.22629408 | 0.005048074 |
| ENSMUSG00000037373 | Ctbp1 | −1.2086398 | 0.004462132 |
| ENSMUSG00000019295 | Tmem129 | 1.20046835 | 0.001191945 |
| ENSMUSG00000048142 | Nat8l | −1.8036409 | 0.002693316 |
| ENSMUSG00000045102 | Poln | −2.85857558 | 0.017188253 |
| ENSMUSG00000001082 | Mfsd10 | 1.02909998 | 0.002330808 |
| ENSMUSG00000051246 | Msantd1 | −5.51686228 | 4.96E−04 |
| ENSMUSG00000044716 | Dok7 | −2.87254201 | 0.018842364 |
| ENSMUSG00000029103 | Lrpap1 | 1.46981946 | 2.80E−04 |
| ENSMUSG00000029097 | Trmt44 | 1.27488947 | 0.01604485 |
| ENSMUSG00000050677 | Ccdc96 | −2.0137731 | 0.011259531 |
| ENSMUSG00000029119 | Man2b2 | 1.78960524 | 5.54E−05 |
| ENSMUSG00000029120 | Ppp2r2c | −5.11934498 | 8.66E−04 |
| ENSMUSG00000063646 | Jakmip1 | −3.93199736 | 4.04E−04 |
| ENSMUSG00000029121 | Crmp1 | −2.41900378 | 0.040902723 |
| ENSMUSG00000048450 | Msx1 | −2.48485377 | 0.016452799 |
| ENSMUSG00000029126 | Nsg1 | −3.95601779 | 1.79E−04 |
| ENSMUSG00000029127 | Zbtb49 | 1.05190891 | 0.014829672 |
| ENSMUSG00000005107 | Slc2a9 | 1.07529253 | 0.033054206 |
| ENSMUSG00000039315 | Clnk | −2.9182218 | 0.006984851 |
| ENSMUSG00000061755 | Bod1l | −1.15564932 | 0.002517947 |
| ENSMUSG00000029082 | Bst1 | −2.89496762 | 7.97E−05 |
| ENSMUSG00000029086 | Prom1 | −1.98262738 | 0.012307951 |
| ENSMUSG00000039706 | Ldb2 | −7.36924907 | 4.70E−04 |
| ENSMUSG00000029088 | Kcnip4 | −3.03914667 | 0.003369598 |
| ENSMUSG00000029189 | Sel1l3 | −4.6396373 | 0.001673663 |
| ENSMUSG00000039178 | Tbc1d19 | 1.17244405 | 0.00161306 |
| ENSMUSG00000029108 | Pcdh7 | −6.69278822 | 0.001250271 |
| ENSMUSG00000090061 | Nwd2 | −4.5332897 | 0.001791146 |
| ENSMUSG00000044827 | Tlr1 | 1.03038361 | 0.005642793 |
| ENSMUSG00000051498 | Tlr6 | 1.00168322 | 0.00822955 |
| ENSMUSG00000054920 | Klhl5 | −1.35714226 | 0.003342892 |
| ENSMUSG00000029223 | Uchl1 | −2.94054751 | 0.001904499 |
| ENSMUSG00000037736 | Limch1 | −4.57405045 | 4.51E−05 |
| ENSMUSG00000037720 | Tmem33 | 1.27795381 | 0.002548331 |
| ENSMUSG00000092060 | Bend4 | −3.36857211 | 3.84E−04 |
| ENSMUSG00000037685 | Atp8a1 | 1.35505229 | 7.51E−04 |
| ENSMUSG00000001260 | Gabrg1 | −3.3322516 | 0.006001732 |
| ENSMUSG00000000560 | Gabra2 | −3.73386093 | 0.001145525 |
| ENSMUSG00000029212 | Gabrb1 | −4.35556994 | 5.90E−05 |
| ENSMUSG00000054892 | Txk | −3.53032852 | 0.000261182 |
| ENSMUSG00000029156 | Sgcb | 1.57045734 | 3.52E−04 |
| ENSMUSG00000062110 | Scfd2 | 1.09828625 | 0.006694603 |
| ENSMUSG00000062960 | Kdr | 1.86099585 | 0.001006029 |
| ENSMUSG00000029233 | Srd5a3 | 1.41007675 | 2.88E−04 |
| ENSMUSG00000036377 | Cracd | −1.83852902 | 0.039263684 |
| ENSMUSG00000059325 | Hopx | −4.20508022 | 6.69E−06 |
| ENSMUSG00000053030 | Spink2 | −3.50856454 | 0.007253497 |
| ENSMUSG00000037605 | Adgrl3 | −4.49964681 | 4.16E−05 |
| ENSMUSG00000029245 | Epha5 | −2.76625861 | 0.020886909 |
| ENSMUSG00000029254 | Stap1 | −2.31662159 | 2.50E−05 |
| ENSMUSG00000067149 | Jchain | 5.3631257 | 8.79E−06 |
| ENSMUSG00000060961 | Slc4a4 | −4.0002841 | 0.001664922 |
| ENSMUSG00000029369 | Afm | −4.02392256 | 0.003870622 |
| ENSMUSG00000029380 | Cxcl1 | 2.30919907 | 3.46E−04 |
| ENSMUSG00000034981 | Parm1 | −2.48389574 | 0.010912008 |
| ENSMUSG00000029417 | Cxcl9 | −1.43117485 | 0.007394085 |
| ENSMUSG00000034855 | Cxcl10 | 1.0056752 | 0.010069226 |
| ENSMUSG00000060183 | Cxcl11 | 2.57015507 | 0.026389201 |
| ENSMUSG00000047963 | Stbd1 | 1.01433777 | 0.032751941 |
| ENSMUSG00000029385 | Ccng2 | 1.91152747 | 2.39E−05 |
| ENSMUSG00000023078 | Cxcl13 | 3.58984271 | 2.00E−06 |
| ENSMUSG00000029335 | Bmp3 | −3.97268216 | 0.005252307 |
| ENSMUSG00000029334 | Prkg2 | −7.42283831 | 5.66E−04 |
| ENSMUSG00000000568 | Hnrnpd | −1.21149817 | 0.001849738 |
| ENSMUSG00000029322 | Plac8 | −2.12926534 | 1.58E−04 |
| ENSMUSG00000029314 | Gpat3 | −1.92994858 | 0.034368151 |
| ENSMUSG00000046709 | Mapk10 | −2.34811358 | 0.040906596 |
| ENSMUSG00000029321 | Slc10a6 | 1.43122057 | 0.025504107 |
| ENSMUSG00000029309 | Sparcl1 | −5.21302653 | 5.04E−06 |
| ENSMUSG00000029304 | Spp1 | −8.9448933 | 1.61E−10 |
| ENSMUSG00000097392 | Thoc2l | −1.60525931 | 0.005688982 |
| ENSMUSG00000034438 | Gbp8 | −1.62080803 | 0.002069729 |
| ENSMUSG00000054720 | Lrrc8c | −1.06945571 | 0.005616789 |
| ENSMUSG00000029287 | Tgfbr3 | −1.70263557 | 8.90E−04 |
| ENSMUSG00000111375 | Btbd8 | 1.36661619 | 0.027801671 |
| ENSMUSG00000029275 | Gfi1 | −2.7474455 | 0.025925671 |
| ENSMUSG00000029263 | Pigg | 1.92024765 | 1.20E−04 |
| ENSMUSG00000033615 | Cplx1 | −5.51040925 | 9.70E−06 |
| ENSMUSG00000013495 | Tmem175 | 1.24526164 | 8.32E−04 |
| ENSMUSG00000029501 | Ankle2 | 1.06246441 | 0.003506659 |
| ENSMUSG00000043323 | Fbrsl1 | 1.08101023 | 0.040082703 |
| ENSMUSG00000033316 | Galnt9 | −8.26366498 | 1.11E−06 |
| ENSMUSG00000029346 | Srrd | 1.1249516 | 0.018133619 |
| ENSMUSG00000042249 | Grk3 | −1.60111453 | 1.07E−04 |
| ENSMUSG00000053334 | Ficd | 1.4031463 | 9.01E−04 |
| ENSMUSG00000054675 | Tmem119 | 2.04447538 | 4.05E−05 |
| ENSMUSG00000048163 | Selplg | 1.87776948 | 1.51E−04 |
| ENSMUSG00000042010 | Acacb | 3.78257773 | 0.001737541 |
| ENSMUSG00000066952 | Myo1h | −2.35010943 | 0.046488116 |
| ENSMUSG00000029524 | Sirt4 | 1.01898126 | 0.01935246 |
| ENSMUSG00000067274 | Rplp0 | −1.13231314 | 0.00255695 |
| ENSMUSG00000041638 | Gcn1 | 1.39725668 | 0.001074591 |
| ENSMUSG00000029516 | Cit | −1.91675209 | 0.001036863 |
| ENSMUSG00000041548 | Hspb8 | −6.0015008 | 8.53E−05 |
| ENSMUSG00000066900 | Suds3 | −1.72233746 | 0.001240128 |
| ENSMUSG00000032959 | Pebp1 | −1.22937895 | 9.96E−04 |
| ENSMUSG00000029598 | Plbd2 | 1.65394628 | 2.37E−04 |
| ENSMUSG00000029603 | Dtx1 | −3.20034838 | 0.009763865 |
| ENSMUSG00000032661 | Oas3 | −1.23073325 | 0.00372568 |
| ENSMUSG00000029608 | Rph3a | −5.12301223 | 0.001536652 |
| ENSMUSG00000064267 | Hvcn1 | 1.69411903 | 5.78E−05 |
| ENSMUSG00000038593 | Tctn1 | 1.63116541 | 4.49E−04 |
| ENSMUSG00000049686 | Orai1 | 1.28074495 | 0.006558543 |
| ENSMUSG00000029477 | Morn3 | −3.20032131 | 9.25E−04 |
| ENSMUSG00000038384 | Setd1b | −2.05450518 | 3.94E−05 |
| ENSMUSG00000029414 | Kntc1 | −1.46218263 | 0.018367179 |
| ENSMUSG00000049241 | Hcar1 | 7.31657689 | 3.64E−04 |
| ENSMUSG00000000915 | Hip1r | −1.13506413 | 0.018339871 |
| ENSMUSG00000029406 | Pitpnm2 | −3.2098199 | 0.013138407 |
| ENSMUSG00000049327 | Kmt5a | −1.23244595 | 0.002716506 |
| ENSMUSG00000038023 | Atp6v0a2 | 1.46416773 | 2.13E−04 |
| ENSMUSG00000038011 | Dnah10 | −2.73175867 | 0.008565918 |
| ENSMUSG00000037979 | Ccdc92 | −2.17170974 | 0.036196071 |
| ENSMUSG00000037962 | Rflna | −4.44646896 | 8.01E−04 |
| ENSMUSG00000029478 | Ncor2 | −1.15206501 | 0.005601918 |
| ENSMUSG00000008348 | Ubc | −2.07070044 | 3.00E−05 |
| ENSMUSG00000029416 | Slc15a4 | 1.7290941 | 1.78E−04 |
| ENSMUSG00000029432 | Nipsnap2 | −1.68440971 | 1.53E−04 |
| ENSMUSG00000025538 | Sumf2 | 1.3670832 | 4.33E−04 |
| ENSMUSG00000025534 | Gusb | 1.30875222 | 7.18E−04 |
| ENSMUSG00000056310 | Tyw1 | 1.21537542 | 9.94E−04 |
| ENSMUSG00000029673 | Auts2 | −2.66769511 | 0.003806315 |
| ENSMUSG00000005374 | Tbl2 | 1.18424699 | 0.003504303 |
| ENSMUSG00000040013 | Fkbp6 | 2.94198013 | 0.012037844 |
| ENSMUSG00000039959 | Hip1 | −1.71114088 | 1.12E−04 |
| ENSMUSG00000005514 | Por | 1.31007793 | 5.18E−04 |
| ENSMUSG00000004951 | Hspb1 | 1.37454618 | 6.59E−04 |
| ENSMUSG00000039747 | Orai2 | 1.14826156 | 0.005272402 |
| ENSMUSG00000001739 | Cldn15 | −1.76738467 | 0.033282023 |
| ENSMUSG00000004846 | Plod3 | 1.09944947 | 0.001824835 |
| ENSMUSG00000023328 | Ache | 1.31322594 | 0.002536292 |
| ENSMUSG00000029714 | Gigyf1 | −1.33614596 | 0.007141629 |
| ENSMUSG00000029713 | Gnb2 | −1.33238387 | 3.87E−04 |
| ENSMUSG00000029712 | Actl6b | −3.71143842 | 0.004231744 |
| ENSMUSG00000046245 | Pilra | −1.10045774 | 0.008441349 |
| ENSMUSG00000066684 | Pilrb1 | −2.18473503 | 3.93E−04 |
| ENSMUSG00000066682 | Pilrb2 | −2.29320852 | 9.47E−05 |
| ENSMUSG00000056966 | Gjc3 | −4.52508458 | 0.001737747 |
| ENSMUSG00000037007 | Zfp113 | 1.0374896 | 0.019771458 |
| ENSMUSG00000047592 | Nxpe5 | −2.43520544 | 0.010110768 |
| ENSMUSG00000029510 | Gpc2 | 1.06994176 | 0.040694279 |
| ENSMUSG00000109713 | Pvrig | 1.76321471 | 0.001725694 |
| ENSMUSG00000025854 | Fam20c | 1.16613433 | 0.001390613 |
| ENSMUSG00000056413 | Adap1 | −1.11222973 | 0.003669905 |
| ENSMUSG00000044197 | Gpr146 | 1.23529792 | 6.32E−04 |
| ENSMUSG00000036687 | Tmem184a | −1.86427399 | 0.007490398 |
| ENSMUSG00000000148 | Brat1 | 1.10167597 | 0.003542887 |
| ENSMUSG00000056493 | Foxk1 | −1.42404023 | 0.007241515 |
| ENSMUSG00000039533 | Mmd2 | −6.43287815 | 3.51E−06 |
| ENSMUSG00000029587 | Zfp12 | 1.08306673 | 0.003063406 |
| ENSMUSG00000039206 | Daglb | 2.08841131 | 1.24E−05 |
| ENSMUSG00000001847 | Rac1 | −1.24503192 | 0.001648916 |
| ENSMUSG00000038970 | Lmtk2 | −1.62594561 | 0.005137936 |
| ENSMUSG00000029623 | Pdap1 | −1.10950971 | 0.003126904 |
| ENSMUSG00000029636 | Wasf3 | −3.89488289 | 2.80E−04 |
| ENSMUSG00000042817 | Flt3 | −2.12658501 | 1.34E−05 |
| ENSMUSG00000029658 | Wdr95 | −3.04411265 | 0.005548525 |
| ENSMUSG00000047735 | Samd9l | −1.1142806 | 0.001593444 |
| ENSMUSG00000044156 | Hepacam2 | −2.33911032 | 3.84E−05 |
| ENSMUSG00000004631 | Sgce | 1.48254247 | 4.56E−04 |
| ENSMUSG00000029759 | Pon3 | 1.73495015 | 9.10E−05 |
| ENSMUSG00000042607 | Asb4 | −3.46629505 | 0.001666063 |
| ENSMUSG00000019577 | Pdk4 | −2.79985334 | 0.006918645 |
| ENSMUSG00000029757 | Dync1i1 | −4.14388262 | 8.63E−04 |
| ENSMUSG00000029571 | Tmem106b | 1.43242901 | 6.78E−04 |
| ENSMUSG00000029569 | Tmem168 | 1.47696982 | 1.29E−04 |
| ENSMUSG00000029534 | St7 | 1.40677726 | 0.005397658 |
| ENSMUSG00000029669 | Tspan12 | −4.30038266 | 2.52E−04 |
| ENSMUSG00000029672 | Fam3c | 1.08827776 | 0.002978015 |
| ENSMUSG00000068748 | Ptprz1 | −4.41715622 | 7.88E−05 |
| ENSMUSG00000029695 | Aass | −3.58188923 | 0.002841368 |
| ENSMUSG00000048022 | Tmem229a | −1.87256799 | 0.005978989 |
| ENSMUSG00000001424 | Snd1 | 1.08222728 | 0.003329486 |
| ENSMUSG00000029767 | Calu | 1.34455128 | 8.92E−04 |
| ENSMUSG00000029769 | Ccdc136 | −2.91780526 | 0.00357381 |
| ENSMUSG00000090685 | Atp6v1fnb | 2.55527566 | 0.012654681 |
| ENSMUSG00000001763 | Tspan33 | −1.08501105 | 0.030375816 |
| ENSMUSG00000001761 | Smo | 1.17804974 | 0.003779818 |
| ENSMUSG00000039629 | Strip2 | −3.1826367 | 1.18E−04 |
| ENSMUSG00000029782 | Tmem209 | 1.42964091 | 8.24E−04 |
| ENSMUSG00000018999 | Slc35b4 | 1.37371463 | 2.68E−04 |
| ENSMUSG00000029761 | Cald1 | −3.4285861 | 0.008127334 |
| ENSMUSG00000057137 | Tmem140 | 1.10405657 | 0.006075188 |
| ENSMUSG00000029838 | Ptn | −3.30754636 | 1.90E−04 |
| ENSMUSG00000038648 | Creb312 | 1.20776486 | 0.003123546 |
| ENSMUSG00000029833 | Trim24 | −1.37776568 | 0.003292599 |
| ENSMUSG00000029823 | Luc7l2 | −1.52277841 | 1.04E−04 |
| ENSMUSG00000071537 | Klrg2 | 2.32678004 | 0.002024603 |
| ENSMUSG00000029925 | Tbxas1 | 1.41032667 | 3.35E−04 |
| ENSMUSG00000029923 | Rab19 | −1.22965263 | 0.009905798 |
| ENSMUSG00000091313 | Fth-ps2 | −3.21688246 | 8.91E−04 |
| ENSMUSG00000029915 | Clec5a | 1.04372385 | 0.004933219 |
| ENSMUSG00000068587 | Mgam | −3.19706046 | 0.010063916 |
| ENSMUSG00000076490 | Trbc1 | −3.87959035 | 1.82E−04 |
| ENSMUSG00000076498 | Trbc2 | −2.85585177 | 0.013429755 |
| ENSMUSG00000029869 | Ephb6 | −1.54245708 | 0.029220823 |
| ENSMUSG00000029859 | Epha1 | −2.05681489 | 0.001099276 |
| ENSMUSG00000039419 | Cntnap2 | −1.73724727 | 0.027868316 |
| ENSMUSG00000029686 | Cul1 | 1.24654966 | 4.90E−04 |
| ENSMUSG00000025823 | Pdia4 | 1.72180224 | 7.23E−05 |
| ENSMUSG00000045466 | Zfp956 | 1.14561689 | 0.008726501 |
| ENSMUSG00000042810 | Krba1 | 1.10755518 | 0.010003176 |
| ENSMUSG00000107476 | Zfp862-ps | 1.27175663 | 0.003492856 |
| ENSMUSG00000064262 | Gimap8 | −3.0263845 | 0.002196047 |
| ENSMUSG00000054435 | Gimap4 | −2.98965638 | 0.001220831 |
| ENSMUSG00000047867 | Gimap6 | 1.48184878 | 2.63E−04 |
| ENSMUSG00000043931 | Gimap7 | −2.41177733 | 0.010167067 |
| ENSMUSG00000044867 | Gimap1os | −2.05485066 | 0.022004021 |
| ENSMUSG00000090019 | Gimap1 | 1.12283012 | 0.003706008 |
| ENSMUSG00000039264 | Gimap3 | −3.0379271 | 2.31E−04 |
| ENSMUSG00000050786 | Ccdc126 | 1.70834336 | 7.93E−04 |
| ENSMUSG00000029832 | Nfe2l3 | −1.88404064 | 0.020319765 |
| ENSMUSG00000038236 | Hoxa7 | −1.78239125 | 0.048636678 |
| ENSMUSG00000086040 | Wipf3 | −4.47270176 | 8.53E−04 |
| ENSMUSG00000019124 | Scrn1 | −4.33995751 | 4.20E−05 |
| ENSMUSG00000038074 | Fkbp14 | 1.84531999 | 9.06E−04 |
| ENSMUSG00000038065 | Mturn | −1.43112366 | 0.012368509 |
| ENSMUSG00000058446 | Znrf2 | −1.28014154 | 0.007664289 |
| ENSMUSG00000002797 | Ggct | −1.17110705 | 0.009326629 |
| ENSMUSG00000029778 | Adcyap1r1 | −8.45429359 | 9.55E−05 |
| ENSMUSG00000004347 | Pde1c | −2.62054863 | 0.00646491 |
| ENSMUSG00000029781 | Fkbp9 | 1.21807534 | 8.56E−04 |
| ENSMUSG00000071419 | Rps15-ps2 | −1.31662345 | 0.040614286 |
| ENSMUSG00000029802 | Abcg2 | 1.8862119 | 5.56E−04 |
| ENSMUSG00000055430 | Nap1l5 | −4.65767088 | 1.46E−04 |
| ENSMUSG00000044162 | Tnip3 | −2.67160414 | 6.32E−06 |
| ENSMUSG00000049001 | Ndnf | −1.46614765 | 0.042112871 |
| ENSMUSG00000029913 | Prdm5 | 1.52174536 | 0.034633438 |
| ENSMUSG00000018341 | Il12rb2 | 1.50174623 | 0.005639882 |
| ENSMUSG00000051397 | Tacstd2 | −3.09171135 | 2.92E−04 |
| ENSMUSG00000076532 | Igkv4-91 | 5.44520852 | 4.71E−04 |
| ENSMUSG00000092746 | Rn7s6 | −1.43884562 | 0.001705844 |
| ENSMUSG00000076563 | Igkv5-48 | 9.16769618 | 4.92E−04 |
| ENSMUSG00000076564 | Igkv12-46 | 6.56790335 | 6.54E−05 |
| ENSMUSG00000076577 | Igkv8-30 | 10.485393 | 3.62E−05 |
| ENSMUSG00000094930 | Igkv6-25 | 7.20198835 | 1.77E−04 |
| ENSMUSG00000076609 | Igkc | 3.8063527 | 1.13E−05 |
| ENSMUSG00000052852 | Reep1 | −2.32657737 | 0.013085747 |
| ENSMUSG00000056091 | St3gal5 | 1.4050854 | 3.69E−04 |
| ENSMUSG00000053460 | Ggcx | 1.13621565 | 0.003980097 |
| ENSMUSG00000056666 | Retsat | 1.72698684 | 1.13E−04 |
| ENSMUSG00000079523 | Tmsb10 | −1.93594125 | 1.48E−05 |
| ENSMUSG00000052861 | Dnah6 | −4.15674781 | 7.50E−04 |
| ENSMUSG00000063063 | Ctnna2 | −3.71948695 | 0.005797496 |
| ENSMUSG00000035104 | Eva1a | 1.35186858 | 5.18E−04 |
| ENSMUSG00000033720 | Sfxn5 | −3.81783308 | 3.88E−05 |
| ENSMUSG00000030000 | Add2 | −3.97433368 | 0.002122262 |
| ENSMUSG00000029999 | Tgfa | 1.4662204 | 0.001321075 |
| ENSMUSG00000029998 | Pcyox1 | 1.82982816 | 4.22E−05 |
| ENSMUSG00000033508 | Asprv1 | −5.76370955 | 9.53E−06 |
| ENSMUSG00000033420 | Antxr1 | −2.81752131 | 0.007791977 |
| ENSMUSG00000030054 | Gp9 | 2.25793246 | 1.10E−04 |
| ENSMUSG00000030062 | Rpn1 | 1.26969357 | 4.78E−04 |
| ENSMUSG00000030082 | Sec61a1 | 1.3072802 | 3.79E−04 |
| ENSMUSG00000030084 | Plxna1 | −1.1993672 | 0.030592636 |
| ENSMUSG00000030088 | Aldh1l1 | −1.92206707 | 2.33E−04 |
| ENSMUSG00000034312 | Iqsec1 | −1.01364969 | 0.011024615 |
| ENSMUSG00000030095 | Tmem43 | 1.10305076 | 0.001500915 |
| ENSMUSG00000030098 | Grip2 | −2.29550146 | 0.007778088 |
| ENSMUSG00000030022 | Adamts9 | −6.74198858 | 9.40E−04 |
| ENSMUSG00000030029 | Lrig1 | −2.40974268 | 0.006832813 |
| ENSMUSG00000030064 | Frmd4b | 1.17747713 | 0.002028885 |
| ENSMUSG00000084319 | Tpt1-ps3 | −1.09194969 | 0.005936175 |
| ENSMUSG00000035378 | Shq1 | 1.20999031 | 0.006802517 |
| ENSMUSG00000005362 | Crbn | 1.12031957 | 0.0018463 |
| ENSMUSG00000030101 | Sumf1 | 1.3973928 | 2.65E−04 |
| ENSMUSG00000030102 | Itpr1 | −1.96132363 | 3.46E−05 |
| ENSMUSG00000030103 | Bhlhe40 | −1.18574586 | 0.001099874 |
| ENSMUSG00000030105 | Arl8b | 1.03745719 | 0.004192952 |
| ENSMUSG00000030104 | Edem1 | 1.63743898 | 6.72E−04 |
| ENSMUSG00000030257 | Srgap3 | −1.88714135 | 6.35E−04 |
| ENSMUSG00000051256 | Jagn1 | 1.06169285 | 0.008512622 |
| ENSMUSG00000030286 | Emc3 | 1.32831412 | 3.29E−04 |
| ENSMUSG00000030307 | Slc6a11 | −7.78771586 | 8.00E−04 |
| ENSMUSG00000030310 | Slc6a1 | −4.59912324 | 0.002521446 |
| ENSMUSG00000009394 | Syn2 | −3.50100648 | 0.007968502 |
| ENSMUSG00000030317 | Timp4 | −6.49062372 | 1.03E−05 |
| ENSMUSG00000000440 | Pparg | −1.59222921 | 5.65E−04 |
| ENSMUSG00000042389 | Tsen2 | 1.06098427 | 0.013953898 |
| ENSMUSG00000059900 | Tmem40 | −1.65789444 | 0.02147327 |
| ENSMUSG00000030123 | Plxnd1 | −3.6116596 | 7.56E−06 |
| ENSMUSG00000030126 | Tmcc1 | −1.27132093 | 4.99E−04 |
| ENSMUSG00000048489 | Depp1 | −1.5488936 | 0.023320847 |
| ENSMUSG00000004988 | Fxyd4 | −2.04427245 | 0.011369705 |
| ENSMUSG00000030134 | Rasgef1a | −1.83771756 | 0.025166974 |
| ENSMUSG00000030145 | Zfp248 | 1.05411314 | 0.047659311 |
| ENSMUSG00000030172 | Erc1 | −1.06259552 | 0.032452157 |
| ENSMUSG00000004902 | Slc25a18 | −4.10108531 | 5.12E−05 |
| ENSMUSG00000030137 | Tuba8 | −2.93619825 | 0.001838447 |
| ENSMUSG00000030108 | Slc6a13 | −3.17753372 | 5.69E−04 |
| ENSMUSG00000108083 | Mug4-ps | −3.52190318 | 0.00691599 |
| ENSMUSG00000007458 | M6pr | 1.09416525 | 0.001842897 |
| ENSMUSG00000040649 | Rimklb | −8.14049457 | 9.00E−05 |
| ENSMUSG00000030117 | Gdf3 | −1.05482949 | 0.015123091 |
| ENSMUSG00000040552 | C3ar1 | 1.58426226 | 2.30E−04 |
| ENSMUSG00000049037 | Clec4a1 | −1.75900765 | 4.96E−05 |
| ENSMUSG00000030147 | Clec4b1 | −2.62223214 | 5.42E−05 |
| ENSMUSG00000067767 | Clec4b2 | −2.98414587 | 0.005857011 |
| ENSMUSG00000030144 | Clec4d | −3.79078834 | 6.69E−06 |
| ENSMUSG00000030142 | Clec4e | −2.3256623 | 1.31E−04 |
| ENSMUSG00000004270 | Lpcat3 | 1.61564218 | 6.14E−05 |
| ENSMUSG00000004263 | Atn1 | −2.05836137 | 0.032305698 |
| ENSMUSG00000004267 | Eno2 | −2.39187797 | 0.002427159 |
| ENSMUSG00000038390 | Gpr162 | −1.61443763 | 0.039877739 |
| ENSMUSG00000030124 | Lag3 | 1.6367373 | 1.58E−04 |
| ENSMUSG00000030122 | Ptms | −1.20435165 | 0.00130604 |
| ENSMUSG00000067714 | Lpar5 | 1.82577691 | 7.39E−05 |
| ENSMUSG00000038213 | Tapbpl | 1.15459527 | 0.001715805 |
| ENSMUSG00000030336 | Cd27 | −1.16896028 | 0.023973558 |
| ENSMUSG00000030340 | Scnn1a | −2.82539843 | 6.17E−05 |
| ENSMUSG00000030342 | Cd9 | 1.81970213 | 5.29E−04 |
| ENSMUSG00000001930 | Vwf | −1.72999843 | 0.002578449 |
| ENSMUSG00000030345 | Dyrk4 | 1.2086319 | 0.005830775 |
| ENSMUSG00000030347 | D6Wsu163e | 1.03837261 | 0.002397481 |
| ENSMUSG00000038028 | Tigar | −1.02800168 | 0.025659286 |
| ENSMUSG00000000184 | Ccnd2 | −2.4903912 | 1.85E−06 |
| ENSMUSG00000030352 | Tspan9 | 1.32046588 | 0.001955751 |
| ENSMUSG00000030359 | Pzp | −6.62349539 | 0.001057431 |
| ENSMUSG00000047228 | A2ml1 | −3.44948457 | 0.007914114 |
| ENSMUSG00000030361 | Klrb1a | −1.71580042 | 0.012921538 |
| ENSMUSG00000030325 | Klrb1c | −1.79239234 | 0.002023688 |
| ENSMUSG00000079298 | Klrb1b | −1.7570803 | 7.73E−04 |
| ENSMUSG00000090164 | BC035044 | 1.0228751 | 0.007529117 |
| ENSMUSG00000030365 | Clec2i | −1.57845425 | 3.19E−04 |
| ENSMUSG00000030154 | Klrb1f | −3.87786775 | 9.23E−05 |
| ENSMUSG00000030157 | Clec2d | −1.77465363 | 0.021394051 |
| ENSMUSG00000053063 | Clec12a | −1.66837859 | 2.16E−04 |
| ENSMUSG00000046080 | Clec9a | −2.50875765 | 7.61E−04 |
| ENSMUSG00000050241 | Klre1 | −3.98682515 | 2.35E−05 |
| ENSMUSG00000030165 | Klrd1 | −2.48352257 | 3.17E−06 |
| ENSMUSG00000030149 | Klrk1 | −2.07784829 | 1.22E−04 |
| ENSMUSG00000052736 | Klrc2 | −3.08905658 | 7.73E−04 |
| ENSMUSG00000030167 | Klrc1 | −3.16468889 | 0.007473389 |
| ENSMUSG00000067610 | Klri1 | −3.31155138 | 3.39E−04 |
| ENSMUSG00000043932 | Klri2 | −2.3283436 | 0.001529372 |
| ENSMUSG00000014543 | Klra17 | −1.83116567 | 0.001683054 |
| ENSMUSG00000079852 | Klra4 | −2.72230001 | 0.008413635 |
| ENSMUSG00000089727 | Klra8 | −3.18825493 | 3.16E−04 |
| ENSMUSG00000072721 | Klra14-ps | −4.49971486 | 0.001106133 |
| ENSMUSG00000033024 | Klra9 | −4.47865803 | 9.62E−05 |
| ENSMUSG00000067599 | Klra7 | −2.57612203 | 0.010169722 |
| ENSMUSG00000030178 | Klra13-ps | −3.40629518 | 1.07E−04 |
| ENSMUSG00000067591 | Klra3 | −3.08810869 | 0.01043399 |
| ENSMUSG00000030187 | Klra2 | −2.11491703 | 1.65E−05 |
| ENSMUSG00000032899 | Styk1 | −2.81938181 | 0.003983352 |
| ENSMUSG00000030189 | Ybx3 | −2.00712557 | 3.21E−04 |
| ENSMUSG00000030203 | Dusp16 | −1.26735647 | 7.62E−04 |
| ENSMUSG00000098318 | Lockd | −1.9378051 | 0.010855807 |
| ENSMUSG00000030208 | Emp1 | −2.68863378 | 5.55E−04 |
| ENSMUSG00000030209 | Grin2b | −2.08832938 | 0.019562039 |
| ENSMUSG00000030214 | Plbd1 | −1.85016948 | 2.68E−05 |
| ENSMUSG00000060032 | H2aj | −1.02657319 | 0.0030728 |
| ENSMUSG00000064330 | Pde6h | −1.8432682 | 0.045001281 |
| ENSMUSG00000015766 | Eps8 | −1.23903026 | 0.00120748 |
| ENSMUSG00000008540 | Mgst1 | −1.79386194 | 5.33E−04 |
| ENSMUSG00000030226 | Lmo3 | −4.81726918 | 8.72E−04 |
| ENSMUSG00000080921 | Rpl38-ps2 | −1.07936336 | 0.017601806 |
| ENSMUSG00000030247 | Kcnj8 | −3.57072204 | 0.006169314 |
| ENSMUSG00000041540 | Sox5 | −2.56305776 | 7.38E−04 |
| ENSMUSG00000043541 | Casc1 | −2.08314574 | 0.009310155 |
| ENSMUSG00000040234 | Tm7sf3 | 1.27346653 | 0.005408033 |
| ENSMUSG00000030313 | Dennd5b | −3.54647449 | 0.001371522 |
| ENSMUSG00000003452 | Bicd1 | −1.94797476 | 0.005424035 |
| ENSMUSG00000078817 | Nlrp12 | −3.0691632 | 0.002155889 |
| ENSMUSG00000078816 | Prkcg | −3.05300245 | 6.17E−04 |
| ENSMUSG00000053338 | Tarm1 | −3.03784791 | 6.02E−04 |
| ENSMUSG00000019734 | Tmc4 | −1.05957093 | 0.045839625 |
| ENSMUSG00000058818 | Pirb | −1.02093489 | 0.002342251 |
| ENSMUSG00000081665 | Pira1 | −1.25726793 | 0.019767846 |
| ENSMUSG00000089942 | Pira2 | −1.73198719 | 0.003331791 |
| ENSMUSG00000074417 | Pira12 | −2.33812438 | 2.61E−04 |
| ENSMUSG00000030427 | Lilra6 | −2.43652556 | 1.46E−04 |
| ENSMUSG00000055541 | Lair1 | 1.30273039 | 0.002969812 |
| ENSMUSG00000030428 | Ttyh1 | −1.65027527 | 0.00232035 |
| ENSMUSG00000062524 | Ncr1 | −2.24477536 | 0.002705643 |
| ENSMUSG00000019254 | Ppp1r12c | −1.27967491 | 5.08E−04 |
| ENSMUSG00000045282 | Tmem86b | −1.27756627 | 0.014630313 |
| ENSMUSG00000035390 | Brsk1 | −4.57308482 | 8.04E−04 |
| ENSMUSG00000046456 | Tmem150b | −2.02308014 | 0.003941338 |
| ENSMUSG00000074406 | Zfp628 | −1.5653109 | 0.017107745 |
| ENSMUSG00000035285 | Nat14 | −1.90423104 | 0.005812694 |
| ENSMUSG00000055150 | Zfp78 | 1.16206346 | 0.037409522 |
| ENSMUSG00000002265 | Peg3 | −1.52842341 | 0.039331928 |
| ENSMUSG00000051527 | Usp29 | −5.05789872 | 8.27E−04 |
| ENSMUSG00000063535 | Zfp773 | 1.76294959 | 6.38E−04 |
| ENSMUSG00000066838 | Zfp772 | 1.07544095 | 0.012433998 |
| ENSMUSG00000030393 | Zik1 | 1.25157997 | 0.01483009 |
| ENSMUSG00000034071 | Zfp551 | 1.90209203 | 0.007359859 |
| ENSMUSG00000060397 | Zfp128 | 1.3278221 | 0.007645064 |
| ENSMUSG00000054715 | Zscan22 | 1.0181145 | 0.00923892 |
| ENSMUSG00000004500 | Zfp324 | 1.10075761 | 0.006701957 |
| ENSMUSG00000041571 | Selenow | −1.7353383 | 1.42E−04 |
| ENSMUSG00000041375 | Ccdc9 | −1.02126606 | 0.009370662 |
| ENSMUSG00000001918 | Slc1a5 | −1.39170924 | 6.25E−04 |
| ENSMUSG00000048920 | Fkrp | 1.18011157 | 0.001935227 |
| ENSMUSG00000078794 | Dact3 | −1.99885456 | 0.030599746 |
| ENSMUSG00000043017 | Ptgir | −1.76599172 | 0.006157127 |
| ENSMUSG00000019370 | Calm3 | −1.10903614 | 0.001947171 |
| ENSMUSG00000070802 | Pnma8b | −4.24456062 | 0.002223168 |
| ENSMUSG00000030413 | Pglyrp1 | 2.23556209 | 1.58E−04 |
| ENSMUSG00000044030 | Irf2bp1 | 1.11341601 | 0.014773826 |
| ENSMUSG00000030410 | Dmwd | −2.45196246 | 0.007043267 |
| ENSMUSG00000030409 | Dmpk | −3.93441143 | 0.002142482 |
| ENSMUSG00000030407 | Qpctl | 1.48097057 | 5.41E−04 |
| ENSMUSG00000040811 | Eml2 | −1.52645326 | 0.040260109 |
| ENSMUSG00000030401 | Rtn2 | −2.39288079 | 0.004488742 |
| ENSMUSG00000040714 | Klc3 | −3.10787317 | 0.00949795 |
| ENSMUSG00000002981 | Clptm1 | 1.53262025 | 1.19E−04 |
| ENSMUSG00000002992 | Apoc2 | −6.48980618 | 0.001248022 |
| ENSMUSG00000074336 | Apoc4 | −1.3181001 | 0.01192301 |
| ENSMUSG00000062300 | Nectin2 | 1.98707258 | 4.07E−05 |
| ENSMUSG00000055305 | Zfp93 | 1.24619054 | 0.041383724 |
| ENSMUSG00000050605 | Zfp61 | 1.11781214 | 0.03507673 |
| ENSMUSG00000055826 | Tescl | 4.11840925 | 0.002846426 |
| ENSMUSG00000052212 | Cd177 | −2.39441917 | 2.87E−06 |
| ENSMUSG00000054169 | Ceacam10 | −2.1442513 | 0.02983696 |
| ENSMUSG00000057177 | Gsk3a | −2.26817504 | 0.002392334 |
| ENSMUSG00000040703 | Cyp2s1 | −2.9216156 | 0.00287476 |
| ENSMUSG00000003752 | Itpkc | 1.68284542 | 0.007018496 |
| ENSMUSG00000011751 | Sptbn4 | −1.72807872 | 0.02046224 |
| ENSMUSG00000003363 | Pld3 | 1.71196418 | 1.13E−04 |
| ENSMUSG00000078779 | Zfp59 | 1.38503928 | 0.009488005 |
| ENSMUSG00000037337 | Map4k1 | −1.12645835 | 0.003162946 |
| ENSMUSG00000030592 | Ryr1 | −2.15526058 | 0.001006395 |
| ENSMUSG00000030589 | Rasgrp4 | −2.79226943 | 2.74E−06 |
| ENSMUSG00000037239 | Spred3 | −2.64486886 | 0.004504056 |
| ENSMUSG00000046410 | Kcnk6 | 1.42451099 | 7.84E−04 |
| ENSMUSG00000030588 | Yif1b | 1.13205306 | 0.002029008 |
| ENSMUSG00000074227 | Spint2 | −2.00183794 | 8.76E−04 |
| ENSMUSG00000046185 | Zfp84 | 1.09699331 | 0.002127154 |
| ENSMUSG00000011427 | Zfp790 | 1.24110131 | 0.001867839 |
| ENSMUSG00000050855 | Zfp940 | 1.22350633 | 0.014428486 |
| ENSMUSG00000001794 | Capns1 | −1.59093833 | 7.20E−04 |
| ENSMUSG00000013921 | Clip3 | −2.80927637 | 6.81E−04 |
| ENSMUSG00000036882 | Arhgap33 | −2.26180384 | 0.048119686 |
| ENSMUSG00000006307 | Kmt2b | −1.2997756 | 0.004078266 |
| ENSMUSG00000060962 | Dmkn | −2.63582548 | 0.029439362 |
| ENSMUSG00000051314 | Ffar2 | −3.30296681 | 3.89E−04 |
| ENSMUSG00000030577 | Cd22 | 1.17881468 | 0.001462158 |
| ENSMUSG00000100029 | D7Ertd128e | 1.35452345 | 0.031972236 |
| ENSMUSG00000001247 | Lsr | −1.65234665 | 0.002643777 |
| ENSMUSG00000009687 | Fxyd5 | −1.69417121 | 2.32E−04 |
| ENSMUSG00000036578 | Fxyd7 | −1.98357471 | 0.046670632 |
| ENSMUSG00000036570 | Fxyd1 | −3.73740181 | 8.66E−04 |
| ENSMUSG00000060402 | Chst8 | 1.04143131 | 0.007376276 |
| ENSMUSG00000043671 | Dpy19l3 | 1.21291569 | 0.012534831 |
| ENSMUSG00000021217 | Tshz3 | −3.56734135 | 0.006274109 |
| ENSMUSG00000030424 | Zfp939 | 1.16338405 | 0.016478158 |
| ENSMUSG00000068959 | Zfp619 | 1.03094335 | 0.039231708 |
| ENSMUSG00000092193 | Cd9-ps | 2.69390616 | 0.001753204 |
| ENSMUSG00000012640 | Zfp715 | 1.19659356 | 0.002879479 |
| ENSMUSG00000039013 | Siglecf | 1.64343387 | 1.87E−04 |
| ENSMUSG00000030468 | Siglecg | −1.42474495 | 8.85E−04 |
| ENSMUSG00000004612 | Nkg7 | −3.29586406 | 3.67E−05 |
| ENSMUSG00000004609 | Cd33 | 1.14970247 | 0.00299518 |
| ENSMUSG00000056592 | Zfp658 | 1.32869447 | 7.14E−04 |
| ENSMUSG00000064023 | Klk8 | 1.3232373 | 0.002926217 |
| ENSMUSG00000060177 | Klk1b22 | 10.5207246 | 1.72E−06 |
| ENSMUSG00000038738 | Shank1 | −2.45841767 | 5.09E−04 |
| ENSMUSG00000030731 | Syt3 | 1.48507498 | 0.00374701 |
| ENSMUSG00000008193 | Spib | −2.22987112 | 6.55E−04 |
| ENSMUSG00000002204 | Napsa | −2.36866412 | 3.94E−06 |
| ENSMUSG00000030739 | Myh14 | −4.82242814 | 5.20E−04 |
| ENSMUSG00000007783 | Cpt1c | −4.03150019 | 3.44E−04 |
| ENSMUSG00000046574 | Prr12 | −1.22803526 | 0.009859203 |
| ENSMUSG00000030792 | Dkkl1 | −1.76125921 | 0.033791208 |
| ENSMUSG00000030798 | Cd37 | 1.1410288 | 0.001414796 |
| ENSMUSG00000003863 | Ppfia3 | −1.79851721 | 0.024943973 |
| ENSMUSG00000030824 | Nucb1 | 1.56418502 | 1.37E−04 |
| ENSMUSG00000059824 | Dbp | 1.47103171 | 0.001280574 |
| ENSMUSG00000054161 | Fam83e | −2.57100589 | 0.004351817 |
| ENSMUSG00000003271 | Sult2b1 | −5.09510463 | 6.39E−04 |
| ENSMUSG00000058743 | Kcnj14 | 3.9449002 | 2.92E−05 |
| ENSMUSG00000040212 | Emp3 | −1.51904544 | 0.001037662 |
| ENSMUSG00000030835 | Nomo1 | 1.13868197 | 0.004446774 |
| ENSMUSG00000040026 | Saa3 | −2.17310638 | 0.011803006 |
| ENSMUSG00000010307 | Tmem86a | 1.19890967 | 8.89E−04 |
| ENSMUSG00000030854 | Ptpn5 | −2.28969613 | 0.048802903 |
| ENSMUSG00000093973 | Mrgpra2a | −2.25236993 | 0.017024391 |
| ENSMUSG00000096719 | Mrgpra2b | −2.60125101 | 0.002315395 |
| ENSMUSG00000030471 | Zdhhc13 | 1.12556707 | 0.001259994 |
| ENSMUSG00000046179 | E2f8 | −1.72384952 | 0.007619581 |
| ENSMUSG00000063297 | Luzp2 | −4.25679608 | 3.39E−05 |
| ENSMUSG00000051504 | Siglech | 1.81934238 | 3.00E−04 |
| ENSMUSG00000033790 | Tubgcp5 | 1.2263609 | 5.53E−04 |
| ENSMUSG00000102252 | Snrpn | −5.18857945 | 4.31E−06 |
| ENSMUSG00000033585 | Ndn | −7.84422089 | 4.69E−04 |
| ENSMUSG00000052040 | Klf13 | −2.28018654 | 8.39E−04 |
| ENSMUSG00000030519 | Apba2 | −8.52507453 | 1.44E−04 |
| ENSMUSG00000078681 | Tm2d3 | 1.22318044 | 0.005467696 |
| ENSMUSG00000074071 | Fam169b | −4.18158073 | 0.003429494 |
| ENSMUSG00000097530 | Kansl2-ps | 1.64714393 | 0.041134067 |
| ENSMUSG00000030605 | Mfge8 | 1.17496174 | 9.21E−04 |
| ENSMUSG00000039194 | Rlbp1 | −4.80935049 | 5.34E−04 |
| ENSMUSG00000039062 | Anpep | −2.04164915 | 0.018717912 |
| ENSMUSG00000030532 | Hddc3 | 2.3363654 | 1.76E−04 |
| ENSMUSG00000030536 | Iqgap1 | −1.4247947 | 0.001196844 |
| ENSMUSG00000025584 | Pde8a | −1.63986219 | 0.003779141 |
| ENSMUSG00000025586 | Cpeb1 | −1.07267244 | 0.01311963 |
| ENSMUSG00000045795 | Whamm | −1.9811972 | 8.29E−04 |
| ENSMUSG00000025813 | Homer2 | −3.45196151 | 0.001198442 |
| ENSMUSG00000025104 | Hdgfl3 | −1.21097372 | 0.011615397 |
| ENSMUSG00000070469 | Adamtsl3 | −2.03219407 | 0.042777293 |
| ENSMUSG00000001741 | Il16 | 1.10572649 | 0.001395287 |
| ENSMUSG00000011154 | Cfap161 | −1.9650085 | 0.023724242 |
| ENSMUSG00000038503 | Mesd | 1.34509411 | 6.31E−04 |
| ENSMUSG00000038459 | Abhd17c | −1.15655662 | 0.011931364 |
| ENSMUSG00000015709 | Arnt2 | −2.31280801 | 1.89E−04 |
| ENSMUSG00000049583 | Grm5 | −4.67113018 | 0.00129236 |
| ENSMUSG00000030560 | Ctsc | 1.42743436 | 7.14E−04 |
| ENSMUSG00000039428 | Tmem135 | 1.33558668 | 3.56E−04 |
| ENSMUSG00000049791 | Fzd4 | 2.13393795 | 1.34E−04 |
| ENSMUSG00000030621 | Me3 | −3.22492064 | 0.006161109 |
| ENSMUSG00000062235 | Btf3-ps5 | 7.98631212 | 1.82E−04 |
| ENSMUSG00000030616 | Sytl2 | −8.61571745 | 1.19E−04 |
| ENSMUSG00000052572 | Dlg2 | −2.2731948 | 0.014794776 |
| ENSMUSG00000035713 | Usp35 | −1.07458895 | 0.003494183 |
| ENSMUSG00000051727 | Kctd14 | −3.38719964 | 4.63E−05 |
| ENSMUSG00000074006 | Omp | −7.08009598 | 5.05E−04 |
| ENSMUSG00000030760 | Acer3 | 1.06789998 | 0.009663475 |
| ENSMUSG00000049580 | Tsku | 3.92256585 | 6.34E−05 |
| ENSMUSG00000015957 | Wnt11 | −4.15804599 | 0.003716725 |
| ENSMUSG00000052396 | Mogat2 | −2.49650229 | 0.013079186 |
| ENSMUSG00000055407 | Map6 | −2.53113801 | 0.01613425 |
| ENSMUSG00000096606 | Tpbgl | 2.22937217 | 0.00162479 |
| ENSMUSG00000030737 | Slco2b1 | 1.81876938 | 1.45E−04 |
| ENSMUSG00000058761 | Rnf169 | −1.05163628 | 0.003966689 |
| ENSMUSG00000035165 | Kcne3 | −2.31705962 | 1.40E−04 |
| ENSMUSG00000051048 | P4ha3 | −4.56701951 | 0.001287206 |
| ENSMUSG00000030708 | Dnajb13 | −1.36343672 | 0.003720415 |
| ENSMUSG00000030701 | Plekhb1 | −5.41950456 | 5.25E−07 |
| ENSMUSG00000008318 | Relt | −1.24373096 | 9.66E−04 |
| ENSMUSG00000032875 | Arhgef17 | −3.01845498 | 0.002803098 |
| ENSMUSG00000032860 | P2ry2 | 3.05216133 | 1.17E−04 |
| ENSMUSG00000030691 | Fchsd2 | 1.13175707 | 0.0012318 |
| ENSMUSG00000030688 | Stard10 | −2.25446238 | 0.00691439 |
| ENSMUSG00000092517 | Art2a | −5.77030322 | 6.18E−05 |
| ENSMUSG00000001829 | Clpb | 1.01056603 | 0.004119937 |
| ENSMUSG00000078630 | Tomt | 1.07681496 | 0.044329531 |
| ENSMUSG00000073968 | Trim68 | 1.20697134 | 0.002446157 |
| ENSMUSG00000072244 | Trim6 | −3.5705804 | 0.002079733 |
| ENSMUSG00000060441 | Trim5 | −6.08318702 | 1.67E−07 |
| ENSMUSG00000066258 | Trim12a | −8.21503109 | 9.32E−09 |
| ENSMUSG00000090215 | Trim34b | 2.98968023 | 1.59E−05 |
| ENSMUSG00000052749 | Trim30b | −2.07153462 | 7.52E−05 |
| ENSMUSG00000057596 | Trim30d | −4.96900666 | 4.89E−08 |
| ENSMUSG00000037060 | Cavin3 | −3.8270457 | 5.82E−04 |
| ENSMUSG00000037049 | Smpd1 | 1.43024298 | 2.82E−04 |
| ENSMUSG00000030894 | Tpp1 | 1.79285759 | 5.09E−05 |
| ENSMUSG00000086513 | Gvin-ps1 | 3.20001959 | 7.80E−05 |
| ENSMUSG00000045868 | Gvin1 | −3.02649585 | 6.01E−05 |
| ENSMUSG00000098934 | Gvin-ps4 | −3.5705804 | 0.002079733 |
| ENSMUSG00000053541 | Gvin-ps6 | 2.97142854 | 6.77E−06 |
| ENSMUSG00000073902 | Gvin3 | −7.3542078 | 1.09E−07 |
| ENSMUSG00000051041 | Olfml1 | −2.94155313 | 0.006293599 |
| ENSMUSG00000048330 | Ric3 | 1.70841542 | 2.76E−04 |
| ENSMUSG00000036111 | Lmo1 | −2.19125624 | 0.038165062 |
| ENSMUSG00000034825 | Nrip3 | −2.98273246 | 0.001225691 |
| ENSMUSG00000047554 | Tmem41b | 1.16741831 | 0.003458909 |
| ENSMUSG00000030772 | Dkk3 | −3.32763441 | 0.001108835 |
| ENSMUSG00000030770 | Parva | −4.84981976 | 5.03E−05 |
| ENSMUSG00000038156 | Spon1 | −2.84119666 | 7.70E−04 |
| ENSMUSG00000030754 | Copb1 | 1.01913021 | 0.002392584 |
| ENSMUSG00000058420 | Syt17 | −6.82291742 | 8.50E−04 |
| ENSMUSG00000042246 | Tmc7 | 2.08805593 | 2.57E−05 |
| ENSMUSG00000033917 | Gde1 | 1.08890062 | 0.001592901 |
| ENSMUSG00000073856 | Iqck | 1.67825813 | 0.048598105 |
| ENSMUSG00000030905 | Crym | −3.76728003 | 3.38E−04 |
| ENSMUSG00000035064 | Eef2k | 1.24026441 | 0.002313719 |
| ENSMUSG00000030748 | Il4ra | 1.18960657 | 0.00196411 |
| ENSMUSG00000030745 | Il21r | 1.16392268 | 0.00162062 |
| ENSMUSG00000030742 | Lat | −1.85095642 | 0.019617596 |
| ENSMUSG00000030674 | Qprt | 1.05446374 | 0.028878645 |
| ENSMUSG00000051457 | Spn | −2.44801395 | 1.85E−05 |
| ENSMUSG00000042492 | Tbc1d10b | −1.07633887 | 0.004703195 |
| ENSMUSG00000000486 | Septin1 | −2.32663573 | 0.001056479 |
| ENSMUSG00000030830 | Itgal | −1.69227617 | 4.08E−05 |
| ENSMUSG00000045757 | Zfp764 | 1.0688556 | 0.007525811 |
| ENSMUSG00000042308 | Setd1a | −1.50259801 | 0.002020076 |
| ENSMUSG00000030806 | Stx1b | −3.29901933 | 5.13E−04 |
| ENSMUSG00000096145 | Vkorc1 | 1.19172242 | 0.009950021 |
| ENSMUSG00000030795 | Fus | −1.22106308 | 7.89E−04 |
| ENSMUSG00000030781 | Slc5a2 | −2.0726169 | 0.040636645 |
| ENSMUSG00000030780 | Rusf1 | 1.14507749 | 0.002304583 |
| ENSMUSG00000048170 | Mcmbp | −1.08590677 | 0.016744162 |
| ENSMUSG00000030849 | Fgfr2 | −4.60482986 | 0.00268314 |
| ENSMUSG00000030956 | Fam53b | 1.08040383 | 0.005744339 |
| ENSMUSG00000073805 | Insyn2a | −1.97184232 | 0.001606763 |
| ENSMUSG00000031004 | Mki67 | −1.60593134 | 4.58E−04 |
| ENSMUSG00000015981 | Stk32c | −3.33582961 | 0.009768881 |
| ENSMUSG00000025475 | Adgra1 | −7.54991244 | 4.74E−04 |
| ENSMUSG00000025473 | Adam8 | −1.15701613 | 0.002102319 |
| ENSMUSG00000060591 | Ifitm2 | −1.73113547 | 2.68E−04 |
| ENSMUSG00000025491 | Ifitm1 | −3.24812332 | 4.90E−06 |
| ENSMUSG00000025492 | Ifitm3 | −1.15570805 | 0.021548659 |
| ENSMUSG00000059108 | Ifitm6 | −2.37119767 | 8.21E−04 |
| ENSMUSG00000054065 | Pkp3 | −1.87683703 | 7.16E−04 |
| ENSMUSG00000025494 | Sigirr | −1.41414033 | 0.004839087 |
| ENSMUSG00000025495 | Ptdss2 | 1.3821416 | 3.65E−04 |
| ENSMUSG00000060240 | Cend1 | −3.01397599 | 0.001444772 |
| ENSMUSG00000025510 | Cd151 | 1.49915149 | 3.10E−04 |
| ENSMUSG00000025511 | Tspan4 | 1.86596428 | 1.18E−04 |
| ENSMUSG00000053046 | Brsk2 | −2.13686659 | 0.018397139 |
| ENSMUSG00000007891 | Ctsd | 1.79394167 | 0.001021396 |
| ENSMUSG00000018819 | Lsp1 | −1.02882471 | 0.004408187 |
| ENSMUSG00000061723 | Tnnt3 | −3.23325953 | 0.001079997 |
| ENSMUSG00000037706 | Cd81 | 1.64240333 | 5.66E−04 |
| ENSMUSG00000037664 | Cdkn1c | 1.15957593 | 0.037325845 |
| ENSMUSG00000037613 | Tnfrsf23 | 1.16169547 | 0.004295073 |
| ENSMUSG00000037606 | Osbpl5 | −2.6247578 | 0.007762144 |
| ENSMUSG00000048965 | Mrgpre | 1.87065615 | 4.91E−04 |
| ENSMUSG00000058454 | Dhcr7 | 1.50320325 | 2.23E−04 |
| ENSMUSG00000037541 | Shank2 | −7.00812624 | 7.53E−04 |
| ENSMUSG00000031078 | Cttn | −3.71169332 | 3.85E−04 |
| ENSMUSG00000037519 | Ppfia1 | −1.03229673 | 0.008603503 |
| ENSMUSG00000040624 | Plekhg1 | −2.99375439 | 2.08E−04 |
| ENSMUSG00000019767 | Ccdc170 | −2.20409765 | 0.007766653 |
| ENSMUSG00000096054 | Syne1 | −2.7274363 | 1.07E−05 |
| ENSMUSG00000019775 | Rgs17 | −2.86749392 | 0.019315206 |
| ENSMUSG00000039981 | Zc3h12d | −2.07548533 | 2.74E−04 |
| ENSMUSG00000015755 | Tab2 | −1.03662853 | 0.004795392 |
| ENSMUSG00000015305 | Sash1 | −1.94695806 | 0.004180443 |
| ENSMUSG00000019820 | Utrn | −1.82529168 | 1.47E−04 |
| ENSMUSG00000039232 | Stx11 | −1.51442042 | 0.00250405 |
| ENSMUSG00000019810 | Fuca2 | 1.24775567 | 0.001066792 |
| ENSMUSG00000015501 | Hivep2 | −1.15848828 | 0.003401899 |
| ENSMUSG00000039835 | Nhsl1 | −3.66102085 | 0.001229474 |
| ENSMUSG00000019851 | Perp | 1.01314688 | 0.026231973 |
| ENSMUSG00000019850 | Tnfaip3 | 1.63921078 | 0.003848798 |
| ENSMUSG00000020009 | Ifngr1 | 1.15326416 | 0.003121671 |
| ENSMUSG00000071369 | Map3k5 | −1.14311311 | 0.002364868 |
| ENSMUSG00000019992 | Mtfr2 | −1.48598189 | 0.027132564 |
| ENSMUSG00000019990 | Pde7b | −2.46671538 | 0.001099217 |
| ENSMUSG00000019970 | Sgk1 | 1.78907903 | 1.07E−04 |
| ENSMUSG00000075297 | H60b | −1.29910745 | 0.046645729 |
| ENSMUSG00000097295 | Hmgb1-ps8 | −1.66443654 | 0.004279076 |
| ENSMUSG00000010461 | Eya4 | 1.07786159 | 0.024473101 |
| ENSMUSG00000037455 | Slc18b1 | 1.77763204 | 6.79E−04 |
| ENSMUSG00000020010 | Vnn3 | −1.08022854 | 0.036817675 |
| ENSMUSG00000037370 | Enpp1 | 1.30958359 | 0.002795537 |
| ENSMUSG00000019987 | Arg1 | −2.02376443 | 0.014675715 |
| ENSMUSG00000039166 | Akap7 | −1.19394988 | 0.007427309 |
| ENSMUSG00000051354 | Samd3 | −3.08124205 | 0.003377628 |
| ENSMUSG00000019899 | Lama2 | −2.7835466 | 0.007654876 |
| ENSMUSG00000038916 | Soga3 | −3.55566976 | 0.001411131 |
| ENSMUSG00000039485 | Tspyl4 | −2.19007289 | 0.002954813 |
| ENSMUSG00000019777 | Hdac2 | 1.34578803 | 0.002400718 |
| ENSMUSG00000019843 | Fyn | −1.45546073 | 7.41E−04 |
| ENSMUSG00000019842 | Traf3ip2 | −1.72444535 | 0.016966001 |
| ENSMUSG00000063428 | Ddo | −1.86633669 | 0.015446255 |
| ENSMUSG00000019818 | Cd164 | 1.65847337 | 1.74E−04 |
| ENSMUSG00000048756 | Foxo3 | −1.02916424 | 0.014247748 |
| ENSMUSG00000047139 | Cd24a | −2.12259876 | 1.33E−05 |
| ENSMUSG00000019866 | Crybg1 | −1.23405384 | 0.008787781 |
| ENSMUSG00000038151 | Prdm1 | 1.39959704 | 9.55E−04 |
| ENSMUSG00000019861 | Gopc | 1.06345375 | 0.012528281 |
| ENSMUSG00000047669 | Msl3l2 | 1.06203709 | 0.02321012 |
| ENSMUSG00000050953 | Gja1 | −2.7317721 | 0.002387016 |
| ENSMUSG00000019877 | Serinc1 | 1.51027133 | 4.14E−04 |
| ENSMUSG00000019874 | Fabp7 | −7.01529969 | 1.28E−05 |
| ENSMUSG00000058537 | AW822073 | −3.2046735 | 0.012791405 |
| ENSMUSG00000038010 | Ccdc138 | −1.24606807 | 0.035455025 |
| ENSMUSG00000019916 | P4ha1 | 1.63942251 | 1.10E−04 |
| ENSMUSG00000057337 | Chst3 | 2.37440716 | 0.008856402 |
| ENSMUSG00000012819 | Cdh23 | 1.16112507 | 0.001298417 |
| ENSMUSG00000020101 | Vsir | 1.3618465 | 6.81E−04 |
| ENSMUSG00000020100 | Slc29a3 | 1.73175861 | 8.20E−05 |
| ENSMUSG00000020097 | Sgpl1 | 1.71475122 | 1.86E−04 |
| ENSMUSG00000059901 | Adamts14 | −2.51523143 | 0.009034714 |
| ENSMUSG00000037202 | Prf1 | −2.93825128 | 2.14E−04 |
| ENSMUSG00000061488 | Rpl27a-ps1 | −1.68515246 | 0.049769434 |
| ENSMUSG00000020092 | Pald1 | 1.37712165 | 8.09E−04 |
| ENSMUSG00000020085 | Aifm2 | −1.27039784 | 0.015337908 |
| ENSMUSG00000020083 | Fam241b | −2.10392151 | 0.017822056 |
| ENSMUSG00000037031 | Tspan15 | −5.01817365 | 8.86E−04 |
| ENSMUSG00000036955 | Kifbp | 1.55337563 | 0.003759422 |
| ENSMUSG00000036875 | Dna2 | −1.04698643 | 0.005246661 |
| ENSMUSG00000037855 | Zfp365 | −3.58243779 | 4.95E−04 |
| ENSMUSG00000037846 | Rtkn2 | −1.61667677 | 0.040764931 |
| ENSMUSG00000069601 | Ank3 | −2.59942374 | 0.007916603 |
| ENSMUSG00000048701 | Ccdc6 | −1.28787067 | 0.007413853 |
| ENSMUSG00000037747 | Phyhipl | −6.93782793 | 5.74E−05 |
| ENSMUSG00000014329 | Bicc1 | −2.30158039 | 0.016951424 |
| ENSMUSG00000009681 | Bcr | −1.377252 | 0.012467802 |
| ENSMUSG00000020178 | Adora2a | −2.28183099 | 0.008093494 |
| ENSMUSG00000033427 | Upb1 | −1.25149745 | 0.034603124 |
| ENSMUSG00000046807 | Lrrc75b | −3.79228657 | 0.00629102 |
| ENSMUSG00000000901 | Mmp11 | 2.59393905 | 0.005612158 |
| ENSMUSG00000049422 | Chchd10 | −1.76442061 | 7.62E−04 |
| ENSMUSG00000033208 | S100b | −5.33306033 | 2.08E−07 |
| ENSMUSG00000001436 | Slc19a1 | 1.81392676 | 1.73E−04 |
| ENSMUSG00000000290 | Itgb2 | 1.01976772 | 0.003833828 |
| ENSMUSG00000009291 | Pttg1ip | 1.47256836 | 2.17E−04 |
| ENSMUSG00000051652 | Lrrc3 | 1.58274259 | 1.61E−04 |
| ENSMUSG00000032763 | Ilvbl | 1.20766063 | 6.57E−04 |
| ENSMUSG00000023175 | Bsg | 1.151332 | 0.001595324 |
| ENSMUSG00000020323 | Prss57 | −2.9760487 | 0.002040258 |
| ENSMUSG00000035863 | Palm | −1.87367379 | 0.007611718 |
| ENSMUSG00000057729 | Prtn3 | −3.20584446 | 5.06E−04 |
| ENSMUSG00000020125 | Elane | −3.40994796 | 3.14E−04 |
| ENSMUSG00000013858 | Tmem259 | −1.34684009 | 4.95E−04 |
| ENSMUSG00000004665 | Cnn2 | −1.37458301 | 3.23E−04 |
| ENSMUSG00000003068 | Stk11 | −1.4207893 | 5.24E−04 |
| ENSMUSG00000035640 | Cbarp | −1.72307987 | 0.017227256 |
| ENSMUSG00000003345 | Csnk1g2 | −1.26452745 | 0.004511308 |
| ENSMUSG00000003344 | Btbd2 | −1.05955848 | 0.022104544 |
| ENSMUSG00000020190 | Mknk2 | −1.5211765 | 1.38E−04 |
| ENSMUSG00000035206 | Sppl2b | 1.15226257 | 0.00279907 |
| ENSMUSG00000048240 | Gng7 | −1.0653722 | 0.039150522 |
| ENSMUSG00000085779 | Atcayos | −6.78101435 | 7.72E−04 |
| ENSMUSG00000034949 | Zfr2 | −1.50722885 | 0.033524008 |
| ENSMUSG00000020238 | Ncln | 1.33902179 | 7.46E−04 |
| ENSMUSG00000044199 | S1pr4 | −1.75721555 | 0.001760411 |
| ENSMUSG00000034792 | Gna15 | 1.40886836 | 2.05E−04 |
| ENSMUSG00000054452 | Tle5 | −1.28832463 | 0.003124385 |
| ENSMUSG00000020263 | Appl2 | 1.02006888 | 0.002980247 |
| ENSMUSG00000046841 | Ckap4 | −1.67468348 | 5.84E−04 |
| ENSMUSG00000020037 | Rfx4 | −4.12762971 | 8.79E−04 |
| ENSMUSG00000035529 | Prdm4 | 1.01505476 | 0.022148061 |
| ENSMUSG00000020044 | Timp3 | −3.39559797 | 9.09E−04 |
| ENSMUSG00000056366 | Fabp3-ps1 | −1.38758311 | 0.02942352 |
| ENSMUSG00000058934 | Igf1os | 2.25658165 | 0.014671839 |
| ENSMUSG00000035311 | Gnptab | 1.00496532 | 0.013558777 |
| ENSMUSG00000058589 | Anks1b | −4.07472673 | 0.001866726 |
| ENSMUSG00000020017 | Hal | −2.12578101 | 0.033568977 |
| ENSMUSG00000074785 | Plxnc1 | −1.52969931 | 0.00153585 |
| ENSMUSG00000090035 | Galnt4 | 1.26815945 | 0.00117584 |
| ENSMUSG00000090665 | Gad1-ps | −1.00369822 | 0.042683781 |
| ENSMUSG00000019966 | Kitl | 1.27443967 | 0.002536476 |
| ENSMUSG00000036676 | Tmtc3 | 1.39534941 | 0.001227664 |
| ENSMUSG00000036019 | Tmtc2 | −3.7455997 | 0.00478851 |
| ENSMUSG00000053825 | Ppfia2 | −2.98092742 | 0.004720882 |
| ENSMUSG00000035864 | Syt1 | −1.87820483 | 0.043958142 |
| ENSMUSG00000035798 | Zdhhc17 | 1.03154439 | 0.018950697 |
| ENSMUSG00000006764 | Tph2 | −6.62349539 | 0.001057431 |
| ENSMUSG00000064181 | Rab3ip | 1.26023505 | 0.008230045 |
| ENSMUSG00000060181 | Slc35e3 | 1.07895841 | 0.041512592 |
| ENSMUSG00000055170 | Ifng | −2.37057853 | 0.00826349 |
| ENSMUSG00000034707 | Gns | 2.19457423 | 1.47E−04 |
| ENSMUSG00000025795 | Rassf3 | −1.99685386 | 8.89E−05 |
| ENSMUSG00000020105 | Lrig3 | 1.48675512 | 0.03363365 |
| ENSMUSG00000006736 | Tspan31 | 1.33926987 | 3.23E−04 |
| ENSMUSG00000040462 | Os9 | 1.48998088 | 1.37E−04 |
| ENSMUSG00000074657 | Kif5a | −4.93859051 | 1.06E−04 |
| ENSMUSG00000025409 | Mbd6 | −1.30669982 | 0.003518076 |
| ENSMUSG00000040054 | Baz2a | −1.13032937 | 0.003410469 |
| ENSMUSG00000025383 | Il23a | −2.55224101 | 0.016062353 |
| ENSMUSG00000018166 | Erbb3 | −1.94038264 | 0.020907745 |
| ENSMUSG00000047090 | Tmem198b | 1.47501892 | 7.26E−04 |
| ENSMUSG00000034833 | Tespa1 | −2.30286871 | 0.00213883 |
| ENSMUSG00000004567 | Mcoln1 | 1.22267528 | 6.05E−04 |
| ENSMUSG00000012705 | Retn | −3.71796625 | 0.004919718 |
| ENSMUSG00000013974 | Mcemp1 | −2.65693987 | 7.02E−06 |
| ENSMUSG00000089665 | Fcor | −2.4368731 | 0.003676643 |
| ENSMUSG00000074491 | Clec4g | −4.62165119 | 3.47E−04 |
| ENSMUSG00000031494 | Cd209a | −1.91023994 | 4.62E−05 |
| ENSMUSG00000040165 | Cd209c | −2.91799733 | 1.82E−05 |
| ENSMUSG00000002948 | Map2k7 | −1.0506784 | 0.008771139 |
| ENSMUSG00000048644 | Ctxn1 | −3.24455601 | 0.001261411 |
| ENSMUSG00000001300 | Efnb2 | −1.70993477 | 0.04868901 |
| ENSMUSG00000040459 | Arglu1 | −1.84640054 | 8.14E−04 |
| ENSMUSG00000040396 | Abhd13 | 1.66767871 | 9.36E−04 |
| ENSMUSG00000031497 | Tnfsf13b | 1.11988441 | 0.001516097 |
| ENSMUSG00000031503 | Col4a2 | −2.39802054 | 0.01174841 |
| ENSMUSG00000031444 | F10 | −2.30455429 | 8.06E−05 |
| ENSMUSG00000038515 | Grtp1 | −1.03906591 | 0.049859897 |
| ENSMUSG00000038497 | Tmco3 | 1.17135405 | 0.001045307 |
| ENSMUSG00000031451 | Gas6 | 1.15471893 | 0.001954802 |
| ENSMUSG00000026317 | Cln8 | 1.09629435 | 0.002344625 |
| ENSMUSG00000071176 | Arhgef10 | −2.05400985 | 0.003545167 |
| ENSMUSG00000055675 | Kbtbd11 | −2.61372992 | 8.44E−04 |
| ENSMUSG00000060924 | Csmd1 | −6.66615666 | 9.87E−04 |
| ENSMUSG00000063362 | Alg11 | 1.13650635 | 0.0015689 |
| ENSMUSG00000037492 | Zmat4 | −2.64622928 | 0.022320675 |
| ENSMUSG00000031553 | Adam3 | 1.22539684 | 0.018763661 |
| ENSMUSG00000031555 | Adam9 | 1.43618875 | 2.35E−04 |
| ENSMUSG00000031565 | Fgfr1 | −1.59033997 | 0.002867487 |
| ENSMUSG00000037260 | Hgsnat | 1.6340059 | 1.95E−04 |
| ENSMUSG00000031483 | Erlin2 | 1.50992673 | 2.07E−04 |
| ENSMUSG00000031486 | Adgra2 | −2.50580737 | 0.01601058 |
| ENSMUSG00000031488 | Rab11fip1 | −1.52937396 | 6.94E−04 |
| ENSMUSG00000039328 | Rnf122 | 1.07539641 | 0.005073563 |
| ENSMUSG00000046152 | Fut10 | 1.80381528 | 6.10E−05 |
| ENSMUSG00000062991 | Nrg1 | −5.23888717 | 9.72E−04 |
| ENSMUSG00000049184 | Purg | −2.34008406 | 0.007603054 |
| ENSMUSG00000009630 | Ppp2cb | −1.10884906 | 0.01335201 |
| ENSMUSG00000031584 | Gsr | −1.95658291 | 1.14E−04 |
| ENSMUSG00000031532 | Saraf | 1.41890587 | 2.99E−04 |
| ENSMUSG00000031523 | Dlc1 | 1.22981359 | 0.014920571 |
| ENSMUSG00000039470 | Zdhhc2 | −4.41018511 | 0.002365879 |
| ENSMUSG00000031591 | Asah1 | 1.58397208 | 3.85E−04 |
| ENSMUSG00000079057 | Cyp4v3 | 1.63278667 | 5.96E−05 |
| ENSMUSG00000031639 | Tlr3 | 1.91142897 | 1.90E−04 |
| ENSMUSG00000031626 | Sorbs2 | −3.47798059 | 9.06E−04 |
| ENSMUSG00000038173 | Enpp6 | −2.8266589 | 0.001905155 |
| ENSMUSG00000031561 | Tenm3 | −3.990965 | 0.00115724 |
| ENSMUSG00000031521 | Aga | 1.51218724 | 7.95E−04 |
| ENSMUSG00000039396 | Neil3 | −1.25325169 | 0.045050038 |
| ENSMUSG00000031520 | Vegfc | −1.71117619 | 0.028786874 |
| ENSMUSG00000031517 | Gpm6a | −2.83782209 | 5.68E−04 |
| ENSMUSG00000031613 | Hpgd | 1.08022595 | 0.012791669 |
| ENSMUSG00000031610 | Scrg1 | −3.69861449 | 1.92E−04 |
| ENSMUSG00000031647 | Mfap3l | −1.18103075 | 0.031407333 |
| ENSMUSG00000058056 | Palld | −2.18879082 | 5.18E−04 |
| ENSMUSG00000037852 | Cpe | −5.10883744 | 1.21E−06 |
| ENSMUSG00000031604 | Msmo1 | 1.33746595 | 0.002596236 |
| ENSMUSG00000031605 | Klhl2 | −1.8438173 | 0.001514347 |
| ENSMUSG00000036356 | Csgalnact1 | −3.96143772 | 0.002536329 |
| ENSMUSG00000015568 | Lpl | 1.83026259 | 8.99E−05 |
| ENSMUSG00000036306 | Lzts1 | −2.03564737 | 0.039482359 |
| ENSMUSG00000031862 | Atp13a1 | 1.29322632 | 7.39E−04 |
| ENSMUSG00000002341 | Ncan | −9.60722621 | 4.10E−05 |
| ENSMUSG00000087408 | Cers1 | 1.95764823 | 0.03787696 |
| ENSMUSG00000058301 | Upf1 | −1.02965765 | 0.016034976 |
| ENSMUSG00000110622 | Iqcn | −1.42909794 | 0.005028958 |
| ENSMUSG00000046295 | Ankle1 | −1.49672581 | 0.008725972 |
| ENSMUSG00000034863 | Ano8 | −1.52698846 | 0.038216274 |
| ENSMUSG00000034845 | Plvap | 1.98521985 | 0.021173897 |
| ENSMUSG00000043664 | Tmem221 | 1.66405458 | 2.75E−04 |
| ENSMUSG00000043243 | Niban3 | 1.00868966 | 0.044381563 |
| ENSMUSG00000070000 | Fcho1 | −1.03207708 | 0.030902663 |
| ENSMUSG00000003484 | Cyp4f18 | −1.70746058 | 8.25E−05 |
| ENSMUSG00000062007 | Hsh2d | −2.33734338 | 0.001777448 |
| ENSMUSG00000048148 | Nwd1 | −4.37436987 | 4.77E−04 |
| ENSMUSG00000034472 | Rasd2 | −3.20474705 | 0.005049621 |
| ENSMUSG00000037148 | Arhgap10 | −2.32580115 | 0.004335558 |
| ENSMUSG00000031681 | Smad1 | 1.20356774 | 0.016357954 |
| ENSMUSG00000031715 | Smarca5 | −1.09158848 | 0.00301317 |
| ENSMUSG00000031714 | Gab1 | 1.39309857 | 0.001205949 |
| ENSMUSG00000047747 | Rnf150 | −1.31204192 | 0.001712745 |
| ENSMUSG00000031710 | Ucp1 | −2.83799143 | 0.00785573 |
| ENSMUSG00000035151 | Elmod2 | 1.41239431 | 5.15E−04 |
| ENSMUSG00000002885 | Adgre5 | −1.45108431 | 1.59E−04 |
| ENSMUSG00000013033 | Adgrl1 | −1.39530914 | 0.024176221 |
| ENSMUSG00000005469 | Prkaca | −1.37862522 | 0.005547811 |
| ENSMUSG00000079003 | Samd1 | −2.29492266 | 0.002034816 |
| ENSMUSG00000037103 | Dcaf15 | −1.30351485 | 0.003155326 |
| ENSMUSG00000012889 | Podnl1 | −4.20589295 | 0.002780029 |
| ENSMUSG00000008129 | Brme1 | 1.6255511 | 0.037125207 |
| ENSMUSG00000003814 | Calr | 1.22411956 | 0.001050663 |
| ENSMUSG00000053693 | Mast1 | −1.98096785 | 0.013032679 |
| ENSMUSG00000005142 | Man2b1 | 1.71560265 | 1.40E−04 |
| ENSMUSG00000031703 | Itfg1 | 1.56858048 | 7.57E−05 |
| ENSMUSG00000045333 | Zfp423 | −4.83142063 | 0.00108051 |
| ENSMUSG00000055994 | Nod2 | −1.57261962 | 0.009957785 |
| ENSMUSG00000031740 | Mmp2 | 1.76600815 | 1.08E−04 |
| ENSMUSG00000033192 | Lpcat2 | 1.6436492 | 1.18E−04 |
| ENSMUSG00000031748 | Gnao1 | −2.91445512 | 3.07E−04 |
| ENSMUSG00000031760 | Mt3 | −4.76521412 | 6.07E−07 |
| ENSMUSG00000031770 | Herpud1 | 1.04852365 | 0.002587552 |
| ENSMUSG00000031779 | Ccl22 | −2.11825051 | 8.33E−04 |
| ENSMUSG00000031780 | Ccl17 | −4.20971905 | 2.75E−06 |
| ENSMUSG00000061577 | Adgrg5 | −1.19385617 | 0.034067011 |
| ENSMUSG00000031785 | Adgrg1 | 1.90752479 | 3.85E−05 |
| ENSMUSG00000060470 | Adgrg3 | −2.18863329 | 0.002202466 |
| ENSMUSG00000046707 | Csnk2a2 | −1.12440493 | 0.003383896 |
| ENSMUSG00000036564 | Ndrg4 | −4.34863333 | 2.94E−05 |
| ENSMUSG00000036534 | Slc38a7 | 1.35724502 | 6.78E−04 |
| ENSMUSG00000031880 | Rrad | 1.46176639 | 0.001102992 |
| ENSMUSG00000054320 | Lrrc36 | −2.12452603 | 0.035233636 |
| ENSMUSG00000014846 | Tppp3 | −1.60449163 | 1.11E−04 |
| ENSMUSG00000035237 | Lcat | −2.37943236 | 0.010498921 |
| ENSMUSG00000017765 | Slc12a4 | 1.10357685 | 0.006788532 |
| ENSMUSG00000031898 | Dpep3 | −4.76198987 | 0.001258912 |
| ENSMUSG00000053687 | Dpep2 | −2.27185702 | 9.45E−05 |
| ENSMUSG00000031903 | Pla2g15 | 1.90852796 | 2.93E−05 |
| ENSMUSG00000031907 | Zfp90 | 1.07540011 | 0.004491173 |
| ENSMUSG00000000303 | Cdh1 | −3.49368809 | 1.99E−04 |
| ENSMUSG00000003849 | Nqo1 | −1.93885837 | 0.019863605 |
| ENSMUSG00000031723 | Txnl4b | −1.1123272 | 0.040752957 |
| ENSMUSG00000031722 | Hp | −3.01856158 | 1.26E−06 |
| ENSMUSG00000048827 | Pkd1l3 | −2.31519132 | 0.007985967 |
| ENSMUSG00000003657 | Calb2 | −4.1084715 | 0.001445424 |
| ENSMUSG00000033763 | Mtss2 | −9.03293307 | 1.62E−05 |
| ENSMUSG00000003316 | Glg1 | 1.04524168 | 0.003069054 |
| ENSMUSG00000033596 | Rfwd3 | −1.28170965 | 6.79E−04 |
| ENSMUSG00000031951 | Tmem231 | 1.03897055 | 0.046042485 |
| ENSMUSG00000052557 | Gan | −1.37129634 | 0.003110332 |
| ENSMUSG00000034390 | Cmip | −1.93720108 | 4.80E−05 |
| ENSMUSG00000031825 | Crispld2 | −2.17706403 | 0.004618823 |
| ENSMUSG00000040010 | Slc7a5 | 1.2833378 | 0.019935643 |
| ENSMUSG00000025316 | Banp | 1.00376235 | 0.00328366 |
| ENSMUSG00000006362 | Cbfa2t3 | −2.36651078 | 5.01E−06 |
| ENSMUSG00000035569 | Ankrd11 | −1.14203608 | 0.001063962 |
| ENSMUSG00000001065 | Zfp276 | 1.14091956 | 0.002375483 |
| ENSMUSG00000062380 | Tubb3 | −2.11258033 | 0.010537898 |
| ENSMUSG00000039960 | Rhou | −5.40088381 | 1.91E−04 |
| ENSMUSG00000019478 | Rab4a | 1.10865238 | 0.019905631 |
| ENSMUSG00000031974 | Abcb10 | 1.03697312 | 0.010148616 |
| ENSMUSG00000031980 | Agt | −4.22548768 | 5.09E−04 |
| ENSMUSG00000037300 | Ttc13 | 1.58603671 | 1.11E−04 |
| ENSMUSG00000031987 | Egln1 | −1.5391373 | 8.59E−04 |
| ENSMUSG00000050930 | Map10 | 1.11334479 | 0.035081178 |
| ENSMUSG00000033998 | Kcnk1 | −2.22494413 | 0.033499817 |
| ENSMUSG00000025812 | Pard3 | −3.84272466 | 3.95E−04 |
| ENSMUSG00000025810 | Nrp1 | 1.40106891 | 4.81E−04 |
| ENSMUSG00000025809 | Itgb1 | 1.12055322 | 0.001603318 |
| ENSMUSG00000096904 | Lamtor3-ps | 1.57676682 | 0.018558383 |
| ENSMUSG00000025279 | Dnase1l3 | −3.21670052 | 4.75E−04 |
| ENSMUSG00000021750 | Fam107a | −6.37133076 | 5.34E−07 |
| ENSMUSG00000021775 | Nr1d2 | 1.12599734 | 0.003874589 |
| ENSMUSG00000058317 | Ube2e2 | −1.54558569 | 0.030382666 |
| ENSMUSG00000021806 | Nid2 | 1.81973091 | 3.73E−04 |
| ENSMUSG00000021815 | Mss51 | −2.15803317 | 0.006866076 |
| ENSMUSG00000021816 | Ppp3cb | −1.27483634 | 0.00541675 |
| ENSMUSG00000039357 | Fut11 | 1.52414725 | 2.69E−04 |
| ENSMUSG00000039308 | Ndst2 | 1.50464074 | 2.85E−04 |
| ENSMUSG00000021822 | Plau | 1.05136986 | 0.010698378 |
| ENSMUSG00000063142 | Kcnma1 | 1.57030641 | 0.0028335 |
| ENSMUSG00000055538 | Zcchc24 | −1.26405877 | 0.00132273 |
| ENSMUSG00000021879 | Dnah12 | −2.69873314 | 1.70E−04 |
| ENSMUSG00000006526 | Stimate | 1.27813437 | 0.008823555 |
| ENSMUSG00000006522 | Itih3 | −3.94473698 | 3.72E−04 |
| ENSMUSG00000021900 | Btd | 1.36376993 | 4.56E−04 |
| ENSMUSG00000021913 | Ogdhl | −3.98105945 | 1.80E−04 |
| ENSMUSG00000041673 | Lrrc18 | −1.48606133 | 0.019609226 |
| ENSMUSG00000037824 | Tspan14 | 1.22893211 | 0.001155843 |
| ENSMUSG00000072601 | Ear1 | −1.63112158 | 0.027508316 |
| ENSMUSG00000072599 | Ear-ps2 | −2.1074062 | 0.024434431 |
| ENSMUSG00000072596 | Ear2 | −2.24283506 | 0.001125957 |
| ENSMUSG00000037759 | Ptger2 | −2.50030954 | 0.001131034 |
| ENSMUSG00000037712 | Fermt2 | −5.21371797 | 2.16E−04 |
| ENSMUSG00000037697 | Ddhd1 | −1.7635879 | 2.38E−04 |
| ENSMUSG00000055128 | Cgrrf1 | 1.00908312 | 0.009556793 |
| ENSMUSG00000050335 | Lgals3 | −1.72491407 | 6.05E−05 |
| ENSMUSG00000036339 | Tmem260 | 1.46453504 | 7.45E−04 |
| ENSMUSG00000051969 | Tlr11 | −2.11586672 | 0.002211352 |
| ENSMUSG00000035953 | Pip4p1 | 1.28251075 | 0.002805633 |
| ENSMUSG00000021876 | Rnase4 | 1.2956139 | 0.004550099 |
| ENSMUSG00000004558 | Ndrg2 | −4.28583318 | 8.39E−08 |
| ENSMUSG00000093044 | Snord8 | −1.66316232 | 0.032329452 |
| ENSMUSG00000076867 | Trdv4 | −6.85165381 | 8.65E−04 |
| ENSMUSG00000000958 | Slc7a7 | 1.49033597 | 2.63E−04 |
| ENSMUSG00000000957 | Mmp14 | 1.18157536 | 0.002364718 |
| ENSMUSG00000022175 | Lrp10 | 1.46126904 | 0.001619685 |
| ENSMUSG00000022178 | Ajuba | 1.31835119 | 0.016332761 |
| ENSMUSG00000052435 | Cebpe | −3.06605268 | 8.97E−04 |
| ENSMUSG00000022180 | Slc7a8 | 1.97726845 | 5.04E−05 |
| ENSMUSG00000072494 | Ppp1r3e | −3.39880882 | 0.002781127 |
| ENSMUSG00000089682 | Bcl2l2 | −1.46912298 | 0.019056287 |
| ENSMUSG00000022194 | Pabpn1 | −1.07015949 | 0.009278948 |
| ENSMUSG00000022199 | Slc22a17 | 1.28548425 | 6.35E−04 |
| ENSMUSG00000040759 | Cmtm5 | −2.96608907 | 0.00331665 |
| ENSMUSG00000022208 | Jph4 | −2.76372614 | 0.023370055 |
| ENSMUSG00000002324 | Rec8 | −2.10622397 | 0.021898783 |
| ENSMUSG00000002320 | Tm9sf1 | 1.22051851 | 9.96E−04 |
| ENSMUSG00000046908 | Ltb4r1 | −2.6231007 | 1.50E−05 |
| ENSMUSG00000022225 | Cma1 | −2.43883675 | 0.033831618 |
| ENSMUSG00000079186 | Gzmc | −4.40518412 | 3.65E−06 |
| ENSMUSG00000015437 | Gzmb | −3.8000111 | 1.30E−06 |
| ENSMUSG00000040055 | Gjb6 | −3.35319972 | 4.37E−04 |
| ENSMUSG00000021983 | Atp8a2 | 1.88478004 | 3.33E−05 |
| ENSMUSG00000060548 | Tnfrsf19 | −3.74481401 | 0.003519612 |
| ENSMUSG00000035184 | Fam124a | −6.42575516 | 0.001385579 |
| ENSMUSG00000014547 | Wdfy2 | 1.00177892 | 0.007191689 |
| ENSMUSG00000075571 | Defb30 | −3.94061282 | 0.004135321 |
| ENSMUSG00000021939 | Ctsb | 1.67158779 | 5.66E−04 |
| ENSMUSG00000035067 | Xkr6 | −1.24496069 | 0.034970983 |
| ENSMUSG00000021978 | Extl3 | 1.07449064 | 0.003320535 |
| ENSMUSG00000022037 | Clu | −3.05648946 | 1.16E−05 |
| ENSMUSG00000022040 | Ephx2 | −1.62335118 | 0.040865737 |
| ENSMUSG00000022041 | Chrna2 | −2.07655325 | 0.024985502 |
| ENSMUSG00000044447 | Dock5 | −2.27290257 | 5.76E−05 |
| ENSMUSG00000022055 | Nefl | −4.40741284 | 1.55E−04 |
| ENSMUSG00000098713 | Rps2-ps5 | −1.80932509 | 0.030067078 |
| ENSMUSG00000033644 | Piwil2 | −2.76429777 | 0.00311564 |
| ENSMUSG00000022096 | Hr | −3.82612791 | 4.22E−04 |
| ENSMUSG00000022102 | Dok2 | −1.61512712 | 5.10E−04 |
| ENSMUSG00000022103 | Gfra2 | −4.09890101 | 1.07E−04 |
| ENSMUSG00000033446 | Lpar6 | 1.04941639 | 0.003656511 |
| ENSMUSG00000022108 | Itm2b | 1.44319771 | 0.002114936 |
| ENSMUSG00000021997 | Lrrc63 | −1.89740615 | 0.028473284 |
| ENSMUSG00000046523 | Kctd4 | −2.4297915 | 0.006603715 |
| ENSMUSG00000052584 | Serp2 | −3.08756601 | 0.010353749 |
| ENSMUSG00000035566 | Pcdh17 | −1.79822655 | 0.019211776 |
| ENSMUSG00000022021 | Diaph3 | −2.04621721 | 0.006357123 |
| ENSMUSG00000055421 | Pcdh9 | −4.48394856 | 1.53E−04 |
| ENSMUSG00000055639 | Dach1 | −7.40528205 | 3.15E−04 |
| ENSMUSG00000072294 | Klf12 | 1.61237875 | 6.24E−04 |
| ENSMUSG00000033060 | Lmo7 | −3.28175303 | 0.011694837 |
| ENSMUSG00000022126 | Acod1 | −2.94266015 | 7.77E−06 |
| ENSMUSG00000022125 | Cln5 | 1.32006443 | 3.66E−04 |
| ENSMUSG00000022123 | Scel | −1.81857464 | 0.036267204 |
| ENSMUSG00000053253 | Ndfip2 | 1.41468731 | 0.001218569 |
| ENSMUSG00000022114 | Spry2 | −3.38499241 | 0.0010145 |
| ENSMUSG00000089726 | Mir17hg | −1.10149908 | 0.040364786 |
| ENSMUSG00000022112 | Gpc5 | −3.80845019 | 0.001406001 |
| ENSMUSG00000058571 | Gpc6 | −2.1294864 | 0.016084025 |
| ENSMUSG00000022131 | Gpr180 | 1.74027324 | 5.28E−05 |
| ENSMUSG00000022132 | Cldn10 | −2.54982661 | 0.004144666 |
| ENSMUSG00000042156 | Dzip1 | −1.87391471 | 0.017279866 |
| ENSMUSG00000025555 | Farp1 | −1.61008954 | 0.009651849 |
| ENSMUSG00000063410 | Stk24 | −1.54039828 | 7.19E−04 |
| ENSMUSG00000051212 | Gpr183 | 1.62331279 | 5.28E−04 |
| ENSMUSG00000025544 | Tm9sf2 | 1.61306105 | 2.62E−04 |
| ENSMUSG00000041650 | Pcca | 1.054609 | 0.003220733 |
| ENSMUSG00000025551 | Fgf14 | −4.13923106 | 0.002380056 |
| ENSMUSG00000025887 | Casp12 | 1.21577392 | 0.039083092 |
| ENSMUSG00000050578 | Mmp13 | −1.65614142 | 0.038273111 |
| ENSMUSG00000005800 | Mmp8 | −1.50980583 | 0.001170135 |
| ENSMUSG00000053110 | Yap1 | −3.68358407 | 0.002160069 |
| ENSMUSG00000031802 | Phxr4 | −1.56330786 | 9.54E−04 |
| ENSMUSG00000032009 | Sesn3 | −2.16889543 | 3.18E−04 |
| ENSMUSG00000037419 | Endod1 | 1.87104529 | 2.24E−05 |
| ENSMUSG00000043943 | Naalad2 | 2.23681258 | 1.45E−05 |
| ENSMUSG00000058192 | Zfp846 | 1.13621115 | 0.006853369 |
| ENSMUSG00000037405 | Icam1 | 1.50655132 | 1.97E−04 |
| ENSMUSG00000045087 | S1pr5 | −1.50862604 | 6.48E−04 |
| ENSMUSG00000096472 | Cdkn2d | −1.20453632 | 7.58E−04 |
| ENSMUSG00000057193 | Slc44a2 | 1.1607646 | 0.001380127 |
| ENSMUSG00000057191 | AB124611 | −1.91599535 | 3.45E−05 |
| ENSMUSG00000032182 | Yipf2 | 1.11237378 | 0.004665245 |
| ENSMUSG00000074476 | Spc24 | −1.56366528 | 0.005425819 |
| ENSMUSG00000019066 | Rab3d | −1.13335224 | 0.005218528 |
| ENSMUSG00000003402 | Prkcsh | 1.07769525 | 0.001956327 |
| ENSMUSG00000003410 | Elavl3 | −2.96141384 | 0.003388427 |
| ENSMUSG00000001348 | Acp5 | −1.18404855 | 0.003409213 |
| ENSMUSG00000043067 | Dpy19l1 | −1.0339158 | 0.014992648 |
| ENSMUSG00000031990 | Jam3 | 1.59361453 | 4.39E−04 |
| ENSMUSG00000059974 | Ntm | −6.01508827 | 6.30E−06 |
| ENSMUSG00000031993 | Snx19 | 1.3889484 | 0.001103484 |
| ENSMUSG00000031995 | St14 | 1.41348788 | 2.79E−04 |
| ENSMUSG00000031996 | Aplp2 | 1.32440209 | 4.44E−04 |
| ENSMUSG00000032036 | Kirrel3 | −3.48284944 | 0.006440761 |
| ENSMUSG00000032042 | Srpr | 1.23922655 | 6.71E−04 |
| ENSMUSG00000032101 | Ddx25 | −6.43074561 | 0.001418032 |
| ENSMUSG00000032116 | Stt3a | 1.50209201 | 1.25E−04 |
| ENSMUSG00000032118 | Fez1 | −4.15695113 | 2.09E−04 |
| ENSMUSG00000035934 | Pknox2 | −3.09135362 | 0.010749818 |
| ENSMUSG00000032122 | Slc37a2 | 1.20804011 | 6.70E−04 |
| ENSMUSG00000046240 | Hepacam | −4.44850364 | 2.46E−04 |
| ENSMUSG00000053310 | Nrgn | −2.6510449 | 0.001935561 |
| ENSMUSG00000001942 | Siae | 1.5758972 | 9.45E−05 |
| ENSMUSG00000059252 | Tpt1-ps5 | −1.28368413 | 0.02312023 |
| ENSMUSG00000038112 | AW551984 | −6.48980618 | 0.001248022 |
| ENSMUSG00000032021 | Crtam | −3.21808348 | 0.007451392 |
| ENSMUSG00000049313 | Sorl1 | −1.42389631 | 1.92E−04 |
| ENSMUSG00000032018 | Sc5d | 1.11919681 | 0.002205534 |
| ENSMUSG00000032012 | Nectin1 | −2.75529663 | 0.002047992 |
| ENSMUSG00000032011 | Thy1 | −3.45101111 | 0.00134169 |
| ENSMUSG00000032105 | Pdzd3 | −2.86457365 | 0.010972655 |
| ENSMUSG00000032120 | C2cd2l | 1.33977316 | 0.001567508 |
| ENSMUSG00000032123 | Dpagt1 | 1.37265585 | 3.22E−04 |
| ENSMUSG00000032115 | Hyou1 | 1.26060334 | 0.019620144 |
| ENSMUSG00000002028 | Kmt2a | −1.0319313 | 0.002524153 |
| ENSMUSG00000048534 | Jaml | −2.52564031 | 2.08E−05 |
| ENSMUSG00000070304 | Scn2b | −5.77704071 | 5.03E−05 |
| ENSMUSG00000046480 | Scn4b | −3.03422396 | 0.003596928 |
| ENSMUSG00000032076 | Cadm1 | 1.58371834 | 9.92E−05 |
| ENSMUSG00000044229 | Nxpe4 | −1.48652777 | 8.03E−04 |
| ENSMUSG00000066687 | Zbtb16 | −1.91335484 | 0.001695427 |
| ENSMUSG00000039542 | Ncam1 | −4.04000791 | 1.91E−04 |
| ENSMUSG00000032064 | Dixdc1 | −2.01643388 | 0.032581757 |
| ENSMUSG00000032060 | Cryab | −2.14552848 | 1.74E−04 |
| ENSMUSG00000032059 | Alg9 | 1.48031737 | 1.81E−04 |
| ENSMUSG00000060594 | Layn | 1.72555169 | 0.010209306 |
| ENSMUSG00000034487 | Poglut3 | 2.01754322 | 5.56E−04 |
| ENSMUSG00000055069 | Rab39 | 1.2954131 | 7.43E−04 |
| ENSMUSG00000041986 | Elmod1 | −3.56560096 | 0.005904996 |
| ENSMUSG00000032281 | Acsbg1 | −5.41499741 | 8.64E−06 |
| ENSMUSG00000032285 | Dnaja4 | 1.45578165 | 0.037084552 |
| ENSMUSG00000035878 | Hykk | 1.36991839 | 0.019335994 |
| ENSMUSG00000032322 | Pstpip1 | −1.86726209 | 3.09E−05 |
| ENSMUSG00000032324 | Tspan3 | 1.74670945 | 1.06E−04 |
| ENSMUSG00000070298 | Trcg1 | −1.85152968 | 0.028258427 |
| ENSMUSG00000040722 | Scamp5 | 1.35762745 | 6.89E−04 |
| ENSMUSG00000040188 | Scamp2 | 1.31381614 | 4.73E−04 |
| ENSMUSG00000035914 | Cd276 | 2.19016428 | 4.20E−05 |
| ENSMUSG00000032336 | Nptn | 1.04135599 | 0.013999112 |
| ENSMUSG00000074269 | Rec114 | −1.10828683 | 0.035724647 |
| ENSMUSG00000090516 | Rps11-ps1 | −1.53519036 | 8.50E−04 |
| ENSMUSG00000025232 | Hexa | 1.67084387 | 3.24E−04 |
| ENSMUSG00000074259 | Gramd2 | −3.37883626 | 0.002000914 |
| ENSMUSG00000032254 | Kif23 | −1.39496164 | 0.002364953 |
| ENSMUSG00000032252 | Glce | 1.24608316 | 0.009479251 |
| ENSMUSG00000041729 | Coro2b | −1.9252015 | 0.017334963 |
| ENSMUSG00000034263 | Ints14 | 1.07052607 | 0.002396173 |
| ENSMUSG00000033629 | Hacd3 | 1.62513864 | 1.01E−04 |
| ENSMUSG00000041696 | Rasl12 | −6.60520644 | 0.001149495 |
| ENSMUSG00000040204 | Pclaf | −2.0422187 | 6.48E−04 |
| ENSMUSG00000032380 | Dapk2 | −2.78112847 | 0.004572561 |
| ENSMUSG00000032375 | Aph1b | −1.08724201 | 0.008637021 |
| ENSMUSG00000053040 | Aph1c | −1.05243184 | 0.009523151 |
| ENSMUSG00000032231 | Anxa2 | −1.63089033 | 1.54E−04 |
| ENSMUSG00000054693 | Adam10 | 1.6192494 | 7.75E−05 |
| ENSMUSG00000032204 | Aqp9 | −2.48676158 | 3.33E−04 |
| ENSMUSG00000013584 | Aldh1a2 | −2.78308754 | 0.001896453 |
| ENSMUSG00000032232 | Cgnl1 | −3.71111011 | 0.004780169 |
| ENSMUSG00000032216 | Nedd4 | −2.45497799 | 5.97E−04 |
| ENSMUSG00000034910 | Pygo1 | −8.34597954 | 7.24E−05 |
| ENSMUSG00000034563 | Ccpg1 | 1.01059642 | 0.004868579 |
| ENSMUSG00000079469 | Pigb | 1.03918089 | 0.004632967 |
| ENSMUSG00000062151 | Unc13c | −4.51315345 | 0.00047659 |
| ENSMUSG00000032186 | Tmod2 | −4.62567978 | 1.33E−05 |
| ENSMUSG00000032181 | Scg3 | −5.40252003 | 1.02E−05 |
| ENSMUSG00000032348 | Gsta4 | −1.77299621 | 0.048762265 |
| ENSMUSG00000070291 | Ddx43 | 1.09828154 | 0.009786915 |
| ENSMUSG00000049624 | Slc17a5 | 1.34615785 | 3.94E−04 |
| ENSMUSG00000032328 | Tmem30a | 1.15431892 | 0.003970021 |
| ENSMUSG00000032418 | Me1 | −3.4826963 | 4.15E−04 |
| ENSMUSG00000033419 | Snap91 | −6.36230069 | 4.75E−04 |
| ENSMUSG00000032420 | Nt5e | −1.53127782 | 0.045530391 |
| ENSMUSG00000032422 | Snx14 | 1.06407726 | 0.002464871 |
| ENSMUSG00000032353 | Tmed3 | 1.00963254 | 0.010048989 |
| ENSMUSG00000032356 | Rasgrf1 | −1.61282159 | 0.022561907 |
| ENSMUSG00000032359 | Ctsh | 1.17953108 | 0.0035288 |
| ENSMUSG00000031129 | Slc9a9 | 1.47782202 | 1.99E−04 |
| ENSMUSG00000032412 | Atp1b3 | 1.08542027 | 0.003947522 |
| ENSMUSG00000043587 | Pxylp1 | −3.15218943 | 6.15E−05 |
| ENSMUSG00000046402 | Rbp1 | −3.5141499 | 3.28E−04 |
| ENSMUSG00000032549 | Rab6b | −1.00121739 | 0.009919847 |
| ENSMUSG00000032553 | Srprb | 1.33462981 | 4.55E−04 |
| ENSMUSG00000032554 | Trf | 1.37048945 | 0.000467727 |
| ENSMUSG00000032561 | Acpp | −3.10268417 | 6.83E−04 |
| ENSMUSG00000032570 | Atp2c1 | 1.69206072 | 5.52E−05 |
| ENSMUSG00000032571 | Pik3r4 | 1.30221884 | 0.001298207 |
| ENSMUSG00000045322 | Tlr9 | 1.15799065 | 0.034821632 |
| ENSMUSG00000023495 | Pcbp4 | −1.61471558 | 0.016524915 |
| ENSMUSG00000032578 | Cish | −1.18729699 | 0.047871371 |
| ENSMUSG00000010045 | Tmem115 | 1.34413994 | 3.90E−04 |
| ENSMUSG00000010047 | Hyal2 | 1.49820014 | 5.63E−04 |
| ENSMUSG00000010064 | Slc38a3 | −3.0893601 | 0.004149542 |
| ENSMUSG00000042106 | Inka1 | 1.04527054 | 0.007119033 |
| ENSMUSG00000032589 | Bsn | 1.05059044 | 0.037314995 |
| ENSMUSG00000039952 | Dag1 | 1.89956027 | 8.87E−05 |
| ENSMUSG00000032609 | Klhdc8b | 1.52353343 | 1.24E−04 |
| ENSMUSG00000052911 | Lamb2 | 2.39487073 | 7.51E−05 |
| ENSMUSG00000032601 | Prkar2a | −1.45641162 | 5.88E−04 |
| ENSMUSG00000038357 | Camp | −3.41114169 | 1.18E−06 |
| ENSMUSG00000032482 | Cspg5 | −4.08364058 | 3.84E−04 |
| ENSMUSG00000098973 | Mir6236 | 1.06088551 | 0.02311128 |
| ENSMUSG00000032484 | Ngp | −2.78788029 | 3.72E−06 |
| ENSMUSG00000032491 | Nradd | 1.65518604 | 4.73E−04 |
| ENSMUSG00000049555 | Tmie | −7.17113095 | 5.11E−04 |
| ENSMUSG00000032494 | Tdgf1 | −3.63057029 | 8.38E−04 |
| ENSMUSG00000032496 | Ltf | −2.18932892 | 2.47E−05 |
| ENSMUSG00000043953 | Ccr12 | 1.43886835 | 6.31E−04 |
| ENSMUSG00000046785 | Epm2aip1 | 1.20709773 | 0.001931767 |
| ENSMUSG00000032503 | Arpp21 | −3.27583065 | 8.38E−04 |
| ENSMUSG00000032507 | Fbxl2 | −2.70887667 | 0.009094891 |
| ENSMUSG00000032431 | Crtap | 1.71295132 | 6.14E−04 |
| ENSMUSG00000045594 | Glb1 | 1.95408312 | 1.18E−05 |
| ENSMUSG00000032434 | Cmtm6 | 1.64276215 | 1.46E−04 |
| ENSMUSG00000032437 | Stt3b | 1.40253026 | 5.24E−04 |
| ENSMUSG00000056880 | Gadl1 | 1.64709674 | 0.004843792 |
| ENSMUSG00000032440 | Tgfbr2 | 1.89351105 | 7.42E−05 |
| ENSMUSG00000047409 | Ctdspl | −1.34680048 | 0.015642282 |
| ENSMUSG00000038775 | Vill | −1.70098143 | 0.001123233 |
| ENSMUSG00000010660 | Plcd1 | 1.04589729 | 0.00471412 |
| ENSMUSG00000036737 | Oxsr1 | −1.33734095 | 0.004985941 |
| ENSMUSG00000035769 | Xylb | 1.22133594 | 0.0019092 |
| ENSMUSG00000042787 | Exog | 1.83388057 | 2.87E−05 |
| ENSMUSG00000052336 | Cx3cr1 | 1.85286169 | 1.77E−04 |
| ENSMUSG00000032517 | Mobp | −6.02171463 | 2.81E−05 |
| ENSMUSG00000041608 | Entpd3 | −3.67947939 | 0.003197288 |
| ENSMUSG00000032530 | Lyzl4 | 1.59711162 | 0.00596213 |
| ENSMUSG00000013419 | Zfp651 | −1.27710652 | 0.033918813 |
| ENSMUSG00000032523 | Hhatl | 6.97170861 | 6.17E−04 |
| ENSMUSG00000066235 | Pomgnt2 | 2.256149 | 9.32E−05 |
| ENSMUSG00000037949 | Ano10 | 1.53996076 | 1.47E−04 |
| ENSMUSG00000111063 | Zfp660 | 2.20864486 | 0.02094291 |
| ENSMUSG00000066233 | Tmem42 | 1.06371737 | 0.030479752 |
| ENSMUSG00000035202 | Lars2 | −1.6211137 | 2.83E−04 |
| ENSMUSG00000025240 | Sacm1l | 1.06178751 | 0.00232315 |
| ENSMUSG00000029530 | Ccr9 | −1.51827406 | 0.019927377 |
| ENSMUSG00000048521 | Cxcr6 | −3.30813539 | 6.72E−04 |
| ENSMUSG00000049103 | Ccr2 | −2.4934935 | 1.23E−06 |
| ENSMUSG00000079227 | Ccr5 | 1.41772982 | 3.60E−04 |
| ENSMUSG00000082658 | Fau-ps2 | −2.75300891 | 0.010349856 |
| ENSMUSG00000020432 | Tcn2 | 1.95264441 | 3.90E−05 |
| ENSMUSG00000058755 | Osm | 1.30107992 | 8.85E−04 |
| ENSMUSG00000034175 | Rhbdd3 | 1.00838015 | 0.010729581 |
| ENSMUSG00000020484 | Xbp1 | 1.0337665 | 0.00278709 |
| ENSMUSG00000020482 | Ccdc117 | 1.05890832 | 0.04039108 |
| ENSMUSG00000004394 | Tmed4 | 1.1564367 | 0.001026943 |
| ENSMUSG00000041164 | Zmiz2 | −1.23670209 | 0.001134562 |
| ENSMUSG00000020437 | Myo1g | −1.2178303 | 7.18E−04 |
| ENSMUSG00000020431 | Adcy1 | −3.03162379 | 0.004836601 |
| ENSMUSG00000020407 | Upp1 | −1.63379468 | 0.037338523 |
| ENSMUSG00000020193 | Zpbp | 2.03093138 | 0.013954794 |
| ENSMUSG00000020122 | Egfr | 1.54544703 | 0.044552109 |
| ENSMUSG00000016984 | Etaa1 | 1.07911626 | 0.025026582 |
| ENSMUSG00000044066 | Cep68 | 1.33387149 | 6.29E−04 |
| ENSMUSG00000056342 | Usp34 | −1.02025054 | 0.003537419 |
| ENSMUSG00000000861 | Bcl11a | −1.94097403 | 1.08E−04 |
| ENSMUSG00000032878 | Ccdc85a | −4.3969424 | 1.95E−04 |
| ENSMUSG00000081695 | Rpsa-ps5 | −2.86562874 | 0.00524438 |
| ENSMUSG00000020315 | Sptbn1 | −1.01716808 | 0.013466877 |
| ENSMUSG00000020282 | Rhbdf1 | 1.50877888 | 0.005774906 |
| ENSMUSG00000040711 | Sh3pxd2b | −1.41510644 | 5.12E−04 |
| ENSMUSG00000044949 | Ubtd2 | 1.55717869 | 0.015459936 |
| ENSMUSG00000069911 | Insyn2b | −1.08168055 | 0.010766465 |
| ENSMUSG00000020330 | Hmmr | −1.01934067 | 0.037238051 |
| ENSMUSG00000019189 | Rnf145 | 1.08763392 | 0.004854955 |
| ENSMUSG00000011256 | Adam19 | −2.58934898 | 2.91E−05 |
| ENSMUSG00000020340 | Cyfip2 | −1.62323321 | 8.66E−05 |
| ENSMUSG00000020395 | Itk | −3.58814791 | 6.89E−04 |
| ENSMUSG00000020399 | Havcr2 | 1.44414611 | 2.51E−04 |
| ENSMUSG00000040365 | Trim41 | −1.21712577 | 0.005443344 |
| ENSMUSG00000078922 | Tgtp1 | −1.20720369 | 0.024976321 |
| ENSMUSG00000078920 | Ifi47 | −1.13348174 | 0.002400182 |
| ENSMUSG00000020362 | Cnot6 | −1.04046815 | 0.003311545 |
| ENSMUSG00000044807 | Zfp354c | 1.11824056 | 0.022308283 |
| ENSMUSG00000048728 | Zfp454 | 2.34789555 | 0.006069819 |
| ENSMUSG00000020364 | Zfp354a | 1.56127258 | 0.002317431 |
| ENSMUSG00000063564 | Col23a1 | −1.80733265 | 0.016598876 |
| ENSMUSG00000001053 | N4bp3 | −1.42041626 | 0.007050285 |
| ENSMUSG00000020349 | Ppp2ca | −1.1944731 | 0.003036589 |
| ENSMUSG00000020388 | Pdlim4 | 1.06862946 | 0.003112019 |
| ENSMUSG00000020333 | Acsl6 | −4.18786184 | 1.51E−04 |
| ENSMUSG00000049588 | Ccdc69 | −1.38396069 | 0.00748478 |
| ENSMUSG00000085531 | Slc36a3os | −2.60638356 | 0.002457607 |
| ENSMUSG00000020264 | Slc36a2 | 1.49596406 | 0.006552743 |
| ENSMUSG00000018593 | Sparc | 1.66869458 | 4.34E−04 |
| ENSMUSG00000020524 | Gria1 | −6.72879993 | 3.87E−05 |
| ENSMUSG00000020522 | Mfap3 | 1.1977509 | 7.70E−04 |
| ENSMUSG00000020520 | Galnt10 | 1.56313293 | 0.00645881 |
| ENSMUSG00000078853 | Igtp | 1.2890312 | 5.17E−04 |
| ENSMUSG00000069874 | Irgm2 | 1.00277882 | 0.011055146 |
| ENSMUSG00000005417 | Mprip | −1.00531114 | 0.004457645 |
| ENSMUSG00000043648 | Pld6 | −2.04432327 | 0.031434814 |
| ENSMUSG00000032633 | Flcn | 1.10092169 | 0.002166671 |
| ENSMUSG00000042650 | Alkbh5 | −1.33938055 | 0.010128781 |
| ENSMUSG00000018931 | Natd1 | 1.05334254 | 0.002599163 |
| ENSMUSG00000042529 | Kcnj12 | 2.18724162 | 0.012274125 |
| ENSMUSG00000018500 | Adora2b | −1.26907086 | 0.026365357 |
| ENSMUSG00000042298 | Ttc19 | −1.2768367 | 0.00644814 |
| ENSMUSG00000018501 | Ncor1 | −1.17956024 | 0.001200894 |
| ENSMUSG00000014177 | Tvp23b | 1.02873193 | 0.007759065 |
| ENSMUSG00000018217 | Pmp22 | 1.7698206 | 1.73E−04 |
| ENSMUSG00000070407 | Hs3st3b1 | 1.43592111 | 0.007885492 |
| ENSMUSG00000033389 | Arhgap44 | −2.42540201 | 0.022134083 |
| ENSMUSG00000069844 | Sco1 | 1.09521692 | 0.008584888 |
| ENSMUSG00000055775 | Myh8 | −4.51056647 | 0.002279564 |
| ENSMUSG00000033066 | Gas7 | −2.18826202 | 0.003798205 |
| ENSMUSG00000048329 | Mfsd61 | −2.59626238 | 0.015913647 |
| ENSMUSG00000020900 | Myh10 | −1.08483527 | 0.01020508 |
| ENSMUSG00000020893 | Per1 | −1.00906368 | 0.005853 |
| ENSMUSG00000023781 | Hes7 | −2.65860087 | 0.010809424 |
| ENSMUSG00000018474 | Chd3 | −1.54352251 | 3.27E−04 |
| ENSMUSG00000045377 | Tmem88 | −1.85042223 | 0.002640163 |
| ENSMUSG00000018476 | Kdm6b | −2.28268892 | 4.27E−05 |
| ENSMUSG00000005237 | Dnah2 | −1.64312168 | 0.013699513 |
| ENSMUSG00000041329 | Atp1b2 | −4.75377183 | 1.53E−04 |
| ENSMUSG00000069835 | Sat2 | 1.15328407 | 0.025771889 |
| ENSMUSG00000018774 | Cd68 | 1.62494746 | 7.09E−04 |
| ENSMUSG00000085890 | Tnfsf13os | −2.02972217 | 0.002061423 |
| ENSMUSG00000018750 | Zbtb4 | −1.15091089 | 0.004763607 |
| ENSMUSG00000051790 | Nlgn2 | −2.03770236 | 0.012225098 |
| ENSMUSG00000001588 | Acap1 | −2.48389087 | 1.88E−04 |
| ENSMUSG00000040963 | Asgr2 | −1.99958385 | 0.010421693 |
| ENSMUSG00000040950 | Mgl2 | −1.63370867 | 7.67E−05 |
| ENSMUSG00000000318 | Clec10a | −1.57185622 | 1.17E−04 |
| ENSMUSG00000060600 | Eno3 | −1.17870605 | 0.001064934 |
| ENSMUSG00000020821 | Kif1c | −2.03185365 | 0.001103634 |
| ENSMUSG00000081968 | Rpl23a-ps2 | −2.58979575 | 0.004486756 |
| ENSMUSG00000040620 | Dhx33 | 1.07867069 | 0.003042264 |
| ENSMUSG00000018442 | Derl2 | 1.00757492 | 0.004088356 |
| ENSMUSG00000069830 | Nlrp1a | 1.28960319 | 0.002574614 |
| ENSMUSG00000020811 | Wscd1 | 1.41924019 | 6.94E−04 |
| ENSMUSG00000057778 | Cyb5d2 | 1.35095239 | 4.91E−04 |
| ENSMUSG00000020787 | P2rx1 | 1.47905343 | 0.003508013 |
| ENSMUSG00000005949 | Ctns | 1.32318343 | 4.09E−04 |
| ENSMUSG00000005951 | Shpk | 1.01242967 | 0.035790514 |
| ENSMUSG00000020774 | Aspa | −2.74521012 | 0.002746985 |
| ENSMUSG00000038807 | Rap1gap2 | −1.04701918 | 0.005616622 |
| ENSMUSG00000000282 | Mnt | −1.05644298 | 0.007623669 |
| ENSMUSG00000038351 | Sgsm2 | −1.33152034 | 0.007472442 |
| ENSMUSG00000045287 | Rtn4rl1 | 1.78771587 | 6.82E−05 |
| ENSMUSG00000085148 | Mir22hg | −1.66181066 | 1.53E−04 |
| ENSMUSG00000020846 | Rflnb | −2.8387292 | 9.85E−05 |
| ENSMUSG00000000686 | Abhd15 | 1.0301892 | 0.006319487 |
| ENSMUSG00000017453 | Pipox | −6.50432042 | 0.001431649 |
| ENSMUSG00000044122 | Proca1 | −1.9245998 | 0.008818622 |
| ENSMUSG00000002055 | Spag5 | −1.89782973 | 0.004611101 |
| ENSMUSG00000017390 | Aldoc | −4.84157662 | 1.75E−07 |
| ENSMUSG00000002058 | Unc119 | −2.0971139 | 1.65E−05 |
| ENSMUSG00000020829 | Slc46a1 | 1.65816622 | 5.70E−05 |
| ENSMUSG00000037278 | Tmem97 | 1.00942715 | 0.008510234 |
| ENSMUSG00000078771 | Evi2a | 1.03363889 | 0.004272228 |
| ENSMUSG00000017639 | Rab11fip4 | −1.76821336 | 0.001775481 |
| ENSMUSG00000020702 | Ccl1 | −7.0472111 | 8.70E−04 |
| ENSMUSG00000054404 | Slfn5 | −1.03333178 | 0.005506626 |
| ENSMUSG00000078763 | Slfn1 | −3.33457637 | 1.50E−06 |
| ENSMUSG00000000204 | Slfn4 | −3.39614196 | 2.93E−06 |
| ENSMUSG00000087107 | Al662270 | −1.37683389 | 0.00258316 |
| ENSMUSG00000065715 | Snord7 | −2.54416808 | 0.017898704 |
| ENSMUSG00000020682 | Mmp28 | 1.46126236 | 0.018126269 |
| ENSMUSG00000035042 | Ccl5 | −1.72769789 | 9.54E−04 |
| ENSMUSG00000000982 | Ccl3 | 1.16986054 | 0.002405799 |
| ENSMUSG00000018930 | Ccl4 | 1.43076561 | 5.65E−04 |
| ENSMUSG00000069792 | Wfdc17 | −1.2031283 | 0.00723541 |
| ENSMUSG00000000983 | Wfdc18 | −2.43031206 | 0.007939649 |
| ENSMUSG00000051748 | Wfdc21 | −3.54809373 | 1.56E−06 |
| ENSMUSG00000082806 | Rpl13-ps1 | −1.82266631 | 0.014837844 |
| ENSMUSG00000086199 | Bcas3os1 | −1.45753856 | 0.027026961 |
| ENSMUSG00000085208 | Brip1os | 1.12636692 | 0.00324104 |
| ENSMUSG00000020495 | Smg8 | 1.07522063 | 0.00728245 |
| ENSMUSG00000007646 | Rad51c | −1.43363386 | 0.029219854 |
| ENSMUSG00000020486 | Septin4 | −1.38606813 | 0.012032773 |
| ENSMUSG00000034177 | Rnf43 | −3.07156022 | 0.001319301 |
| ENSMUSG00000065420 | Mir142b | −1.9355297 | 0.010568674 |
| ENSMUSG00000034156 | Tspoap1 | −3.61431465 | 3.02E−05 |
| ENSMUSG00000009350 | Mpo | −1.77303581 | 0.027035018 |
| ENSMUSG00000018378 | Cuedc1 | −4.03033445 | 5.86E−04 |
| ENSMUSG00000069769 | Msi2 | −1.14172064 | 0.019678104 |
| ENSMUSG00000018428 | Akap1 | 1.14533133 | 0.007300812 |
| ENSMUSG00000069763 | Tmem100 | 1.54125736 | 4.16E−04 |
| ENSMUSG00000020541 | Tom1l1 | 1.09399761 | 0.002550159 |
| ENSMUSG00000056158 | Car10 | −6.82703775 | 7.70E−04 |
| ENSMUSG00000037573 | Tob1 | −1.06669727 | 0.027320416 |
| ENSMUSG00000020864 | Ankrd40 | −1.00741263 | 0.007891541 |
| ENSMUSG00000020865 | Abcc3 | 1.7946439 | 1.14E−04 |
| ENSMUSG00000039084 | Chad | 1.40710826 | 0.047247404 |
| ENSMUSG00000020869 | Lrrc59 | 1.14411497 | 0.007893122 |
| ENSMUSG00000039055 | Eme1 | −1.25583209 | 0.034929233 |
| ENSMUSG00000038976 | Ppp1r9b | −1.6181907 | 3.04E−04 |
| ENSMUSG00000047181 | Samd14 | −1.9411173 | 0.015994963 |
| ENSMUSG00000001507 | Itga3 | −2.17475081 | 0.019919496 |
| ENSMUSG00000038692 | Hoxb4 | −3.78308633 | 0.001200775 |
| ENSMUSG00000057058 | Skap1 | −2.31429945 | 0.004793492 |
| ENSMUSG00000001444 | Tbx21 | −3.27856817 | 0.006161493 |
| ENSMUSG00000038517 | Tbkbp1 | −1.30516519 | 0.007120628 |
| ENSMUSG00000018547 | Pip4k2b | −1.52900846 | 0.005046392 |
| ENSMUSG00000017417 | Plxdc1 | 1.68321169 | 3.41E−04 |
| ENSMUSG00000017400 | Stac2 | −5.10118656 | 5.78E−04 |
| ENSMUSG00000061718 | Ppp1r1b | −3.19261594 | 4.40E−04 |
| ENSMUSG00000018167 | Stard3 | 1.40342354 | 2.51E−04 |
| ENSMUSG00000018168 | Ikzf3 | −1.54843187 | 0.009785288 |
| ENSMUSG00000020889 | Nr1d1 | 2.19809785 | 3.90E−05 |
| ENSMUSG00000038013 | Wipf2 | −1.07195158 | 0.007099712 |
| ENSMUSG00000037992 | Rara | −2.57009874 | 4.15E−06 |
| ENSMUSG00000037944 | Ccr7 | −1.48564414 | 0.010936016 |
| ENSMUSG00000006931 | P3h4 | 1.80916898 | 8.78E−04 |
| ENSMUSG00000048732 | Klhl11 | 1.49474345 | 0.022131108 |
| ENSMUSG00000001751 | Naglu | 1.93984658 | 2.04E−05 |
| ENSMUSG00000017167 | Cntnap1 | −1.65057548 | 0.037440394 |
| ENSMUSG00000034993 | Vat1 | −1.36979133 | 0.013960321 |
| ENSMUSG00000001313 | Rnd2 | −2.49973741 | 5.17E−04 |
| ENSMUSG00000003518 | Dusp3 | −1.01942188 | 0.021514336 |
| ENSMUSG00000017309 | Cd300lg | −2.60098992 | 4.18E−05 |
| ENSMUSG00000034793 | G6pc3 | 1.02342803 | 0.003310039 |
| ENSMUSG00000059995 | Atxn7l3 | −1.25533397 | 0.009028972 |
| ENSMUSG00000006575 | Rundc3a | −2.21576215 | 0.006292826 |
| ENSMUSG00000034708 | Grn | 1.7550878 | 2.16E−04 |
| ENSMUSG00000020926 | Adam11 | −1.92935713 | 0.026542124 |
| ENSMUSG00000020932 | Gfap | −6.3923573 | 6.29E−07 |
| ENSMUSG00000048878 | Hexim1 | −1.65544506 | 2.51E−04 |
| ENSMUSG00000018411 | Mapt | −4.24184622 | 9.89E−05 |
| ENSMUSG00000020695 | Mrc2 | 1.80885663 | 6.37E−04 |
| ENSMUSG00000019590 | Cyb561 | −2.79920519 | 0.001023199 |
| ENSMUSG00000020681 | Ace | −1.53167224 | 8.70E−04 |
| ENSMUSG00000078622 | Ccdc47 | 1.31580726 | 3.58E−04 |
| ENSMUSG00000040592 | Cd79b | 1.37431948 | 8.27E−04 |
| ENSMUSG00000001029 | Icam2 | −1.02597044 | 0.043431005 |
| ENSMUSG00000040481 | Bptf | −1.04171731 | 0.002259662 |
| ENSMUSG00000085811 | Cep112it | −2.15381036 | 0.021254552 |
| ENSMUSG00000041920 | Slc16a6 | 1.19079496 | 7.00E−04 |
| ENSMUSG00000020604 | Arsg | 1.99880921 | 1.35E−05 |
| ENSMUSG00000041797 | Abca9 | 1.59961386 | 2.06E−04 |
| ENSMUSG00000041695 | Kcnj2 | 1.41559834 | 0.001270131 |
| ENSMUSG00000041674 | BC006965 | −6.975535 | 5.76E−04 |
| ENSMUSG00000000567 | Sox9 | −4.8508006 | 4.00E−05 |
| ENSMUSG00000041654 | Slc39a11 | 1.12557005 | 0.001594646 |
| ENSMUSG00000018661 | Cog1 | 1.09261106 | 0.004171358 |
| ENSMUSG00000057322 | Rpl38 | −1.07277738 | 0.005007601 |
| ENSMUSG00000034714 | Ttyh2 | 1.44692468 | 5.37E−04 |
| ENSMUSG00000083237 | Btf3-ps13 | −3.75361205 | 0.001622627 |
| ENSMUSG00000034641 | Cd300ld | −1.61580194 | 1.82E−04 |
| ENSMUSG00000020732 | Rab37 | −1.72054487 | 0.036418109 |
| ENSMUSG00000047798 | Cd300lf | −1.07087133 | 0.001826771 |
| ENSMUSG00000045980 | Tmem104 | 1.33739278 | 7.10E−04 |
| ENSMUSG00000034586 | Hid1 | −1.71481792 | 0.029839106 |
| ENSMUSG00000016940 | Kctd2 | −1.49356368 | 0.003300338 |
| ENSMUSG00000048442 | Smim5 | −1.9532584 | 5.74E−04 |
| ENSMUSG00000020773 | Trim47 | 1.5572691 | 0.005012233 |
| ENSMUSG00000020802 | Ube20 | −1.14005281 | 0.020599548 |
| ENSMUSG00000020818 | Mfsd11 | 1.31829471 | 3.59E−04 |
| ENSMUSG00000059248 | Septin9 | −1.35213436 | 0.001975684 |
| ENSMUSG00000025571 | Tnrc6c | −1.26634557 | 7.38E−04 |
| ENSMUSG00000025574 | Tk1 | −1.04015178 | 0.007398295 |
| ENSMUSG00000017716 | Birc5 | −1.30811526 | 0.006567196 |
| ENSMUSG00000053113 | Socs3 | 1.31932382 | 0.009156059 |
| ENSMUSG00000033987 | Dnah17 | −1.29634754 | 0.049776101 |
| ENSMUSG00000033880 | Lgals3bp | 2.03928801 | 9.63E−05 |
| ENSMUSG00000025575 | Cant1 | 1.08902229 | 0.00316104 |
| ENSMUSG00000025576 | Rbfox3 | −1.97543143 | 0.019655388 |
| ENSMUSG00000025579 | Gaa | 1.91981912 | 6.27E−05 |
| ENSMUSG00000061306 | Slc38a10 | 1.61498928 | 2.28E−04 |
| ENSMUSG00000025386 | Pde6g | −1.9779711 | 0.030125968 |
| ENSMUSG00000025130 | P4hb | 1.3239161 | 5.71E−04 |
| ENSMUSG00000025134 | Alyref | −1.79290484 | 8.11E−04 |
| ENSMUSG00000025141 | Myadml2 | 1.68120557 | 0.002155383 |
| ENSMUSG00000025163 | Cd7 | −1.40432922 | 6.30E−04 |
| ENSMUSG00000025169 | Ogfod3 | 1.22925721 | 0.018924718 |
| ENSMUSG00000039294 | Cybc1 | 1.20116167 | 7.24E−04 |
| ENSMUSG00000000056 | Narf | 1.19059625 | 8.44E−04 |
| ENSMUSG00000021215 | Net1 | −1.35889254 | 0.002210659 |
| ENSMUSG00000000078 | Klf6 | 1.09263761 | 0.00679932 |
| ENSMUSG00000021196 | Pfkp | −1.68277246 | 8.19E−05 |
| ENSMUSG00000021303 | Gng4 | −4.92805587 | 6.76E−04 |
| ENSMUSG00000041297 | Cdk13 | −1.10328644 | 0.008809309 |
| ENSMUSG00000076757 | Trgc4 | −4.76127415 | 6.43E−04 |
| ENSMUSG00000002808 | Epdr1 | −3.99732853 | 0.00189007 |
| ENSMUSG00000047462 | Gpr141b | −2.37947818 | 0.004657446 |
| ENSMUSG00000053101 | Gpr141 | −1.93651061 | 5.02E−05 |
| ENSMUSG00000021326 | Trim27 | 1.01220021 | 0.020675805 |
| ENSMUSG00000054931 | Zkscan4 | 1.49345369 | 0.001983522 |
| ENSMUSG00000058773 | H1f5 | −2.41095649 | 0.007237994 |
| ENSMUSG00000006179 | Prss16 | −2.57012513 | 0.011548416 |
| ENSMUSG00000069301 | H2ac11 | −2.01247973 | 0.023144886 |
| ENSMUSG00000058385 | H2bc8 | 1.39066156 | 0.001587289 |
| ENSMUSG00000019132 | BC005537 | −1.80489941 | 5.53E−05 |
| ENSMUSG00000021340 | Gpld1 | −1.68020268 | 0.038051771 |
| ENSMUSG00000048978 | Nrsn1 | −4.61440724 | 4.97E−05 |
| ENSMUSG00000021342 | Prl | −10.0616436 | 2.56E−06 |
| ENSMUSG00000044734 | Serpinb1a | −2.42217538 | 1.72E−05 |
| ENSMUSG00000079049 | Serpinb1c | −1.97300928 | 0.017440756 |
| ENSMUSG00000042842 | Serpinb6b | −2.07419095 | 0.001452858 |
| ENSMUSG00000021403 | Serpinb9b | −2.22719693 | 0.013495412 |
| ENSMUSG00000059288 | Cdyl | −1.16187459 | 0.020043596 |
| ENSMUSG00000021423 | Ly86 | 1.34755713 | 0.006271025 |
| ENSMUSG00000038991 | Txndc5 | 1.48318474 | 0.002604612 |
| ENSMUSG00000021363 | Mak | −2.94229702 | 0.013609331 |
| ENSMUSG00000021362 | Gcm2 | 2.25750299 | 0.026319936 |
| ENSMUSG00000091264 | Smim13 | −1.15647766 | 0.025998707 |
| ENSMUSG00000021365 | Nedd9 | −1.24322287 | 6.32E−04 |
| ENSMUSG00000021367 | Edn1 | 1.73297317 | 0.001442729 |
| ENSMUSG00000051335 | Gfod1 | −2.07600315 | 0.005338659 |
| ENSMUSG00000044164 | Rnf182 | −3.52838406 | 0.004475862 |
| ENSMUSG00000015396 | Cd83 | 1.58848555 | 1.33E−04 |
| ENSMUSG00000050954 | Zfp169 | 1.50758702 | 0.003134154 |
| ENSMUSG00000038025 | Phf2 | −1.51232685 | 0.011151651 |
| ENSMUSG00000038014 | Fam120a | −1.09037934 | 0.002750927 |
| ENSMUSG00000021384 | Susd3 | 1.16464505 | 0.002630091 |
| ENSMUSG00000021451 | Sema4d | 1.24216401 | 0.001407159 |
| ENSMUSG00000047842 | Diras2 | −2.85582258 | 0.024542847 |
| ENSMUSG00000025867 | Cplx2 | −5.12848682 | 1.90E−04 |
| ENSMUSG00000043183 | Simc1 | 1.14764253 | 0.005165668 |
| ENSMUSG00000025873 | Faf2 | 1.12360978 | 0.001494002 |
| ENSMUSG00000021484 | Lman2 | 1.01287119 | 0.003141533 |
| ENSMUSG00000074886 | Grk6 | −2.15326034 | 7.96E−05 |
| ENSMUSG00000034675 | Dbn1 | −2.19286162 | 0.0031216 |
| ENSMUSG00000021493 | Pdlim7 | −2.79973212 | 1.33E−04 |
| ENSMUSG00000021495 | Fam193b | −1.34072658 | 4.61E−04 |
| ENSMUSG00000021501 | Caml | 1.32388462 | 4.12E−04 |
| ENSMUSG00000021497 | Txndc15 | 1.16424046 | 0.001104299 |
| ENSMUSG00000014164 | Klhl3 | 1.33127253 | 0.040280241 |
| ENSMUSG00000007836 | Hnrnpa0 | −1.23408481 | 0.006343023 |
| ENSMUSG00000055254 | Ntrk2 | −3.17457877 | 0.001238579 |
| ENSMUSG00000021556 | Golm1 | 2.10625008 | 1.45E−05 |
| ENSMUSG00000021559 | Dapk1 | −1.14591788 | 0.004415518 |
| ENSMUSG00000021466 | Ptch1 | 1.09750952 | 0.025135738 |
| ENSMUSG00000021477 | Ctsl | 1.77612005 | 5.16E−04 |
| ENSMUSG00000038212 | Mfsd14b | 1.20191055 | 9.82E−04 |
| ENSMUSG00000021518 | Ptdss1 | 1.22184237 | 6.41E−04 |
| ENSMUSG00000058900 | Rsl1 | 1.32801789 | 0.003816251 |
| ENSMUSG00000051037 | Zfp455 | 1.37333012 | 0.010608827 |
| ENSMUSG00000078995 | Zfp456 | 1.33730855 | 0.00920802 |
| ENSMUSG00000078994 | Zfp429 | 1.1039607 | 0.021100037 |
| ENSMUSG00000059839 | Zfp874b | 1.21744737 | 0.014787894 |
| ENSMUSG00000048280 | Zfp738 | 1.28166489 | 0.021799813 |
| ENSMUSG00000049538 | Adamts16 | 1.36153888 | 0.001140115 |
| ENSMUSG00000021608 | Lpcat1 | 1.16770167 | 0.003623548 |
| ENSMUSG00000017756 | Slc12a7 | 1.00748032 | 0.010132572 |
| ENSMUSG00000021573 | Tppp | 1.14891377 | 0.003581957 |
| ENSMUSG00000021583 | Erap1 | 1.22080872 | 7.91E−04 |
| ENSMUSG00000021589 | Rhobtb3 | −4.03299485 | 0.001500889 |
| ENSMUSG00000021592 | Arsk | 1.41381937 | 9.98E−04 |
| ENSMUSG00000093668 | Pou5f2 | −1.61975864 | 0.00854536 |
| ENSMUSG00000069170 | Adgrv1 | −2.21567923 | 0.010192742 |
| ENSMUSG00000035840 | Lysmd3 | 1.0275195 | 0.004751765 |
| ENSMUSG00000034488 | Edil3 | −1.92929731 | 0.025748605 |
| ENSMUSG00000021614 | Vcan | −3.18548249 | 0.001563919 |
| ENSMUSG00000021703 | Serinc5 | 1.02720336 | 0.025598241 |
| ENSMUSG00000042082 | Arsb | 2.0092655 | 3.83E−05 |
| ENSMUSG00000045312 | Lhfpl2 | 1.83247732 | 5.04E−05 |
| ENSMUSG00000021687 | Scamp1 | 1.50596837 | 1.53E−04 |
| ENSMUSG00000048376 | F2r | 1.83913668 | 0.013912869 |
| ENSMUSG00000021675 | F2rl2 | −3.42002279 | 7.28E−04 |
| ENSMUSG00000109561 | Ankrd31 | −6.55107183 | 0.001140645 |
| ENSMUSG00000021665 | Hexb | 2.12544549 | 1.06E−04 |
| ENSMUSG00000021662 | Arhgef28 | −3.44588144 | 0.003181464 |
| ENSMUSG00000052727 | Map1b | −1.97692869 | 0.00455935 |
| ENSMUSG00000021639 | Gtf2h2 | 1.01437288 | 0.006661296 |
| ENSMUSG00000021638 | Ocln | 2.50384786 | 0.00236834 |
| ENSMUSG00000021636 | Marveld2 | 1.03691404 | 0.024276877 |
| ENSMUSG00000048924 | Ccdc125 | −1.65540167 | 0.001419491 |
| ENSMUSG00000021629 | Slc30a5 | 1.64102814 | 5.97E−05 |
| ENSMUSG00000021720 | Rnf180 | 1.39505151 | 4.02E−04 |
| ENSMUSG00000032846 | Zswim6 | −1.27120351 | 0.007763413 |
| ENSMUSG00000021697 | Depdc1b | −1.35871098 | 0.037838315 |
| ENSMUSG00000046006 | Gapt | −1.83971403 | 0.001004861 |
| ENSMUSG00000021756 | Il6st | 1.8160142 | 5.78E−05 |
| ENSMUSG00000050377 | Il31ra | −1.54410873 | 0.008515294 |
| ENSMUSG00000021759 | Plpp1 | 2.09307057 | 1.55E−04 |
| ENSMUSG00000021760 | Gpx8 | −1.80341687 | 0.03775938 |
| ENSMUSG00000023132 | Gzma | −3.0349147 | 2.72E−05 |
| ENSMUSG00000042284 | Itga1 | −3.05278524 | 0.010323013 |
| ENSMUSG00000021728 | Emb | −2.04008161 | 1.47E−05 |
| ENSMUSG00000021730 | Hcn1 | −3.2046735 | 0.012791405 |
| ENSMUSG00000025451 | Paip1 | −1.38952185 | 0.002467424 |
| ENSMUSG00000095930 | Nim1k | −1.36533591 | 0.011283898 |
| ENSMUSG00000020654 | Adcy3 | 1.96963481 | 6.56E−04 |
| ENSMUSG00000020647 | Ncoa1 | −1.12011081 | 0.003350142 |
| ENSMUSG00000020639 | Pfn4 | −3.35017901 | 0.001879378 |
| ENSMUSG00000020635 | Fkbp1b | −1.01584149 | 0.019365556 |
| ENSMUSG00000020592 | Sdc1 | −1.54862423 | 0.035178539 |
| ENSMUSG00000020621 | Rdh14 | 1.18088386 | 0.006462834 |
| ENSMUSG00000051235 | Gen1 | −3.06591305 | 0.002536109 |
| ENSMUSG00000020601 | Trib2 | −3.38214811 | 0.00546043 |
| ENSMUSG00000020591 | Ntsr2 | −5.34879294 | 7.74E−05 |
| ENSMUSG00000020571 | Pdia6 | 1.06956881 | 0.002477967 |
| ENSMUSG00000069755 | Zfp125 | 1.14658047 | 0.021538501 |
| ENSMUSG00000052593 | Adam17 | 1.58092931 | 1.46E−04 |
| ENSMUSG00000062563 | Cys1 | −3.3823042 | 0.008373831 |
| ENSMUSG00000020649 | Rrm2 | −1.074573 | 0.013291357 |
| ENSMUSG00000020646 | Mboat2 | −7.64435532 | 3.08E−04 |
| ENSMUSG00000020644 | Id2 | 1.04300392 | 0.003542078 |
| ENSMUSG00000020642 | Rnf144a | −2.72086796 | 5.19E−05 |
| ENSMUSG00000063632 | Sox11 | −3.86177046 | 0.003551819 |
| ENSMUSG00000061911 | Myt1l | −4.45869265 | 0.00144165 |
| ENSMUSG00000043061 | Tmem18 | 1.50079127 | 0.004455842 |
| ENSMUSG00000002997 | Prkar2b | −2.87997343 | 0.00185063 |
| ENSMUSG00000020573 | Pik3cg | 1.03361513 | 0.006784063 |
| ENSMUSG00000020570 | Sypl | 1.22055149 | 0.009103406 |
| ENSMUSG00000079143 | Atxn7l1os1 | −2.27324707 | 0.010855641 |
| ENSMUSG00000085664 | Atxn7l1os2 | −1.76998754 | 0.03944556 |
| ENSMUSG00000020590 | Snx13 | 1.09950973 | 0.004302429 |
| ENSMUSG00000036169 | Sostdc1 | 1.87600035 | 0.037012156 |
| ENSMUSG00000050103 | Agmo | 1.62501993 | 4.94E−04 |
| ENSMUSG00000036095 | Dgkb | −8.2061286 | 7.47E−05 |
| ENSMUSG00000071342 | Lsmem1 | −3.20755702 | 4.58E−04 |
| ENSMUSG00000089371 | Mir1938 | 1.96174727 | 0.021610765 |
| ENSMUSG00000035954 | Dock4 | 1.07101268 | 0.008262549 |
| ENSMUSG00000020598 | Nrcam | −4.4028621 | 8.83E−04 |
| ENSMUSG00000046314 | Stxbp6 | −2.44127875 | 2.36E−04 |
| ENSMUSG00000020954 | Strn3 | −1.11143224 | 0.004838795 |
| ENSMUSG00000061603 | Akap6 | −8.23712605 | 8.29E−04 |
| ENSMUSG00000021010 | Npas3 | −3.078095 | 0.01111431 |
| ENSMUSG00000066491 | Cox6c2 | −1.55566925 | 0.04589042 |
| ENSMUSG00000035329 | Fbxo33 | −1.07688671 | 0.016860626 |
| ENSMUSG00000034912 | Mdga2 | −3.20407727 | 0.001706411 |
| ENSMUSG00000043998 | Mgat2 | 1.3154472 | 4.30E−04 |
| ENSMUSG00000044147 | Arf6 | −1.61300878 | 1.53E−04 |
| ENSMUSG00000034761 | Map4k5 | 1.22052152 | 0.004004418 |
| ENSMUSG00000021066 | Atl1 | 1.01901421 | 0.047079892 |
| ENSMUSG00000021067 | Sav1 | −1.64245228 | 0.029338997 |
| ENSMUSG00000021071 | Trim9 | −2.53916393 | 0.008430892 |
| ENSMUSG00000021072 | Tmx1 | 1.18106404 | 0.00546135 |
| ENSMUSG00000021087 | Rtn1 | 1.02056181 | 0.003285892 |
| ENSMUSG00000034501 | Pcnx4 | 1.08315153 | 0.022954273 |
| ENSMUSG00000063450 | Syne2 | −1.32303311 | 0.002186686 |
| ENSMUSG00000052609 | Plekhg3 | −1.87094959 | 0.001469428 |
| ENSMUSG00000021062 | Rab15 | −2.23269496 | 0.010410392 |
| ENSMUSG00000021120 | Pigh | 1.56289671 | 4.01E−04 |
| ENSMUSG00000021125 | Arg2 | −2.23133468 | 0.002213217 |
| ENSMUSG00000066441 | Rdh11 | 1.55536903 | 2.21E−04 |
| ENSMUSG00000021123 | Rdh12 | −1.17209686 | 0.016591125 |
| ENSMUSG00000032705 | Exd2 | 1.25417539 | 7.96E−04 |
| ENSMUSG00000048833 | Slc39a9 | 1.38643503 | 4.25E−04 |
| ENSMUSG00000021133 | Susd6 | 1.12061044 | 0.004463925 |
| ENSMUSG00000021135 | Slc10a1 | −2.08200845 | 0.001575941 |
| ENSMUSG00000042734 | Ttc9 | 1.08747338 | 0.037241032 |
| ENSMUSG00000042724 | Map3k9 | −1.46991116 | 0.005203825 |
| ENSMUSG00000021219 | Rgs6 | −6.36505386 | 6.36E−05 |
| ENSMUSG00000021221 | Dpf3 | −4.30875179 | 0.002699417 |
| ENSMUSG00000019969 | Psen1 | 1.46770086 | 1.34E−04 |
| ENSMUSG00000021224 | Numb | 1.34030323 | 3.95E−04 |
| ENSMUSG00000090843 | Heatr4 | −2.98188888 | 0.008239237 |
| ENSMUSG00000057265 | Bbof1 | −3.33886509 | 0.004717142 |
| ENSMUSG00000019235 | Rps6kl1 | −1.7718411 | 0.013741436 |
| ENSMUSG00000021250 | Fos | 1.35092507 | 0.003085206 |
| ENSMUSG00000034271 | Jdp2 | −1.3129437 | 0.00811788 |
| ENSMUSG00000021253 | Tgfb3 | −4.09922507 | 2.51E−04 |
| ENSMUSG00000021257 | Angel1 | 2.5256067 | 1.54E−04 |
| ENSMUSG00000034126 | Pomt2 | 1.57172976 | 9.50E−04 |
| ENSMUSG00000066392 | Nrxn3 | −2.56956435 | 0.004756866 |
| ENSMUSG00000007682 | Dio2 | −7.32073846 | 1.87E−05 |
| ENSMUSG00000061533 | Cep128 | −1.73250958 | 4.44E−04 |
| ENSMUSG00000020964 | Sel1l | 1.22167664 | 0.001533694 |
| ENSMUSG00000045404 | Kcnk13 | 1.88854849 | 2.11E−05 |
| ENSMUSG00000033530 | Ttc7b | −1.64996727 | 0.001416104 |
| ENSMUSG00000047415 | Gpr68 | −2.47196666 | 7.46E−05 |
| ENSMUSG00000021182 | Ccdc88c | −1.66910499 | 8.04E−04 |
| ENSMUSG00000021190 | Lgmn | 1.64437352 | 2.56E−04 |
| ENSMUSG00000021200 | Asb2 | 1.71819386 | 9.37E−05 |
| ENSMUSG00000066363 | Serpina3f | −1.68853438 | 0.028186316 |
| ENSMUSG00000041481 | Serpina3g | −2.00461276 | 8.43E−04 |
| ENSMUSG00000021091 | Serpina3n | −6.1732952 | 4.32E−04 |
| ENSMUSG00000054150 | Syne3 | −2.35283695 | 0.00706864 |
| ENSMUSG00000021281 | Tnfaip2 | −1.05594071 | 0.019698237 |
| ENSMUSG00000037686 | Aspg | −4.38294424 | 0.001269996 |
| ENSMUSG00000037679 | Inf2 | −1.16688106 | 0.024090477 |
| ENSMUSG00000072825 | Cep170b | −1.56417491 | 0.042381324 |
| ENSMUSG00000052160 | Pld4 | 1.158079 | 0.002173348 |
| ENSMUSG00000021298 | Gpr132 | −2.71289991 | 8.39E−05 |
| ENSMUSG00000021144 | Mta1 | −1.29690384 | 0.015340844 |
| ENSMUSG00000006360 | Crip1 | −1.47990531 | 2.72E−04 |
| ENSMUSG00000095079 | Igha | 4.43697658 | 3.75E−05 |
| ENSMUSG00000076620 | Ighj2 | 1.61940348 | 0.02333173 |
| ENSMUSG00000095571 | Ighv5-17 | 5.86530271 | 2.37E−04 |
| ENSMUSG00000093838 | Ighv3-1 | 8.68058912 | 3.79E−04 |
| ENSMUSG00000076695 | Ighv1-18 | 8.55387561 | 8.62E−05 |
| ENSMUSG00000104452 | Ighv8-8 | 8.60653907 | 8.30E−05 |
| ENSMUSG00000041992 | Rapgef5 | 1.01921063 | 0.02917689 |
| ENSMUSG00000021175 | Cdca71 | −1.25348552 | 0.011701077 |
| ENSMUSG00000018581 | Dnah11 | −3.78443502 | 0.004133184 |
| ENSMUSG00000064373 | Selenop | 1.13767114 | 0.006733539 |
| ENSMUSG00000003882 | Il7r | 1.17421733 | 0.019572421 |
| ENSMUSG00000044224 | Dnajc21 | −1.46270151 | 0.004687548 |
| ENSMUSG00000022197 | Pdzd2 | −1.48937983 | 0.013666768 |
| ENSMUSG00000052253 | Zfp622 | 1.02271025 | 0.007207962 |
| ENSMUSG00000046034 | Otulin | −1.46557131 | 0.002610762 |
| ENSMUSG00000056069 | Otulinl | 1.40995403 | 4.65E−04 |
| ENSMUSG00000022240 | Ctnnd2 | −1.227601 | 0.006569868 |
| ENSMUSG00000022237 | Ankrd33b | −1.63283117 | 0.007001939 |
| ENSMUSG00000022261 | Sdc2 | −1.85510965 | 0.039894924 |
| ENSMUSG00000039007 | Cpq | 1.76799299 | 2.31E−04 |
| ENSMUSG00000022257 | Laptm4b | 1.90195835 | 9.74E−05 |
| ENSMUSG00000022323 | Rida | −1.12865443 | 0.005570521 |
| ENSMUSG00000022283 | Pabpc1 | −1.26597067 | 0.001362763 |
| ENSMUSG00000051359 | Ncald | −4.7429958 | 2.10E−04 |
| ENSMUSG00000022296 | Baalc | −4.17107918 | 7.07E−04 |
| ENSMUSG00000022305 | Lrp12 | 1.6550375 | 2.84E−04 |
| ENSMUSG00000022309 | Angpt1 | −4.58665747 | 8.33E−04 |
| ENSMUSG00000038725 | Pkhd1l1 | −3.28175303 | 0.011694837 |
| ENSMUSG00000022340 | Sybu | −2.46339814 | 0.012852954 |
| ENSMUSG00000022311 | Csmd3 | 1.01316778 | 0.002536168 |
| ENSMUSG00000038679 | Trps1 | −2.03455832 | 1.62E−05 |
| ENSMUSG00000022314 | Rad21 | −1.27964232 | 0.003839175 |
| ENSMUSG00000061731 | Ext1 | −1.47672467 | 0.001238074 |
| ENSMUSG00000022425 | Enpp2 | 1.25619928 | 0.012004148 |
| ENSMUSG00000060429 | Sntb1 | −2.13023085 | 0.009869974 |
| ENSMUSG00000022365 | Derl1 | 1.37455988 | 2.61E−04 |
| ENSMUSG00000022357 | Klhl38 | 1.38269582 | 0.011035495 |
| ENSMUSG00000022346 | Myc | 1.09650145 | 0.003047722 |
| ENSMUSG00000055748 | Gsdmc4 | −2.86447308 | 0.008623221 |
| ENSMUSG00000022376 | Adcy8 | 1.80791805 | 0.030053382 |
| ENSMUSG00000005125 | Ndrg1 | −2.84639532 | 3.13E−05 |
| ENSMUSG00000013846 | St3gal1 | −1.07635494 | 0.013360735 |
| ENSMUSG00000036698 | Ago2 | −1.28608417 | 7.94E−04 |
| ENSMUSG00000022594 | Lynx1 | −2.89119503 | 0.003065569 |
| ENSMUSG00000044678 | Ly6k | 2.74060042 | 0.007863794 |
| ENSMUSG00000022586 | Ly6i | −1.36509374 | 0.006029311 |
| ENSMUSG00000079018 | Ly6c1 | −2.04267888 | 0.004354732 |
| ENSMUSG00000022584 | Ly6c2 | −1.92590844 | 4.97E−05 |
| ENSMUSG00000102051 | Ly6a2 | −2.82391035 | 9.71E−06 |
| ENSMUSG00000022582 | Ly6g | −1.93116318 | 0.001011441 |
| ENSMUSG00000103906 | Tigd5 | −1.83207541 | 0.047179891 |
| ENSMUSG00000050846 | Zfp623 | 1.10717151 | 0.008296508 |
| ENSMUSG00000022568 | Scrib | −1.87893736 | 1.81E−04 |
| ENSMUSG00000075590 | Nrbp2 | −2.28463923 | 0.037779181 |
| ENSMUSG00000022565 | Plec | −1.89637699 | 2.36E−05 |
| ENSMUSG00000049653 | Spatc1 | −6.20849132 | 0.002011476 |
| ENSMUSG00000022561 | Gpaa1 | 1.02423466 | 0.002515807 |
| ENSMUSG00000022560 | Slc52a2 | 1.06695955 | 0.003182719 |
| ENSMUSG00000063354 | Slc39a4 | 1.83006782 | 1.36E−04 |
| ENSMUSG00000019080 | Mfsd3 | 1.13411349 | 0.005830374 |
| ENSMUSG00000071716 | Apol7e | −7.39972117 | 3.73E−04 |
| ENSMUSG00000056656 | Apol8 | −2.18539267 | 0.019406447 |
| ENSMUSG00000016552 | Foxred2 | −1.01056667 | 0.0412317 |
| ENSMUSG00000005716 | Pvalb | −6.0541052 | 2.16E−04 |
| ENSMUSG00000044986 | Tst | −2.89493245 | 4.31E−04 |
| ENSMUSG00000033287 | Kctd17 | −3.06855293 | 0.001716669 |
| ENSMUSG00000068227 | Il2rb | −3.19535574 | 1.15E−04 |
| ENSMUSG00000033170 | Card10 | −1.57306227 | 0.003181075 |
| ENSMUSG00000068220 | Lgals1 | −1.23340608 | 6.18E−04 |
| ENSMUSG00000033006 | Sox10 | −1.79393422 | 0.039533982 |
| ENSMUSG00000009035 | Tmem184b | 1.49800728 | 2.12E−04 |
| ENSMUSG00000022415 | Syngr1 | 1.31400862 | 0.0020377 |
| ENSMUSG00000042351 | Grap2 | −1.86725772 | 0.010597732 |
| ENSMUSG00000022404 | Slc25a17 | 1.12840247 | 0.001461107 |
| ENSMUSG00000047108 | Dnajb7 | 2.09378518 | 0.022858144 |
| ENSMUSG00000042109 | Csdc2 | −4.87044182 | 7.66E−05 |
| ENSMUSG00000068105 | Tnfrsf13c | −1.87271897 | 0.016066018 |
| ENSMUSG00000068101 | Cenpm | −1.24533889 | 0.026100867 |
| ENSMUSG00000068099 | Smim45 | −8.58172755 | 7.02E−05 |
| ENSMUSG00000022456 | Septin3 | −2.6964002 | 8.73E−04 |
| ENSMUSG00000041852 | Tcf20 | −1.71051271 | 0.002635205 |
| ENSMUSG00000048755 | Mcat | 1.05870603 | 0.02292269 |
| ENSMUSG00000016763 | Scube1 | 1.81926326 | 0.004435823 |
| ENSMUSG00000018865 | Sult4a1 | −2.02502109 | 0.010406702 |
| ENSMUSG00000022438 | Parvb | −1.4559139 | 0.004097939 |
| ENSMUSG00000062760 | Shisal1 | −4.35152097 | 0.002793607 |
| ENSMUSG00000022432 | Smc1b | −1.70883435 | 0.036427015 |
| ENSMUSG00000035900 | Gramd4 | −1.50173132 | 0.001459183 |
| ENSMUSG00000035845 | Alg12 | 1.0216849 | 0.014651393 |
| ENSMUSG00000035805 | Mlc1 | −4.32922329 | 5.91E−06 |
| ENSMUSG00000035757 | Selenoo | 1.63716175 | 0.013588811 |
| ENSMUSG00000036606 | Plxnb2 | 1.34217173 | 0.001084497 |
| ENSMUSG00000015377 | Dennd6b | 1.02578641 | 0.034600985 |
| ENSMUSG00000022614 | Lmf2 | 1.11429056 | 0.002567468 |
| ENSMUSG00000022619 | Mapk8ip2 | −3.81493631 | 0.006230883 |
| ENSMUSG00000022620 | Arsa | 1.02082668 | 0.00361802 |
| ENSMUSG00000022629 | Kif21a | −3.081253 | 0.001885781 |
| ENSMUSG00000036273 | Lrrk2 | −2.36232519 | 2.81E−05 |
| ENSMUSG00000055022 | Cntn1 | −5.97600768 | 4.37E−06 |
| ENSMUSG00000036218 | Pdzrn4 | 2.23907572 | 0.01925598 |
| ENSMUSG00000045608 | Dbx2 | −3.52085042 | 0.007024736 |
| ENSMUSG00000022468 | Endou | −3.66944614 | 0.005306268 |
| ENSMUSG00000022469 | Rapgef3 | −2.38598313 | 0.048999007 |
| ENSMUSG00000022475 | Hdac7 | −2.95213336 | 1.19E−04 |
| ENSMUSG00000022479 | Vdr | −2.82919757 | 0.002276143 |
| ENSMUSG00000033065 | Pfkm | −1.04657593 | 0.024638001 |
| ENSMUSG00000090129 | Olfr287 | −4.69663988 | 8.25E−04 |
| ENSMUSG00000022987 | Zfp641 | 1.24608851 | 0.022251167 |
| ENSMUSG00000022992 | Kansl2 | 1.08863997 | 0.00174374 |
| ENSMUSG00000003352 | Cacnb3 | −3.46945686 | 0.001024055 |
| ENSMUSG00000054855 | Rnd1 | −2.26219142 | 0.001023234 |
| ENSMUSG00000003355 | Fkbp11 | 1.92410695 | 0.033737422 |
| ENSMUSG00000059213 | Ddn | −5.15794166 | 0.00198367 |
| ENSMUSG00000048154 | Kmt2d | −1.23637878 | 0.001106321 |
| ENSMUSG00000023030 | Slc11a2 | 1.17493746 | 0.001349269 |
| ENSMUSG00000044636 | Csrnp2 | 1.13419595 | 0.015154113 |
| ENSMUSG00000037185 | Krt80 | −1.73414562 | 0.001440078 |
| ENSMUSG00000023039 | Krt7 | −2.65318889 | 0.004456316 |
| ENSMUSG00000023046 | Igfbp6 | −2.0050128 | 0.006223143 |
| ENSMUSG00000001281 | Itgb7 | −1.81829483 | 4.55E−05 |
| ENSMUSG00000001288 | Rarg | −2.26416817 | 3.99E−04 |
| ENSMUSG00000023047 | Amhr2 | −2.59962816 | 0.03235144 |
| ENSMUSG00000009575 | Cbx5 | −1.03010848 | 0.003927205 |
| ENSMUSG00000058794 | Nfe2 | −1.87253024 | 3.52E−04 |
| ENSMUSG00000063234 | Gpr84 | 2.14216409 | 2.07E−05 |
| ENSMUSG00000022489 | Pde1b | −1.07796564 | 0.005102357 |
| ENSMUSG00000022490 | Ppp1r1a | −2.34535262 | 0.011904927 |
| ENSMUSG00000022534 | Mefv | −3.26229119 | 2.98E−05 |
| ENSMUSG00000049871 | Nlrc3 | −1.18123083 | 0.026913941 |
| ENSMUSG00000022519 | Srl | 1.47556803 | 0.008777304 |
| ENSMUSG00000023143 | Nagpa | 1.27969731 | 4.11E−04 |
| ENSMUSG00000057880 | Abat | −5.5705532 | 7.65E−06 |
| ENSMUSG00000022710 | Usp7 | −1.33219972 | 3.58E−04 |
| ENSMUSG00000022504 | Ciita | −2.33224493 | 8.42E−06 |
| ENSMUSG00000038055 | Dexi | −1.87868918 | 0.001975012 |
| ENSMUSG00000022498 | Txndc11 | 1.24291213 | 0.001091362 |
| ENSMUSG00000022496 | Tnfrsf17 | 1.34746773 | 0.006791988 |
| ENSMUSG00000044117 | Bmerb1 | −4.3229582 | 3.10E−05 |
| ENSMUSG00000060657 | Marf1 | −1.29278533 | 0.030496228 |
| ENSMUSG00000022792 | Yars2 | 1.66985873 | 0.001898203 |
| ENSMUSG00000026181 | Ppm1f | 1.10378127 | 0.002080976 |
| ENSMUSG00000022769 | Sdf2l1 | 1.1292846 | 0.003296544 |
| ENSMUSG00000022763 | Aifm3 | −8.04666708 | 7.36E−04 |
| ENSMUSG00000003166 | Dgcr2 | 1.01734131 | 0.005363601 |
| ENSMUSG00000072214 | Septin5 | −2.81531907 | 0.009059463 |
| ENSMUSG00000105906 | Iglc1 | −1.75203375 | 0.024486163 |
| ENSMUSG00000105547 | Iglc3 | −1.80993941 | 0.048885692 |
| ENSMUSG00000076934 | Iglv1 | −1.93191017 | 0.037049315 |
| ENSMUSG00000041205 | Map6d1 | −2.85214062 | 0.011492947 |
| ENSMUSG00000022818 | Cyp2ab1 | −2.4753305 | 0.005141921 |
| ENSMUSG00000033809 | Alg3 | 1.09189381 | 0.002876181 |
| ENSMUSG00000022853 | Ehhadh | 1.11202379 | 0.016074342 |
| ENSMUSG00000044626 | Liph | 2.01148809 | 8.48E−05 |
| ENSMUSG00000033581 | Igf2bp2 | 1.07925999 | 0.013173368 |
| ENSMUSG00000022861 | Dgkg | −2.60438598 | 1.75E−04 |
| ENSMUSG00000022887 | Masp1 | 2.67046237 | 7.40E−05 |
| ENSMUSG00000038168 | P3h2 | 1.17088303 | 0.005828525 |
| ENSMUSG00000022512 | Cldn1 | −1.42821115 | 0.009855853 |
| ENSMUSG00000022523 | Fgf12 | −6.90754569 | 9.61E−04 |
| ENSMUSG00000022525 | Plaat1 | −7.55271264 | 4.08E−04 |
| ENSMUSG00000022528 | Hes1 | −1.34668876 | 0.002959575 |
| ENSMUSG00000047434 | Xxylt1 | 1.04822698 | 0.02140737 |
| ENSMUSG00000022548 | Apod | −2.58371399 | 0.004554416 |
| ENSMUSG00000046598 | Bdh1 | −5.48487578 | 7.54E−04 |
| ENSMUSG00000075269 | Bex6 | −1.67104443 | 0.009916754 |
| ENSMUSG00000035764 | Fbxo45 | −1.08137391 | 0.035167653 |
| ENSMUSG00000022791 | Tnk2 | −2.993088 | 5.88E−04 |
| ENSMUSG00000075254 | Heg1 | −2.53285741 | 3.53E−05 |
| ENSMUSG00000022833 | Ccdc14 | 1.07347226 | 0.029293092 |
| ENSMUSG00000022840 | Adcy5 | −2.54341035 | 0.014188141 |
| ENSMUSG00000034473 | Sec22a | 1.06842118 | 0.003388719 |
| ENSMUSG00000022844 | Pdia5 | 1.04061583 | 0.00988608 |
| ENSMUSG00000059657 | Stfa2l1 | −4.21493392 | 4.50E−05 |
| ENSMUSG00000079597 | Cstdc4 | −5.22149624 | 9.92E−05 |
| ENSMUSG00000071562 | Stfa1 | −6.48980618 | 0.001248022 |
| ENSMUSG00000022901 | Cd86 | 1.516887 | 2.69E−04 |
| ENSMUSG00000034206 | Polq | −1.73000363 | 0.029293968 |
| ENSMUSG00000002845 | Tmem39a | 1.13249252 | 0.001560027 |
| ENSMUSG00000022793 | B4galt4 | 1.19668328 | 0.00267676 |
| ENSMUSG00000049436 | Upk1b | 2.31812118 | 1.13E−05 |
| ENSMUSG00000061080 | Lsamp | −3.44584568 | 1.01E−04 |
| ENSMUSG00000047261 | Gap43 | −5.81218877 | 4.15E−05 |
| ENSMUSG00000081824 | Ndufs5-ps | 2.13918896 | 4.42E−04 |
| ENSMUSG00000071552 | Tigit | −3.60493095 | 0.005226103 |
| ENSMUSG00000022664 | Slc35a5 | 1.55359139 | 1.23E−04 |
| ENSMUSG00000052013 | Btla | −1.1567824 | 0.011512654 |
| ENSMUSG00000022661 | Cd200 | −1.74659599 | 0.021899355 |
| ENSMUSG00000022658 | Tagln3 | −5.29134737 | 7.19E−06 |
| ENSMUSG00000022657 | Cd96 | −2.18872886 | 0.028053158 |
| ENSMUSG00000061100 | Retnla | −4.8265376 | 1.00E−06 |
| ENSMUSG00000022651 | Retnlg | −2.80047465 | 3.83E−05 |
| ENSMUSG00000033031 | Cip2a | −1.24570948 | 0.035538489 |
| ENSMUSG00000092009 | Myh15 | −2.19748813 | 0.022629049 |
| ENSMUSG00000022636 | Alcam | −1.67154226 | 2.90E−04 |
| ENSMUSG00000022604 | Cep97 | 1.16535396 | 0.006758973 |
| ENSMUSG00000046748 | Tmem45a2 | −3.39238509 | 0.009125359 |
| ENSMUSG00000022747 | St3gal6 | 1.50083635 | 3.32E−04 |
| ENSMUSG00000022742 | Cpox | 1.29590538 | 0.027014393 |
| ENSMUSG00000022744 | Cldnd1 | 1.09529365 | 0.002124092 |
| ENSMUSG00000022912 | Pros1 | 2.03443352 | 1.75E−05 |
| ENSMUSG00000047141 | Zfp654 | 1.1546492 | 0.003060654 |
| ENSMUSG00000032932 | Hspa13 | 1.50410883 | 2.21E−04 |
| ENSMUSG00000022867 | Usp25 | −1.01173811 | 0.009831157 |
| ENSMUSG00000022762 | Ncam2 | −1.64873544 | 0.04531574 |
| ENSMUSG00000053062 | Jam2 | 2.01045608 | 2.54E−05 |
| ENSMUSG00000022893 | Adamts1 | 2.17838737 | 1.42E−04 |
| ENSMUSG00000022894 | Adamts5 | −3.97463691 | 0.005186338 |
| ENSMUSG00000002489 | Tiam1 | −1.23745726 | 0.003218477 |
| ENSMUSG00000022982 | Sod1 | −1.39173695 | 0.001326178 |
| ENSMUSG00000046160 | Olig1 | −4.88225706 | 0.001313833 |
| ENSMUSG00000022971 | Ifnar2 | 1.09868289 | 0.002808388 |
| ENSMUSG00000039763 | Dnajc28 | 1.15596451 | 0.001661559 |
| ENSMUSG00000040820 | Hlcs | −1.08025446 | 0.012664717 |
| ENSMUSG00000023147 | Get1 | 1.21801686 | 9.00E−04 |
| ENSMUSG00000074892 | B3galt5 | 1.70541233 | 1.05E−04 |
| ENSMUSG00000000157 | Itgb21 | −2.57546306 | 6.84E−05 |
| ENSMUSG00000050272 | Dscam | −3.27046528 | 0.010286928 |
| ENSMUSG00000005251 | Ripk4 | −3.05459807 | 0.003095156 |
| ENSMUSG00000023800 | Tiam2 | −1.49180759 | 0.001074489 |
| ENSMUSG00000015659 | Serac1 | 1.58807591 | 0.001492986 |
| ENSMUSG00000041831 | Sytl3 | −3.08519839 | 0.00419513 |
| ENSMUSG00000052397 | Ezr | −1.26555217 | 0.001350911 |
| ENSMUSG00000094724 | Rnaset2b | 1.12954963 | 0.00337292 |
| ENSMUSG00000033450 | Tagap | 1.34381013 | 8.69E−04 |
| ENSMUSG00000095687 | Rnaset2a | 1.10841631 | 0.003804501 |
| ENSMUSG00000055945 | Prr18 | −2.96462861 | 0.001631284 |
| ENSMUSG00000023827 | Agpat4 | −1.10440948 | 0.006922155 |
| ENSMUSG00000068036 | Afdn | −1.18533385 | 0.004219021 |
| ENSMUSG00000014773 | Dll1 | 1.47134383 | 0.030405216 |
| ENSMUSG00000047786 | Lix1 | −1.20230925 | 0.022526975 |
| ENSMUSG00000052270 | Fpr2 | −2.91459781 | 1.15E−04 |
| ENSMUSG00000063383 | Zfp947 | −1.99934767 | 0.015869206 |
| ENSMUSG00000024124 | Prss30 | −1.54584106 | 0.022598719 |
| ENSMUSG00000032855 | Pkd1 | 1.06640174 | 0.004642532 |
| ENSMUSG00000041130 | Zfp598 | −1.2472791 | 0.007512048 |
| ENSMUSG00000090101 | Snhg9 | −2.29295048 | 0.012214034 |
| ENSMUSG00000002500 | Rpl3l | −3.32443668 | 4.07E−04 |
| ENSMUSG00000075705 | Msrb1 | −2.62517995 | 6.37E−06 |
| ENSMUSG00000024168 | Tmem204 | 1.9888381 | 2.31E−05 |
| ENSMUSG00000036636 | Clcn7 | 1.23648699 | 9.32E−04 |
| ENSMUSG00000015127 | Unkl | 1.85549282 | 0.003678552 |
| ENSMUSG00000035521 | Gnptg | 1.15800429 | 0.002822688 |
| ENSMUSG00000056399 | Prss34 | 1.94870963 | 0.038635546 |
| ENSMUSG00000002279 | Lmf1 | 1.32358892 | 5.10E−04 |
| ENSMUSG00000025739 | Gng13 | −4.18881917 | 0.001331078 |
| ENSMUSG00000025738 | Fbxl16 | −3.8477113 | 3.42E−04 |
| ENSMUSG00000024182 | Axin1 | −1.07185637 | 0.010156353 |
| ENSMUSG00000024186 | Rgs11 | 1.89582039 | 2.58E−04 |
| ENSMUSG00000024190 | Dusp1 | 1.63330623 | 0.001828708 |
| ENSMUSG00000001576 | Ergic1 | 1.14797061 | 0.002095758 |
| ENSMUSG00000057789 | Bak1 | −2.23796645 | 0.003765415 |
| ENSMUSG00000024210 | Ip6k3 | −2.05652079 | 0.001907599 |
| ENSMUSG00000044857 | Lemd2 | −1.18000106 | 0.003409901 |
| ENSMUSG00000024213 | Nudt3 | −1.21655572 | 0.022054277 |
| ENSMUSG00000056692 | Ilrun | −1.22543957 | 0.001872555 |
| ENSMUSG00000080731 | Rps2-ps9 | −1.95511452 | 0.014069963 |
| ENSMUSG00000062859 | Tcp11 | −1.6079148 | 0.033145265 |
| ENSMUSG00000007570 | Fance | 1.08252138 | 0.007267591 |
| ENSMUSG00000004864 | Mapk13 | −3.1090293 | 2.55E−04 |
| ENSMUSG00000063952 | Brpf3 | −1.40979247 | 0.003057664 |
| ENSMUSG00000064147 | Rab44 | −2.84385251 | 3.18E−04 |
| ENSMUSG00000024011 | Pi16 | −3.0273891 | 7.72E−05 |
| ENSMUSG00000024012 | Mtch1 | −1.39438638 | 0.002606353 |
| ENSMUSG00000024013 | Fgd2 | 1.23907891 | 9.47E−04 |
| ENSMUSG00000024014 | Pim1 | −2.57266152 | 6.70E−04 |
| ENSMUSG00000033826 | Dnah8 | −3.76191777 | 8.47E−04 |
| ENSMUSG00000042345 | Ubash3a | −2.8613578 | 0.008928653 |
| ENSMUSG00000024033 | Rsph1 | −2.68908889 | 0.006417385 |
| ENSMUSG00000041119 | Pde9a | −7.95366641 | 8.07E−04 |
| ENSMUSG00000024002 | Brd4 | −1.21258348 | 0.001678987 |
| ENSMUSG00000079563 | Pglyrp2 | −2.59523446 | 0.013262971 |
| ENSMUSG00000091586 | Cyp4f17 | −1.84389515 | 0.016207714 |
| ENSMUSG00000048440 | Cyp4f16 | −1.1651521 | 0.001117975 |
| ENSMUSG00000002308 | Cd320 | 1.05982822 | 0.012865157 |
| ENSMUSG00000079553 | Kifc1 | −1.40830658 | 0.049678305 |
| ENSMUSG00000024308 | Tapbp | 1.01015871 | 0.004843672 |
| ENSMUSG00000024327 | Slc39a7 | 1.24371976 | 0.004637631 |
| ENSMUSG00000037548 | H2-DMb2 | −1.5585755 | 9.22E−05 |
| ENSMUSG00000073421 | H2-Ab1 | −1.99186381 | 7.31E−05 |
| ENSMUSG00000036594 | H2-Aa | −1.67052173 | 2.56E−04 |
| ENSMUSG00000060586 | H2-Eb1 | −2.01831061 | 2.91E−05 |
| ENSMUSG00000034786 | Gpsm3 | −1.20535952 | 8.55E−04 |
| ENSMUSG00000034673 | Pbx2 | −1.91885105 | 4.43E−04 |
| ENSMUSG00000015452 | Ager | −1.68244317 | 0.036625494 |
| ENSMUSG00000015476 | Prrt1 | −2.08898289 | 0.037242196 |
| ENSMUSG00000091971 | Hspa1a | 1.39019887 | 5.17E−04 |
| ENSMUSG00000007036 | Abhd16a | 1.00735667 | 0.004458788 |
| ENSMUSG00000043311 | D17H6S53E | −1.89644694 | 0.003926256 |
| ENSMUSG00000024399 | Ltb | −1.72982955 | 4.04E−04 |
| ENSMUSG00000024403 | Atp6v1g2 | −1.62966761 | 0.00377195 |
| ENSMUSG00000067235 | H2-Q10 | −2.40918511 | 2.77E−05 |
| ENSMUSG00000003534 | Ddr1 | −2.31388553 | 0.003300445 |
| ENSMUSG00000016283 | H2-M2 | −2.4166001 | 4.96E−04 |
| ENSMUSG00000090894 | Olfr110 | 1.3222955 | 0.005648537 |
| ENSMUSG00000090675 | Olfr111 | 1.27844775 | 0.00964651 |
| ENSMUSG00000023915 | Tnfrsf21 | 1.58165485 | 1.18E−04 |
| ENSMUSG00000023963 | Cyp39a1 | −3.61397219 | 2.13E−04 |
| ENSMUSG00000039601 | Rcan2 | −6.45609265 | 8.05E−07 |
| ENSMUSG00000039153 | Runx2 | −1.86056399 | 0.001367058 |
| ENSMUSG00000096847 | Tmem151b | −3.97403153 | 0.003365054 |
| ENSMUSG00000037089 | Slc35b2 | 1.41345485 | 3.07E−04 |
| ENSMUSG00000023942 | Slc29a1 | 1.30407885 | 4.28E−04 |
| ENSMUSG00000023953 | Polh | 1.11360327 | 0.02631325 |
| ENSMUSG00000015597 | Zfp318 | −1.73752224 | 3.19E−04 |
| ENSMUSG00000015605 | Srf | −2.1205593 | 2.12E−04 |
| ENSMUSG00000064043 | Trerf1 | −2.39964951 | 1.13E−05 |
| ENSMUSG00000042265 | Trem1 | −2.13682332 | 9.11E−05 |
| ENSMUSG00000041754 | Trem3 | −2.77525316 | 7.22E−05 |
| ENSMUSG00000051682 | Treml4 | −1.3789247 | 9.24E−04 |
| ENSMUSG00000023992 | Trem2 | 1.1171515 | 0.004897034 |
| ENSMUSG00000040260 | Daam2 | −3.15182587 | 0.001602464 |
| ENSMUSG00000010592 | Dazl | −3.34191142 | 0.002764336 |
| ENSMUSG00000023927 | Satb1 | −1.00673449 | 0.019024601 |
| ENSMUSG00000017831 | Rab5a | 1.02341608 | 0.015725051 |
| ENSMUSG00000023940 | Sgo1 | −1.17842154 | 0.049205255 |
| ENSMUSG00000032915 | Adgre4 | −1.2193526 | 0.002414078 |
| ENSMUSG00000037095 | Lrg1 | −3.3931606 | 5.33E−06 |
| ENSMUSG00000024201 | Kdm4b | 1.36422605 | 0.002438738 |
| ENSMUSG00000110218 | Tincr | −1.96147253 | 0.016569505 |
| ENSMUSG00000024206 | Rfx2 | −2.43354005 | 0.002854271 |
| ENSMUSG00000007670 | Khsrp | −1.36506179 | 0.002208438 |
| ENSMUSG00000046329 | Slc25a23 | −1.24337326 | 0.010793161 |
| ENSMUSG00000062591 | Tubb4a | −4.36771137 | 2.43E−04 |
| ENSMUSG00000005824 | Tnfsf14 | −3.14272815 | 5.28E−04 |
| ENSMUSG00000024164 | C3 | −1.15282415 | 0.002882739 |
| ENSMUSG00000004730 | Adgre1 | 1.27282536 | 8.54E−04 |
| ENSMUSG00000024228 | Nudt12 | 2.14298556 | 0.011664957 |
| ENSMUSG00000024085 | Man2a1 | −1.09998668 | 0.005932676 |
| ENSMUSG00000024098 | Twsg1 | 1.27720231 | 0.00190983 |
| ENSMUSG00000023460 | Rab12 | −1.0602094 | 0.013180921 |
| ENSMUSG00000033278 | Ptprm | 1.91547459 | 6.48E−05 |
| ENSMUSG00000003279 | Dlgap1 | −3.26643804 | 2.94E−04 |
| ENSMUSG00000024053 | Emilin2 | −1.70402188 | 9.37E−05 |
| ENSMUSG00000093289 | Snord92 | −2.24337459 | 0.01195891 |
| ENSMUSG00000024063 | Lbh | −1.45104349 | 9.21E−04 |
| ENSMUSG00000054469 | Lclat1 | 1.0036471 | 0.021193899 |
| ENSMUSG00000024066 | Xdh | −1.78004114 | 9.90E−05 |
| ENSMUSG00000024069 | Slc30a6 | 1.15773308 | 0.003267965 |
| ENSMUSG00000024072 | Yipf4 | 1.48342661 | 8.11E−04 |
| ENSMUSG00000001870 | Ltbp1 | −4.04537176 | 0.002343756 |
| ENSMUSG00000061062 | Hdac1-ps | 2.02768321 | 1.52E−04 |
| ENSMUSG00000036368 | Rmdn2 | 2.40421388 | 0.000412869 |
| ENSMUSG00000024245 | Tmem178 | 2.32147285 | 0.003613634 |
| ENSMUSG00000024120 | Lrpprc | 1.12242534 | 0.002218696 |
| ENSMUSG00000024140 | Epas1 | −3.58733118 | 1.75E−04 |
| ENSMUSG00000024150 | Mcfd2 | 1.09633537 | 0.003259894 |
| ENSMUSG00000036557 | Stpg4 | −3.34067263 | 0.009720195 |
| ENSMUSG00000045394 | Epcam | −1.93882926 | 0.02452206 |
| ENSMUSG00000005371 | Fbxo11 | −1.05325462 | 0.015627941 |
| ENSMUSG00000024109 | Nrxn1 | −4.47765615 | 1.64E−04 |
| ENSMUSG00000024231 | Cul2 | 1.01845202 | 0.004570506 |
| ENSMUSG00000024236 | Svil | −1.02384165 | 0.008182526 |
| ENSMUSG00000036103 | Colec12 | 1.11701241 | 0.006272356 |
| ENSMUSG00000049411 | Tmem241 | 1.28433373 | 8.97E−04 |
| ENSMUSG00000057766 | Ankrd29 | −3.79583722 | 0.004472108 |
| ENSMUSG00000036225 | Kctd1 | −1.4825196 | 0.04530768 |
| ENSMUSG00000071867 | Mrpl27-ps | −2.06687934 | 0.031981043 |
| ENSMUSG00000024411 | Aqp4 | −4.70112146 | 6.72E−06 |
| ENSMUSG00000056124 | B4galt6 | −1.28386571 | 0.006388757 |
| ENSMUSG00000045215 | Asxl3 | −4.51685926 | 0.001890948 |
| ENSMUSG00000041923 | Nol4 | −5.13884357 | 0.001437692 |
| ENSMUSG00000024302 | Dtna | −5.29484852 | 1.16E−05 |
| ENSMUSG00000024270 | Slc39a6 | 1.06605878 | 0.003404712 |
| ENSMUSG00000039616 | Mocos | −1.29335344 | 0.009930923 |
| ENSMUSG00000024268 | Celf4 | −1.96499622 | 0.003405243 |
| ENSMUSG00000057455 | Rit2 | −3.90253668 | 0.001030612 |
| ENSMUSG00000024261 | Syt4 | −3.85053568 | 0.004460062 |
| ENSMUSG00000024395 | Lims2 | −6.82703775 | 7.70E−04 |
| ENSMUSG00000038128 | Camk4 | −1.78274213 | 0.033698461 |
| ENSMUSG00000024357 | Sil1 | 1.2713363 | 4.89E−04 |
| ENSMUSG00000073599 | Ecscr | 1.79366828 | 4.32E−05 |
| ENSMUSG00000024349 | Sting1 | 1.87416548 | 4.51E−05 |
| ENSMUSG00000024347 | Psd2 | −2.26252251 | 0.003513131 |
| ENSMUSG00000043991 | Pura | −1.34685104 | 0.010819587 |
| ENSMUSG00000046727 | Cystm1 | −1.81203885 | 0.002998808 |
| ENSMUSG00000051439 | Cd14 | 1.18443735 | 0.001396 |
| ENSMUSG00000073591 | Pcdhb22 | 1.56828185 | 0.004612282 |
| ENSMUSG00000103332 | Pcdhga2 | 1.44562227 | 0.022792929 |
| ENSMUSG00000104346 | Pcdhga3 | 2.95618935 | 0.003179094 |
| ENSMUSG00000103677 | Pcdhga4 | 3.03360443 | 1.84E−04 |
| ENSMUSG00000102748 | Pcdhgb2 | 3.26866043 | 3.34E−06 |
| ENSMUSG00000103567 | Pcdhga5 | 2.38106068 | 0.002047342 |
| ENSMUSG00000103585 | Pcdhgb4 | 1.50978989 | 0.002057121 |
| ENSMUSG00000023036 | Pcdhgc4 | −2.84910889 | 0.002027801 |
| ENSMUSG00000102543 | Pcdhgc5 | −2.85316983 | 0.004647702 |
| ENSMUSG00000024456 | Diaph1 | −1.98381762 | 2.57E−05 |
| ENSMUSG00000024425 | Ndfip1 | 1.31296162 | 4.48E−04 |
| ENSMUSG00000036452 | Arhgap26 | −1.31285816 | 7.41E−04 |
| ENSMUSG00000036585 | Fgf1 | −1.50928601 | 4.09E−04 |
| ENSMUSG00000024487 | Yipf5 | 1.09273363 | 0.002906786 |
| ENSMUSG00000024500 | Ppp2r2b | −1.78130721 | 0.033225887 |
| ENSMUSG00000024502 | Jakmip2 | −3.60493095 | 0.005226103 |
| ENSMUSG00000071856 | Mcc | −1.45583745 | 0.013109083 |
| ENSMUSG00000033949 | Trim36 | 1.2743263 | 8.21E−04 |
| ENSMUSG00000033022 | Cdo1 | −2.14408528 | 8.71E−04 |
| ENSMUSG00000019647 | Sema6a | −3.1759248 | 0.001912224 |
| ENSMUSG00000059040 | Eno1b | 3.24651682 | 1.91E−06 |
| ENSMUSG00000024529 | Lox | 1.28298888 | 0.002084216 |
| ENSMUSG00000024534 | Sncaip | 1.38621387 | 4.48E−04 |
| ENSMUSG00000024590 | Lmnb1 | −1.14419014 | 0.016208406 |
| ENSMUSG00000069372 | Ctxn3 | −7.17881664 | 5.30E−04 |
| ENSMUSG00000050875 | Minar2 | −3.63239885 | 0.007223224 |
| ENSMUSG00000024610 | Cd74 | −1.7273088 | 3.06E−04 |
| ENSMUSG00000093327 | Mir5107 | −1.57604102 | 0.005338357 |
| ENSMUSG00000024617 | Camk2a | −2.02662872 | 0.012769078 |
| ENSMUSG00000024621 | Csf1r | 2.10260793 | 9.58E−05 |
| ENSMUSG00000045094 | Arhgef37 | −1.78532478 | 0.009782896 |
| ENSMUSG00000024579 | Pcyox1l | 1.31926098 | 0.00102056 |
| ENSMUSG00000033032 | Afap1l1 | 1.00353773 | 0.007867817 |
| ENSMUSG00000039529 | Atp8b1 | 1.2844322 | 0.04883629 |
| ENSMUSG00000041891 | Lman1 | 1.38974389 | 3.34E−04 |
| ENSMUSG00000024525 | Impa2 | −1.27137545 | 0.018740423 |
| ENSMUSG00000024544 | Ldlrad4 | 1.37725966 | 2.77E−04 |
| ENSMUSG00000024511 | Rab27b | −6.55577707 | 0.00111484 |
| ENSMUSG00000024513 | Mbd2 | −1.44889623 | 0.003953747 |
| ENSMUSG00000024558 | Mapk4 | −3.83970391 | 4.14E−04 |
| ENSMUSG00000025429 | Pstpip2 | −1.19448227 | 0.007796071 |
| ENSMUSG00000024548 | Setbp1 | −2.08695163 | 0.003395823 |
| ENSMUSG00000024566 | Atp9b | 1.0346989 | 0.003928917 |
| ENSMUSG00000041607 | Mbp | −3.93020464 | 8.19E−05 |
| ENSMUSG00000024647 | Cbln2 | −4.76628191 | 0.001104556 |
| ENSMUSG00000023066 | Rttn | 1.0162352 | 0.015737672 |
| ENSMUSG00000034028 | Cd226 | −2.43617634 | 0.00369028 |
| ENSMUSG00000024614 | Tmx3 | 1.05623009 | 0.008185361 |
| ENSMUSG00000036908 | Unc93b1 | 1.2569996 | 0.001225608 |
| ENSMUSG00000037263 | Aldh3b3 | −2.73611715 | 0.006507096 |
| ENSMUSG00000045826 | Ptprcap | −2.0192649 | 4.03E−04 |
| ENSMUSG00000040247 | Tbc1d10c | −2.02682948 | 6.20E−05 |
| ENSMUSG00000040663 | Clcf1 | 1.03477602 | 0.006383485 |
| ENSMUSG00000024858 | Grk2 | −1.0199041 | 0.002693058 |
| ENSMUSG00000067889 | Sptbn2 | −2.43491628 | 0.003226697 |
| ENSMUSG00000083282 | Ctsf | 1.6644146 | 7.74E−05 |
| ENSMUSG00000006463 | Zdhhc24 | 1.44831636 | 0.001810242 |
| ENSMUSG00000024891 | Slc29a2 | 1.62863904 | 0.013226133 |
| ENSMUSG00000047379 | B4gat1 | 1.5508368 | 1.14E−04 |
| ENSMUSG00000061451 | Tmem151a | −1.56525947 | 0.04220251 |
| ENSMUSG00000024910 | Ctsw | −1.56570902 | 0.002264835 |
| ENSMUSG00000024909 | Efemp2 | 1.3124455 | 7.85E−04 |
| ENSMUSG00000092341 | Malat1 | −1.89456571 | 1.23E−04 |
| ENSMUSG00000092274 | Neat1 | −2.39384273 | 3.97E−05 |
| ENSMUSG00000024818 | Slc25a45 | 1.07315282 | 0.001577723 |
| ENSMUSG00000045664 | Cdc42ep2 | −1.22761816 | 0.011501308 |
| ENSMUSG00000024942 | Capn1 | −1.15771159 | 0.02450988 |
| ENSMUSG00000024769 | Cdc42bpg | −4.39143695 | 2.70E−04 |
| ENSMUSG00000032648 | Pygm | −1.9121891 | 0.002021155 |
| ENSMUSG00000032946 | Rasgrp2 | −2.26687625 | 1.02E−05 |
| ENSMUSG00000047787 | Flrt1 | −4.24284635 | 0.003063517 |
| ENSMUSG00000024758 | Rtn3 | 1.17294136 | 0.001336679 |
| ENSMUSG00000010095 | Slc3a2 | 1.13799249 | 0.001467119 |
| ENSMUSG00000071659 | Hnrnpul2 | −1.32216928 | 0.00217254 |
| ENSMUSG00000071658 | Gng3 | −2.07015686 | 0.005228581 |
| ENSMUSG00000069833 | Ahnak | −2.72629543 | 5.25E−06 |
| ENSMUSG00000024654 | Asrgl1 | −2.83948763 | 1.55E−06 |
| ENSMUSG00000024665 | Fads2 | −3.01903126 | 4.64E−04 |
| ENSMUSG00000035735 | Dagla | 1.06552849 | 0.015900234 |
| ENSMUSG00000034445 | Cyb561a3 | 1.02066303 | 0.00244998 |
| ENSMUSG00000024669 | Cd5 | 2.30661071 | 3.24E−04 |
| ENSMUSG00000034659 | Tmem109 | 1.06498544 | 0.001677191 |
| ENSMUSG00000024730 | Ms4a8a | −4.4994817 | 1.44E−04 |
| ENSMUSG00000024675 | Ms4a4c | −1.56547336 | 5.29E−04 |
| ENSMUSG00000056290 | Ms4a4b | −3.05714138 | 2.22E−05 |
| ENSMUSG00000079419 | Ms4a6c | −1.30227656 | 5.01E−04 |
| ENSMUSG00000099757 | BE692007 | −1.10850538 | 0.012906169 |
| ENSMUSG00000024681 | Ms4a3 | −3.80579902 | 4.98E−05 |
| ENSMUSG00000039982 | Dtx4 | 1.14468426 | 0.003413421 |
| ENSMUSG00000024695 | Zfp91 | −1.28107932 | 0.001204797 |
| ENSMUSG00000038843 | Gcnt1 | 1.50841021 | 1.69E−04 |
| ENSMUSG00000036192 | Rorb | −8.66974506 | 3.55E−05 |
| ENSMUSG00000024659 | Anxa1 | −2.07147854 | 1.82E−05 |
| ENSMUSG00000053279 | Aldh1a1 | −6.01098879 | 2.36E−05 |
| ENSMUSG00000058624 | Gda | −2.87664629 | 7.86E−06 |
| ENSMUSG00000047368 | Abhd17b | −1.88671692 | 0.009954238 |
| ENSMUSG00000052387 | Trpm3 | −2.37637207 | 0.011386731 |
| ENSMUSG00000033863 | Klf9 | −2.00391767 | 0.001183521 |
| ENSMUSG00000033207 | Mamdc2 | 1.59657771 | 5.65E−04 |
| ENSMUSG00000024867 | Pip5k1b | −4.19017912 | 0.001750785 |
| ENSMUSG00000051490 | Foxd4 | −3.35200034 | 0.001243866 |
| ENSMUSG00000032702 | Kank1 | −7.94451374 | 3.00E−04 |
| ENSMUSG00000038658 | Ric1 | −1.30535072 | 0.015093146 |
| ENSMUSG00000046324 | Ermp1 | 1.02901321 | 0.007097648 |
| ENSMUSG00000024810 | Il33 | −4.08402006 | 3.07E−04 |
| ENSMUSG00000024896 | Minpp1 | 1.81210334 | 2.21E−04 |
| ENSMUSG00000024899 | Papss2 | −2.25155799 | 0.00236385 |
| ENSMUSG00000013662 | Atad1 | 1.16377087 | 0.002502607 |
| ENSMUSG00000024774 | Ankrd22 | −2.8703579 | 0.002230631 |
| ENSMUSG00000035783 | Acta2 | −3.09347865 | 9.22E−04 |
| ENSMUSG00000024781 | Lipa | 1.28265888 | 4.74E−04 |
| ENSMUSG00000067297 | Ifit1bl2 | −2.80378564 | 0.008893475 |
| ENSMUSG00000033610 | Pank1 | −1.38404555 | 0.027181227 |
| ENSMUSG00000024798 | Htr7 | −7.06552601 | 4.94E−04 |
| ENSMUSG00000087579 | Hectd2os | −2.99968591 | 0.011646057 |
| ENSMUSG00000067279 | Ppp1r3c | −4.74299027 | 2.91E−07 |
| ENSMUSG00000012443 | Kif11 | −1.00118541 | 0.046779168 |
| ENSMUSG00000048612 | Myof | −1.39803275 | 0.007503054 |
| ENSMUSG00000054200 | Ffar4 | −1.96896309 | 0.004975284 |
| ENSMUSG00000024998 | Plce1 | −3.8681879 | 2.87E−04 |
| ENSMUSG00000055044 | Pdlim1 | −1.52764033 | 0.003389281 |
| ENSMUSG00000025006 | Sorbs1 | −3.25406035 | 3.71E−06 |
| ENSMUSG00000025008 | Tctn3 | 1.39649546 | 9.01E−04 |
| ENSMUSG00000048120 | Entpd1 | 1.42601086 | 5.14E−04 |
| ENSMUSG00000025016 | Tm9sf3 | 1.16403382 | 0.003807334 |
| ENSMUSG00000049670 | Morn4 | −3.38400557 | 8.82E−04 |
| ENSMUSG00000025178 | Pi4k2a | −1.25442066 | 0.001265975 |
| ENSMUSG00000018821 | Avpi1 | −1.28510536 | 0.008623686 |
| ENSMUSG00000044345 | Marveld1 | −2.21834709 | 6.84E−04 |
| ENSMUSG00000040414 | Slc25a28 | −1.33758103 | 0.002569211 |
| ENSMUSG00000025194 | Abcc2 | −3.44316096 | 0.002605492 |
| ENSMUSG00000037071 | Scd1 | −4.26905197 | 1.01E−04 |
| ENSMUSG00000037126 | Psd | −1.42905041 | 0.006078265 |
| ENSMUSG00000025231 | Sufu | 1.07216405 | 0.004671451 |
| ENSMUSG00000025035 | Arl3 | −1.01627741 | 0.016978901 |
| ENSMUSG00000064105 | Cnnm2 | 1.44121812 | 0.011621801 |
| ENSMUSG00000025041 | Nt5c2 | 1.01864756 | 0.002840831 |
| ENSMUSG00000025050 | Pcgf6 | 1.40956586 | 0.003249457 |
| ENSMUSG00000006435 | Neurl1a | 1.29688567 | 0.004918087 |
| ENSMUSG00000025060 | Slk | −1.89631131 | 3.28E−05 |
| ENSMUSG00000025064 | Col17a1 | −2.06827039 | 0.036810783 |
| ENSMUSG00000097636 | Mirt1 | −2.97912894 | 1.22E−05 |
| ENSMUSG00000034765 | Dusp5 | −1.71403089 | 0.008277565 |
| ENSMUSG00000043639 | Rbm20 | −2.05894871 | 0.030341418 |
| ENSMUSG00000024978 | Gpam | 1.69909293 | 0.001688571 |
| ENSMUSG00000099746 | Ppnr | −4.48961606 | 0.002165417 |
| ENSMUSG00000035283 | Adrb1 | −1.55030698 | 0.001397772 |
| ENSMUSG00000025083 | Afap1l2 | −2.51047566 | 0.032137178 |
| ENSMUSG00000054843 | Atrnl1 | −1.70298055 | 0.029363546 |
| ENSMUSG00000025089 | Gfra1 | −4.89258027 | 8.78E−04 |
| ENSMUSG00000041362 | Shtn1 | −2.44596347 | 2.63E−05 |
| ENSMUSG00000003228 | Grk5 | −2.23793112 | 3.02E−04 |

TABLE 2
Summary of human samples included in the study.

| | N for | N for | Age | Sex | PMI |
|---|---|---|---|---|---|
| | ELISA | IF | (years ± SD) | (F/M) | (min ± SD) |
| Normal (CDR = 0) | 11 | 5 | 81.1 ± 9.6 | 6/5 | 787 ± 425 |
| MCI (CDR = 0.5) | 10 | 9 | 82.6 ± 9.4 | 6/5 | 730 ± 425 |
| AD (CDR > 1) | 11 | 8 | 82.7 ± 9.5 | 7/5 | 533 ± 237 |

PMI, Post-mortem Interval (min);
CDR, Clinical Dementia Rating;
IF, Immunofluorescence staining

Samples matched for age and se were analyzed for OPN levels as measured by ELISA of frozen samples followed by immunofluorescence staining for Iba-1/CD11c/OPN (Fixed, parrafin embedded sections).

TABLE 3
Characterization of frozen and fixed human samples.

| | Subject | | | PMI | | Used for | Used | Plaque | Tangle |
|---|---|---|---|---|---|---|---|---|---|
| | number | Age | Sex | (min) | CDR | ELISA | for IF | rating¹ | rating¹ |
| Normal | 23983 | 79 | Male | 964 | 0 | + | | 1 | 0 |
| | 36472 | 89 | Female | 353 | 0 | + | | 1 | 0 |
| | 57242 | 85 | Female | 320 | 0 | + | + | 1 | 0 |
| | 61911 | 95 | Female | 456 | 0 | + | | 0 | 0 |
| | 80875 | 83 | Male | 834 | 0 | + | | 1 | 0 |
| | 201539 | 85 | Male | 488 | 0 | + | | 3 | 1 |
| | 395695 | 91 | Female | 290 | 0 | + | + | 0 | 0 |
| | 503571 | 81 | Female | 1364 | 0 | + | | 0 | 0 |
| | 576228 | 72 | Male | 986 | 0 | + | + | 0 | 0 |
| | 808460 | 66 | Male | 1390 | 0 | + | + | 0 | 0 |
| | 921781 | 66 | Female | 1211 | 0 | + | + | 0 | 0 |
| MCI | 13090 | 71 | Male | 1285 | 0.5 | + | + | 1 | 0 |
| | 24420 | 83 | Male | 740 | 0.5 | + | | 0 | 0 |
| | 27409 | 68 | Female | 1155 | 0.5 | + | + | 0 | 0 |
| | 38519 | 92 | Female | 225 | 0.5 | + | + | 3 | 3 |
| | 46426 | 68 | Male | 225 | 0.5 | + | | 0 | 0 |
| | 83284 | 89 | Female | 495 | 0.5 | + | + | 0 | 0 |
| | 271140 | 89 | Male | 776 | 0.5 | + | + | 1 | 0 |
| | 582757 | 87 | Male | 925 | 0.5 | + | + | 0 | 0 |
| | 604571 | 93 | Female | 248 | 0.5 | + | + | 1 | 0 |
| | 754833 | 82 | Female | 553 | 0.5 | + | + | 5 | 3 |
| | 852223 | 87 | Female | 1398 | 0.5 | | + | 3 | 1 |
| AD | 5323 | 91 | Female | 420 | 2 | + | | 5 | 5 |
| | 29188 | 91 | Female | 285 | 2 | + | + | 3 | 1 |
| | 1100 | 84 | Female | 480 | 3 | + | | 3 | 1 |
| | 64891 | 68 | Male | 1075 | 3 | + | + | 5 | 5 |
| | 74931 | 79 | Female | 390 | 3 | + | + | 5 | 5 |
| | 354049 | 85 | Male | 392 | 3 | + | + | 5 | 1 |
| | 639732 | 69 | Female | 500 | 3 | + | + | 5 | 5 |
| | 762124 | 72 | Male | 375 | 3 | | + | 3 | 5 |
| | 782872 | 99 | Female | 360 | 3 | + | | 1 | 0 |
| | 31742 | 87 | Female | 885 | 4 | + | + | 5 | 5 |
| | 39413 | 87 | Male | 540 | 5 | + | + | 5 | 5 |
| | 524179 | 80 | Male | 688 | 5 | + | | 5 | 5 |

¹Determined postmortem by a neuropathologist before storage of samples in the Mount Sinai brain bank.
PMI: Post-mortem Interval (min);
CDR: Clinical Dementia Rating;
IF: Immunofluorescence staining

1. A compound that can cross a blood brain barrier in a patient, comprising
an active agent comprising an anti-osteopontin antibody, an anti-CD11b antibody or an αVβ3 inhibitor conjugated to a bridge, a linker, a carrier agent, or a combination thereof.

2-4. (canceled)

5. The compound of claim 1, wherein the αVβ3 inhibitor comprises a cyclic RGD-containing small molecule, including cilengitide or a derivative thereof.

6. (canceled)

7. The compound of claim 1, wherein the anti-osteopontin or anti-CD11b antibody comprises a monospecific, bispecific, or multispecific antibody.

8. The compound of claim 1, wherein the anti-osteopontin or anti-CD11b antibody comprises a monoclonal antibody, Fab, F(ab′)2, Fab′ single chain antibody, Fv, single chain, mono-specific antibody, bi-specific antibody, tri-specific antibody, multi-valent antibody, chimeric antibody, canine-human chimeric antibody, chimeric antibody, humanized antibody, human antibody, CDR-grafted antibody, shark antibody, nanobody, camelid antibody, microbody, intrabody, de-fucosylated antibody, or any combination or derivative thereof.

9. The compound of claim 1, wherein the anti-osteopontin or anti-CD11b antibody comprises a single chain antibody.

10-13. (canceled)

14. The compound of claim 1, wherein the carrier agent comprises a cell-penetrating peptide or a cell-targeting peptide.

15. (canceled)

16. The compound of claim 14, wherein the carrier agent comprises TFFYGGSRGKRNNFKTEEY (Angiopep-2; SEQ ID NO: 1), D-Lys6-LHRH, CNGRCG (SEQ ID NO: 4), PGA, LHRH (SEQ ID NO: 5), DRDDS (SEQ ID NO: 6), D-γ-E-γ-E-γ-E-E (SEQ ID NO: 7), GSH, HSTPSSP (SEQ ID NO: 8), DSSLFAL (SEQ ID NO: 9), YGRKKRRQRRRPPQQ (SEQ ID NO: 10), LLIILRRRIRKQAHAHSK (SEQ ID NO: 11), RRLSYSRRRF (SEQ ID NO: 12) or any combination thereof.

17-19. (canceled)

20. The compound of claim 1, wherein the bridge comprises positively charged amino acids.

21. (canceled)

22. The compound of claim 20, wherein the bridge comprises arginine, lysine, histidine or combinations thereof.

23-26. (canceled)

27. The compound of claim 1, wherein the linker comprises amino acids that comprise a flexible linker.

28-31. (canceled)

32. The compound of claim 27, wherein the flexible linker comprises small, polar amino acids or non-polar amino acids.

33-35. (canceled)

36. The compound of claim 27, wherein the flexible linker comprises 6-aminohexanoic acid (Ahx), 2-aminoethoxy acetic acid (AEA), 5-aminovaleric acid (Ava), 8-amino-3,6-dioxaoctanoic acid (PEG2 or AEEA) or 12-amino-4,7,10-trioxadodecanoic acid (PEG3).

37-38. (canceled)

39. The compound of claim 1, additionally comprising a conjugate that connects the active agent to the bridge, linker or carrier agent.

40. The compound of claim 39, wherein the conjugate comprises an —N-hydroxysuccinimide-ester (—NHS ester).

41. (canceled)

42. A pharmaceutical composition for treating a neurodegenerative disease in a patient, comprising the compound of claim 1.

43-51. (canceled)

52. A method for treating Alzheimer's disease in a patient, comprising administering the compound of claim 1 to the patient.

53-57. (canceled)

58. The method of claim 52, wherein the compound is administered intranasally.

59-89. (canceled)

90. The compound of claim 1, wherein the compound has a linear arrangement, in order: active agent, bridge, linker and carrier agent.


SEQUENCE LISTING (20 sequences)

SEQ ID NO: 1 - protein, 19 aa, synthetic construct
TFFYGGSRGKRNNFKTEEY

SEQ ID NO: 2 - protein, 317 aa, Rattus norvegicus
MRLAVVCFCLFGLASCLPVKVAEFGSSEEKAHYSKHSDAVATWLKPDPSQKQNLLAPQNSVSSEETDDFKQETLPSNSNESHDHMDDDDDDDDDGDHAESEDSVNSDESDESHHSDESDESFTASTQADVLTPIAPTVDVPDGRGDSLAYGLRSKSRSFPVSDEQYPDATDEDLTSRMKSQESDEAIKVIPVAQRLSVPSDQDSNGKTSHESSQLDEPSVETHSLEQSKEYKQRASHESTEQSDAIDSAEKPDAIDSAERSDAIDSQASSKASLEHQSHEFHSHEDKLVLDPKSKEDDRYLKFRISHELESSSSEVN

SEQ ID NO: 3 - skipped

SEQ ID NO: 4 - protein, 6 aa, synthetic construct
CNGRCG

SEQ ID NO: 5 - protein, 4 aa, synthetic construct
LHRH

SEQ ID NO: 6 - protein, 5 aa, synthetic construct
DRDDS

SEQ ID NO: 7 - protein, 5 aa, synthetic construct
  MOD_RES 2..4: note = Gamma-Glutamic acid
DEEEE

SEQ ID NO: 8 - protein, 7 aa, synthetic construct
HSTPSSP

SEQ ID NO: 9 - protein, 7 aa, synthetic construct
DSSLFAL

SEQ ID NO: 10 - protein, 15 aa, synthetic construct
YGRKKRRQRRRPPQQ

SEQ ID NO: 11 - protein, 18 aa, synthetic construct
LLIILRRRIRKQAHAHSK

SEQ ID NO: 12 - protein, 10 aa, synthetic construct
RRLSYSRRRF

SEQ ID NO: 13 - protein, 4 aa, synthetic construct
  MOD_RES 1: note = Acetylated-Tyrosine
  MOD_RES 4: note = Aspartic acid-Aldehyde
YVAD

SEQ ID NO: 14 - protein, 5 aa, synthetic construct
GGGGS

SEQ ID NO: 15 - protein, 18 aa, synthetic construct
KESGSVSSEQLAQFRSLD

SEQ ID NO: 16 - protein, 14 aa, synthetic construct
EGKSSGSGSESKST

SEQ ID NO: 17 - protein, 8 aa, synthetic construct
GGGGGGGG

SEQ ID NO: 18 - protein, 12 aa, synthetic construct
GSAGSAAGSGEF

SEQ ID NO: 19 - protein, 20 aa, synthetic construct
GGGGSGGGGSGGGGSGGGGS

SEQ ID NO: 20 - protein, 22 aa, synthetic construct
  SITE 3: note = 6-aminohexanoic acid
  MOD_RES 22: note = Amidated Tyrosine
KKXTFFYGGSRGKRNNFKTEEY